(19) United States
(12) Patent Application Publication
(10) Pub. No.: US 2026/0007704 A1
(43) Pub. Date: Jan. 8, 2026

(54) MICROBIAL CONSORTIA

(71) Applicant: KANVAS BIOSCIENCES, INC., Monmouth Junction, NJ (US)

(72) Inventors: Robert Lee SWEM, Monmouth Junction, NJ (US); Joshua J. HAMILTON, Monmouth Junction, NJ (US); James BERLEMAN, Monmouth Junction, NJ (US); Kyle JACOBY, Monmouth Junction, NJ (US); Ariana M. CIGLAR, Monmouth Junction, NJ (US); Anupreet PARMAR, Monmouth Junction, NJ (US); Aditya BHALLA, Monmouth Junction, NJ (US); Pawan KUMAR, Monmouth Junction, NJ (US)

(73) Assignee: KANVAS BIOSCIENCES, INC., Monmouth Junction, NJ (US)

(21) Appl. No.: 19/324,836

(22) Filed: Sep. 10, 2025

Related U.S. Application Data
(63) Continuation of application No. PCT/US24/19341, filed on Mar. 11, 2024.
(60) Provisional application No. 63/451,442, filed on Mar. 10, 2023.

(51) Int. Cl. A61K 35/742; A61K 35/745; A61K 35/747; A61K 45/06; A61P 1/00

(52) U.S. Cl. CPC A61K 35/742; A61K 35/745; A61K 35/747; A61K 45/06; A61P 1/00

(57) ABSTRACT

The present disclosure provides microbial consortia, and methods of making and using the same, comprising a complete microbiome replacement capable of stable engraftment in the gastrointestinal tract for the treatment of IBD, ulcerative colitis, and Crohn's disease.

### CROSS-REFERENCE TO RELATED APPLICATIONS

The instant application is a continuation of International Patent Application No. PCT/US24/19341, filed Mar. 11, 2024, which claims priority to U.S. Provisional Patent Application No. 63/451,442, filed Mar. 10, 2023, and to U.S. Provisional Patent Application No. 63/469,080, filed May 26, 2023, the content of each of which is incorporated in its entirety, and to each of which priority is claimed.

### SEQUENCE LISTING

The instant application contains a Sequence Listing which has been submitted electronically in XML format and is hereby incorporated by reference in its entirety. Said XML copy, created on Sep. 10, 2025, is named 0915920133.xml, and is 393,169 bytes in size.

### FIELD OF THE INVENTION

The present disclosure relates to microbial consortia for the treatment of dysbiosis of the gut and/or irritable bowel disease.

### BACKGROUND

The gastrointestinal tract comprises various biological niches along its longitudinal length having different physical, chemical, and nutrient compositions. As a consequence of these diverse conditions, specific microbial communities are established within a particular biological niche. The microbial species comprising a specific microbial community are highly responsive to their local environment and produce an array of bioactive molecules that facilitate host engraftment, inter-microbial communication, nutrient metabolism, and inclusion or exclusion of competing microbial species. Adding further complexity, there is substantial diversity of microbial species and strains in the human gastrointestinal tract between individuals, which is attributed to a number of factors including genetics, diet, antibiotic and antifungal use, surgical intervention (e.g., gastric by-pass/bowel resection), presence of inflammatory bowel disease and/or irritable bowel syndrome, and other environmental influences. However, despite this interindividual diversity, the functional attributes of the varying human gut microbiota are relatively consistent among healthy adults and comprise core metabolic pathways involved in carbohydrate metabolism, amino acid metabolism, fermentation, and oxidative phosphorylation.

Modulation of microbial species in the gastrointestinal tract through the use of antibiotics, antifungals, and more recently, fecal microbial transplantation (“FMT”), have been approaches clinically investigated for the treatment and/or prevention of certain diseases and disorders. For example, Dodd et al. (Nature, 2007, 551: 648-652) have proposed FMT as a therapeutic to modulate the levels of aromatic amino acid metabolites in the serum of gnotobiotic mice, which affect intestinal permeability and systemic immunity.

As a modality for treating various diseases and/or conditions, there is a need for microbial compositions comprising a plurality of microbial species having improved therapeutic efficacy and an ability to efficiently engraft in a host, grow, and metabolize pathogenic substrates to non-pathogenic metabolic products within the various biological niches of the gastrointestinal tract and within the diverse gastrointestinal environments of different individuals. Furthermore, there is an unmet need for a treatment of diseases using a complex microbial community that can engraft and function symbiotically in the human gastrointestinal tract to degradation of a disease-associated metabolic substrate.

### SUMMARY OF THE INVENTION

The present disclosure provides methods for preventing, decreasing, and/or treating dysbiosis in a subject. In certain embodiments, the methods comprise administering an effective amount of a microbial consortium or a pharmaceutical composition thereof comprising
a) Clostridium citroniae, Bacteroides salyersiae, Blautia obeum, Parabacteroides merdae, Parabacteroides distasonis, Anaerostipes hadrus, Lachnospiraceae sp. FBI00033, Eubacterium eligens, Bifidobacterium dentium, Blautia wexlerae, Fusicatenibacter saccharivorans, Bacteroides nordii, Dorea formicigenerans, Dorea longicatena, Bacteroides stercorirosoris, Bifidobacterium longum, Bacteroides kribbi, Lachnospiraceae sp. FBI00071, Bacteroides thetaiotaomicron, Clostridium clostridioforme, Clostridium scindens, Roseburia hominis, Clostridium fessum, Coprococcus comes, Blautia faecis, Hungatella hathewayi, Bacteroides stercoris, Collinsella aerofaciens, Hungatella effluvii, Bifidobacterium adolescentis, Bifidobacterium catenulatum, Lactobacillus rogosae, Bacteroides faecis, Bacteroides finegoldii, Clostridiaceae sp. FBI00191, Ruminococcus faecis, Lachnoclostridium pacaense, Clostridium bolteae, Longicatena caecimuris, Eggerthella lenta, Blautia massiliensis, Bacteroides xylanisolvens, Bacteroides vulgatus, Megasphaera massiliensis, Butyricimonas faecihominis, Eisenbergiella tayi, Acidaminococcus intestini, Emergencia timonensis, Bifidobacterium pseudocatenulatum, Eubacterium hallii, Anaerofustis stercorihominis, Eubacterium ventriosum, Blautia hydrogenotrophica, and Lachnospiraceae sp. FBI00290, or a functional equivalent thereof; or
b) FBI00001, FBI00002, FBI00010, FBI00013, FBI00029, FBI00032, FBI00033, FBI00034, FBI00043, FBI00044, FBI00048, FBI00050, FBI00051, FBI00057, FBI00059, FBI00060, FBI00070, FBI00071, FBI00076, FBI00079, FBI00087, FBI00093, FBI00102, FBI00109, FBI00117, FBI00120, FBI00125, FBI00127, FBI00128, FBI00145, FBI00162, FBI00174, FBI00184, FBI00190, FBI00191, FBI00194, FBI00198, FBI00199, FBI00200, FBI00201, FBI00205, FBI00206, FBI00211, FBI00220, FBI00221, FBI00236, FBI00245, FBI00248, FBI00251, FBI00254, FBI00267, FBI00278, FBI00288, and FBI00290, or a functional equivalent thereof.

In certain embodiments, the microbial consortium or a pharmaceutical composition thereof further comprises:
a) Acutalibacter timonensis, Alistipes onderdonkii, Bacteroides uniformis, Eubacterium rectale, Alistipes timonensis, Bacteroides kribbi, Coprococcus eutactus, Bilophila wadsworthia, Bacteroides caccae, Alistipes shahii, Parasutterella excrementihominis, Paraprevotella clara, Sutterella wadsworthensis, Sutterella massiliensis, Porphyromonas asaccharolytica, Ruminococcus bromii, Monoglobus pectinilyticus, Ruminococcaceae sp. FBI00097, Gordonibacter pamelaeae, Bacteroides uniformis, Gordonibacter pamelaeae, Bacteroides fragilis, Phascolarctobacterium faecium, Monoglobus pectinilyticus, Clostridium aldenense, Ruthenibacterium lactatiformans, Bacteroides ovatus, Bifidobacterium bifidum, Anaerotruncus massiliensis, Clostridium aldenense, Sutterella wadsworthensis, Catabacter hongkongensis, Alistipes senegalensis, Ruminococcaceae sp. FBI00233, Alistipes shahii, Dielma fastidiosa, Eubacterium siraeum, Faecalibacterium prausnitzii, Turicibacter sanguinis, Eubacterium rectale, Bacteroides caccae, Methanobrevibacter smithii, Barnesiella intestinihominis, Alistipes onderdonkii, and Methanobrevibacter smithii, or a functional equivalent thereof;
b) Bifidobacterium adolescentis, Bifidobacterium longum, Bifidobacterium pseudocatenulatum, Bacteroides thetaiotaomicron, Coprococcus comes, Fusicatenibacter saccharivorans, Eggerthella lenta, Eubacterium eligens, Bacteroides xylanisolvens, Lactobacillus rogosae, Clostridium citroniae, Collinsella aerofaciens, Blautia obeum, Eggerthella lenta, Blautia wexlerae, Lachnoclostridium pacaense, Bacteroides vulgatus, Parabacteroides merdae, Dorea formicigenerans, Ruminococcus faecis, Roseburia hominis, Anaerostipes hadrus, Bifidobacterium adolescentis, Bifidobacterium pseudocatenulatum, Clostridium bolteae, Eisenbergiella tayi, Dorea longicatena, Eggerthella lenta, Bacteroides stercoris, Hungatella hathewayi, and Bacteroides xylanisolvens, or a functional equivalent thereof; and/or
c) Alistipes putredinis, Dialister succinatiphilus, Akkermansia muciniphila, Ruminococcus bromii, Dialister invisus, Bacteroides massiliensis, Bilophila wadsworthia, Holdemanella biformis, Parasutterella excrementihominis, Alistipes sp. FBI00180, Bacteroides coprocola, Alistipes sp. FBI00238, Alistipes putredinis, Eubacterium xylanophilum, and Senegalimassilia anaerobia, or a functional equivalent thereof.

In certain embodiments, the microbial consortium or a pharmaceutical composition thereof further comprises:
a) FBI00004, FBI00012, FBI00015, FBI00018, FBI00019, FBI00021, FBI00038, FBI00040, FBI00046, FBI00061, FBI00066, FBI00075, FBI00077, FBI00080, FBI00081, FBI00085, FBI00092, FBI00097, FBI00099, FBI00112, FBI00132, FBI00137, FBI00140, FBI00149, FBI00151, FBI00176, FBI00189, FBI00197, FBI00208, FBI00212, FBI00224, FBI00226, FBI00229, FBI00233, FBI00235, FBI00237, FBI00243, FBI00244, FBI00258, FBI00260, FBI00263, FBI00270, FBI00273, FBI00277, and FBI00292, or a functional equivalent thereof;
b) FBI00009, FBI00011, FBI00016, FBI00020, FBI00025, FBI00027, FBI00030, FBI00047, FBI00052, FBI00053, FBI00056, FBI00062, FBI00078, FBI00096, FBI00104, FBI00110, FBI00111, FBI00113, FBI00115, FBI00116, FBI00123, FBI00124, FBI00126, FBI00135, FBI00147, FBI00159, FBI00167, FBI00170, FBI00232, FBI00255, and FBI00271, or a functional equivalent thereof; and/or
c) FBI00022, FBI00049, FBI00068, FBI00069, FBI00152, FBI00165, FBI00171, FBI00175, FBI00177, FBI00180, FBI00182, FBI00238, FBI00269, FBI00274, and FBI00281, or a functional equivalent thereof.

In certain embodiments, the microbial consortium or a pharmaceutical composition thereof further comprises a first strain of Oxalobacter formigenes or FBI00067. In certain embodiments, the microbial consortium or a pharmaceutical composition thereof further comprises a second strain of Oxalobacter formigenes or FBI00133. In certain embodiments, wherein the microbial consortium or a pharmaceutical composition thereof further comprises a third strain of Oxalobacter formigenes or FBI00289.

In certain embodiments, the microbial consortium or a pharmaceutical composition thereof comprises:
a) Clostridium citroniae, Bacteroides salyersiae, Blautia obeum, Parabacteroides merdae, Parabacteroides distasonis, Anaerostipes hadrus, Lachnospiraceae sp. FBI00033, Eubacterium eligens, Bifidobacterium dentium, Blautia wexlerae, Fusicatenibacter saccharivorans, Bacteroides nordii, Dorea formicigenerans, Dorea longicatena, Bacteroides stercorirosoris, Bifidobacterium longum, Bacteroides kribbi, Lachnospiraceae sp. FBI00071, Bacteroides thetaiotaomicron, Clostridium clostridioforme, Clostridium scindens, Roseburia hominis, Clostridium fessum, Coprococcus comes, Blautia faecis, Hungatella hathewayi, Bacteroides stercoris, Collinsella aerofaciens, Hungatella effluvii, Bifidobacterium adolescentis, Bifidobacterium catenulatum, Lactobacillus rogosae, Bacteroides faecis, Bacteroides finegoldii, Clostridiaceae sp. FBI00191, Ruminococcus faecis, Lachnoclostridium pacaense, Clostridium bolteae, Longicatena caecimuris, Eggerthella lenta, Blautia massiliensis, Bacteroides xylanisolvens, Bacteroides vulgatus, Megasphaera massiliensis, Butyricimonas faecihominis, Eisenbergiella tayi, Acidaminococcus intestini, Emergencia timonensis, Bifidobacterium pseudocatenulatum, Eubacterium hallii, Anaerofustis stercorihominis, Eubacterium ventriosum, Blautia hydrogenotrophica, and Lachnospiraceae sp. FBI00290, or a functional equivalent thereof;
b) Acutalibacter timonensis, Alistipes onderdonkii, Bacteroides uniformis, Eubacterium rectale, Alistipes timonensis, Bacteroides kribbi, Coprococcus eutactus, Bilophila wadsworthia, Bacteroides caccae, Alistipes shahii, Parasutterella excrementihominis, Paraprevotella clara, Sutterella wadsworthensis, Sutterella massiliensis, Porphyromonas asaccharolytica, Ruminococcus bromii, Monoglobus pectinilyticus, Ruminococcaceae sp. FBI00097, Gordonibacter pamelaeae, Bacteroides uniformis, Gordonibacter pamelaeae, Bacteroides fragilis, Phascolarctobacterium faecium, Monoglobus pectinilyticus, Clostridium aldenense, Ruthenibacterium lactatiformans, Bacteroides ovatus, Bifidobacterium bifidum, Anaerotruncus massiliensis, Clostridium aldenense, Sutterella wadsworthensis, Catabacter hongkongensis, Alistipes senegalensis, Ruminococcaceae sp. FBI00233, Alistipes shahii, Dielma fastidiosa, Eubacterium siraeum, Faecalibacterium prausnitzii, Turicibacter sanguinis, Eubacterium rectale, Bacteroides caccae, Methanobrevibacter smithii, Barnesiella intestinihominis, Alistipes onderdonkii, and Methanobrevibacter smithii, or a functional equivalent thereof;
c) Bifidobacterium adolescentis, Bifidobacterium longum, Bifidobacterium pseudocatenulatum, Bacteroides thetaiotaomicron, Coprococcus comes, Fusicatenibacter saccharivorans, Eggerthella lenta, Eubacterium eligens, Bacteroides xylanisolvens, Lactobacillus rogosae, Clostridium citroniae, Collinsella aerofaciens, Blautia obeum, Eggerthella lenta, Blautia wexlerae, Lachnoclostridium pacaense, Bacteroides vulgatus, Parabacteroides merdae, Dorea formicigenerans, Ruminococcus faecis, Roseburia hominis, Anaerostipes hadrus, Bifidobacterium adolescentis, Bifidobacterium pseudocatenulatum, Clostridium bolteae, Eisenbergiella tayi, Dorea longicatena, Eggerthella lenta, Bacteroides stercoris, Hungatella hathewayi, and Bacteroides xylanisolvens, or a functional equivalent thereof; and
d) Alistipes putredinis, Dialister succinatiphilus, Akkermansia muciniphila, Ruminococcus bromii, Dialister invisus, Bacteroides massiliensis, Bilophila wadsworthia, Holdemanella biformis, Parasutterella excrementihominis, Alistipes sp. FBI00180, Bacteroides coprocola, Alistipes sp. FBI00238, Alistipes putredinis, Eubacterium xylanophilum, and Senegalimassilia anaerobia, or a functional equivalent thereof.

In certain embodiments, the microbial consortium or a pharmaceutical composition thereof comprises:
a) FBI00001, FBI00002, FBI00010, FBI00013, FBI00029, FBI00032, FBI00033, FBI00034, FBI00043, FBI00044, FBI00048, FBI00050, FBI00051, FBI00057, FBI00059, FBI00060, FBI00070, FBI00071, FBI00076, FBI00079, FBI00087, FBI00093, FBI00102, FBI00109, FBI00117, FBI00120, FBI00125, FBI00127, FBI00128, FBI00145, FBI00162, FBI00174, FBI00184, FBI00190, FBI00191, FBI00194, FBI00198, FBI00199, FBI00200, FBI00201, FBI00205, FBI00206, FBI00211, FBI00220, FBI00221, FBI00236, FBI00245, FBI00248, FBI00251, FBI00254, FBI00267, FBI00278, FBI00288, and FBI00290, or a functional equivalent thereof;
b) FBI00004, FBI00012, FBI00015, FBI00018, FBI00019, FBI00021, FBI00038, FBI00040, FBI00046, FBI00061, FBI00066, FBI00075, FBI00077, FBI00080, FBI00081, FBI00085, FBI00092, FBI00097, FBI00099, FBI00112, FBI00132, FBI00137, FBI00140, FBI00149, FBI00151, FBI00176, FBI00189, FBI00197, FBI00208, FBI00212, FBI00224, FBI00226, FBI00229, FBI00233, FBI00235, FBI00237, FBI00243, FBI00244, FBI00258, FBI00260, FBI00263, FBI00270, FBI00273, FBI00277, and FBI00292, or a functional equivalent thereof;
c) FBI00009, FBI00011, FBI00016, FBI00020, FBI00025, FBI00027, FBI00030, FBI00047, FBI00052, FBI00053, FBI00056, FBI00062, FBI00078, FBI00096, FBI00104, FBI00110, FBI00111, FBI00113, FBI00115, FBI00116, FBI00123, FBI00124, FBI00126, FBI00135, FBI00147, FBI00159, FBI00167, FBI00170, FBI00232, FBI00255, and FBI00271, or a functional equivalent thereof; and
d) FBI00022, FBI00049, FBI00068, FBI00069, FBI00152, FBI00165, FBI00171, FBI00175, FBI00177, FBI00180, FBI00182, FBI00238, FBI00269, FBI00274, and FBI00281, or a functional equivalent thereof.

In certain embodiments, the microbial consortium or a pharmaceutical composition thereof further comprises a first strain of Oxalobacter formigenes or FBI00067, a second strain of Oxalobacter formigenes or FBI00133, and a third strain of Oxalobacter formigenes or FBI00289. In certain embodiments, the microbial consortium or a pharmaceutical composition thereof further comprises a first strain of Oxalobacter formigenes or FBI00067, a second strain of Oxalobacter formigenes or FBI00133, and a third strain of Oxalobacter formigenes or FBI00289. In certain embodiments, the microbial consortium or a pharmaceutical composition thereof is FB-001 or a functional equivalent thereof. In certain embodiments, the microbial consortium or a pharmaceutical composition thereof is FB-003 or a functional equivalent thereof.

In certain embodiments, the microbial consortium or a pharmaceutical composition thereof increases the microbial diversity of the gastrointestinal tract. In certain embodiments, the microbial consortium or a pharmaceutical composition thereof increases short chain fatty acids (SCFAs). In certain embodiments, the microbial consortium or a pharmaceutical composition thereof increases secondary bile acids. In certain embodiments, the microbial consortium or a pharmaceutical composition thereof decreases bacterial pathogens in the gastrointestinal tract of the subject.

In certain embodiments, the microbial consortium or a pharmaceutical composition thereof comprises between about 5×10⁹ and about 5×10¹² viable cells. In certain embodiments, the microbial consortium or a pharmaceutical composition thereof comprises between about 5×10⁹ and about 5×10¹⁰ viable cells. In certain embodiments, the microbial consortium or a pharmaceutical composition thereof comprises between about 5×10¹¹ and about 5×10¹¹ viable cells. In certain embodiments, the microbial consortium or a pharmaceutical composition thereof comprises between about 5×10¹¹ and about 5×10¹² viable cells. In certain embodiments, the microbial consortium or a pharmaceutical composition thereof comprises up to about 10¹¹ viable cells. In certain embodiments, the microbial consortium or a pharmaceutical composition thereof comprises up to about 10¹² viable cells.

In certain embodiments, the methods comprise administering a loading dose and one or more maintenance doses. In certain embodiments, the loading dose is administered for 1 day, 2 days, 3 days, 4 days, 5 days, 6 days, 7 days, 8 days, 9 days, or 10 days. In certain embodiments, the loading dose is administered for 2-3 days, 3-5 days, 4-6 days, or 5-7 days. In certain embodiments, the one or more maintenance doses are administered for at least 21 days following the last loading dose.

In certain embodiments, the methods further comprise administering an antibacterial agent, an antiviral agent, an antifungal agent, anti-inflammatory agent, an immunosuppressive agent, and/or a prebiotic. In certain embodiments, the antibacterial agent is selected from the group consisting of ciprofloxacin, levaquin, floxin, tequin, avelox, norflox, cephalexin, cefuroxime, cefadroxil, cefazolin, cephalothin, cefaclor, cefamandole, cefoxitin, cefprozil, ceftobiprole, amoxicillin, ampicillin, penicillin V, dicloxacillin, carbenicillin, vancomycin, and methicillin, tetracycline, minocycline, oxytetracycline, doxycycline, ertapenem, doripenem, imipenem/cilastatin, and meropenem. In certain embodiments, the antiviral agent is selected from the group consisting of abacavir, acyclovir, adefovir, amprenavir, atazanavir, cidofovir, darunavir, delavirdine, didanosine, docosanol, efavirenz, elvitegravir, emtricitabine, enfuviltide, etravirine, famciclovir, foscamet, fomivirsen, ganciclovir, indinavir, idoxuridine, lamivudine, lopinavir maraviroc, mk-2048, nelfinavir, nevirapine, nirmatrelvir, penciclovir, raltegravir, rilpivirine, ritonavir, saquinavir, stavudine, tenofovir trifluridine, valaciclovir, valganciclovir, vidarabine, ibacitabine, amantadine, oseltamivir, rimantidine, tipranavir, zalcitabine, zanamivir, and zidovudine. In certain embodiments, the antifungal agent is selected from the group consisting of miconazole, ketoconazole, clotrimazole, econazole, omoconazole, bifonazole, butoconazole, fenticonazole, isoconazole, oxiconazole, sertaconazole, sulconazole, tioconazole, fluconazole, itraconazole, isavuconazole, ravuconazole, posaconazole, voriconazok, terconazole, albaconazole, abafungin, terbinafine, naftifine, butenafine, anidulafungin, caspofungin, micafungin, polygodial, benzoic acid, ciclopirox, tolnaftate, undecylenic acid, flucytosine or 5-fluorocytosine, griseofulvin, and haloprogin. In certain embodiments, the anti-inflammatory and/or immunosuppressive agent is selected from the group consisting of cyclophosphamide, mycophenolate mofetil, corticosteroids, mesalazine, mesalamine, sulfasalazine, sulfasalazine derivatives, cyclosporin A, mercaptopurine, azathiopurine, prednisone, methotrexate, antihistamines, glucocorticoids, epinephrine, theophylline, cromolyn sodium, anti-leukotrienes, anticholinergics, monoclonal anti-IgE, immunomodulatory peptides, immunomodulatory small molecules, immunomodulatory cytokines, immunomodulatory antibodies, and vaccines. In certain embodiments, the prebiotic is selected from the group consisting of amino acids, biotin, fructooligosaccharides, galactooligosaccharides, inulin, lactulose, mannan oligosaccharides, oligofructose-enriched inulin, oligofructose, oligodextrose, tagatose, trans-galactooligosaccharide, and xylooligosaccharides.

In certain embodiments, the microbial consortium or pharmaceutical composition thereof is present in a food product.

The present disclosure also provides methods for restoring microbiome and/or recovering a healthy microbiome in a subject. In certain embodiments, the methods comprise administering an effective amount of a microbial consortium or a pharmaceutical composition thereof comprising:
a) Clostridium citroniae, Bacteroides salyersiae, Blautia obeum, Parabacteroides merdae, Parabacteroides distasonis, Anaerostipes hadrus, Lachnospiraceae sp. FBI00033, Eubacterium eligens, Bifidobacterium dentium, Blautia wexlerae, Fusicatenibacter saccharivorans, Bacteroides nordii, Dorea formicigenerans, Dorea longicatena, Bacteroides stercorirosoris, Bifidobacterium longum, Bacteroides kribbi, Lachnospiraceae sp. FBI00071, Bacteroides thetaiotaomicron, Clostridium clostridioforme, Clostridium scindens, Roseburia hominis, Clostridium fessum, Coprococcus comes, Blautia faecis, Hungatella hathewayi, Bacteroides stercoris, Collinsella aerofaciens, Hungatella effluvii, Bifidobacterium adolescentis, Bifidobacterium catenulatum, Lactobacillus rogosae, Bacteroides faecis, Bacteroides finegoldii, Clostridiaceae sp. FBI00191, Ruminococcus faecis, Lachnoclostridium pacaense, Clostridium bolteae, Longicatena caecimuris, Eggerthella lenta, Blautia massiliensis, Bacteroides xylanisolvens, Bacteroides vulgatus, Megasphaera massiliensis, Butyricimonas faecihominis, Eisenbergiella tayi, Acidaminococcus intestini, Emergencia timonensis, Bifidobacterium pseudocatenulatum, Eubacterium hallii, Anaerofustis stercorihominis, Eubacterium ventriosum, Blautia hydrogenotrophica, and Lachnospiraceae sp. FBI00290, or a functional equivalent thereof; or
b) FBI00001, FBI00002, FBI00010, FBI00013, FBI00029, FBI00032, FBI00033, FBI00034, FBI00043, FBI00044, FBI00048, FBI00050, FBI00051, FBI00057, FBI00059, FBI00060, FBI00070, FBI00071, FBI00076, FBI00079, FBI00087, FBI00093, FBI00102, FBJ00109, FBI00117, FBI00120, FBI00125, FBI00127, FBI00128, FBI00145, FBI00162, FBI00174, FBI00184, FBI00190, FBI00191, FBI00194, FBI00198, FBI00199, FBI00200, FBI00201, FBI00205, FBI00206, FBI00211, FBI00220, FBI00221, FBI00236, FBI00245, FBI00248, FBI00251, FBI00254, FBI00267, FBI00278, FBI00288, and FBI00290, or a functional equivalent thereof.

In certain embodiments, the microbial consortium or a pharmaceutical composition thereof further comprises:
a) Acutalibacter timonensis, Alistipes onderdonkii, Bacteroides uniformis, Eubacterium rectale, Alistipes timonensis, Bacteroides kribbi, Coprococcus eutactus, Bilophila wadsworthia, Bacteroides caccae, Alistipes shahii, Parasutterella excrementihominis, Paraprevotella clara, Sutterella wadsworthensis, Sutterella massiliensis, Porphyromonas asaccharolytica, Ruminococcus bromii, Monoglobus pectinilyticus, Ruminococcaceae sp. FBI00097, Gordonibacter pamelaeae, Bacteroides uniformis, Gordonibacter pamelaeae, Bacteroides fragilis, Phascolarctobacterium faecium, Monoglobus pectinilyticus, Clostridium aldenense, Ruthenibacterium lactatiformans, Bacteroides ovatus, Bifidobacterium bifidum, Anaerotruncus massiliensis, Clostridium aldenense, Sutterella wadsworthensis, Catabacter hongkongensis, Alistipes senegalensis, Ruminococcaceae sp. FBI00233, Alistipes shahii, Dielma fastidiosa, Eubacterium siraeum, Faecalibacterium prausnitzii, Turicibacter sanguinis, Eubacterium rectale, Bacteroides caccae, Methanobrevibacter smithii, Barnesiella intestinihominis, Alistipes onderdonkii, and Methanobrevibacter smithii, or a functional equivalent thereof;
b) Bifidobacterium adolescentis, Bifidobacterium longum, Bifidobacterium pseudocatenulatum, Bacteroides thetaiotaomicron, Coprococcus comes, Fusicatenibacter saccharivorans, Eggerthella lenta, Eubacterium eligens, Bacteroides xylanisolvens, Lactobacillus rogosae, Clostridium citroniae, Collinsella aerofaciens, Blautia obeum, Eggerthella lenta, Blautia wexlerae, Lachnoclostridium pacaense, Bacteroides vulgatus, Parabacteroides merdae, Dorea formicigenerans, Ruminococcus faecis, Roseburia hominis, Anaerostipes hadrus, Bifidobacterium adolescentis, Bifidobacterium pseudocatenulatum, Clostridium bolteae, Eisenbergiella tayi, Dorea longicatena, Eggerthella lenta, Bacteroides stercoris, Hungatella hathewayi, and Bacteroides xylanisolvens, or a functional equivalent thereof; and/or
c) Alistipes putredinis, Dialister succinatiphilus, Akkermansia muciniphila, Ruminococcus bromii, Dialister invisus, Bacteroides massiliensis, Bilophila wadsworthia, Holdemanella biformis, Parasutterella excrementihominis, Alistipes sp. FBI00180, Bacteroides coprocola, Alistipes sp. FBI00238, Alistipes putredinis, Eubacterium xylanophilum, and Senegalimassilia anaerobia, or a functional equivalent thereof.

In certain embodiments, the microbial consortium or a pharmaceutical composition thereof further comprises:
a) FBI00004, FBI00012, FBI00015, FBI00018, FBI00019, FBI00021, FBI00038, FBI00040, FBI00046, FBI00061, FBI00066, FBI00075, FBI00077, FBI00080, FBI00081, FBI00085, FBI00092, FBI00097, FBI00099, FBI00112, FBI00132, FBI00137, FBI00140, FBI00149, FBI00151, FBI00176, FBI00189, FBI00197, FBI00208, FBI00212, FBI00224, FBI00226, FBI00229, FBI00233, FBI00235, FBI00237, FBI00243, FBI00244, FBI00258, FBI00260, FBI00263, FBI00270, FBI00273, FBI00277, and FBI00292, or a functional equivalent thereof;
b) FBI00009, FBI00011, FBI00016, FBI00020, FBI00025, FBI00027, FBI00030, FBI00047, FBI00052, FBI00053, FBI00056, FBI00062, FBI00078, FBI00096, FBI00104, FBI00110, FBI00111, FBI00113, FBI00115, FBI00116, FBI00123, FBI00124, FBI00126, FBI00135, FBI00147, FBI00159, FBI00167, FBI00170, FBI00232, FBI00255, and FBI00271, or a functional equivalent thereof; and/or
c) FBI00022, FBI00049, FBI00068, FBI00069, FBI00152, FBI00165, FBI00171, FBI00175, FBI00177, FBI00180, FBI00182, FBI00238, FBI00269, FBI00274, and FBI00281, or a functional equivalent thereof.

In certain embodiments, the microbial consortium or a pharmaceutical composition thereof further comprises a first strain of Oxalobacter formigenes or FBI00067. In certain embodiments, the microbial consortium or a pharmaceutical composition thereof further comprises a second strain of Oxalobacter formigenes or FBI00133. In certain embodiments, the microbial consortium or a pharmaceutical composition thereof further comprises a third strain of Oxalobacter formigenes or FBI00289.

In certain embodiments, the microbial consortium or a pharmaceutical composition thereof comprises:
a) Clostridium citroniae, Bacteroides salyersiae, Blautia obeum, Parabacteroides merdae, Parabacteroides distasonis, Anaerostipes hadrus, Lachnospiraceae sp. FBI00033, Eubacterium eligens, Bifidobacterium dentium, Blautia wexlerae, Fusicatenibacter saccharivorans, Bacteroides nordii, Dorea formicigenerans, Dorea longicatena, Bacteroides stercorirosoris, Bifidobacterium longum, Bacteroides kribbi, Lachnospiraceae sp. FBI00071, Bacteroides thetaiotaomicron, Clostridium clostridioforme, Clostridium scindens, Roseburia hominis, Clostridium fessum, Coprococcus comes, Blautia faecis, Hungatella hathewayi, Bacteroides stercoris, Collinsella aerofaciens, Hungatella effluvii, Bifidobacterium adolescentis, Bifidobacterium catenulatum, Lactobacillus rogosae, Bacteroides faecis, Bacteroides finegoldii, Clostridiaceae sp. FBI00191, Ruminococcus faecis, Lachnoclostridium pacaense, Clostridium bolteae, Longicatena caecimuris, Eggerthella lenta, Blautia massiliensis, Bacteroides xylanisolvens, Bacteroides vulgatus, Megasphaera massiliensis, Butyricimonas faecihominis, Eisenbergiella tayi, Acidaminococcus intestini, Emergencia timonensis, Bifidobacterium pseudocatenulatum, Eubacterium hallii, Anaerofustis stercorihominis, Eubacterium ventriosum, Blautia hydrogenotrophica, and Lachnospiraceae sp. FBI00290, or a functional equivalent thereof;
b) Acutalibacter timonensis, Alistipes onderdonkii, Bacteroides uniformis, Eubacterium rectale, Alistipes timonensis, Bacteroides kribbi, Coprococcus eutactus, Bilophila wadsworthia, Bacteroides caccae, Alistipes shahii, Parasutterella excrementihominis, Paraprevotella clara, Sutterella wadsworthensis, Sutterella massiliensis, Porphyromonas asaccharolytica, Ruminococcus bromii, Monoglobus pectinilyticus, Ruminococcaceae sp. FBI00097, Gordonibacter pamelaeae, Bacteroides uniformis, Gordonibacter pamelaeae, Bacteroides fragilis, Phascolarctobacterium faecium, Monoglobus pectinilyticus, Clostridium aldenense, Ruthenibacterium lactatiformans, Bacteroides ovatus, Bifidobacterium bifidum, Anaerotruncus massiliensis, Clostridium aldenense, Sutterella wadsworthensis, Catabacter hongkongensis, Alistipes senegalensis, Ruminococcaceae sp. FBI00233, Alistipes shahii, Dielma fastidiosa, Eubacterium siraeum, Faecalibacterium prausnitzii, Turicibacter sanguinis, Eubacterium rectale, Bacteroides caccae, Methanobrevibacter smithii, Barnesiella intestinihominis, Alistipes onderdonkii, and Methanobrevibacter smithii, or a functional equivalent thereof;
c) Bifidobacterium adolescentis, Bifidobacterium longum, Bifidobacterium pseudocatenulatum, Bacteroides thetaiotaomicron, Coprococcus comes, Fusicatenibacter saccharivorans, Eggerthella lenta, Eubacterium eligens, Bacteroides xylanisolvens, Lactobacillus rogosae, Clostridium citroniae, Collinsella aerofaciens, Blautia obeum, Eggerthella lenta, Blautia wexlerae, Lachnoclostridium pacaense, Bacteroides vulgatus, Parabacteroides merdae, Dorea formicigenerans, Ruminococcus faecis, Roseburia hominis, Anaerostipes hadrus, Bifidobacterium adolescentis, Bifidobacterium pseudocatenulatum, Clostridium bolteae, Eisenbergiella tayi, Dorea longicatena, Eggerthella lenta, Bacteroides stercoris, Hungatella hathewayi, and Bacteroides xylanisolvens, or a functional equivalent thereof; and
d) Alistipes putredinis, Dialister succinatiphilus, Akkermansia muciniphila, Ruminococcus bromii, Dialister invisus, Bacteroides massiliensis, Bilophila wadsworthia, Holdemanella biformis, Parasutterella excrementihominis, Alistipes sp. FBI00180, Bacteroides coprocola, Alistipes sp. FBI00238, Alistipes putredinis, Eubacterium xylanophilum, and Senegalimassilia anaerobia, or a functional equivalent thereof.

In certain embodiments, the microbial consortium or a pharmaceutical composition thereof comprises:
a) FBI00001, FBI00002, FBI00010, FBI00013, FBI00029, FBI00032, FBI00033, FBI00034, FBI00043, FBI00044, FBI00048, FBI00050, FBI00051, FBI00057, FBI00059, FBI00060, FBI00070, FBI00071, FBI00076, FBI00079, FBI00087, FBI00093, FBI00102, FBI00109, FBI00117, FBI00120, FBI00125, FBI00127, FBI00128, FBI00145, FBI00162, FBI00174, FBI00184, FBI00190, FBI00191, FBI00194, FBI00198, FBI00199, FBI00200, FBI00201, FBI00205, FBI00206, FBI00211, FBI00220, FBI00221, FBI00236, FBI00245, FBI00248, FBI00251, FBI00254, FBI00267, FBI00278, FBI00288, and FBI00290, or a functional equivalent thereof;
b) FBI00004, FBI00012, FBI00015, FBI00018, FBI00019, FBI00021, FBI00038, FBI00040, FBI00046, FBI00061, FBI00066, FBI00075, FBI00077, FBI00080, FBI00081, FBI00085, FBI00092, FBI00097, FBI00099, FBI00112, FBI00132, FBI00137, FBI00140, FBI00149, FBI00151, FBI00176, FBI00189, FBI00197, FBI00208, FBI00212, FBI00224, FBI00226, FBI00229, FBI00233, FBI00235, FBI00237, FBI00243, FBI00244, FBI00258, FBI00260, FBI00263, FBI00270, FBI00273, FBI00277, and FBI00292, or a functional equivalent thereof;
c) FBI00009, FBI00011, FBI00016, FBI00020, FBI00025, FBI00027, FBI00030, FBI00047, FBI00052, FBI00053, FBI00056, FBI00062, FBI00078, FBI00096, FBI00104, FBI00110, FBI00111, FBI00113, FBI00115, FBI00116, FBI00123, FBI00124, FBI00126, FBI00135, FBI00147, FBI00159, FBI00167, FBI00170, FBI00232, FBI00255, and FBI00271, or a functional equivalent thereof; and
d) FBI00022, FBI00049, FBI00068, FBI00069, FBI00152, FBI00165, FBI00171, FBI00175, FBI00177, FBI00180, FBI00182, FBI00238, FBI00269, FBI00274, and FBI00281, or a functional equivalent thereof.

In certain embodiments, the microbial consortium or a pharmaceutical composition thereof further comprises a first strain of Oxalobacter formigenes or FBI00067, a second strain of Oxalobacter formigenes or FBI00133, and a third strain of Oxalobacter formigenes or FBI00289. In certain embodiments, the microbial consortium or a pharmaceutical composition thereof further comprises a first strain of Oxalobacter formigenes or FBI00067, a second strain of Oxalobacter formigenes or FBI00133, and a third strain of Oxalobacter formigenes or FBI00289. In certain embodiments, the microbial consortium or a pharmaceutical composition thereof is FB-001 or a functional equivalent thereof. In certain embodiments, the microbial consortium or a pharmaceutical composition thereof is FB-003 or a functional equivalent thereof.

In certain embodiments, the microbial consortium or a pharmaceutical composition thereof increases the microbial diversity of the gastrointestinal tract. In certain embodiments, the microbial consortium or a pharmaceutical composition thereof increases short chain fatty acids (SCFAs). In certain embodiments, the microbial consortium or a pharmaceutical composition thereof increases secondary bile acids. In certain embodiments, wherein the microbial consortium or a pharmaceutical composition thereof decreases bacterial pathogens in the gastrointestinal tract of the subject.

In certain embodiments, the microbial consortium or a pharmaceutical composition thereof comprises between about 5×10⁹ and about 5×10¹² viable cells. In certain embodiments, the microbial consortium or a pharmaceutical composition thereof comprises between about 5×10⁹ and about 5×10¹⁰ viable cells. In certain embodiments, the microbial consortium or a pharmaceutical composition thereof comprises between about 5×10¹⁰ and about 5×10¹¹ viable cells. In certain embodiments, the microbial consortium or a pharmaceutical composition thereof comprises between about 5×10¹¹ and about 5×10¹² viable cells. In certain embodiments, the microbial consortium or a pharmaceutical composition thereof comprises up to about 10¹¹ viable cells. In certain embodiments, the microbial consortium or a pharmaceutical composition thereof comprises up to about 10¹² viable cells. In certain embodiments, the method comprises administering a loading dose and one or more maintenance doses. In certain embodiments, the loading dose is administered for 1 day, 2 days, 3 days, 4 days, 5 days, 6 days, 7 days, 8 days, 9 days, or 10 days. In certain embodiments, the loading dose is administered for 2-3 days, 3-5 days, 4-6 days, or 5-7 days. In certain embodiments, the one or more maintenance doses are administered for at least 21 days following the last loading dose.

In certain embodiments, the methods further comprise administering an antibacterial agent, an antiviral agent, an antifungal agent, anti-inflammatory agent, an immunosuppressive agent, and/or a prebiotic. In certain embodiments, the antibacterial agent is selected from the group consisting of ciprofloxacin, levaquin, floxin, tequin, avelox, norflox, cephalexin, cefuroxime, cefadroxil, cefazolin, cephalothin, cefaclor, cefamandole, cefoxitin, cefprozil, ceftobiprole, amoxicillin, ampicillin, penicillin V, dicloxacillin, carbenicillin, vancomycin, and methicillin, tetracycline, minocycline, oxytetracycline, doxycycline, ertapenem, doripenem, imipenem/cilastatin, and meropenem. In certain embodiments, the antiviral agent is selected from the group consisting of abacavir, acyclovir, adefovir, amprenavir, atazanavir, cidofovir, darunavir, delavirdine, didanosine, docosanol, efavirenz, elvitegravir, emtricitabine, enfuviltide, etravirine, famciclovir, foscamet, fomivirsen, ganciclovir, indinavir, idoxuridine, lamivudine, lopinavir maraviroc, mk-2048, nelfinavir, nevirapine, nirmatrelvir, penciclovir, raltegravir, rilpivirine, ritonavir, saquinavir, stavudine, tenofovir trifluridine, valaciclovir, valganciclovir, vidarabine, ibacitabine, amantadine, oseltamivir, rimantidine, tipranavir, zalcitabine, zanamivir, and zidovudine. In certain embodiments, the antifungal agent is selected from the group consisting of miconazole, ketoconazole, clotrimazole, econazole, omoconazole, bifonazole, butoconazole, fenticonazole, isoconazole, oxiconazole, sertaconazole, sulconazole, tioconazole, fluconazole, itraconazole, isavuconazole, ravuconazole, posaconazole, voriconazok, terconazole, albaconazole, abafungin, terbinafine, naftifine, butenafine, anidulafungin, caspofungin, micafungin, polygodial, benzoic acid, ciclopirox, tolnaftate, undecylenic acid, flucytosine or 5-fluorocytosine, griseofulvin, and haloprogin. In certain embodiments, the anti-inflammatory and/or immunosuppressive agent is selected from the group consisting of cyclophosphamide, mycophenolate mofetil, corticosteroids, mesalazine, mesalamine, sulfasalazine, sulfasalazine derivatives, cyclosporin A, mercaptopurine, azathiopurine, prednisone, methotrexate, antihistamines, glucocorticoids, epinephrine, theophylline, cromolyn sodium, anti-leukotrienes, anticholinergics, monoclonal anti-IgE, immunomodulatory peptides, immunomodulatory small molecules, immunomodulatory cytokines, immunomodulatory antibodies, and vaccines. In certain embodiments, the prebiotic is selected from the group consisting of amino acids, biotin, fructooligosaccharides, galactooligosaccharides, inulin, lactulose, mannan oligosaccharides, oligofructose-enriched inulin, oligofructose, oligodextrose, tagatose, trans-galactooligosaccharide, and xylooligosaccharides.

In certain embodiments, the microbial consortium or pharmaceutical composition thereof is present in a food product.

Moreover, the present disclosure provides methods for treating a disease in a subject. In certain embodiments, the methods comprise administering an effective amount of a microbial consortium or a pharmaceutical composition thereof comprising:
a) Clostridium citroniae, Bacteroides salyersiae, Blautia obeum, Parabacteroides merdae, Parabacteroides distasonis, Anaerostipes hadrus, Lachnospiraceae sp. FBI00033, Eubacterium eligens, Bifidobacterium dentium, Blautia wexlerae, Fusicatenibacter saccharivorans, Bacteroides nordii, Dorea formicigenerans, Dorea longicatena, Bacteroides stercorirosoris, Bifidobacterium longum, Bacteroides kribbi, Lachnospiraceae sp. FBI00071, Bacteroides thetaiotaomicron, Clostridium clostridioforme, Clostridium scindens, Roseburia hominis, Clostridium fessum, Coprococcus comes, Blautia faecis, Hungatella hathewayi, Bacteroides stercoris, Collinsella aerofaciens, Hungatella effluvii, Bifidobacterium adolescentis, Bifidobacterium catenulatum, Lactobacillus rogosae, Bacteroides faecis, Bacteroides finegoldii, Clostridiaceae sp. FBI00191, Ruminococcus faecis, Lachnoclostridium pacaense, Clostridium bolteae, Longicatena caecimuris, Eggerthella lenta, Blautia massiliensis, Bacteroides xylanisolvens, Bacteroides vulgatus, Megasphaera massiliensis, Butyricimonas faecihominis, Eisenbergiella tayi, Acidaminococcus intestini, Emergencia timonensis, Bifidobacterium pseudocatenulatum, Eubacterium hallii, Anaerofustis stercorihominis, Eubacterium ventriosum, Blautia hydrogenotrophica, and Lachnospiraceae sp. FBI00290, or a functional equivalent thereof; or
b) FBI00001, FBI00002, FBI00010, FBI00013, FBI00029, FBI00032, FBI00033, FBI00034, FBI00043, FBI00044, FBI00048, FBI00050, FBI00051, FBI00057, FBI00059, FBI00060, FBI00070, FBI00071, FBI00076, FBI00079, FBI00087, FBI00093, FBI00102, FBI00109, FBI00117, FBI00120, FBI00125, FBI00127, FBI00128, FBI00145, FBI00162, FBI00174, FBI00184, FBI00190, FBI00191, FBI00194, FBI00198, FBI00199, FBI00200, FBI00201, FBI00205, FBI00206, FBI00211, FBI00220, FBI00221, FBI00236, FBI00245, FBI00248, FBI00251, FBI00254, FBI00267, FBI00278, FBI00288, and FBI00290, or a functional equivalent thereof.

In certain embodiments, the disease is irritable bowel syndrome, diarrhea, constipation, celiac disease, and leaky gut syndrome, colitis, ulcerative colitis, or Crohn's disease.

In certain embodiments, the microbial consortium or a pharmaceutical composition thereof further comprises:
a) Acutalibacter timonensis, Alistipes onderdonkii, Bacteroides uniformis, Eubacterium rectale, Alistipes timonensis, Bacteroides kribbi, Coprococcus eutactus, Bilophila wadsworthia, Bacteroides caccae, Alistipes shahii, Parasutterella excrementihominis, Paraprevotella clara, Sutterella wadsworthensis, Sutterella massiliensis, Porphyromonas asaccharolytica, Ruminococcus bromii, Monoglobus pectinilyticus, Ruminococcaceae sp. FBI00097, Gordonibacter pamelaeae, Bacteroides uniformis, Gordonibacter pamelaeae, Bacteroides fragilis, Phascolarctobacterium faecium, Monoglobus pectinilyticus, Clostridium aldenense, Ruthenibacterium lactatiformans, Bacteroides ovatus, Bifidobacterium bifidum, Anaerotruncus massiliensis, Clostridium aldenense, Sutterella wadsworthensis, Catabacter hongkongensis, Alistipes senegalensis, Ruminococcaceae sp. FBI00233, Alistipes shahii, Dielma fastidiosa, Eubacterium siraeum, Faecalibacterium prausnitzii, Turicibacter sanguinis, Eubacterium rectale, Bacteroides caccae, Methanobrevibacter smithii, Barnesiella intestinihominis, Alistipes onderdonkii, and Methanobrevibacter smithii, or a functional equivalent thereof;
b) Bifidobacterium adolescentis, Bifidobacterium longum, Bifidobacterium pseudocatenulatum, Bacteroides thetaiotaomicron, Coprococcus comes, Fusicatenibacter saccharivorans, Eggerthella lenta, Eubacterium eligens, Bacteroides xylanisolvens, Lactobacillus rogosae, Clostridium citroniae, Collinsella aerofaciens, Blautia obeum, Eggerthella lenta, Blautia wexlerae, Lachnoclostridium pacaense, Bacteroides vulgatus, Parabacteroides merdae, Dorea formicigenerans, Ruminococcus faecis, Roseburia hominis, Anaerostipes hadrus, Bifidobacterium adolescentis, Bifidobacterium pseudocatenulatum, Clostridium bolteae, Eisenbergiella tayi, Dorea longicatena, Eggerthella lenta, Bacteroides stercoris, Hungatella hathewayi, and Bacteroides xylanisolvens, or a functional equivalent thereof; and/or
c) Alistipes putredinis, Dialister succinatiphilus, Akkermansia muciniphila, Ruminococcus bromii, Dialister invisus, Bacteroides massiliensis, Bilophila wadsworthia, Holdemanella biformis, Parasutterella excrementihominis, Alistipes sp. FBI00180, Bacteroides coprocola, Alistipes sp. FBI00238, Alistipes putredinis, Eubacterium xylanophilum, and Senegalimassilia anaerobia, or a functional equivalent thereof.

In certain embodiments, the microbial consortium or a pharmaceutical composition thereof further comprises:
a) FBI00004, FBI00012, FBI00015, FBI00018, FBI00019, FBI00021, FBI00038, FBI00040, FBI00046, FBI00061, FBI00066, FBI00075, FBI00077, FBI00080, FBI00081, FBI00085, FBI00092, FBI00097, FBI00099, FBI00112, FBI00132, FBI00137, FBI00140, FBI00149, FBI00151, FBI00176, FBI00189, FBI00197, FBI00208, FBI00212, FBI00224, FBI00226, FBI00229, FBI00233, FBI00235, FBI00237, FBI00243, FBI00244, FBI00258, FBI00260, FBI00263, FBI00270, FBI00273, FBI00277, and FBI00292, or a functional equivalent thereof;
b) FBI00009, FBI00011, FBI00016, FBI00020, FBI00025, FBI00027, FBI00030, FBI00047, FBI00052, FBI00053, FBI00056, FBI00062, FBI00078, FBI00096, FBI00104, FBI00110, FBI00111, FBI00113, FBI00115, FBI00116, FBI00123, FBI00124, FBI00126, FBI00135, FBI00147, FBI00159, FBI00167, FBI00170, FBI00232, FBI00255, and FBI00271, or a functional equivalent thereof; and/or
c) FBI00022, FBI00049, FBI00068, FBI00069, FBI00152, FBI00165, FBI00171, FBI00175, FBI00177, FBI00180, FBI00182, FBI00238, FBI00269, FBI00274, and FBI00281, or a functional equivalent thereof.

In certain embodiments, the microbial consortium or a pharmaceutical composition thereof further comprises a first strain of Oxalobacter formigenes or FBI00067.

In certain embodiments, the microbial consortium or a pharmaceutical composition thereof further comprises a second strain of Oxalobacter formigenes or FBI00133.

In certain embodiments, the microbial consortium or a pharmaceutical composition thereof further comprises a third strain of Oxalobacter formigenes or FBI00289.

In certain embodiments, the microbial consortium or a pharmaceutical composition thereof comprises:
a) Clostridium citroniae, Bacteroides salyersiae, Blautia obeum, Parabacteroides merdae, Parabacteroides distasonis, Anaerostipes hadrus, Lachnospiraceae sp. FBI00033, Eubacterium eligens, Bifidobacterium dentium, Blautia wexlerae, Fusicatenibacter saccharivorans, Bacteroides nordii, Dorea formicigenerans, Dorea longicatena, Bacteroides stercorirosoris, Bifidobacterium longum, Bacteroides kribbi, Lachnospiraceae sp. FBI00071, Bacteroides thetaiotaomicron, Clostridium clostridioforme, Clostridium scindens, Roseburia hominis, Clostridium fessum, Coprococcus comes, Blautia faecis, Hungatella hathewayi, Bacteroides stercoris, Collinsella aerofaciens, Hungatella effluvii, Bifidobacterium adolescentis, Bifidobacterium catenulatum, Lactobacillus rogosae, Bacteroides faecis, Bacteroides finegoldii, Clostridiaceae sp. FBI00191, Ruminococcus faecis, Lachnoclostridium pacaense, Clostridium bolteae, Longicatena caecimuris, Eggerthella lenta, Blautia massiliensis, Bacteroides xylanisolvens, Bacteroides vulgatus, Megasphaera massiliensis, Butyricimonas faecihominis, Eisenbergiella tayi, Acidaminococcus intestini, Emergencia timonensis, Bifidobacterium pseudocatenulatum, Eubacterium hallii, Anaerofustis stercorihominis, Eubacterium ventriosum, Blautia hydrogenotrophica, and Lachnospiraceae sp. FBI00290, or a functional equivalent thereof;
b) Acutalibacter timonensis, Alistipes onderdonkii, Bacteroides uniformis, Eubacterium rectale, Alistipes timonensis, Bacteroides kribbi, Coprococcus eutactus, Bilophila wadsworthia, Bacteroides caccae, Alistipes shahii, Parasutterella excrementihominis, Paraprevotella clara, Sutterella wadsworthensis, Sutterella massiliensis, Porphyromonas asaccharolytica, Ruminococcus bromii, Monoglobus pectinilyticus, Ruminococcaceae sp. FBI00097, Gordonibacter pamelaeae, Bacteroides uniformis, Gordonibacter pamelaeae, Bacteroides fragilis, Phascolarctobacterium faecium, Monoglobus pectinilyticus, Clostridium aldenense, Ruthenibacterium lactatiformans, Bacteroides ovatus, Bifidobacterium bifidum, Anaerotruncus massiliensis, Clostridium aldenense, Sutterella wadsworthensis, Catabacter hongkongensis, Alistipes senegalensis, Ruminococcaceae sp. FBI00233, Alistipes shahii, Dielma fastidiosa, Eubacterium siraeum, Faecalibacterium prausnitzii, Turicibacter sanguinis, Eubacterium rectale, Bacteroides caccae, Methanobrevibacter smithii, Barnesiella intestinihominis, Alistipes onderdonkii, and Methanobrevibacter smithii, or a functional equivalent thereof;
c) Bifidobacterium adolescentis, Bifidobacterium longum, Bifidobacterium pseudocatenulatum, Bacteroides thetaiotaomicron, Coprococcus comes, Fusicatenibacter saccharivorans, Eggerthella lenta, Eubacterium eligens, Bacteroides xylanisolvens, Lactobacillus rogosae, Clostridium citroniae, Collinsella aerofaciens, Blautia obeum, Eggerthella lenta, Blautia wexlerae, Lachnoclostridium pacaense, Bacteroides vulgatus, Parabacteroides merdae, Dorea formicigenerans, Ruminococcus faecis, Roseburia hominis, Anaerostipes hadrus, Bifidobacterium adolescentis, Bifidobacterium pseudocatenulatum, Clostridium bolteae, Eisenbergiella tayi, Dorea longicatena, Eggerthella lenta, Bacteroides stercoris, Hungatella hathewayi, and Bacteroides xylanisolvens, or a functional equivalent thereof; and
d) Alistipes putredinis, Dialister succinatiphilus, Akkermansia muciniphila, Ruminococcus bromii, Dialister invisus, Bacteroides massiliensis, Bilophila wadsworthia, Holdemanella biformis, Parasutterella excrementihominis, Alistipes sp. FBI00180, Bacteroides coprocola, Alistipes sp. FBI00238, Alistipes putredinis, Eubacterium xylanophilum, and Senegalimassilia anaerobia, or a functional equivalent thereof.

In certain embodiments, the microbial consortium or a pharmaceutical composition thereof comprises:
a) FBI00001, FBI00002, FBI00010, FBI00013, FBI00029, FBI00032, FBI00033, FBI00034, FBI00043, FBI00044, FBI00048, FBI00050, FBI00051, FBI00057, FBI00059, FBI00060, FBI00070, FBI00071, FBI00076, FBI00079, FBI00087, FBI00093, FBI00102, FBI00109, FBI00117, FBI00120, FBI00125, FBI00127, FBI00128, FBI00145, FBI00162, FBI00174, FBI00184, FBI00190, FBI00191, FBI00194, FBI00198, FBI00199, FBI00200, FBI00201, FBI00205, FBI00206, FBI00211, FBI00220, FBI00221, FBI00236, FBI00245, FBI00248, FBI00251, FBI00254, FBI00267, FBI00278, FBI00288, and FBI00290, or a functional equivalent thereof;
b) FBI00004, FBI00012, FBI00015, FBI00018, FBI00019, FBI00021, FBI00038, FBI00040, FBI00046, FBI00061, FBI00066, FBI00075, FBI00077, FBI00080, FBI00081, FBI00085, FBI00092, FBI00097, FBI00099, FBI00112, FBI00132, FBI00137, FBI00140, FBI00149, FBI00151, FBI00176, FBI00189, FBI00197, FBI00208, FBI00212, FBI00224, FBI00226, FBI00229, FBI00233, FBI00235, FBI00237, FBI00243, FBI00244, FBI00258, FBI00260, FBI00263, FBI00270, FBI00273, FBI00277, and FBI00292, or a functional equivalent thereof;
c) FBI00009, FBI00011, FBI00016, FBI00020, FBI00025, FBI00027, FBI00030, FBI00047, FBI00052, FBI00053, FBI00056, FBI00062, FBI00078, FBI00096, FBI00104, FBI00110, FBI00111, FBI00113, FBI00115, FBI00116, FBI00123, FBI00124, FBI00126, FBI00135, FBI00147, FBI00159, FBI00167, FBI00170, FBI00232, FBI00255, and FBI00271, or a functional equivalent thereof; and
d) FBI00022, FBI00049, FBI00068, FBI00069, FBI00152, FBI00165, FBI00171, FBI00175, FBI00177, FBI00180, FBI00182, FBI00238, FBI00269, FBI00274, and FBI00281, or a functional equivalent thereof.

In certain embodiments, the microbial consortium or a pharmaceutical composition thereof further comprises a first strain of Oxalobacter formigenes or FBI00067, a second strain of Oxalobacter formigenes or FBI00133, and a third strain of Oxalobacter formigenes or FBI00289. In certain embodiments, the microbial consortium or a pharmaceutical composition thereof further comprises a first strain of Oxalobacter formigenes or FBI00067, a second strain of Oxalobacter formigenes or FBI00133, and a third strain of Oxalobacter formigenes or FBI00289. In certain embodiments, the microbial consortium or a pharmaceutical composition thereof is FB-001 or a functional equivalent thereof. In certain embodiments, the microbial consortium or a pharmaceutical composition thereof is FB-003 or a functional equivalent thereof. In certain embodiments, the microbial consortium or a pharmaceutical composition thereof increases the microbial diversity of the gastrointestinal tract. In certain embodiments, the microbial consortium or a pharmaceutical composition thereof increases short chain fatty acids (SCFAs). In certain embodiments, the microbial consortium or a pharmaceutical composition thereof increases secondary bile acids. In certain embodiments, the microbial consortium or a pharmaceutical composition thereof decreases bacterial pathogens in the gastrointestinal tract of the subject.

In certain embodiments, the microbial consortium or a pharmaceutical composition thereof comprises between about 5×10⁹ and about 5×10¹² viable cells. In certain embodiments, the microbial consortium or a pharmaceutical composition thereof comprises between about 5×10⁹ and about 5×10¹⁰ viable cells. In certain embodiments, the microbial consortium or a pharmaceutical composition thereof comprises between about 5×10¹⁰ and about 5×10¹¹ viable cells. In certain embodiments, the microbial consortium or a pharmaceutical composition thereof comprises between about 5×10¹¹ and about 5×10¹² viable cells. In certain embodiments, the microbial consortium or a pharmaceutical composition thereof comprises up to about 10¹¹ viable cells. In certain embodiments, the microbial consortium or a pharmaceutical composition thereof comprises up to about 10¹² viable cells. In certain embodiments, the method comprises administering a loading dose and one or more maintenance doses. In certain embodiments, the loading dose is administered for 1 day, 2 days, 3 days, 4 days, 5 days, 6 days, 7 days, 8 days, 9 days, or 10 days. In certain embodiments, the loading dose is administered for 2-3 days, 3-5 days, 4-6 days, or 5-7 days. In certain embodiments, the one or more maintenance doses are administered for at least 21 days following the last loading dose.

In certain embodiments, the methods further comprise administering an antibacterial agent, an antiviral agent, an antifungal agent, anti-inflammatory agent, an immunosuppressive agent, and/or a prebiotic. In certain embodiments, the antibacterial agent is selected from the group consisting of ciprofloxacin, levaquin, floxin, tequin, avelox, norflox, cephalexin, cefuroxime, cefadroxil, cefazolin, cephalothin, cefaclor, cefamandole, cefoxitin, cefprozil, ceftobiprole, amoxicillin, ampicillin, penicillin V, dicloxacillin, carbenicillin, vancomycin, and methicillin, tetracycline, minocycline, oxytetracycline, doxycycline, ertapenem, doripenem, imipenem/cilastatin, and meropenem. In certain embodiments, the antiviral agent is selected from the group consisting of abacavir, acyclovir, adefovir, amprenavir, atazanavir, cidofovir, darunavir, delavirdine, didanosine, docosanol, efavirenz, elvitegravir, emtricitabine, enfuviltide, etravirine, famciclovir, foscamet, fomivirsen, ganciclovir, indinavir, idoxuridine, lamivudine, lopinavir maraviroc, mk-2048, nelfinavir, nevirapine, nirmatrelvir, penciclovir, raltegravir, rilpivirine, ritonavir, saquinavir, stavudine, tenofovir trifluridine, valaciclovir, valganciclovir, vidarabine, ibacitabine, amantadine, oseltamivir, rimantidine, tipranavir, zalcitabine, zanamivir, and zidovudine. In certain embodiments, the antifungal agent is selected from the group consisting of miconazole, ketoconazole, clotrimazole, econazole, omoconazole, bifonazole, butoconazole, fenticonazole, isoconazole, oxiconazole, sertaconazole, sulconazole, tioconazole, fluconazole, itraconazole, isavuconazole, ravuconazole, posaconazole, voriconazok, terconazole, albaconazole, abafungin, terbinafine, naftifine, butenafine, anidulafungin, caspofungin, micafungin, polygodial, benzoic acid, ciclopirox, tolnaftate, undecylenic acid, flucytosine or 5-fluorocytosine, griseofulvin, and haloprogin. In certain embodiments, the anti-inflammatory and/or immunosuppressive agent is selected from the group consisting of cyclophosphamide, mycophenolate mofetil, corticosteroids, mesalazine, mesalamine, sulfasalazine, sulfasalazine derivatives, cyclosporin A, mercaptopurine, azathiopurine, prednisone, methotrexate, antihistamines, glucocorticoids, epinephrine, theophylline, cromolyn sodium, anti-leukotrienes, anticholinergics, monoclonal anti-IgE, immunomodulatory peptides, immunomodulatory small molecules, immunomodulatory cytokines, immunomodulatory antibodies, and vaccines. In certain embodiments, the prebiotic is selected from the group consisting of amino acids, biotin, fructooligosaccharides, galactooligosaccharides, inulin, lactulose, mannan oligosaccharides, oligofructose-enriched inulin, oligofructose, oligodextrose, tagatose, trans-galactooligosaccharide, and xylooligosaccharides.

In certain embodiments, the microbial consortium or pharmaceutical composition thereof is present in a food product. In certain embodiments, the methods discloses herein further comprise diagnosing IBD in the subject before administration of the microbial consortium or pharmaceutical composition thereof.

The present disclosure further provides a method for decreasing dysbiosis in a subject, the method comprising administering to a patient a therapeutically effective amount of a pharmaceutical composition comprising a Consortia to decrease dysbiosis of the gastrointestinal tract in the patient. In certain embodiments, decreasing dysbiosis comprises engraftment of microbes of the Consortia, increase in the microbial diversity of the gastrointestinal tract, increase in the short chain fatty acids (SCFAs), increase in secondary bile acids, and/or decrease of bacterial pathogens. In certain embodiments, the Consortia is FB-001 or a functional equivalent thereof. In certain embodiments, the Consortia is FB-003 or a functional equivalent thereof.

In addition, the present disclosure provides a method for restoring the microbiome in a patient, the method comprising administering to the patient a therapeutically effective amount of a pharmaceutical composition comprising a Consortia. In certain embodiments, the restoring the microbiome comprises engraftment of microbes of the Consortia, increase in the microbial diversity of the gastrointestinal tract, increase in the short chain fatty acids (SCFAs), increase in secondary bile acids, and/or decrease of bacterial pathogens. In certain embodiments, the Consortia is FB-001 or a functional equivalent thereof. In certain embodiments, the Consortia is FB-003 or a functional equivalent thereof.

The present disclosure provides a method for increasing the recovery of a healthy microbiome in a patient after a dysbiosis inducing event, the method comprising administering to the subject a therapeutically effective amount of a pharmaceutical composition comprising a Consortia. In certain embodiments, the recovery of a healthy microbiome comprises engraftment of microbes of the Consortia, increase in the microbial diversity of the gastrointestinal tract, increase in the short chain fatty acids (SCFAs), increase in secondary bile acids, and/or decrease of bacterial pathogens. In certain embodiments, the Consortia is FB-001 or a functional equivalent thereof. In certain embodiments, the Consortia is FB-003 or a functional equivalent thereof. In certain embodiments, the dysbiosis inducing event is treatment with one or more antibiotics, an infectious disease, or an underlying disease. In certain embodiments, the underlying disease is IBD, colitis, ulcerative colitis, or Crohn's disease.

The present disclosure provides a composition for treating or reducing a severity of at least one symptom of a gastrointestinal disease, disorder or condition associated with a dysbiosis in a patient, the composition comprising a Consortia in an amount effective to populate and optionally engraft in a gastrointestinal tract in the patient. In certain embodiments, the gastrointestinal disease is selected from the group consisting of IBD, colitis, ulcerative colitis, and Crohn's disease. In certain embodiments, the dysbiosis is associated with a decrease in the microbial diversity of the gastrointestinal tract, a decrease in the short chain fatty acids (SCFAs), a decrease in secondary bile acids, and/or an increase of bacterial pathogens. In certain embodiments, the Consortia is FB-001 or a functional equivalent thereof. In certain embodiments, the Consortia is FB-003 or a functional equivalent thereof.

The present disclosure also provides a method of treating or reducing a severity of at least one symptom of a gastrointestinal disease associated with a dysbiosis, the method comprising administering an effective amount of a pharmaceutical composition comprising a Consortia. In certain embodiments, the Consortia is FB-001 or a functional equivalent thereof. In certain embodiments, the Consortia is FB-003 or a functional equivalent thereof.

The present disclosure provides a composition comprising FB-003 or a functional equivalent thereof. The present disclosure provides a method of making FB-003 or a functional equivalent thereof.

The present disclosure provides a method of treating IBD, colitis, ulcerative colitis, or Crohn's disease by administering a Consortia. In certain embodiments, the Consortia is FB-003 or FB-001. The present disclosure provides a method of reducing the symptoms associated with IBD, colitis, ulcerative colitis, or Crohn's disease by administering a Consortia. In certain embodiments, the Consortia is FB-003 or FB-001. The present disclosure provides any method or composition described herein.

### BRIEF DESCRIPTION OF THE DRAWINGS

FIG. 1A shows an exemplary coculture experiment and FIG. 1B shows an exemplary coculture experiment that was modified to yield 100% strain detection following coculture.

FIG. 2A shows the design of the DS buckets for a Consortia and FIG. 2B shows the yield of strains after coculture depending on the inoculum seed.

FIGS. 3A and 3B show examples of different lyophilization excipients.

FIGS. 4A and 4B show examples of different lyophilization excipients and reducing agents.

FIGS. 5A and 5B show examples of different lyophilization excipients.

FIG. 6A is a venn diagram showing the overlapping microbes of five representative consortia designed and disclosed herein. FIG. 6B shows the breakdown of the type of microbe in each of the 5 representative consortia.

FIGS. 7A and 7B show the schematics of the experimental designs of the studies described in Example 5.

FIG. 8 shows that YCFAC+GalNAc is not able to support the growth of Akkermansia.

FIG. 9 shows that Threonine supports the growth of Akkermansia in the absence of GalNAc.

FIG. 10 shows a diagram of the coculture method of manufacture of FB-001 and FB-003.

FIG. 11 shows an overview of the strain isolation and purification process, RCB banking, and RCB identity/purity testing.

FIG. 12 shows a method for generation of master cell banks (MCB).

FIG. 13 shows a phylogenetic tree indicating the taxonomic composition of certain of the consortia disclosed herein, including the FB-001 and FB-003 Consortia.

FIGS. 14A-14C show a table summarizing the strains and species of the microbial consortia disclosed herein.

FIG. 15A shows the effect FB-001 has on reducing gut permeability and FIG. 15B shows the ability of FB-001 to produce short chain fatty acids (SCFA) at a level that is comparable to a normal, healthy gut. Butyrate, a SCFA, is important because it supports gastrointestinal epithelial cell health, energy metabolism and cell signaling to improve barrier function. In this experiment, the O. formigenes did not show activity and/or viability and thus the drug product used in these experiments are a research version of FB-003 (i.e., the strains of FB-001 without O. formigenes).

FIG. 16 shows the manufacturing process used for O. formigenes in the production of the Consortia described herein. Furthermore, DS5-DS7 (i.e., the three O. formigenes drug substances) of FB-001 used this manufacturing process for GMP and non-GMP manufacture.

FIG. 17 shows the manufacturing process used for DS1 in the production of the Consortia described herein. Furthermore, DS1 of FB-001 used this manufacturing process for GMP and non-GMP manufacture.

FIG. 18 shows the manufacturing process used for DS2 in the production of the Consortia described herein. Furthermore, DS2 of FB-001 used this manufacturing process for GMP and non-GMP manufacture.

FIG. 19 shows the manufacturing process used for DS3 in the production of the Consortia described herein. Furthermore, DS3 of FB-001 used this manufacturing process for GMP and non-GMP manufacture.

FIG. 20 shows the manufacturing process used for DS4 in the production of the Consortia described herein. Furthermore, DS4 of FB-001 used this manufacturing process for GMP and non-GMP manufacture.

FIG. 21A shows the experimental design for the in vivo assessment for FB-003 engraftment and metabolic function after antibiotic treatment in mice. FIG. 21B shows that FB-003 has stable engraftment for over 60 days in SPF mice. The “veh” or vehicle used in this experiment was plain media (the term “Media” is used for simplicity in FIG. 21A. “abx” stands for antibiotics. The antibiotics (“antibiotics” in FIG. 21A and “abx” in FIG. 21B) used in this experiment was 0.575 mg/mL enrofloxacin and 1 mg/mL ampicillin that was provided in the drinking water for 11 days. FIG. 21C shows the diverse colonization of of FB-003 based on genus level engraftment. FIG. 21D shows the diverse colonization of of FB-003 based on strain level engraftment. The engraftment of FB-003 shown in FIGS. 21C and 21D is from 1 day to 60 days after dosing SPF mice with FB-003 and shows that engraftment stabilizes approximately after 1 week of dosing.

FIGS. 22A-22C show that FB-004 treatment can rapidly produce a modified SCFA profile in mice. Specifically, the total SCFA recovery following microbiome ablating antibiotic treatment (0.575 mg/mL enrofloxacin and 1 mg/mL ampicillin) is faster with FB-003 than with a vehicle control, that FB-003 can control the SCFA profile of the SPF mice, and that the vehicle (i.e., media) treated mice bloom a dysbiotic community dominated by butyrate producers. FIG. 22A shows the total SCFA (acetate not included) concentrations following antibiotic treatment in mice+/−FB-003 treatment. FIG. 22B shows SCFA levels following antibiotic and FB-003 treatment. FIG. 22C shows SCFA levels following antibiotic and vehicle (i.e, media control) treatment. Comparing FIGS. 22B and 22C show that the ration of SCFAs are different between the FB-003 treatment group and the control group such that butyrate is dominant over proprionate in the control group which is a sign of dysbiosis.

FIG. 23A shows that antibiotics dramatically disrupt the bile acid pool in the gut. FIG. 23B shows that FB-003 rapidly restores balance to the bile acid pool after antibiotic treatment induces dysbiosis.

FIGS. 24A-24F show the design and results of a DSS colitis mouse model experiment. FIG. 24A shows a schematic of the DSS colitis experimental design of the DSS colitis model. FIG. 24B shows the experimental design for sample collection and analysis. FIG. 24C shows the body weight results of 5% DSS+/−FB-003 in the presence of antibiotic pre-treatment. FIG. 24D shows the body weight results of 5% DSS+/−FB-003 in the absence of antibiotic pre-treatment. FIG. 24E shows that antibiotics are required to displace a native mouse microbiome, that antibiotic treatment increases DSS colitis severity, and that FB-003 treatment dramatically improves the clinical scores of the mice (and the experiment also showed a reduced weight loss). FIG. 24F shows that in the no antibiotic setting, FB-003 still shows improvement in the clinical score (reduced weight loss was also observed). For FIGS. 24E and 24F, Total Clinical Score was calculated as Area Under the Curve (AUC). Data is presented as mean±SEM (n=2 or Naïve, n=10 per treatment group). Data was analysed by Ordinary One-Way ANOVA followed by Sidak's multiple comparisons test, the mean of the Abx+FB-003 group was compared to Abx+Vehicle group and the FB-003 treated group compared against the vehicle group. FIG. 24G shows the stool consistency score following 5% DSS-induced ulcerative colitis. Specifically, Stool Consistency Score was calculated as Area Under the Curve (AUC). Data is presented as mean±SEM (n=2 or Naïve, n=10 per treatment group). Data was analysed by Ordinary One-Way ANOVA followed by Sidak's multiple comparisons test, the mean of the Abx+FB-003 group was compared to Abx+Vehicle group and the FB-003 treated group compared against the vehicle group.

FIG. 25 shows that FB-003 improves survival in the DSS colitis model.

FIG. 26 provides a chart that provides references that disclose the function of certain species within the Consortia disclosed herein, including FB-003, Consortia A, and Consortia B consortia.

FIG. 27A shows clinical scores of the colon of mice in the DSS study described in the examples. FIGS. 27B and 27C shows images of the colon of mice treated with FB-003 in contrast to vehicle control.

### DETAILED DESCRIPTION

The present disclosure relates to compositions and methods for engrafting a microbial consortia disclosed herein. The present disclosure is based, in part, on the discovery that the presently disclosed microbial consortia are capable to effectively engraft in a subject and reduce the dysbiosis, restore the microbiome, and recovery the microbiome of a subject. Furthermore, the present disclosure relates methods of treating IBD, colitis, ulcerative colitis, and Crohn's disease comprising administering the microbial consortia disclosed herein. For clarity of description, and not by way of limitation, this section is divided into the subsections outlined below.

### Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

It is understood that aspects and embodiments of the present disclosure described herein include “comprising,” “consisting,” and “consisting essentially of” aspects and embodiments. The terms “comprises” and “comprising” are intended to have the broad meaning ascribed to them in U.S. Patent Law and can mean “includes,” “including” and the like. To facilitate an understanding of the present disclosure, a number of terms and phrases are defined below.

The term “a” and “an” as used herein mean “one or more” and include the plural unless the context is appropriate.

As used herein, the term “microbe” or “microbiota” refers to a microbial organism including, but not limited to, bacteria, archaea, protozoa, and unicellular fungi.

As used herein, the term “active microbes” refers to microbes that express sufficient amounts of one or more metabolic enzymes to metabolize a substrate that causes or contributes to disease in an animal.

As used herein, the term “supportive community” refers to one or more microbial strains that, when administered with an active microbe, enhance one or more characteristics of the active microbe selected from the group consisting of gastrointestinal engraftment, biomass, metabolic substrate metabolism, and longitudinal stability.

As used herein, the term “synthesizing microbe” refers to a microbe that expresses sufficient amounts of one or more enzymes to catalyze the combination of one or more metabolites produced by an active microbe, and one or more fermentation products produced by a fermenting microbe in a gastrointestinal niche.

As used herein, the term “fermenting microbe” refers to a microbe that expresses sufficient amounts of one or more enzymes to catalyze a fermentation reaction in a gastrointestinal niche.

As used herein, the term “longitudinal stability” refers to the ability of one or more microbes, or microbial consortia, to remain engrafted and metabolically active in one of more than one niche of the gastrointestinal tract despite transient or long-term environmental changes to the gastrointestinal niche.

As used herein, the term “metabolism,” “metabolize,” “metabolization,” or variants thereof refers to the biochemical conversion of a metabolic substrate to a metabolic product. In certain embodiments, metabolization includes isomerization.

As used herein, the term “biomass,” refers to the total mass of one or more microbes, or consortia, in a given area or volume.

As used herein, the terms “microbial consortia” and “microbial consortium” are used interchangeably and refer to a mixture of two or more isolated microbial strains that are expanded in culture, wherein one microbial strain in the mixture has a beneficial or desired effect on another microbial strain in the mixture.

As used herein, the term “Consortia” is used as a capitalized term to refer to one or more of the microbial consortia described herein.

As used herein, “dysbiosis” refers to an imbalance in the microbiome homeostasis within internal organs and tissues of a subject (e.g., gut) or on the external organs, tissues, and surfaces of the subject (e.g., skin).

As used herein, the term “gastrointestinal engraftment” or “engraft” or “engraftment” refers to the establishment of one or more microbes, or microbial consortia, in one or more niches of the gastrointestinal tract that, prior to administration of the one or more microbes, or microbial consortia, lacks the one or more microbes, or microbial consortia. For clarity, engraftment refers to the engraftment of one or more microbes administered to a subject. In certain embodiments, the gastrointestinal engraftment can be transient. In certain embodiments, the gastrointestinal engraftment can be persistent.

As used herein, the term “pharmaceutical composition” refers to the combination of an active agent with a carrier, inert or active, making the composition especially suitable for therapeutic use in vivo or ex vivo.

As used herein, the term “pharmaceutically acceptable carrier” refers to any of the standard pharmaceutical carriers, such as phosphate buffered saline solution, water, emulsions (e.g., such as oil/water or water/oil emulsions), and various types of wetting agents. The compositions also can include stabilizers and preservatives. For examples of carriers, stabilizers, and adjuvants, see e.g., Martin, Remington's Pharmaceutical Sciences, 15^{th} Ed. Mack Publ. Co., Easton, PA [1975].

As used herein, the term “effective amount” refers to an amount sufficient to achieve a beneficial or desired result. In certain non-limiting embodiments, an effective amount can be an amount that results in improved gastrointestinal engraftment (e.g., engraftment of one or more of the plurality of active microbes), increased biomass (e.g., of one or more of the plurality of active microbes), increased metabolism, or improved longitudinal stability.

As used herein, “significantly” or “significant” refers to a change or alteration in a measurable parameter to a statistically significant degree as determined in accordance with an appropriate statistically relevant test. For example, in certain non-limiting embodiments, a change or alteration is significant if it is statistically significant in accordance with, e.g., a Student's t-test, chi-square, or Mann Whitney test.

As used herein, the term “standardized substrate metabolization assay” refers to an experimental assay known to persons of ordinary skill in the art used to quantify the amount of substrate converted to a metabolic product.

As used herein, the term “subject” refers to an organism to be treated by the microbial consortium and compositions described herein. Such organisms preferably include, but are not limited to, mammals (e.g., murines, simians, equines, bovines, porcines, canines, felines, and the like), and more preferably include humans.

The term percent “identity” or “sequence identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

When used in reference to 16S rRNA sequences, a “sequence identity” of at least 97% indicates that two microbial strains are likely to belong to the same species, whereas 16S rRNA sequences having less than 97% sequence identity indicate that two microbial strains likely belong to different species, and 16S rRNA sequences having less than 95% sequence identity indicates that two microbial strains likely belong to distinct genera (Stackebrandt E., and Goebel, B. M., Int J Syst Bact, 44 (1994) 846-849.).

As used herein, the terms “functional equivalent” or “functionally equivalent” refers to microbes, microbial consortia, and compositions that share similar or identical role (e.g., metabolism of oxalate). For example, without any limitation, two different microbial consortia that can catalyze high concentration of oxalate are functional equivalent to each other. In certain non-limiting embodiments, a microbe, a microbial consortium, and a composition that is functional equivalent can be based on the characteristic outlined in Table 3 (see Example section).

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

As a general matter, compositions specifying a percentage are by weight unless otherwise specified. Further, if a variable is not accompanied by a definition, then the previous definition of the variable controls.

### Dysbiosis

Microbiomes are present in multiple species (for example, in mammalian subjects) and comprise bacteria, archaea, protists, fungi, and viruses. Traditionally, a microbiome (e.g., a human microbiome) includes several trillions of microbes (e.g., bacteria) from thousands of species and performs functions that can benefit the host organism (e.g., a human subject). For example, the species present in a microbiome benefit the host (e.g., a human subject) by performing useful or necessary functions, such as aiding in the digestion of food in the intestinal tract of the subject, protecting the body from penetration by pathogenic microbes, and promoting immunological development. In certain embodiments, organisms that perform these functions may be referred to as symbiotic or commensal organisms because they exist in the host (e.g., a human subject) without harming and, in some cases, actually benefit the host. In certain embodiments, in dysbiosis the physiological/normal microbiome of the host (e.g., a human subject) is perturbed or damaged, which may lead to a variety of diseases and/or disorders. In certain embodiments, dysbiosis may result, for example, from a loss of beneficial species, loss of microbial diversity, increase in pathogenic organism(s), and/or change in metabolic capacity. In certain embodiments, the species that normally dominate the microbiome become underrepresented (e.g., commensal or symbiotic species) and species which are normally underrepresented (e.g., opportunistic species) become overrepresented. See Petersen et al., “Defining dysbiosis and its influence on host immunity and disease.” Cell Microbiol 2014, July 16 (7), 1024-1033.

In certain embodiments, the compositions and methods described herein reduce dysbiosis. In certain embodiments, the compositions and methods provide for an increase in the abundance of bacterial species beneficial to the microbiome. In certain embodiments, the compositions and methods provide for a decrease in the abundance of pathogenic bacterial species. In certain embodiments, the compositions and methods described herein do not reduce all characteristics of dysbiosis. In certain embodiments, the compositions and methods provide for an increase in the abundance of bacterial species beneficial to the microbiome. In certain embodiments, the compositions and methods provide for an increase in the abundance of bacterial species beneficial to the microbiome but do not increase the diversity of the microbiome. In certain embodiments, the term “decreasing dysbiosis” refers to restoring the microbiota community composition and homeostasis. In certain embodiments, disruptions in the microbiome may allow pathogens within the microbiome or from other sources to colonize, overpopulate, and/or cause disease in the subject. In certain embodiments, dysbiosis is associated with many diseases and/or disorders, including inflammatory bowel disease (IBD), colitis, ulcerative colitis, and Crohn's disease.

In certain embodiments, dysbiosis may be detected and/or monitored by many of a variety of methods, such as stool tests (e.g., identification and/or quantification of microbial populations, enzyme assays, metabolite assays, immune function), and/or hydrogen/methane breath tests. Additional methods for detecting dysbiosis encompassed by the present disclosure can be found in Wei et al., Applied and Environmental Microbiology 87, no. 11 (2021): e00395-21, the content of which is incorporated by reference in its entirety.

In certain embodiments, decreasing dysbiosis involves a change (e.g., an increase or a decrease) in the abundance of one or more populations of bacteria. In certain embodiments, the abundance of bacteria, including the abundance of specific species or strains of bacteria and abundance of a population of bacteria (e.g., bacteria belonging to a particular phylum) can be assessed using any method known to one of skill in the art. In certain embodiments, the abundance of bacteria can be assessed directly or indirectly. In certain embodiments, methods for directly assessing the abundance of bacteria in a sample (e.g., a microbiome or sample thereof) include identifying and quantifying bacterial strains in a fecal sample from the subject. In certain embodiments, methods for indirectly assessing the abundance of bacteria in a sample (e.g., a microbiome or sample thereof) include sequencing of nucleic acid samples (e.g., 16S rRNA gene for a given bacterial species or other bacterial genes) obtained from a fecal sample or a biopsy sample, and detecting and quantifying metabolites associated with specific bacteria (e.g., phospholipid fatty acid metabolism, microbial biomass carbon analysis) in a fecal sample from the subject.

In certain embodiments, the abundance of one or more populations of bacteria in a sample from a subject may be compared to the abundance of the populations of bacteria in a sample from the same subject obtained at another time (e.g., obtained previously or subsequently). In certain embodiments, the abundance of one or more populations of bacteria in a sample from a subject may be compared to the abundance of the populations of bacteria in a sample from a different subject (e.g., a reference subject).

In certain embodiments, the dysbiosis is characterized by an increase in the abundance of microorganisms associated with inflammation and/or disease. In certain embodiments, the dysbiosis is characterized by an increase in the abundance of Proteobacteria. In certain embodiments, the increase in the abundance of microorganisms associated with inflammation and/or disease is relative to the abundance of microorganisms associated with inflammation prior to exposure to an event, referred to as a dysbiosis-inducing event.

In some embodiments, the dysbiosis of a subject is characterized by a decrease in the abundance of microorganism considered to provide one or more beneficial effects to the subject. In certain embodiments, the dysbiosis is characterized by a decrease in the abundance of bacteria of the phylum Bacteroidetes. In certain embodiments, the dysbiosis of the microbiota of a subject is characterized by a decrease in the abundance of bacteria of the phylum Firmicutes. In certain embodiments, the dysbiosis is characterized by a decrease in the abundance of bacteria belonging to Clostridium clusters IV and/or Clostridium clusters XIVa. In certain embodiments, the dysbiosis is characterized by a decrease in the abundance of bacteria belonging to Clostridium cluster XVII. In certain embodiments, the decrease in the abundance of beneficial microorganisms is relative to the abundance of microorganisms associated with inflammation prior to exposure to an event, referred to as a dysbiosis-inducing event, as described herein.

In certain embodiments, the dysbiosis is a dysbiosis of the gastrointestinal microbiota. In certain embodiments, the dysbiosis of the gastrointestinal microbiota is characterized by an increase in the abundance of microorganisms associated with inflammation and/or disease. In certain embodiments, the dysbiosis of the gastrointestinal microbiota is characterized by an increase in the abundance of Proteobacteria. In certain embodiments, the increase in the abundance of microorganisms associated with inflammation and/or disease is relative to the abundance of microorganisms associated with inflammation prior to exposure to an event (e.g., a dysbiosis-inducing event).

In certain embodiments, the dysbiosis of the gastrointestinal microbiota is characterized by a decrease in the abundance of microorganism considered to provide one or more beneficial effects to the subject. In certain embodiments, the dysbiosis of the gastrointestinal microbiota is characterized by a decrease in the abundance of bacteria of the phylum Bacteroidetes. In certain embodiments, the dysbiosis of the gastrointestinal microbiota is characterized by a decrease in the abundance of bacteria of the phylum Firmicutes. In certain embodiments, the dysbiosis of the gastrointestinal microbiota is characterized by a decrease in the abundance of bacteria belonging to Clostridium clusters IV and/or Clostridium clusters XIVa. In certain embodiments, the dysbiosis of the gastrointestinal microbiota is characterized by a decrease in the abundance of bacteria belonging to Clostridium cluster XVII. In certain embodiments, the decrease in the abundance of beneficial microorganisms is relative to the abundance of microorganisms associated with inflammation prior to exposure to an event (e.g., a dysbiosis-inducing event).

In certain embodiments, decreasing dysbiosis results in an increase in the abundance of bacteria of the phylum Bacteroidetes (e.g., bacteria of the genus Bacteroides) relative to the abundance of Bacteroides in the host (e.g., a human subject) (or microbiome thereof) prior to administering the pharmaceutical composition. In certain embodiments, decreasing dysbiosis results in an increase in the abundance of bacteria of the phylum Bacteroidetes (e.g., bacteria of the genus Bacteroides) relative to the abundance of Bacteroides in a reference host (e.g., a human subject, e.g., a reference subject) (or microbiome thereof) who did not receive the pharmaceutical composition. In certain embodiments, decreasing dysbiosis results in an increase in the abundance of one or more bacterial species belonging to the genus Bacteroides. In certain embodiments, decreasing dysbiosis results in an increase in the abundance overall of bacterial species belonging to the genus Bacteroides.

### Biological Niches

Disclosed herein are microbial consortia for administration to an animal (e.g., a human subject) comprising a plurality of active microbes which metabolize a first metabolic substrate. In certain embodiments, the first metabolic substrate causes or contributes to disease in the animal. The microbial consortia disclosed herein further comprise an effective amount of a supportive community of microbes that metabolize one or more metabolites produced by the plurality of active microbes, wherein the one or more metabolites inhibit metabolism of the plurality of active microbes. These microbial consortia are advantageous in having enhanced characteristics when administered to an animal as compared to administration of the plurality of active microbes alone. Enhanced characteristics of the microbial consortia include, for example and without any limitation, improved gastrointestinal engraftment, increased biomass, increased metabolism of the first metabolic substrate, and improved longitudinal stability.

The present disclosure provides microbial consortia capable of engrafting into one or more niches of a gastrointestinal tract. In certain embodiments, the engrafted microbial consortia are capable of metabolizing a substrate that causes or contributes to disease in an animal. These niches comprise specific microbial communities whose composition varies according to a number of environmental factors including, but not limited to, the particular physical compartment of the gastrointestinal tract inhabited by a microbial community, the chemical and physicochemical properties of the environment inhabited, the metabolic substrate composition of the environment inhabited, and other co-inhabiting microbial species.

### Consortia

The present disclosure provides Consortia comprising a plurality of active microbes and an effective amount of a supportive community of microbes.

In certain embodiments, the Consortia comprises a plurality of microbes (e.g., active microbes and supportive community microbes) designated as “Consortia I.” In certain embodiments, the Consortia I comprises Acidaminococcus intestini, Bacteroides stercoris, Blautia hydrogenotrophica, Coprococcus eutactus, Holdemanella biformis, Akkermansia muciniphila, Bacteroides stercoris, Blautia luti, Coprococcus eutactus, Holdemanella biformis, Alistipes finegoldii, Bacteroides thetaiotaomicron, Blautia luti, Desulfovibrio desulfuricans, Hungatella hathewayi, Alistipes onderdonkii, Bacteroides thetaiotaomicron, Blautia luti, Desulfovibrio desulfuricans, Hungatella hathewayi, Alistipes onderdonkii, Bacteroides thetaiotaomicron, Blautia obeum, Dialister invisus, Neglecta timonensis, Alistipes onderdonkii, Bacteroides uniformis, Blautia obeum, Dorea formicigenerans, Oxalobacter formigenes, Alistipes putredinis, Bacteroides uniformis, Blautia obeum, Dorea formicigenerans, Oxalobacter formigenes, Alistipes putredinis, Bacteroides uniformis, Blautia obeum, Dorea formicigenerans, Oxalobacter formigenes, Alistipes senegalensis, Bacteroides uniformis, Blautia wexlerae, Dorea longicatena, Parabacteroides distasonis, Alistipes senegalensis, Bacteroides vulgatus, Blautia wexlerae, Dorea longicatena, Parabacteroides distasonis, Alistipes shahii, Bacteroides vulgatus, Blautia wexlerae, Dorea longicatena, Parabacteroides distasonis, Alistipes shahii, Bacteroides vulgatus, Clostridium aldenense, Eggerthella lenta, Parabacteroides merdae, Alistipes shahii, Bacteroides vulgatus, Clostridium aldenense, Eggerthella lenta, Parabacteroides merdae, Alistipes timonensis, Bacteroides xylanisolvens, Clostridium amygdalinum, Eggerthella lenta, Parabacteroides merdae, Anaerofustis stercorihominis, Bacteroides xylanisolvens, Clostridium bolteae, Eggerthella lenta, Paraprevotella clara, Anaerostipes hadrus, Bacteroides xylanisolvens, Clostridium bolteae, Eubacterium eligens, Parasutterella excrementihominis, Anaerostipes hadrus, Bifidobacterium adolescentis, Clostridium citroniae, Eubacterium eligens, Parasutterella excrementihominis, Anaerotruncus colihominis, Bifidobacterium adolescentis, Clostridium citroniae, Eubacterium eligens, Roseburia hominis, Bacteroides caccae, Bifidobacterium catenulatum, Clostridium scindens, Eubacterium eligens, Roseburia hominis, Bacteroides caccae, Bifidobacterium dentium, Clostridium symbiosum, Eubacterium hallii, Roseburia hominis, Bacteroides cellulosilyticus, Bifidobacterium longum, Clostridium symbiosum, Eubacterium rectale, Roseburia hominis, Bacteroides coprocola, Bifidobacterium longum, Clostridium symbiosum, Eubacterium rectale, Ruminococcus bromii, Bacteroides finegoldii, Bifidobacterium longum, Collinsella aerofaciens, Eubacterium rectale, Ruminococcus bromii, Bacteroides fragilis, Bifidobacterium longum, Collinsella aerofaciens, Eubacterium rectale, Ruminococcus bromii, Bacteroides massiliensis, Bifidobacterium pseudocatenulatum, Collinsella aerofaciens, Eubacterium siraeum, Ruminococcus bromii, Bacteroides massiliensis, Bifidobacterium pseudocatenulatum, Collinsella aerofaciens, Eubacterium ventriosum, Ruminococcus faecis, Bacteroides nordii, Bifidobacterium pseudocatenulatum, Coprococcus comes, Eubacterium xylanophilum, Ruminococcus faecis, Bacteroides oleiciplenus, Blautia faecis, Coprococcus comes, Faecalibacterium prausnitzii, Turicibacter sanguinis, Bacteroides ovatus, Blautia faecis, Coprococcus comes, Faecalibacterium prausnitzii, Bacteroides salyersiae, Blautia faecis, Coprococcus comes, Gordonibacter pamelaeae, Bacteroides stercoris, Blautia faecis, Coprococcus eutactus, and Gordonibacter pamelaeae.

In certain embodiments, the Consortia comprises a plurality of microbes (e.g., active microbes and supportive community microbes) designated as “Consortia II.” In certain embodiments, the Consortia II comprises Akkermansia muciniphila, Bacteroides vulgatus, Clostridium amygdalinum, Eggerthella lenta, Oxalobacter formigenes, Alistipes onderdonkii, Bifidobacterium dentium, Clostridium citroniae, Eubacterium eligens, Parabacteroides distasonis, Alistipes putredinis, Bifidobacterium faecale, Clostridium citroniae, Eubacterium eligens, Parabacteroides distasonis, Alistipes shahii, Bifidobacterium longum, Clostridium scindens, Eubacterium rectale, Parabacteroides merdae, Alistipes timonensis, Bifidobacterium longum, Clostridium symbiosum, Eubacterium rectale, Paraprevotella clara, Bacteroides caccae, Bifidobacterium pseudocatenulatum, Collinsella aerofaciens, Faecalibacterium prausnitzii, Parasutterella excrementihominis, Bacteroides koreensis, Bifidobacterium pseudocatenulatum, Coprococcus comes, Fusicatenibacter saccharivorans, Phascolarctobacterium faecium, Bacteroides kribbi, Bifidobacterium pseudocatenulatum, Coprococcus eutactus, Fusicatenibacter saccharivorans, Phascolarctobacterium faecium, Bacteroides kribbi, Blautia faecis, Desulfovibrio desulfuricans, Gordonibacter pamelaeae, Phascolarctobacterium faecium, Bacteroides nordii, Blautia faecis, Dialister succinatiphilus, Lachnoclostridium pacaense, Roseburia hominis, Bacteroides ovatus, Blautia obeum, Dorea formicigenerans, Lachnospira pectinoschiza, Ruminococcus bromii, Bacteroides salyersiae, Blautia obeum, Dorea longicatena, Monoglobus pectinilyticus, Ruminococcus bromii, Bacteroides thetaiotaomicron, Blautia obeum, Eggerthella lenta, Neglecta timonensis, Ruminococcus faecis, Bacteroides thetaiotaomicron, Blautia wexlerae, Eggerthella lenta, Oxalobacter formigenes, Sutterella massiliensis, Bacteroides uniformis, Blautia wexlerae, Eggerthella lenta, Oxalobacter formigenes, and Sutterella wadsworthensis.

In certain embodiments, the Consortia comprises a plurality of microbes (e.g., active microbes and supportive community microbes) designated as “Consortia III.” In certain embodiments, the Consortia III comprises Akkermansia muciniphila, Bacteroides vulgatus, Clostridium scindens, Eubacterium rectale, Parabacteroides merdae, Anaerotruncus colihominis, Bacteroides vulgatus, Clostridium symbiosum, Eubacterium rectale, Ruminococcus bromii, Bacteroides caccae, Bacteroides vulgatus, Clostridium symbiosum, Eubacterium rectale, Ruminococcus bromii, Bacteroides caccae, Bifidobacterium adolescentis, Clostridium symbiosum, Eubacterium rectale, Ruminococcus bromii, Bacteroides cellulosilyticus, Bifidobacterium adolescentis, Collinsella aerofaciens, Eubacterium siraeum, Ruminococcus bromii, Bacteroides fragilis, Bifidobacterium bifidum, Collinsella aerofaciens, Faecalibacterium prausnitzii, Sutterella wadsworthensis, Bacteroides massiliensis, Bifidobacterium bifidum, Collinsella aerofaciens, Gordonibacter pamelaeae, Sutterella wadsworthensis, Bacteroides massiliensis, Bifidobacterium catenulatum, Collinsella aerofaciens, Gordonibacter pamelaeae, Sutterella wadsworthensis, Bacteroides salyersiae, Bifidobacterium dentium, Coprococcus comes, Hydrogenoanaerobacterium saccharovorans, Bacteroides vulgatus, Bacteroides stercoris, Bifidobacterium longum, Coprococcus comes, Lachnospiraceae sp., Clostridium citroniae, Bacteroides stercoris, Bifidobacterium longum, Coprococcus comes, Lactonifactor longoviformis, Eggerthella lenta, Bacteroides stercoris, Bifidobacterium longum, Desulfovibrio desulfuricans, Neglecta timonensis, Parabacteroides merdae, Bacteroides thetaiotaomicron, Bifidobacterium longum, Desulfovibrio desulfuricans, Oxalobacter formigenes, Parabacteroides merdae, Bacteroides thetaiotaomicron, Bifidobacterium pseudocatenulatum, Dorea longicatena, Oxalobacter formigenes, Eggerthella lenta, Bacteroides thetaiotaomicron, Bifidobacterium pseudocatenulatum, Dorea longicatena, Oxalobacter formigenes, Clostridium citroniae, Bacteroides uniformis, Bifidobacterium pseudocatenulatum, Dorea longicatena, Parabacteroides distasonis, Bacteroides uniformis, Bacteroides uniformis, Citrobacter freundii, Eggerthella lenta, Parabacteroides distasonis, Bacteroides uniformis, Clostridium amygdalinum, Eggerthella lenta, and Parabacteroides distasonis.

In certain embodiments, the Consortia comprises a plurality of microbes (e.g., active microbes and supportive community microbes) designated as “Consortia IV.” In certain embodiments, the Consortia IV comprises Alistipes finegoldii, Bacteroides vulgatus, Clostridium bolteae, Dorea longicatena, Oxalobacter formigenes, Alistipes putredinis, Bacteroides vulgatus, Clostridium bolteae, Eggerthella lenta, Parabacteroides merdae Alistipes putredinis, Bacteroides xylanisolvens, Clostridium citroniae, Eggerthella lenta, Parabacteroides merdae, Anaerotruncus colihominis, Bacteroides xylanisolvens, Clostridium citroniae, Eggerthella lenta, Parabacteroides merdae, Bacteroides caccae, Bacteroides xylanisolvens, Clostridium scindens, Eggerthella lenta, Ruminococcus bromii, Bacteroides cellulosilyticus, Bifidobacterium bifidum, Clostridium symbiosum, Eubacterium eligens, Ruminococcus bromii, Bacteroides coprocola, Bifidobacterium bifidum, Clostridium symbiosum, Eubacterium eligens, Ruminococcus bromii, Bacteroides fragilis, Bifidobacterium catenulatum, Clostridium symbiosum, Eubacterium eligens, Ruminococcus bromii, Bacteroides ovatus, Bifidobacterium dentium, Collinsella aerofaciens, Eubacterium eligens, Sutterella wadsworthensis, Bacteroides salyersiae, Bifidobacterium longum, Collinsella aerofaciens, Eubacterium hallii, Bacteroides stercoris, Bifidobacterium longum, Collinsella aerofaciens, Eubacterium rectale, Bacteroides stercoris, Bifidobacterium longum, Collinsella aerofaciens, Eubacterium siraeum, Bacteroides stercoris, Bifidobacterium longum, Coprococcus comes, Eubacterium ventriosum, Bacteroides thetaiotaomicron, Bifidobacterium pseudocatenulatum, Coprococcus eutactus, Faecalibacterium prausnitzii, Bacteroides thetaiotaomicron, Bifidobacterium pseudocatenulatum, Coprococcus eutactus, Faecalibacterium prausnitzii, Bacteroides thetaiotaomicron, Bifidobacterium pseudocatenulatum, Coprococcus eutactus, Hungatella hathewayi, Bacteroides uniformis, Blautia hydrogenotrophica, Desulfovibrio desulfuricans, Hungatella hathewayi, Bacteroides uniformis, Blautia obeum, Desulfovibrio desulfuricans, Neglecta timonensis, Bacteroides vulgatus, Blautia obeum, Dorea formicigenerans, Oxalobacter formigenes, Bacteroides vulgatus, Clostridium amygdalinum, Dorea longicatena, and Oxalobacter formigenes.

In certain embodiments, the Consortia comprises a plurality of microbes (e.g., active microbes and supportive community microbes) designated as “Consortia V.” In certain embodiments, the Consortia V comprises Acidaminococcus intestini, Bacteroides uniformis, Clostridium citroniae, Eubacterium ventriosum, Phascolarctobacterium faecium, Akkermansia muciniphila, Bacteroides vulgatus, Clostridium clostridioforme, Eubacterium siraeum, Phascolarctobacterium faecium, Alistipes finegoldii, Bacteroides vulgatus, Clostridium scindens, Eubacterium xylanophilum, Phocea massiliensis, Alistipes onderdonkii, Bacteroides xylanisolvens, Clostridium swellfunianum, Faecalibacterium prausnitzii, Phocea massiliensis, Alistipes onderdonkii, Bacteroides xylanisolvens, Clostridium symbiosum, Faecalibacterium prausnitzii, Porphyromonas asaccharolytica, Alistipes putredinis, Barnesiella intestinihominis, Clostridium symbiosum, Faecalicatena contorta, Porphyromonas asaccharolytica, Alistipes putredinis, Bifidobacterium adolescentis, Collinsella aerofaciens, Fusicatenibacter saccharivorans, Roseburia hominis, Alistipes senegalensis, Bifidobacterium adolescentis, Collinsella aerofaciens, Fusicatenibacter saccharivorans, Roseburia hominis, Alistipes senegalensis, Bifidobacterium bifidum, Coprococcus comes, Gordonibacter pamelaeae, Ruminococcus bromii, Alistipes shahii, Bifidobacterium bifidum, Coprococcus comes, Gordonibacter pamelaeae, Ruminococcus bromii, Alistipes timonensis, Bifidobacterium catenulatum, Coprococcus eutactus, Holdemanella biformis, Ruminococcus faecis, Anaerofustis stercorihominis, Bifidobacterium dentium, Coprococcus eutactus, Holdemanella biformis, Ruminococcus faecis, Anaerostipes hadrus, Bifidobacterium faecale, Desulfovibrio desulfuricans, Hungatella effluvii, Ruthenibacterium lactatiformans, Anaerostipes hadrus, Bifidobacterium longum, Desulfovibrio desulfuricans, Hungatella hathewayi, Senegalimassilia anaerobia, Anaerotruncus colihominis, Bifidobacterium longum, Dialister invisus, Hungatella hathewayi, Sutterella massiliensis, Bacteroides caccae, Bifidobacterium pseudocatenulatum, Dialister succinatiphilus, Hydrogenoanaerobacterium saccharovorans, Sutterella wadsworthensis, Bacteroides caccae, Bifidobacterium pseudocatenulatum, Dielma fastidiosa, Lachnoclostridium pacaense, Sutterella wadsworthensis, Bacteroides coprocola, Bifidobacterium pseudocatenulatum, Dorea formicigenerans, Lachnoclostridium pacaense, Turicibacter sanguinis, Bacteroides faecis, Blautia faecis, Dorea formicigenerans, Lachnospira pectinoschiza, Bacteroides finegoldii, Blautia faecis, Dorea longicatena, Lachnospira pectinoschiza, Bacteroides fragilis, Blautia hydrogenotrophica, Dorea longicatena, Lactonifactor longoviformis, Bacteroides koreensis, Blautia luti, Eggerthella lenta, Longicatena caecimuris, Bacteroides koreensis, Blautia obeum, Eggerthella lenta, Megasphaera massiliensis, Bacteroides kribbi, Blautia obeum, Eggerthella lenta, Monoglobus pectinilyticus, Bacteroides kribbi, Blautia wexlerae, Eggerthella lenta, Monoglobus pectinilyticus, Bacteroides massiliensis, Blautia wexlerae, Eisenbergiella tayi, Neglecta timonensis, Bacteroides nordii, Butyricimonas faecihominis, Eisenbergiella tayi, Oxalobacter formigenes, Bacteroides oleiciplenus, Catabacter hongkongensis, Emergencia timonensis, Oxalobacter formigenes, Bacteroides ovatus, Citrobacter freundii, Eubacterium eligens, Oxalobacter formigenes, Bacteroides salyersiae, Clostridium aldenense, Eubacterium eligens, Parabacteroides distasonis, Bacteroides stercoris, Clostridium aldenense, Eubacterium hallii, Parabacteroides merdae, Bacteroides stercoris, Clostridium amygdalinum, Eubacterium oxidoreducens, Parabacteroides merdae, Bacteroides thetaiotaomicron, Clostridium bolteae, Eubacterium rectale, Paraprevotella clara, Bacteroides thetaiotaomicron, Clostridium bolteae, Eubacterium rectale, Parasutterella excrementihominis, Bacteroides uniformis, Clostridium citroniae, Eubacterium ruminantium, and Parasutterella excrementihominis.

In certain embodiments, the Consortia comprises a plurality of microbes (e.g., active microbes and supportive community microbes) designated as “Consortia VI.” In certain embodiments, the Consortia VI comprises Acidaminococcus intestini, Bacteroides stercorirosoris, Butyricimonas sp. FBI00158, Enterococcus casseliflavus, Longicatena caecimuri, Acidaminococcus intestini, Bacteroides stercoris, Catabacter hongkongensis, Enterococcus casseliflavus, Megasphaera massiliensis, Acutalibacter timonensis, Bacteroides stercoris, Citrobacter portucalensis, Enterococcus durans, Methanobrevibacter smithii, Akkermansia muciniphila, Bacteroides thetaiotaomicron, Clostridiaceae sp. FBI00191, Enterococcus durans, Monoglobus pectinilyticus, Alistipes onderdonkii, Bacteroides thetaiotaomicron, Clostridium aldenense, Enterococcus durans, Monoglobus pectinilyticus, Alistipes onderdonkii, Bacteroides uniformis, Clostridium aldenense, Enterococcus faecalis, Oxalobacter formigenes, Alistipes putredinis, Bacteroides uniformis, Clostridium bolteae, Enterococcus faecium, Oxalobacter formigenes, Alistipes putredinis, Bacteroides vulgatus, Clostridium bolteae, Escherichia flexneri, Oxalobacter formigenes, Alistipes senegalensis, Bacteroides vulgatus, Clostridium citroniae, Eubacterium eligens, Parabacteroides distasonis, Alistipes shahii, Bacteroides xylanisolvens, Clostridium citroniae, Eubacterium eligens, Parabacteroides distasonis, Alistipes shahii, Bacteroides xylanisolvens, Clostridium clostridioforme, Eubacterium hallii, Parabacteroides merdae, Alistipes sp. FBI00180, Bacteroides xylanisolvens, Clostridium fessum, Eubacterium rectale, Parabacteroides merdae, Alistipes sp. FBI00238, Barnesiella intestinihominis, Clostridium fessum, Eubacterium rectale, Paraprevotella clara, Alistipes timonensis, Bifidobacterium adolescentis, Clostridium scindens, Eubacterium siraeum, Parasutterella excrementihominis, Anaerofustis stercorihominis, Bifidobacterium adolescentis, Collinsella aerofaciens, Eubacterium ventriosum, Parasutterella excrementihominis, Anaerostipes hadrus, Bifidobacterium adolescentis, Collinsella aerofaciens, Eubacterium xylanophilum, Phascolarctobacterium faecium, Anaerostipes hadrus, Bifidobacterium adolescentis, Coprococcus comes, Faecalibacterium prausnitzii, Phascolarctobacterium faecium, Anaerotruncus massiliensis, Bifidobacterium bifidum, Coprococcus comes, Faecalibacterium prausnitzii, Porphyromonas asaccharolytica, Bacteroides caccae, Bifidobacterium bifidum, Coprococcus eutactus, Faecalicatena contorta, Porphyromonas asaccharolytica, Bacteroides caccae, Bifidobacterium catenulatum, Dialister invisus, Fusicatenibacter saccharivorans, Roseburia hominis, Bacteroides cellulosilyticus, Bifidobacterium dentium, Coprococcus eutactus, Fusicatenibacter saccharivorans, Roseburia hominis, Bacteroides cellulosilyticus, Bifidobacterium longum, Dialister succinatiphilus, Gordonibacter pamelaeae, Ruminococcaceae sp. FBI00097, Bacteroides coprocola, Bifidobacterium longum, Dialister succinatiphilus, Gordonibacter pamelaeae, Ruminococcaceae sp. FBI00097, Bacteroides dorei, Bifidobacterium pseudocatenulatum, Dielma fastidiosa, Holdemanella biformis, Ruminococcaceae sp. FBI00233, Bacteroides dorei, Bifidobacterium pseudocatenulatum, Dorea formicigenerans, Holdemanella biformis, Ruminococcus bromii, Bacteroides faecis, Bilophila wadsworthia, Dorea formicigenerans, Hungatella effluvii, Ruminococcus bromii, Bacteroides finegoldii, Bilophila wadsworthia, Dorea longicatena, Hungatella effluvii, Ruminococcus faecis, Bacteroides fragilis, Blautia faecis, Dorea longicatena, Hungatella effluvii, Ruminococcus faecis, Bacteroides kribbi/Bacteroides koreensis species cluster, Blautia faecis, Eggerthella lenta, Lachnoclostridium pacaense, Ruthenibacterium lactatiformans, Bacteroides kribbi/Bacteroides koreensis species cluster, Blautia hydrogenotrophica, Eggerthella lenta, Lachnoclostridium pacaense, Senegalimassilia anaerobia, Bacteroides kribbi/Bacteroides koreensis species cluster, Blautia luti, Eisenbergiella tayi, Lachnospiraceae sp. FBI00033, Sutterella massiliensis, Bacteroides massiliensis, Blautia massiliensis, Emergencia timonensis, Lachnospiraceae sp. FBI00071, Sutterella wadsworthensis, Bacteroides massiliensis, Blautia obeum, Eisenbergiella tayi, Lachnospiraceae sp. FBI00150, Sutterella wadsworthensis, Bacteroides nordii, Blautia obeum, Enterobacter himalayensis, Lachnospiraceae sp. FBI00290, Turicibacter sanguinis, Bacteroides ovatus, Blautia wexlerae, Enterobacter hormaechei, Lactobacillus rogosae, Bacteroides salyersiae, Blautia wexlerae, Enterococcus casseliflavus, Lactobacillus rogosae, Bacteroides salyersiae, Butyricimonas faecihominis, Enterococcus casseliflavus, and Lactonifactor longoviformis.

In certain embodiments, the Consortia comprises a plurality of microbes (e.g., active microbes and supportive community microbes) designated as “Consortia VII.” In certain embodiments, the Consortia VII comprises Acidaminococcus intestini, Bacteroides thetaiotaomicron, Citrobacter portucalensis, Eubacterium eligens, Oxalobacter formigenes, Acutalibacter timonensis, Bacteroides uniformis, Clostridiaceae sp. FBI00191, Eubacterium hallii, Parabacteroides distasonis, Akkermansia muciniphila, Bacteroides uniformis, Clostridium aldenense, Eubacterium rectale, Parabacteroides merdae, Alistipes onderdonkii, Bacteroides vulgatus, Clostridium aldenense, Eubacterium rectale, Parabacteroides merdae, Alistipes onderdonkii, Bacteroides vulgatus, Clostridium bolteae, Eubacterium siraeum, Paraprevotella clara, Alistipes putredinis, Bacteroides xylanisolvens, Clostridium bolteae, Eubacterium ventriosum, Parasutterella excrementihominis, Alistipes putredinis, Bacteroides xylanisolvens, Clostridium citroniae, Faecalibacterium prausnitzii, Parasutterella excrementihominis, Alistipes senegalensis, Bacteroides xylanisolvens, Clostridium citroniae, Eubacterium xylanophilum, Phascolarctobacterium faecium, Alistipes shahii, Barnesiella intestinihominis, Clostridium clostridioforme, Faecalibacterium prausnitzii, Phascolarctobacterium faecium, Alistipes sp. FBI00180, Bifidobacterium adolescentis, Clostridium fessum, Faecalicatena contorta, Porphyromonas asaccharolytica, Alistipes sp. FBI00238, Bifidobacterium adolescentis, Clostridium fessum, Fusicatenibacter saccharivorans, Porphyromonas asaccharolytica, Alistipes timonensis, Bifidobacterium adolescentis, Clostridium scindens, Fusicatenibacter saccharivorans, Roseburia hominis, Anaerofustis stercorihominis, Bifidobacterium bifidum, Collinsella aerofaciens, Gordonibacter pamelaeae, Roseburia hominis, Anaerostipes hadrus, Bifidobacterium bifidum, Collinsella aerofaciens, Gordonibacter pamelaeae, Ruminococcaceae sp. FBI00097, Anaerostipes hadrus, Bifidobacterium catenulatum, Coprococcus comes, Holdemanella biformis, Ruminococcaceae sp. FBI00097, Anaerotruncus massiliensis, Bifidobacterium dentium, Coprococcus comes, Holdemanella biformis, Ruminococcaceae sp. FBI00233, Bacteroides caccae, Bifidobacterium longum, Coprococcus eutactus, Hungatella effluvii, Ruminococcus bromii, Bacteroides caccae, Bifidobacterium longum, Coprococcus eutactus, Hungatella effluvii, Ruminococcus bromii, Bacteroides coprocola, Bifidobacterium pseudocatenulatum, Dialister invisus, Hungatella effluvii, Ruminococcus faecis, Bacteroides faecis, Bifidobacterium pseudocatenulatum, Dialister succinatiphilus, Lachnoclostridium pacaense, Ruminococcus faecis, Bacteroides finegoldii, Bifidobacterium pseudocatenulatum, Dielma fastidiosa, Lachnoclostridium pacaense, Ruthenibacterium lactatiformans, Bacteroides fragilis, Bilophila wadsworthia, Dorea formicigenerans, Lachnospiraceae sp. FBI00033, Senegalimassilia anaerobia, Bacteroides kribbi/Bacteroides koreensis species cluster, Bilophila wadsworthia, Dorea formicigenerans, Lachnospiraceae sp. FBI00071, Sutterella massiliensis, Bacteroides kribbi/Bacteroides koreensis species cluster, Blautia faecis, Dorea longicatena, Lachnospiraceae sp. FBI00290, Sutterella wadsworthensis, Bacteroides kribbi/Bacteroides koreensis species cluster, Blautia faecis, Dorea longicatena, Lactobacillus rogosae, Sutterella wadsworthensis, Bacteroides massiliensis, Blautia hydrogenotrophica, Eggerthella lenta, Lactobacillus rogosae, Turicibacter sanguinis, Bacteroides nordii, Blautia massiliensis, Eggerthella lenta, Lactonifactor longoviformis, Bacteroides ovatus, Blautia obeum, Eggerthella lenta, Longicatena caecimuris, Bacteroides salyersiae, Blautia obeum, Eggerthella lenta, Megasphaera massiliensis, Bacteroides stercorirosoris, Blautia wexlerae, Eisenbergiella tayi, Monoglobus pectinilyticus, Bacteroides stercoris, Blautia wexlerae, Eisenbergiella tayi, Monoglobus pectinilyticus, Bacteroides stercoris, Butyricimonas faecihominis, Emergencia timonensis, Oxalobacter formigenes, Bacteroides thetaiotaomicron, Catabacter hongkongensis, Eubacterium eligens, and Oxalobacter formigenes.

In certain embodiments, the Consortia comprises a plurality of microbes (e.g., active microbes and supportive community microbes) designated as “Consortia VIII.” In certain embodiments, the Consortia VIII comprises Acidaminococcus intestini, Bacteroides thetaiotaomicron, Butyricimonas faecihominis, Eisenbergiella tayi, Monoglobus pectinilyticus, Acutalibacter timonensis, Bacteroides thetaiotaomicron, Catabacter hongkongensis, Emergencia timonensis, Monoglobus pectinilyticus, Akkermansia muciniphila, Bacteroides uniformis, Citrobacter portucalensis, Eubacterium eligens, Oxalobacter formigenes, Alistipes onderdonkii, Bacteroides uniformis, Clostridiaceae sp. FBI00191, Eubacterium eligens, Oxalobacter formigenes, Alistipes onderdonkii, Bacteroides vulgatus, Clostridium aldenense, Eubacterium hallii, Oxalobacter formigenes, Alistipes putredinis, Bacteroides vulgatus, Clostridium aldenense, Eubacterium rectale, Parabacteroides distasonis, Alistipes putredinis, Bacteroides xylanisolvens, Clostridium bolteae, Eubacterium rectale, Parabacteroides merdae, Alistipes senegalensis, Bacteroides xylanisolvens, Clostridium bolteae, Eubacterium siraeum, Parabacteroides merdae, Alistipes shahii, Bacteroides xylanisolvens, Clostridium citroniae, Eubacterium ventriosum, Paraprevotella clara, Alistipes sp. FBI00180, Barnesiella intestinihominis, Clostridium citroniae, Faecalibacterium prausnitzii, Parasutterella excrementihominis, Alistipes sp. FBI00238, Bifidobacterium adolescentis, Clostridium clostridioforme, Eubacterium xylanophilum, Parasutterella excrementihominis, Alistipes timonensis, Bifidobacterium adolescentis, Clostridium fessum, Faecalibacterium prausnitzii, Phascolarctobacterium faecium, Anaerofustis stercorihominis, Bifidobacterium adolescentis, Clostridium fessum, Faecalicatena contorta, Phascolarctobacterium faecium, Anaerostipes hadrus, Bifidobacterium bifidum, Clostridium scindens, Fusicatenibacter saccharivorans, Porphyromonas asaccharolytica, Anaerostipes hadrus, Bifidobacterium bifidum, Collinsella aerofaciens, Fusicatenibacter saccharivorans, Porphyromonas asaccharolytica, Anaerotruncus massiliensis, Bifidobacterium catenulatum, Collinsella aerofaciens, Gordonibacter pamelaeae, Roseburia hominis, Bacteroides caccae, Bifidobacterium dentium, Coprococcus comes, Gordonibacter pamelaeae, Roseburia hominis, Bacteroides caccae, Bifidobacterium longum, Coprococcus comes, Holdemanella biformis, Ruminococcaceae sp. FBI00097, Bacteroides coprocola, Bifidobacterium longum, Coprococcus eutactus, Holdemanella biformis, Ruminococcaceae sp. FBI00097, Bacteroides faecis, Bifidobacterium pseudocatenulatum, Coprococcus eutactus, Hungatella effluvii, Ruminococcaceae sp. FBI00233, Bacteroides finegoldii, Bifidobacterium pseudocatenulatum, Dialister invisus, Hungatella effluvii, Ruminococcus bromii, Bacteroides fragilis, Bifidobacterium pseudocatenulatum, Dialister succinatiphilus, Hungatella effluvii, Ruminococcus bromii, Bacteroides kribbi/Bacteroides koreensis species cluster, Bilophila wadsworthia, Dielma fastidiosa, Lachnoclostridium pacaense, Ruminococcus faecis, Bacteroides kribbi/Bacteroides koreensis species cluster, Bilophila wadsworthia, Dorea formicigenerans, Lachnoclostridium pacaense, Ruminococcus faecis, Bacteroides kribbi/Bacteroides koreensis species cluster, Blautia faecis, Dorea formicigenerans, Lachnospiraceae sp. FBI00033, Ruthenibacterium lactatiformans, Bacteroides massiliensis, Blautia faecis, Dorea longicatena, Lachnospiraceae sp. FBI00071, Senegalimassilia anaerobia, Bacteroides nordii, Blautia hydrogenotrophica, Dorea longicatena, Lachnospiraceae sp. FBI00290, Sutterella massiliensis, Bacteroides ovatus, Blautia massiliensis, Eggerthella lenta, Lactobacillus rogosae, Sutterella wadsworthensis, Bacteroides salyersiae, Blautia obeum, Eggerthella lenta, Lactobacillus rogosae, Sutterella wadsworthensis, Bacteroides stercorirosoris, Blautia obeum, Eggerthella lenta, Lactonifactor longoviformis, Turicibacter sanguinis, Bacteroides stercoris, Blautia wexlerae, Eggerthella lenta, Longicatena caecimuris, Bacteroides stercoris, Blautia wexlerae, Eisenbergiella tayi, and Megasphaera massiliensis.

In certain embodiments, the Consortia comprises a plurality of microbes (e.g., active microbes and supportive community microbes) designated as “Consortia IX.” In certain embodiments, the Consortia XI comprises Acidaminococcus intestini, Bacteroides thetaiotaomicron, Butyricimonas faecihominis, Eubacterium eligens, Neglecta timonensis, Akkermansia muciniphila, Bacteroides thetaiotaomicron, Catabacter hongkongensis, Eubacterium eligens, Oxalobacter formigenes, Alistipes onderdonkii, Bacteroides uniformis, Clostridiaceae sp. FBI00191, Eubacterium hallii, Oxalobacter formigenes, Alistipes onderdonkii, Bacteroides uniformis, Clostridiales sp. FBI00377, Eubacterium rectale, Oxalobacter formigenes, Alistipes putredinis, Bacteroides vulgatus, Clostridium aldenense, Eubacterium rectale, Parabacteroides distasonis, Alistipes putredinis, Bacteroides vulgatus, Clostridium aldenense, Eubacterium siraeum, Parabacteroides distasonis, Alistipes senegalensis, Bacteroides xylanisolvens, Clostridium bolteae, Eubacterium ventriosum, Parabacteroides merdae, Alistipes shahii, Bacteroides xylanisolvens, Clostridium bolteae, Eubacterium xylanophilum, Parabacteroides merdae, Alistipes shahii, Bacteroides xylanisolvens, Clostridium citroniae, Faecalibacterium prausnitzii, Paraprevotella clara, Alistipes sp. FBI00180, Barnesiella intestinihominis, Clostridium citroniae, Fusicatenibacter saccharivorans, Parasutterella excrementihominis, Alistipes sp. FBI00238, Bifidobacterium adolescentis, Clostridium clostridioforme, Fusicatenibacter saccharivorans, Parasutterella excrementihominis, Alistipes timonensis, Bifidobacterium adolescentis, Clostridium fessum, Gordonibacter pamelaeae, Phascolarctobacterium faecium, Anaerofustis stercorihominis, Bifidobacterium adolescentis, Clostridium scindens, Gordonibacter pamelaeae, Porphyromonas asaccharolytica, Anaerostipes hadrus, Bifidobacterium bifidum, Collinsella aerofaciens, Holdemanella biformis, Porphyromonas asaccharolytica, Anaerostipes hadrus, Bifidobacterium catenulatum, Collinsella aerofaciens, Hungatella effluvii, Roseburia hominis, Anaerotruncus massiliensis, Bifidobacterium dentium, Coprococcus comes, Hungatella effluvii, Roseburia hominis, Bacteroides caccae, Bifidobacterium longum, Coprococcus comes, Hungatella effluvii, Ruminococcaceae sp. FBI00082, FBI00097, Bacteroides caccae, Bifidobacterium longum, Coprococcus eutactus, Lachnoclostridium pacaense, Ruminococcaceae sp. FBI00233, Bacteroides coprocola, Bifidobacterium pseudocatenulatum, Dialister invisus, Lachnoclostridium pacaense, Ruminococcus bromii, Bacteroides faecis, Bifidobacterium pseudocatenulatum, Dialister succinatiphilus, Lachnospiraceae sp. FBI00033, Ruminococcus bromii, Bacteroides finegoldii, Bifidobacterium pseudocatenulatum, Dielma fastidiosa, Lachnospiraceae sp. FBI00071, Ruminococcus faecis, Bacteroides fragilis, Bilophila wadsworthia, Dorea formicigenerans, Lachnospiraceae sp. FBI00290, Ruminococcus faecis, Bacteroides kribbi/Bacteroides koreensis species cluster, Bilophila wadsworthia, Dorea formicigenerans, Lactobacillus rogosae, Ruthenibacterium lactatiformans, Bacteroides kribbi/Bacteroides koreensis species cluster, Blautia faecis, Dorea longicatena, Lactobacillus rogosae, Senegalimassilia anaerobia, Bacteroides massiliensis, Blautia faecis, Dorea longicatena, Longicatena caecimuris, Sutterella massiliensis, Bacteroides nordii, Blautia hydrogenotrophica, Eggerthella lenta, Megasphaera massiliensis, Sutterella wadsworthensis, Bacteroides ovatus, Blautia massiliensis, Eggerthella lenta, Methanobrevibacter smithii, Sutterella wadsworthensis, Bacteroides salyersiae, Blautia obeum, Eggerthella lenta, Methanobrevibacter smithii, Turicibacter sanguinis, Bacteroides stercorirosoris, Blautia obeum, Eggerthella lenta, Monoglobus pectinilyticus, Bacteroides stercoris, Blautia wexlerae, Eisenbergiella tayi, Monoglobus pectinilyticus, Bacteroides stercoris, Blautia wexlerae, Eisenbergiella tayi, and Neglecta timonensis.

In certain embodiments, the Consortia comprises a plurality of microbes (e.g., active microbes and supportive community microbes) designated as “Consortia X.” In certain embodiments, the Consortia X comprises Acidaminococcus intestini, Bacteroides thetaiotaomicron, Butyricimonas faecihominis, Eubacterium eligens, Monoglobus pectinilyticus, Akkermansia muciniphila, Bacteroides thetaiotaomicron, Catabacter hongkongensis, Eubacterium eligens, Neglecta timonensis, Alistipes onderdonkii, Bacteroides uniformis, Clostridiaceae sp. FBI00191, Eubacterium hallii, Neglecta timonensis, Alistipes onderdonkii, Bacteroides uniformis, Clostridiales sp. FBI00377, Eubacterium rectale, Oxalobacter formigenes, Alistipes putredinis, Bacteroides vulgatus, Clostridium aldenense, Eubacterium rectale, Oxalobacter formigenes, Alistipes putredinis, Bacteroides vulgatus, Clostridium aldenense, Eubacterium siraeum, Oxalobacter formigenes, Alistipes senegalensis, Bacteroides xylanisolvens, Clostridium bolteae, Eubacterium ventriosum, Parabacteroides distasonis, Alistipes shahii, Bacteroides xylanisolvens, Clostridium bolteae, Eubacterium xylanophilum, Parabacteroides distasonis, Alistipes shahii, Bacteroides xylanisolvens, Clostridium citroniae, Faecalibacterium prausnitzii, Parabacteroides merdae, Alistipes sp. FBI00180, Barnesiella intestinihominis, Clostridium citroniae, Faecalibacterium prausnitzii, Parabacteroides merdae, Alistipes sp. FBI00238, Bifidobacterium adolescentis, Clostridium clostridioforme, Fusicatenibacter saccharivorans, Paraprevotella clara, Alistipes timonensis, Bifidobacterium adolescentis, Clostridium fessum, Fusicatenibacter saccharivorans, Parasutterella excrementihominis, Anaerofustis stercorihominis, Bifidobacterium adolescentis, Clostridium scindens, Gordonibacter pamelaeae, Parasutterella excrementihominis, Anaerostipes hadrus, Bifidobacterium bifidum, Collinsella aerofaciens, Gordonibacter pamelaeae, Phascolarctobacterium faecium, Anaerostipes hadrus, Bifidobacterium catenulatum, Collinsella aerofaciens, Holdemanella biformis, Porphyromonas asaccharolytica, Anaerotruncus massiliensis, Bifidobacterium dentium, Coprococcus comes, Holdemanella biformis, Porphyromonas asaccharolytica, Bacteroides caccae, Bifidobacterium longum, Coprococcus comes, Hungatella effluvii, Roseburia hominis, Bacteroides caccae, Bifidobacterium longum, Coprococcus eutactus, Hungatella effluvii, Roseburia hominis, Bacteroides coprocola, Bifidobacterium pseudocatenulatum, Dialister invisus, Hungatella effluvii, Ruminococcaceae sp. FBI00082 FBI00097, Bacteroides faecis, Bifidobacterium pseudocatenulatum, Dialister succinatiphilus, Lachnoclostridium pacaense, Ruminococcaceae sp. FBI00233, Bacteroides finegoldii, Bifidobacterium pseudocatenulatum, Dielma fastidiosa, Lachnoclostridium pacaense, Ruminococcus bromii, Bacteroides fragilis, Bilophila wadsworthia, Dorea formicigenerans, Lachnospiraceae sp. FBI00033, Ruminococcus bromii, Bacteroides kribbi/Bacteroides koreensis species cluster, Bilophila wadsworthia, Dorea formicigenerans, Lachnospiraceae sp. FBI00071, Ruminococcus faecis, Bacteroides kribbi/Bacteroides koreensis species cluster, Blautia faecis, Dorea longicatena, Lachnospiraceae sp. FBI00290, Ruminococcus faecis, Bacteroides massiliensis, Blautia faecis, Dorea longicatena, Lactobacillus rogosae, Ruthenibacterium lactatiformans, Bacteroides nordii, Blautia hydrogenotrophica, Eggerthella lenta, Lactobacillus rogosae, Senegalimassilia anaerobia, Bacteroides ovatus, Blautia massiliensis, Eggerthella lenta, Longicatena caecimuris, Sutterella massiliensis, Bacteroides salyersiae, Blautia obeum, Eggerthella lenta, Megasphaera massiliensis, Sutterella wadsworthensis, Bacteroides stercorirosoris, Blautia obeum, Eggerthella lenta, Methanobrevibacter smithii, Sutterella wadsworthensis, Bacteroides stercoris, Blautia wexlerae, Eisenbergiella tayi, Methanobrevibacter smithii, Turicibacter sanguinis, Bacteroides stercoris, Blautia wexlerae, Eisenbergiella tayi, and Monoglobus pectinilyticus.

In certain embodiments, the Consortia comprises a plurality of microbes (e.g., active microbes and supportive community microbes) designated as “Consortia XI.” In certain embodiments, the Consortia XI comprises Acidaminococcus intestini, Bifidobacterium longum, Fusicatenibacter saccharivorans, Bacteroides xylanisolvens, Clostridium bolteae, Akkermansia muciniphila, Bilophila wadsworthia, Gordonibacter pamelaeae, Turicibacter sanguinis, Collinsella aerofaciens, Alistipes onderdonkii, Blautia hydrogenotrophica, Hungatella effluvii, Bifidobacterium adolescentis, Coprococcus comes, Alistipes putredinis, Blautia massiliensis, Lachnoclostridium pacaense, Bifidobacterium pseudocatenulatum, Dorea formicigenerans, Alistipes senegalensis, Blautia obeum, Lachnospiraceae sp. FBI00033, Blautia faecis, Dorea longicatena, Alistipes shahii, Blautia wexlerae, Lachnospiraceae sp. FBI00071, Clostridium citroniae, Eggerthella lenta, Alistipes sp. FBI00180, Butyricimonas faecihominis, Lachnospiraceae sp. FBI00290, Faecalibacterium prausnitzii, Eggerthella lenta, Alistipes sp. FBI00238, Catabacter hongkongensis, Lactobacillus rogosae, Holdemanella biformis, Eggerthella lenta, Alistipes timonensis, Clostridiaceae sp. FBI00191, Longicatena caecimuris, Bacteroides xylanisolvens, Eisenbergiella tayi, Anaerofustis stercorihominis, Clostridiales sp. FBI00377, Megasphaera massiliensis, Bifidobacterium adolescentis, Eubacterium eligens, Anaerostipes hadrus, Clostridium aldenense, Methanobrevibacter smithii, Bifidobacterium pseudocatenulatum, Eubacterium rectale, Anaerotruncus massiliensis, Clostridium bolteae, Monoglobus pectinilyticus, Blautia faecis, Fusicatenibacter saccharivorans, Bacteroides caccae, Clostridium clostridioforme, Neglecta timonensis, Alistipes onderdonkii, Gordonibacter pamelaeae, Bacteroides coprocola, Clostridium fessum, Oxalobacter formigenes, Clostridium citroniae, Hungatella effluvia, Bacteroides faecis, Clostridium scindens, Oxalobacter formigenes, Alistipes putredinis, Hungatella effluvia, Bacteroides finegoldii, Collinsella aerofaciens, Oxalobacter formigenes, Alistipes shahii, Lachnoclostridium pacaense, Bacteroides fragilis, Coprococcus comes, Parabacteroides distasonis, Anaerostipes hadrus, Lactobacillus rogosae, Bacteroides kribbi/Bacteroides koreensis species cluster, Coprococcus eutactus, Parabacteroides merdae, Bacteroides caccae, Methanobrevibacter smithii, Bacteroides massiliensis, Dialister invisus, Paraprevotella clara, Bacteroides kribbi/Bacteroides koreensis species cluster, Monoglobus pectinilyticus, Bacteroides nordii, Dialister succinatiphilus, Parasutterella excrementihominis, Bacteroides stercoris, Neglecta timonensis, Bacteroides ovatus, Dielma fastidiosa, Phascolarctobacterium faecium, Bacteroides thetaiotaomicron, Parabacteroides distasonis, Bacteroides salyersiae, Dorea formicigenerans, Porphyromonas asaccharolytica, Bacteroides uniformis, Parabacteroides merdae, Bacteroides stercorirosoris, Dorea longicatena, Roseburia hominis, Bacteroides vulgatus, Parasutterella excrementihominis, Bacteroides stercoris, Eggerthella lenta, Ruminococcaceae sp. FBI00082 FBI00097, Bacteroides xylanisolvens, Porphyromonas asaccharolytica, Bacteroides thetaiotaomicron, Eisenbergiella tayi, Ruminococcaceae sp. FBI00233, Bifidobacterium adolescentis, Roseburia hominis, Bacteroides uniformis, Eubacterium eligens, Ruminococcus bromii, Bifidobacterium longum, Ruminococcus bromii, Bacteroides vulgatus, Eubacterium hallii, Ruminococcus faecis, Bifidobacterium pseudocatenulatum, Ruminococcus faecis, Barnesiella intestinihominis, Eubacterium rectale, Ruthenibacterium lactatiformans, Bilophila wadsworthia, Sutterella wadsworthensis, Bifidobacterium bifidum, Eubacterium siraeum, Senegalimassilia anaerobia, Blautia obeum, Bifidobacterium catenulatum, Eubacterium ventriosum, Sutterella massiliensis, Blautia wexlerae, Bifidobacterium dentium, Eubacterium xylanophilum, Sutterella wadsworthensis, and Clostridium aldenense.

In certain embodiments, the Consortia comprises a plurality of microbes (e.g., active microbes and supportive community microbes) designated as “Consortia XII.” In certain embodiments, the Consortia XII comprises Acidaminococcus intestini, Bacteroides uniformis, Clostridium bolteae, Faecalibacterium prausnitzii, Parasutterella excrementihominis, Akkermansia muciniphila, Bacteroides vulgatus, Clostridium bolteae, Faecalibacterium prausnitzii, Phascolarctobacterium faecium, Alistipes onderdonkii, Bacteroides vulgatus, Clostridium citroniae, Fusicatenibacter saccharivorans, Phascolarctobacterium faecium, Alistipes onderdonkii, Bacteroides xylanisolvens, Clostridium citroniae, Fusicatenibacter saccharivorans, Porphyromonas asaccharolytica, Alistipes putredinis, Bacteroides xylanisolvens, Clostridium clostridioforme, Gordonibacter pamelaeae, Porphyromonas asaccharolytica, Alistipes putredinis, Bacteroides xylanisolvens, Clostridium fessum, Gordonibacter pamelaeae, Roseburia hominis, Alistipes senegalensis, Barnesiella intestinihominis, Clostridium fessum, Holdemanella biformis, Roseburia hominis, Alistipes shahii, Bifidobacterium adolescentis, Clostridium scindens, Holdemanella biformis, Ruminococcaceae sp. FBI00082 FBI00097, Alistipes shahii, Bifidobacterium adolescentis, Collinsella aerofaciens, Hungatella effluvii, Ruminococcaceae sp. FBI00082 FBI00097, Alistipes sp. FBI00180, Bifidobacterium adolescentis, Collinsella aerofaciens, Hungatella effluvii, Ruminococcaceae sp. FBI00233, Alistipes sp. FBI00238, Bifidobacterium bifidum, Coprococcus comes, Hungatella effluvii, Ruminococcus bromii, Alistipes timonensis, Bifidobacterium bifidum, Coprococcus comes, Lachnoclostridium pacaense, Ruminococcus bromii, Anaerofustis stercorihominis, Bifidobacterium catenulatum, Coprococcus eutactus, Lachnoclostridium pacaense, Ruminococcus faecis, Anaerostipes hadrus, Bifidobacterium dentium, Coprococcus eutactus, Lachnospiraceae sp. FBI00033, Ruminococcus faecis, Anaerostipes hadrus, Bifidobacterium longum, Dialister invisus, Lachnospiraceae sp. FBI00071, Ruthenibacterium lactatiformans, Anaerotruncus massiliensis, Bifidobacterium longum, Dialister succinatiphilus, Lachnospiraceae sp. FBI00290, Senegalimassilia anaerobia, Bacteroides caccae, Bifidobacterium pseudocatenulatum, Dielma fastidiosa, Lactobacillus rogosae, Sutterella massiliensis, Bacteroides caccae, Bifidobacterium pseudocatenulatum, Dorea formicigenerans, Lactobacillus rogosae, Sutterella wadsworthensis, Bacteroides coprocola, Bifidobacterium pseudocatenulatum, Dorea formicigenerans, Longicatena caecimuris, Sutterella wadsworthensis, Bacteroides faecis, Bilophila wadsworthia, Dorea longicatena, Megasphaera massiliensis, Turicibacter sanguinis, Bacteroides finegoldii, Bilophila wadsworthia, Dorea longicatena, Methanobrevibacter smithii, Bacteroides thetaiotaomicron, Bacteroides fragilis, Blautia faecis, Eggerthella lenta, Methanobrevibacter smithii, Bacteroides uniformis, Bacteroides kribbi/Bacteroides koreensis species cluster, Blautia faecis, Eggerthella lenta, Monoglobus pectinilyticus, Clostridium aldenense, Bacteroides kribbi/Bacteroides koreensis species cluster, Blautia hydrogenotrophica, Eggerthella lenta, Monoglobus pectinilyticus, Clostridium aldenense, Bacteroides kribbi/Bacteroides koreensis species cluster, Blautia massiliensis, Eggerthella lenta, Neglecta timonensis, Eubacterium ventriosum, Bacteroides massiliensis, Blautia obeum, Eisenbergiella tayi, Neglecta timonensis, Eubacterium xylanophilum, Bacteroides nordii, Blautia obeum, Eisenbergiella tayi, Oxalobacter formigenes, Paraprevotella clara, Bacteroides ovatus, Blautia wexlerae, Eubacterium eligens, Oxalobacter formigenes, Parasutterella excrementihominis, Bacteroides salyersiae, Blautia wexlerae, Eubacterium eligens, Oxalobacter formigenes, Bacteroides thetaiotaomicron, Bacteroides stercorirosoris, Butyricimonas faecihominis, Eubacterium hallii, Parabacteroides distasonis, Clostridiales sp. FBI00377, Bacteroides stercoris, Catabacter hongkongensis, Eubacterium rectale, Parabacteroides distasonis, Eubacterium siraeum, Bacteroides stercoris, Clostridiaceae sp. FBI00191, Eubacterium rectale, Parabacteroides merdae, and Parabacteroides merdae.

In certain embodiments, the Consortia comprises a plurality of microbes (e.g., active microbes and supportive community microbes) designated as “Consortia XIII.” In certain embodiments, the Consortia XIII comprises Acidaminococcus intestini, Bacteroides thetaiotaomicron, Butyricimonas faecihominis, Eubacterium eligens, Monoglobus pectinilyticus, Akkermansia muciniphila, Bacteroides thetaiotaomicron, Catabacter hongkongensis, Eubacterium eligens, Neglecta timonensis, Alistipes onderdonkii, Bacteroides uniformis, Clostridiaceae sp. FBI00191, Eubacterium hallii, Neglecta timonensis, Alistipes onderdonkii, Bacteroides uniformis, Clostridiales sp. FBI00377, Eubacterium rectale, Oxalobacter formigenes, Alistipes putredinis, Bacteroides vulgatus, Clostridium aldenense, Eubacterium rectale, Oxalobacter formigenes, Alistipes putredinis, Bacteroides vulgatus, Clostridium aldenense, Eubacterium siraeum, Oxalobacter formigenes, Alistipes senegalensis, Bacteroides xylanisolvens, Clostridium bolteae, Eubacterium ventriosum, Parabacteroides distasonis, Alistipes shahii, Bacteroides xylanisolvens, Clostridium bolteae, Eubacterium xylanophilum, Parabacteroides distasonis, Alistipes shahii, Bacteroides xylanisolvens, Clostridium citroniae, Faecalibacterium prausnitzii, Parabacteroides merdae, Alistipes sp. FBI00180, Barnesiella intestinihominis, Clostridium citroniae, Faecalibacterium prausnitzii, Parabacteroides merdae, Alistipes sp. FBI00238, Bifidobacterium adolescentis, Clostridium clostridioforme, Fusicatenibacter saccharivorans, Paraprevotella clara, Alistipes timonensis, Bifidobacterium adolescentis, Clostridium fessum, Fusicatenibacter saccharivorans, Parasutterella excrementihominis, Anaerofustis stercorihominis, Bifidobacterium adolescentis, Clostridium scindens, Gordonibacter pamelaeae, Parasutterella excrementihominis, Anaerostipes hadrus, Bifidobacterium bifidum, Collinsella aerofaciens, Gordonibacter pamelaeae, Phascolarctobacterium faecium, Anaerostipes hadrus, Bifidobacterium catenulatum, Collinsella aerofaciens, Holdemanella biformis, Porphyromonas asaccharolytica, Anaerotruncus massiliensis, Bifidobacterium dentium, Coprococcus comes, Holdemanella biformis, Porphyromonas asaccharolytica, Bacteroides caccae, Bifidobacterium longum, Coprococcus comes, Hungatella effluvii, Roseburia hominis, Bacteroides caccae, Bifidobacterium longum, Coprococcus eutactus, Hungatella effluvii, Roseburia hominis, Bacteroides coprocola, Bifidobacterium pseudocatenulatum, Dialister invisus, Hungatella effluvii, Ruminococcaceae sp. FBI00082 FBI00097, Bacteroides faecis, Bifidobacterium pseudocatenulatum, Dialister succinatiphilus, Lachnoclostridium pacaense, Ruminococcaceae sp. FBI00233, Bacteroides finegoldii, Bifidobacterium pseudocatenulatum, Dielma fastidiosa, Lachnoclostridium pacaense, Ruminococcus bromii, Bacteroides fragilis, Bilophila wadsworthia, Dorea formicigenerans, Lachnospiraceae sp. FBI00033, Ruminococcus bromii, Bacteroides kribbi/Bacteroides koreensis species cluster, Bilophila wadsworthia, Dorea formicigenerans, Lachnospiraceae sp. FBI00071, Ruminococcus faecis, Bacteroides kribbi/Bacteroides koreensis species cluster, Blautia faecis, Dorea longicatena, Lachnospiraceae sp. FBI00290, Ruminococcus faecis, Bacteroides massiliensis, Blautia faecis, Dorea longicatena, Lactobacillus rogosae, Ruthenibacterium lactatiformans, Bacteroides nordii, Blautia hydrogenotrophica, Eggerthella lenta, Lactobacillus rogosae, Senegalimassilia anaerobia, Bacteroides ovatus, Blautia massiliensis, Eggerthella lenta, Longicatena caecimuris, Sutterella massiliensis, Bacteroides salyersiae, Blautia obeum, Eggerthella lenta, Megasphaera massiliensis, Sutterella wadsworthensis, Bacteroides stercorirosoris, Blautia obeum, Eggerthella lenta, Methanobrevibacter smithii, Sutterella wadsworthensis, Bacteroides stercoris, Blautia wexlerae, Eisenbergiella tayi, Methanobrevibacter smithii, Turicibacter sanguinis, Bacteroides stercoris, Blautia wexlerae, Eisenbergiella tayi, and Monoglobus pectinilyticus.

In certain embodiments, the Consortia comprises a plurality of microbes (e.g., active microbes and supportive community microbes) designated as “Consortia XIV.” In certain embodiments, the Consortia XIV comprises Acidaminococcus intestini, Bacteroides uniformis, Clostridium citroniae, Eubacterium siraeum, Parasutterella excrementihominis, Akkermansia muciniphila, Bacteroides uniformis, Clostridium citroniae, Eubacterium ruminantium, Parasutterella excrementihominis, Alistipes finegoldii, Bacteroides vulgatus, Clostridium clostridioforme, Eubacterium ventriosum, Phascolarctobacterium faecium, Alistipes onderdonkii, Bacteroides vulgatus, Clostridium scindens, Eubacterium xylanophilum, Phascolarctobacterium faecium, Alistipes onderdonkii, Bacteroides xylanisolvens, Clostridium swellfunianum, Faecalibacterium prausnitzii, Phocea massiliensis, Alistipes putredinis, Bacteroides xylanisolvens, Clostridium symbiosum, Faecalibacterium prausnitzii, Phocea massiliensis, Alistipes putredinis, Barnesiella intestinihominis, Clostridium symbiosum, Fusicatenibacter saccharivorans, Porphyromonas asaccharolytica, Alistipes senegalensis, Bifidobacterium adolescentis, Collinsella aerofaciens, Fusicatenibacter saccharivorans, Porphyromonas asaccharolytica, Alistipes senegalensis, Bifidobacterium adolescentis, Collinsella aerofaciens, Gordonibacter pamelaeae, Roseburia hominis, Alistipes shahii, Bifidobacterium bifidum, Coprococcus comes, Gordonibacter pamelaeae, Roseburia hominis, Alistipes shahii, Bifidobacterium bifidum, Coprococcus comes, Holdemanella biformis, Ruminococcus bromii, Alistipes timonensis, Bifidobacterium catenulatum, Coprococcus eutactus, Holdemanella biformis, Ruminococcus bromii, Anaerofustis stercorihominis, Bifidobacterium dentium, Coprococcus eutactus, Hungatella effluvii, Ruminococcus faecis, Anaerostipes hadrus, Bifidobacterium faecale, Desulfovibrio desulfuricans, Hungatella hathewayi, Ruminococcus faecis, Anaerostipes hadrus, Bifidobacterium longum, Desulfovibrio desulfuricans, Hungatella hathewayi, Ruthenibacterium lactatiformans, Anaerotruncus colihominis, Bifidobacterium longum, Dialister invisus, Hydrogenoanaerobacterium saccharovorans, Senegalimassilia anaerobia, Bacteroides caccae, Bifidobacterium pseudocatenulatum, Dialister succinatiphilus, Lachnoclostridium pacaense, Sutterella massiliensis, Bacteroides caccae, Bifidobacterium pseudocatenulatum, Dielma fastidiosa, Lachnoclostridium pacaense, Sutterella wadsworthensis, Bacteroides coprocola, Bifidobacterium pseudocatenulatum, Dorea formicigenerans, Lachnospira pectinoschiza, Sutterella wadsworthensis, Bacteroides faecis, Blautia faecis, Dorea formicigenerans, Lachnospira pectinoschiza, Turicibacter sanguinis, Bacteroides finegoldii, Blautia faecis, Dorea longicatena, Longicatena caecimuris, Bacteroides stercoris, Bacteroides fragilis, Blautia hydrogenotrophica, Dorea longicatena, Megasphaera massiliensis, Bacteroides stercoris, Bacteroides koreensis, Blautia luti, Eggerthella lenta, Methanobrevibacter smithii, Bacteroides thetaiotaomicron, Bacteroides koreensis, Blautia obeum, Eggerthella lenta, Methanobrevibacter smithii, Bacteroides thetaiotaomicron, Bacteroides kribbi, Blautia obeum, Eggerthella lenta, Monoglobus pectinilyticus, Clostridium aldenense, Bacteroides kribbi, Blautia wexlerae, Eggerthella lenta, Monoglobus pectinilyticus, Clostridium aldenense, Bacteroides massiliensis, Blautia wexlerae, Eisenbergiella tayi, Neglecta timonensis, Clostridium bolteae, Bacteroides nordii, Butyricimonas faecihominis, Eisenbergiella tayi, Oxalobacter formigenes, Clostridium bolteae, Bacteroides oleiciplenus, Catabacter hongkongensis, Emergencia timonensis, Oxalobacter formigenes, Parabacteroides merdae, Bacteroides ovatus, Citrobacter freundii, Eubacterium eligens, Oxalobacter formigenes, Parabacteroides merdae, Bacteroides salyersiae, Clostridiaceae sp., Eubacterium eligens, Parabacteroides distasonis, Paraprevotella clara, Eubacterium rectale, Eubacterium rectale, Eubacterium hallii, Parabacteroides distasonis, and Eubacterium oxidoreducens.

In certain embodiments, the Consortia comprises a plurality of microbes (e.g., active microbes and supportive community microbes) designated as “Consortia XV.” In certain embodiments, the Consortia XV comprises Acidaminococcus intestini, Bacteroides thetaiotaomicron, Butyricimonas faecihominis, Eubacterium eligens, Monoglobus pectinilyticus, Akkermansia muciniphila, Bacteroides thetaiotaomicron, Catabacter hongkongensis, Eubacterium eligens, Neglecta timonensis, Alistipes onderdonkii, Bacteroides uniformis, Clostridiaceae sp. FBI00191, Eubacterium hallii, Neglecta timonensis, Alistipes onderdonkii, Bacteroides uniformis, Clostridiales sp. FBI00377, Eubacterium rectale, Oxalobacter formigenes, Alistipes putredinis, Bacteroides vulgatus, Clostridium aldenense, Eubacterium rectale, Oxalobacter formigenes, Alistipes putredinis, Bacteroides vulgatus, Clostridium aldenense, Eubacterium siraeum, Oxalobacter formigenes, Alistipes senegalensis, Bacteroides xylanisolvens, Clostridium bolteae, Eubacterium ventriosum, Parabacteroides distasonis, Alistipes shahii, Bacteroides xylanisolvens, Clostridium bolteae, Eubacterium xylanophilum, Parabacteroides distasonis, Alistipes shahii, Bacteroides xylanisolvens, Clostridium citroniae, Faecalibacterium prausnitzii, Parabacteroides merdae, Alistipes sp. FBI00180, Barnesiella intestinihominis, Clostridium citroniae, Faecalibacterium prausnitzii, Parabacteroides merdae, Alistipes sp. FBI00238, Bifidobacterium adolescentis, Clostridium clostridioforme, Fusicatenibacter saccharivorans, Paraprevotella clara, Alistipes timonensis, Bifidobacterium adolescentis, Clostridium fessum, Fusicatenibacter saccharivorans, Parasutterella excrementihominis, Anaerofustis stercorihominis, Bifidobacterium adolescentis, Clostridium scindens, Gordonibacter pamelaeae, Parasutterella excrementihominis, Anaerostipes hadrus, Bifidobacterium bifidum, Collinsella aerofaciens, Gordonibacter pamelaeae, Phascolarctobacterium faecium, Anaerostipes hadrus, Bifidobacterium catenulatum, Collinsella aerofaciens, Holdemanella biformis, Porphyromonas asaccharolytica, Anaerotruncus massiliensis, Bifidobacterium dentium, Coprococcus comes, Holdemanella biformis, Porphyromonas asaccharolytica, Bacteroides caccae, Bifidobacterium longum, Coprococcus comes, Hungatella effluvii, Roseburia hominis, Bacteroides caccae, Bifidobacterium longum, Coprococcus eutactus, Hungatella effluvii, Roseburia hominis, Bacteroides coprocola, Bifidobacterium pseudocatenulatum, Dialister invisus, Hungatella effluvii, Ruminococcaceae sp. FBI00082 FBI00097, Bacteroides faecis, Bifidobacterium pseudocatenulatum, Dialister succinatiphilus, Lachnoclostridium pacaense, Ruminococcaceae sp. FBI00233, Bacteroides finegoldii, Bifidobacterium pseudocatenulatum, Dielma fastidiosa, Lachnoclostridium pacaense, Ruminococcus bromii, Bacteroides fragilis, Bilophila wadsworthia, Dorea formicigenerans, Lachnospiraceae sp. FBI00033, Ruminococcus bromii, Bacteroides kribbi/Bacteroides koreensis species cluster, Bilophila wadsworthia, Dorea formicigenerans, Lachnospiraceae sp. FBI00071, Ruminococcus faecis, Bacteroides kribbi/Bacteroides koreensis species cluster, Blautia faecis, Dorea longicatena, Lachnospiraceae sp. FBI00290, Ruminococcus faecis, Bacteroides massiliensis, Blautia faecis, Dorea longicatena, Lactobacillus rogosae, Ruthenibacterium lactatiformans, Bacteroides nordii, Blautia hydrogenotrophica, Eggerthella lenta, Lactobacillus rogosae, Senegalimassilia anaerobia, Bacteroides ovatus, Blautia massiliensis, Eggerthella lenta, Longicatena caecimuris, Sutterella massiliensis, Bacteroides salyersiae, Blautia obeum, Eggerthella lenta, Megasphaera massiliensis, Sutterella wadsworthensis, Bacteroides stercorirosoris, Blautia obeum, Eggerthella lenta, Methanobrevibacter smithii, Sutterella wadsworthensis, Bacteroides stercoris, Blautia wexlerae, Eisenbergiella tayi, Methanobrevibacter smithii, Turicibacter sanguinis, Bacteroides stercoris, Blautia wexlerae, Eisenbergiella tayi, and Monoglobus pectinilyticus.

In certain embodiments, the Consortia comprises a plurality of microbes (e.g., active microbes and supportive community microbes) designated as “Consortia XVI.” In certain embodiments, the Consortia XVI comprises Acidaminococcus intestini, Bacteroides uniformis, Clostridium citroniae, Eubacterium ventriosum, Parasutterella excrementihominis, Akkermansia muciniphila, Bacteroides uniformis, Clostridium clostridioforme, Eubacterium siraeum, Phascolarctobacterium faecium, Alistipes finegoldii, Bacteroides vulgatus, Clostridium scindens, Eubacterium xylanophilum, Phascolarctobacterium faecium, Alistipes onderdonkii, Bacteroides vulgatus, Clostridium swellfunianum, Faecalibacterium prausnitzii, Phocea massiliensis, Alistipes onderdonkii, Bacteroides xylanisolvens, Clostridium symbiosum, Faecalibacterium prausnitzii, Phocea massiliensis, Alistipes putredinis, Bacteroides xylanisolvens, Clostridium symbiosum, Fusicatenibacter saccharivorans, Porphyromonas asaccharolytica, Alistipes putredinis, Barnesiella intestinihominis, Collinsella aerofaciens, Fusicatenibacter saccharivorans, Porphyromonas asaccharolytica, Alistipes senegalensis, Bifidobacterium adolescentis, Collinsella aerofaciens, Gordonibacter pamelaeae, Roseburia hominis, Alistipes senegalensis, Bifidobacterium adolescentis, Coprococcus comes, Gordonibacter pamelaeae, Roseburia hominis, Alistipes shahii, Bifidobacterium bifidum, Coprococcus comes, Holdemanella biformis, Ruminococcus bromii, Alistipes shahii, Bifidobacterium bifidum, Coprococcus eutactus, Holdemanella biformis, Ruminococcus bromii, Alistipes timonensis, Bifidobacterium catenulatum, Coprococcus eutactus, Hungatella effluvii, Ruminococcus faecis, Anaerofustis stercorihominis, Bifidobacterium dentium, Desulfovibrio desulfuricans, Hungatella hathewayi, Ruminococcus faecis, Anaerostipes hadrus, Bifidobacterium faecale, Desulfovibrio desulfuricans, Hungatella hathewayi, Ruthenibacterium lactatiformans, Anaerostipes hadrus, Bifidobacterium longum, Dialister invisus, Hydrogenoanaerobacterium saccharovorans, Senegalimassilia anaerobia, Anaerotruncus colihominis, Bifidobacterium longum, Dialister succinatiphilus, Lachnoclostridium pacaense, Sutterella massiliensis, Bacteroides caccae, Bifidobacterium pseudocatenulatum, Dielma fastidiosa, Lachnoclostridium pacaense, Sutterella wadsworthensis, Bacteroides caccae, Bifidobacterium pseudocatenulatum, Dorea formicigenerans, Lachnospira pectinoschiza, Sutterella wadsworthensis, Bacteroides coprocola, Bifidobacterium pseudocatenulatum, Dorea formicigenerans, Lachnospira pectinoschiza, Turicibacter sanguinis, Bacteroides faecis, Blautia faecis, Dorea longicatena, Longicatena caecimuris, Bacteroides stercoris, Bacteroides finegoldii, Blautia faecis, Dorea longicatena, Megasphaera massiliensis, Bacteroides stercoris, Bacteroides fragilis, Blautia hydrogenotrophica, Eggerthella lenta, Methanobrevibacter smithii, Bacteroides thetaiotaomicron, Bacteroides koreensis, Blautia luti, Eggerthella lenta, Methanobrevibacter smithii, Bacteroides thetaiotaomicron, Bacteroides koreensis, Blautia obeum, Eggerthella lenta, Monoglobus pectinilyticus, Clostridium aldenense, Bacteroides kribbi, Blautia obeum, Eggerthella lenta, Monoglobus pectinilyticus, Clostridium bolteae, Bacteroides kribbi, Blautia wexlerae, Eisenbergiella tayi, Neglecta timonensis, Clostridium bolteae, Bacteroides massiliensis, Blautia wexlerae, Eisenbergiella tayi, Oxalobacter formigenes, Clostridium citroniae, Bacteroides nordii, Butyricimonas faecihominis, Emergencia timonensis, Oxalobacter formigenes, Eubacterium oxidoreducens, Bacteroides oleiciplenus, Catabacter hongkongensis, Eubacterium eligens, Oxalobacter formigenes, Eubacterium rectale, Bacteroides ovatus, Clostridiaceae sp., Eubacterium eligens, Parabacteroides distasonis, Eubacterium rectale, Bacteroides salyersiae, Clostridium aldenense, Eubacterium hallii, Parabacteroides distasonis, Eubacterium ruminantium, Parasutterella excrementihominis, Paraprevotella clara, Parabacteroides merdae, and Parabacteroides merdae.

In certain embodiments, the Consortia comprises a plurality of microbes (e.g., active microbes and supportive community microbes) designated as “Consortia XVII.” In certain embodiments, the Consortia XVII comprises Acidaminococcus intestini, Bacteroides uniformis, Clostridium bolteae, Faecalicatena contorta, Roseburia hominis, Acutalibacter timonensis, Bacteroides vulgatus, Clostridium citroniae, Fusicatenibacter saccharivorans, Roseburia hominis, Akkermansia muciniphila, Bacteroides vulgatus, Clostridium citroniae, Fusicatenibacter saccharivorans, Ruminococcaceae sp. FBI00097, Alistipes onderdonkii, Bacteroides xylanisolvens, Clostridium clostridioforme, Gordonibacter pamelaeae, Ruminococcaceae sp. FBI0009, Alistipes onderdonkii, Bacteroides xylanisolvens, Clostridium fessum, Gordonibacter pamelaeae, Ruminococcaceae sp. FBI00233, Alistipes putredinis, Bacteroides xylanisolvens, Clostridium fessum, Holdemanella biformis, Ruminococcus bromii, Alistipes putredinis, Barnesiella intestinihominis, Clostridium scindens, Holdemanella biformis, Ruminococcus bromii, Alistipes senegalensis, Bifidobacterium adolescentis, Collinsella aerofaciens, Hungatella effluvii, Ruminococcus faecis, Alistipes shahii, Bifidobacterium adolescentis, Collinsella aerofaciens, Hungatella effluvii, Ruminococcus faecis, Alistipes sp. FBI00180, Bifidobacterium adolescentis, Coprococcus comes, Hungatella effluvii, Ruthenibacterium lactatiformans, Alistipes sp. FBI00238, Bifidobacterium bifidum, Coprococcus comes, Lachnoclostridium pacaense, Senegalimassilia anaerobia, Alistipes timonensis, Bifidobacterium bifidum, Coprococcus eutactus, Lachnoclostridium pacaense, Sutterella massiliensis, Anaerofustis stercorihominis, Bifidobacterium catenulatum, Coprococcus eutactus, Lachnospiraceae sp. FBI00033, Sutterella wadsworthensis, Anaerostipes hadrus, Bifidobacterium dentium, Dialister invisus, Lachnospiraceae sp. FBI00071, Sutterella wadsworthensis, Anaerostipes hadrus, Bifidobacterium longum, Dialister succinatiphilus, Lachnospiraceae sp. FBI00290, Turicibacter sanguinis, Anaerotruncus massiliensis, Bifidobacterium longum, Dielma fastidiosa, Lactobacillus rogosae, Bacteroides stercoris, Bacteroides caccae, Bifidobacterium pseudocatenulatum, Dorea formicigenerans, Lactobacillus rogosae, Bacteroides stercoris, Bacteroides caccae, Bifidobacterium pseudocatenulatum, Dorea formicigenerans, Lactonifactor longoviformis, Bacteroides thetaiotaomicron, Bacteroides coprocola, Bilophila wadsworthia, Dorea longicatena, Longicatena caecimuris, Bacteroides thetaiotaomicron, Bacteroides faecis, Bilophila wadsworthia, Dorea longicatena, Megasphaera massiliensis, Bacteroides uniformis, Bacteroides finegoldii, Blautia faecis, Eggerthella lenta, Monoglobus pectinilyticus, Citrobacter portucalensis, Bacteroides fragilis, Blautia faecis, Eggerthella lenta, Monoglobus pectinilyticus, Clostridiaceae sp. FBI00191, Bacteroides kribbi/Bacteroides koreensis species cluster, Blautia hydrogenotrophica, Eisenbergiella tayi, Oxalobacter formigenes, Clostridium aldenense, Bacteroides kribbi/Bacteroides koreensis species cluster, Blautia massiliensis, Eisenbergiella tayi, Oxalobacter formigenes, Clostridium aldenense, Bacteroides kribbi/Bacteroides koreensis species cluster, Blautia obeum, Emergencia timonensis, Oxalobacter formigenes, Clostridium bolteae, Bacteroides massiliensis, Blautia obeum, Eubacterium eligens, Parabacteroides distasonis, Eubacterium siraeum, Bacteroides nordii, Blautia wexlerae, Eubacterium eligens, Parabacteroides merdae, Eubacterium ventriosum, Bacteroides ovatus, Blautia wexlerae, Eubacterium hallii, Parabacteroides merdae, Eubacterium xylanophilum, Bacteroides salyersiae, Butyricimonas faecihominis, Eubacterium rectale, Paraprevotella clara, Faecalibacterium prausnitzii, Bacteroides stercorirosoris, Catabacter hongkongensis, Eubacterium rectale, Parasutterella excrementihominis, Faecalibacterium prausnitzii, Phascolarctobacterium faecium, Phascolarctobacterium faecium, Porphyromonas asaccharolytica, Parasutterella excrementihominis, and Porphyromonas asaccharolytica.

In certain embodiments, the Consortia comprises a plurality of microbes (e.g., active microbes and supportive community microbes) designated as “Consortia XVIII.” In certain embodiments, the Consortia XVIII comprises Bacteroides caccae, Bifidobacterium longum, Clostridium scindens, Eggerthella lenta, Parabacteroides merdae, Bacteroides salyersiae, Bifidobacterium pseudocatenulatum, Clostridium symbiosum, Eggerthella lenta, Ruminococcus bromii, Bacteroides thetaiotaomicron, Bifidobacterium pseudocatenulatum, Collinsella aerofaciens, Faecalibacterium prausnitzii, Ruminococcus bromii, Bacteroides thetaiotaomicron, Bifidobacterium pseudocatenulatum, Desulfovibrio desulfuricans, Neglecta timonensis, Bacteroides vulgatus, Clostridium amygdalinum, Dorea longicatena, Oxalobacter formigenes, Bifidobacterium dentium, Clostridium citroniae, Eggerthella lenta, Oxalobacter formigenes, Bifidobacterium longum, Clostridium citroniae, Eggerthella lenta, and Oxalobacter formigenes.

In certain embodiments, the Consortia comprises a plurality of microbes (e.g., active microbes and supportive community microbes) designated as “Consortia XIX.” In certain embodiments, the Consortia XIX comprises Acidaminococcus intestini, Bacteroides stercoris, Blautia wexlerae, Eisenbergiella tayi, Monoglobus pectinilyticus, Acutalibacter timonensis, Bacteroides stercoris, Blautia wexlerae, Eisenbergiella tayi, Monoglobus pectinilyticus, Akkermansia muciniphila, Bacteroides thetaiotaomicron, Butyricimonas faecihominis, Emergencia timonensis, Parabacteroides distasonis, Alistipes onderdonkii, Bacteroides thetaiotaomicron, Catabacter hongkongensis, Eubacterium eligens, Oxalobacter formigenes, Alistipes onderdonkii, Bacteroides uniformis, Clostridiaceae sp. FBI00191, Eubacterium eligens, Oxalobacter formigenes, Alistipes putredinis, Bacteroides uniformis, Clostridium aldenense, Eubacterium hallii, Oxalobacter formigenes, Alistipes putredinis, Bacteroides vulgatus, Clostridium aldenense, Eubacterium rectale, Parabacteroides distasonis, Alistipes senegalensis, Bacteroides vulgatus, Clostridium bolteae, Eubacterium rectale, Parabacteroides merdae, Alistipes shahii, Bacteroides xylanisolvens, Clostridium bolteae, Eubacterium siraeum, Parabacteroides merdae, Alistipes shahii, Bacteroides xylanisolvens, Clostridium citroniae, Eubacterium ventriosum, Paraprevotella clara, Alistipes sp. FBI00180, Bacteroides xylanisolvens, Clostridium citroniae, Eubacterium xylanophilum, Parasutterella excrementihominis, Alistipes sp. FBI00238, Barnesiella intestinihominis, Clostridium clostridioforme, Faecalibacterium prausnitzii, Parasutterella excrementihominis, Alistipes timonensis, Bifidobacterium adolescentis, Clostridium fessum, Fusicatenibacter saccharivorans, Phascolarctobacterium faecium, Anaerofustis stercorihominis, Bifidobacterium adolescentis, Clostridium scindens, Fusicatenibacter saccharivorans, Porphyromonas asaccharolytica, Anaerostipes hadrus, Bifidobacterium bifidum, Collinsella aerofaciens, Gordonibacter pamelaeae, Roseburia hominis, Anaerostipes hadrus, Bifidobacterium catenulatum, Collinsella aerofaciens, Gordonibacter pamelaeae, Roseburia hominis, Anaerotruncus massiliensis, Bifidobacterium dentium, Coprococcus comes, Holdemanella biformis, Ruminococcaceae sp. FBI00097, Bacteroides caccae, Bifidobacterium longum, Coprococcus comes, Hungatella effluvii, Ruminococcaceae sp. FBI00233, Bacteroides caccae, Bifidobacterium longum, Coprococcus eutactus, Hungatella effluvii, Ruminococcus bromii, Bacteroides coprocola, Bifidobacterium pseudocatenulatum, Dialister invisus, Hungatella hathewayi, Ruminococcus bromii, Bacteroides faecis, Bifidobacterium pseudocatenulatum, Dialister succinatiphilus, Lachnoclostridium pacaense, Ruminococcus faecis, Bacteroides finegoldii, Bifidobacterium pseudocatenulatum, Dielma fastidiosa, Lachnoclostridium pacaense, Ruminococcus faecis, Bacteroides fragilis, Bifidobacterium adolescentis, Dorea formicigenerans, Lachnospiraceae sp. FBI00033, Ruthenibacterium lactatiformans, Bacteroides kribbi, Bilophila wadsworthia, Dorea formicigenerans, Lachnospiraceae sp. FBI00071, Senegalimassilia anaerobia, Bacteroides kribbi, Bilophila wadsworthia, Dorea longicatena, Lachnospiraceae sp. FBI00290, Sutterella massiliensis, Bacteroides massiliensis, Blautia faecis, Dorea longicatena, Lactobacillus rogosae, Sutterella wadsworthensis, Bacteroides nordii, Blautia hydrogenotrophica, Eggerthella lenta, Lactobacillus rogosae, Sutterella wadsworthensis, Bacteroides ovatus, Blautia massiliensis, Eggerthella lenta, Longicatena caecimuris, Turicibacter sanguinis, Bacteroides salyersiae, Blautia obeum, Eggerthella lenta, Megasphaera massiliensis, Bacteroides stercorirosoris, Blautia obeum, Eggerthella lenta, and Methanobrevibacter smithii.

In certain embodiments, the Consortia comprises the microbiota comprised in Consortia I. In certain embodiments, the Consortia comprises the microbiota comprised in Consortia II. In certain embodiments, the Consortia comprises the microbiota comprised in Consortia III. In certain embodiments, the Consortia comprises the microbiota comprised in Consortia IV. In certain embodiments, the Consortia comprises the microbiota comprised in Consortia V. In certain embodiments, the Consortia comprises the microbiota comprised in Consortia VI. In certain embodiments, the Consortia comprises the microbiota comprised in Consortia VII. In certain embodiments, the Consortia comprises the microbiota comprised in Consortia VIII. In certain embodiments, the Consortia comprises the microbiota comprised in Consortia IX. In certain embodiments, the Consortia comprises the microbiota comprised in Consortia X. In certain embodiments, the Consortia comprises the microbiota comprised in Consortia XI. In certain embodiments, the Consortia comprises the microbiota comprised in Consortia XII. In certain embodiments, the Consortia comprises the microbiota comprised in Consortia XIII. In certain embodiments, the Consortia comprises the microbiota comprised in Consortia XIV. In certain embodiments, the Consortia comprises the microbiota comprised in Consortia XV. In certain embodiments, the Consortia comprises the microbiota comprised in Consortia XVI. In certain embodiments, the Consortia comprises the microbiota comprised in Consortia XVII. In certain embodiments, the Consortia comprises the microbiota comprised in Consortia XVIII. In certain embodiments, the Consortia comprises the microbiota comprised in Consortia XIX.

In certain embodiments, the Consortia comprises the microbiota that are at least 97% or at least 98% identical to those listed in Consortia I. In certain embodiments, the Consortia comprises the microbiota that are at least 97% or at least 98% identical to those listed in Consortia II. In certain embodiments, the Consortia comprises the microbiota that are at least 97% or at least 98% identical to those listed in Consortia III. In certain embodiments, the Consortia comprises the microbiota that are at least 97% or at least 98% identical to those listed in Consortia IV. In certain embodiments, the Consortia comprises the microbiota that are at least 97% or at least 98% identical to those listed in Consortia V. In certain embodiments, the Consortia comprises the microbiota that are at least 97% or at least 98% identical to those listed in Consortia VI. In certain embodiments, the Consortia comprises the microbiota that are at least 97% or at least 98% identical to those listed in Consortia VII. In certain embodiments, the Consortia comprises the microbiota that are at least 97% or at least 98% identical to those listed in Consortia VIII. In certain embodiments, the Consortia comprises the microbiota that are at least 97% or at least 98% identical to those listed in Consortia IX. In certain embodiments, the Consortia comprises the microbiota that are at least 97% or at least 98% identical to those listed in Consortia X. In certain embodiments, the Consortia comprises the microbiota that are at least 97% or at least 98% identical to those listed in Consortia XI. In certain embodiments, the Consortia comprises the microbiota that are at least 97% or at least 98% identical to those listed in Consortia XII. In certain embodiments, the Consortia comprises the microbiota that are at least 97% or at least 98% identical to those listed in Consortia XIII. In certain embodiments, the Consortia comprises the microbiota that are at least 97% or at least 98% identical to those listed in Consortia XIV. In certain embodiments, the Consortia comprises the microbiota that are at least 97% or at least 98% identical to those listed in Consortia XV. In certain embodiments, the Consortia comprises the microbiota that are at least 97% or at least 98% identical to those listed in Consortia XVI. In certain embodiments, the Consortia comprises the microbiota that are at least 97% or at least 98% identical to those listed in Consortia XVII. In certain embodiments, the Consortia comprises the microbiota that are at least 97% or at least 98% identical to those listed in Consortia XVIII. In certain embodiments, the Consortia comprises the microbiota that are at least 97% or at least 98% identical to those listed in Consortia XIX.

In certain embodiments, the Consortia comprises at least 146 microbes, at least 147 microbes, or at least 148 microbes of Consortia I. In certain embodiments, the Consortia comprises at least 72 microbes, at least 73 microbes, or at least 74 microbes of Consortia II. In certain embodiments, the Consortia comprises at least 85 microbes, at least 86 microbes, or at least 87 microbes of Consortia III. In certain embodiments, the Consortia comprises at least 84 microbes, at least 85 microbes, or at least 86 microbes of Consortia IV. In certain embodiments, the Consortia comprises at least 153 microbes, at least 154 microbes, or at least 155 microbes of Consortia V. In certain embodiments, the Consortia comprises at least 176 microbes, at least 177 microbes, or at least 178 microbes of Consortia VI. In certain embodiments, the Consortia comprises at least 153 microbes, at least 154 microbes, or at least 155 microbes of Consortia VII. In certain embodiments, the Consortia comprises at least 153 microbes, at least 154 microbes, or at least 155 microbes of Consortia VIII. In certain embodiments, the Consortia comprises at least 148 microbes, at least 149 microbes, or at least 150 microbes of Consortia IX. In certain embodiments, the Consortia comprises at least 149 microbes, at least 150 microbes, or at least 151 microbes of Consortia X. In certain embodiments, the Consortia comprises at least 147 microbes, at least 148 microbes, or at least 149 microbes of Consortia XI. In certain embodiments, the Consortia comprises at least 155 microbes, at least 156 microbes, or at least 157 microbes of Consortia XII. In certain embodiments, the Consortia comprises at least 149 microbes, at least 150 microbes, or at least 151 microbes of Consortia XIII. In certain embodiments, the Consortia comprises at least 155 microbes, at least 156 microbes, or at least 157 microbes of Consortia XIV. In certain embodiments, the Consortia comprises at least 149 microbes, at least 150 microbes, or at least 151 microbes of Consortia XV. In certain embodiments, the Consortia comprises at least 153 microbes, at least 154 microbes, or at least 155 microbes of Consortia XVI. In certain embodiments, the Consortia comprises at least 150 microbes, at least 151 microbes, or at least 152 microbes of Consortia XVII. In certain embodiments, the Consortia comprises at least 29 microbes, or at least 30 microbes of Consortia XVIII. In certain embodiments, the Consortia comprises at least 143 microbes, at least 144 microbes, or at least 145 microbes of Consortia XIX.

In certain embodiments, a microbial consortium described herein comprises a microbial strain having a relative abundance of approximately 90%, 80%, 70%, 60%, 50%, 40%, 30%, 20%, 10%, 5%, 1%, 0.1%, 0.01%, 0.001%, 0.0001%, 0.00001%, or 0.000001% of the total microbial consortium. In certain embodiments, the relative abundance of a microbial strain is determined by metagenomic sequencing and calculated as the percentage of reads that are classified as an identified microbial strain, divided by the genome size. In certain embodiments, the relative abundance of a microbial strain of the present disclosure is determined by metagenomic shotgun sequencing.

In certain embodiments, the Consortia comprises the microbiota set forth in Table 1. In certain embodiments, the Consortia comprises the microbiota that are at least 97% or at least 98% identical to those listed in Table 1. In certain embodiments, the Consortia comprises a plurality of microbes (e.g., active microbes and supportive community microbes) designated as “FB-001.” In certain embodiments, the Consortia comprises a plurality of microbes listed in Table 1. In certain embodiments, the Consortia comprises at least 143 microbes, at least 144 microbes, or at least 145 microbes of FB-001. Table 1 is provided below:

TABLE 1
FB-001 Drug Substances

| Drug Substance (DS) | | |
|---|---|---|
| (aka CoCulture, CoC) | Strain ID | Strain Species |
| DS1 | FBI00001 | Clostridium citroniae |
| | FBI00002 | Bacteroides salyersiae |
| | FBI00010 | Blautia obeum |
| | FBI00013 | Parabacteroides merdae |
| | FBI00029 | Parabacteroides distasonis |
| | FBI00032 | Anaerostipes hadrus |
| | FBI00033 | Lachnospiraceae sp. FBI00033 |
| | FBI00034 | Eubacterium eligens |
| | FBI00043 | Bifidobacterium dentium |
| | FBI00044 | Blautia wexlerae |
| | FBI00048 | Fusicatenibacter saccharivorans |
| | FBI00050 | Bacteroides nordii |
| | FBI00051 | Dorea formicigenerans |
| | FBI00057 | Dorea longicatena |
| | FBI00059 | Bacteroides stercorirosoris |
| | FBI00060 | Bifidobacterium longum |
| | FBI00070 | Bacteroides kribbi |
| | FBI00071 | Lachnospiraceae sp. FBI00071 |
| | FBI00076 | Bacteroides thetaiotaomicron |
| | FBI00079 | Clostridium clostridioforme |
| | FBI00087 | Clostridium scindens |
| | FBI00093 | Roseburia hominis |
| | FBI00102 | Clostridium fessum |
| | FBI00109 | Coprococcus comes |
| | FBI00117 | Blautia faecis |
| | FBI00120 | Hungatella hathewayi |
| | FBI00125 | Bacteroides stercoris |
| | FBI00127 | Collinsella aerofaciens |
| | FBI00128 | Hungatella effluvia |
| | FBI00145 | Bifidobacterium adolescentis |
| | FBI00162 | Bifidobacterium catenulatum |
| | FBI00174 | Lactobacillus rogosae |
| | FBI00184 | Bacteroides faecis |
| | FBI00190 | Bacteroides finegoldii |
| | FBI00191 | Clostridiaceae sp. FBI00191 |
| | FBI00194 | Ruminococcus faecis |
| | FBI00198 | Lachnoclostridium pacaense |
| | FBI00199 | Clostridium bolteae |
| | FBI00200 | Longicatena caecimuris |
| | FBI00201 | Eggerthella lenta |
| | FBI00205 | Blautia massiliensis |
| | FBI00206 | Bacteroides xylanisolvens |
| | FBI00211 | Bacteroides vulgatus |
| | FBI00220 | Megasphaera massiliensis |
| | FBI00221 | Butyricimonas faecihominis |
| | FBI00236 | Eisenbergiella tayi |
| | FBI00245 | Acidaminococcus intestini |
| | FBI00248 | Emergencia timonensis |
| | FBI00251 | Bifidobacterium pseudocatenulatum |
| | FBI00254 | Eubacterium hallii |
| | FBI00267 | Anaerofustis stercorihominis |
| | FBI00278 | Eubacterium ventriosum |
| | FBI00288 | Blautia hydrogenotrophica |
| | FBI00290 | Lachnospiraceae sp. FBI00290 |
| DS2 | FBI00004 | Acutalibacter timonensis |
| | FBI00012 | Alistipes onderdonkii |
| | FBI00015 | Bacteroides uniformis |
| | FBI00018 | Eubacterium rectale |
| | FBI00019 | Alistipes timonensis |
| | FBI00021 | Bacteroides kribbi |
| | FBI00038 | Coprococcus eutactus |
| | FBI00040 | Bilophila wadsworthia |
| | FBI00046 | Bacteroides caccae |
| | FBI00061 | Alistipes shahii |
| | FBI00066 | Parasutterella excrementihominis |
| | FBI00075 | Paraprevotella clara |
| | FBI00077 | Sutterella wadsworthensis |
| | FBI00080 | Sutterella massiliensis |
| | FBI00081 | Porphyromonas asaccharolytica |
| | FBI00085 | Ruminococcus bromii |
| | FBI00092 | Monoglobus pectinilyticus |
| | FBI00097 | Ruminococcaceae sp. FBI00097 |
| | FBI00099 | Gordonibacter pamelaeae |
| | FBI00112 | Bacteroides uniformis |
| | FBI00132 | Gordonibacter pamelaeae |
| | FBI00137 | Bacteroides fragilis |
| | FBI00140 | Phascolarctobacterium faecium |
| | FBI00149 | Monoglobus pectinilyticus |
| | FBI00151 | Clostridium aldenense |
| | FBI00176 | Ruthenibacterium lactatiformans |
| | FBI00189 | Bacteroides ovatus |
| | FBI00197 | Bifidobacterium bifidum |
| | FBI00208 | Anaerotruncus massiliensis |
| | FBI00212 | Clostridium aldenense |
| | FBI00224 | Sutterella wadsworthensis |
| | FBI00226 | Catabacter hongkongensis |
| | FBI00229 | Alistipes senegalensis |
| | FBI00233 | Ruminococcaceae sp. FBI00233 |
| | FBI00235 | Alistipes shahii |
| | FBI00237 | Dielma fastidiosa |
| | FBI00243 | Eubacterium siraeum |
| | FBI00244 | Faecalibacterium prausnitzii |
| | FBI00258 | Turicibacter sanguinis |
| | FBI00260 | Eubacterium rectale |
| | FBI00263 | Bacteroides caccae |
| | FBI00270 | Methanobrevibacter smithii |
| | FBI00273 | Barnesiella intestinihominis |
| | FBI00277 | Alistipes onderdonkii |
| | FBI00292 | Methanobrevibacter smithii |
| DS3 | FBI00009 | Bifidobacterium adolescentis |
| | FBI00011 | Bifidobacterium longum |
| | FBI00016 | Bifidobacterium pseudocatenulatum |
| | FBI00020 | Bacteroides thetaiotaomicron |
| | FBI00025 | Coprococcus comes |
| | FBI00027 | Fusicatenibacter saccharivorans |
| | FBI00030 | Eggerthella lenta |
| | FBI00047 | Eubacterium eligens |
| | FBI00052 | Bacteroides xylanisolvens |
| | FBI00053 | Lactobacillus rogosae |
| | FBI00056 | Clostridium citroniae |
| | FBI00062 | Collinsella aerofaciens |
| | FBI00078 | Blautia obeum |
| | FBI00096 | Eggerthella lenta |
| | FBI00104 | Blautia wexlerae |
| | FBI00110 | Lachnoclostridium pacaense |
| | FBI00111 | Bacteroides vulgatus |
| | FBI00113 | Parabacteroides merdae |
| | FBI00115 | Dorea formicigenerans |
| | FBI00116 | Ruminococcus faecis |
| | FBI00123 | Roseburia hominis |
| | FBI00124 | Anaerostipes hadrus |
| | FBI00126 | Bifidobacterium adolescentis |
| | FBI00135 | Bifidobacterium pseudocatenulatum |
| | FBI00147 | Clostridium bolteae |
| | FBI00159 | Eisenbergiella tayi |
| | FBI00167 | Dorea longicatena |
| | FBI00170 | Eggerthella lenta |
| | FBI00232 | Bacteroides stercoris |
| | FBI00255 | Hungatella hathewayi |
| | FBI00271 | Bacteroides xylanisolvens |
| DS4 | FBI00022 | Alistipes putredinis |
| | FBI00049 | Dialister succinatiphilus |
| | FBI00068 | Akkermansia muciniphila |
| | FBI00069 | Ruminococcus bromii |
| | FBI00152 | Dialister invisus |
| | FBI00165 | Bacteroides massiliensis |
| | FBI00171 | Bilophila wadsworthia |
| | FBI00175 | Holdemanella biformis |
| | FBI00177 | Parasutterella excrementihominis |
| | FBI00180 | Alistipes sp. FBI00180 |
| | FBI00182 | Bacteroides coprocola |
| | FBI00238 | Alistipes sp. FBI00238 |
| | FBI00269 | Alistipes putredinis |
| | FBI00274 | Eubacterium xylanophilum |
| | FBI00281 | Senegalimassilia anaerobia |
| DS5 (DS-OF1) | FBI00067 | Oxalobacter formigenes |
| DS6 (DS-OF2) | FBI00133 | Oxalobacter formigenes |
| DS7 (DS-OF3) | FBI00289 | Oxalobacter formigenes |

In certain embodiments, the Consortia is FB-003 as set forth in Table 2. In certain embodiments, the Consortia comprises the microbiota that are at least 97% or at least 98% identical to those listed in Table 2. In certain embodiments, the Consortia comprises a plurality of microbes (e.g., active microbes and supportive community microbes) designated as “FB-003.” In certain embodiments, the Consortia comprises a plurality of microbes listed in Table 2. In certain embodiments, the Consortia comprises at least 140 microbes, at least 141 microbes, or at least 142 microbes of FB-001. Table 2 is provided below:

TABLE 2
FB-003 Drug Substances

| Drug Substance (DS) | | |
|---|---|---|
| (aka CoCulture, CoC) | Strain ID | Strain Species |
| DS1 | Same as Table 1 | Same as Table 1 |
| DS2 | Same as Table 1 | Same as Table 1 |
| DS3 | Same as Table 1 | Same as Table 1 |
| DS4 | Same as Table 1 | Same as Table 1 |

In certain embodiments, the Consortia comprises the microbiota that are at least 97% or at least 98% identical to those listed in any of Consortia I-XIX or Tables 1 or 2.

In certain embodiments, the Consortia comprises a plurality of microbes (e.g., active microbes and supportive community microbes) designated as “Consortia A.” In certain embodiments, Consortia A comprises one or more strains of each of the species Anaerofustis stercorihominis, Anaerostipes hadrus, Bacteroides kribbi, Bacteroides stercoris, Bacteroides thetaiotaomicron, Bacteroides vulgatus, Bacteroides xylanisolvens, Bifidobacterium adolescentis, Bifidobacterium catenulatum, Bifidobacterium dentinum, Bifidobacterium longum, Bifidobacterium pseudocatenulatum, Blautia obeum, Blautia wexlerae, Butyricimonas faecihominis, Clostridium bolteae, Clostridium citroniae, Clostridium scindens, Collinsella aerofaciens, Coprococcus comes, Dorea formicigenerans, Dorea longicatena, Eggerthella lenta, Emergencia timonensis, Eubacterium eligens, Eubacterium hallii, Eubacterium ventriosum, Hungatella hathewayi, Parabacteroides distasonis, Parabacteroides merdae, Roseburia hominis, Ruminococcus faecis, Alistipes onderdonkii, Alistipes senegalensis, Alistipes shahii, Alistipes timonensis, Anaerotruncus massiliensis, Bacteroides caccae, Bacteroides fragilis, Bacteroides ovatus, Bacteroides uniformis, Bifidobacterium bifidum, Bilophila wadsworthia, Catabacter hongkongensis, Clostridium aldenense, Coprococcus eutactus, Dielma fastidiosa, Eubacterium rectale, Eubacterium siraeum, Faecalibacterium prausnitzii, Gordonibacter pamelaeae, Methanobrevibacter smithii, Monoglobus pectinilyticus, Paraprevotella clara, Ruminococcus bromii, Ruthenibacterium lactatiformans, Sutterella wadsworthensis, Akkermansia muciniphila, Bacteroides massiliensis, Dialister succinatiphilus, Eubacterium xylanophilum, Acidaminococcus intestine, Bacteroides faecis, Blautia faecis, Blautia hydrogenotrophica, Blautia massiliensis, Eisenbergiella tayi, Fusicatenibacter saccharivorans, Megasphaera massiliensis, Parasutterella excrementihominis, Phascolarctobacterium faecium, Ruminococcaceae sp. FBI00097, Ruminococcaceae sp. FBI00233, Sutterella massiliensis, Alistipes putredinis, Holdemanella biformis, Bifidobacterium dentium, Bacteroides finegoldii, Bacteroides nordii, Bacteroides salyersiae, Bacteroides stercorirosoris, Clostridiaceae sp. FBI00191, Clostridium clostridioforme, Clostridium fessum, Hungatella effluvia, Lachnoclostridium pacaense, Lachnospiraceae sp. FBI00033, Lachnospiraceae sp. FBI00071, Lachnospiraceae sp. FBI00290, Lactobacillus rogosae, Longicatena caecimuris, Barnesiella intestinihominis, Porphyromonas asaccharolytica, Acutalibacter timonensis, Turicibacter sanguinis, Alistipes sp. FBI00238, Bacteroides coprocola, Dialister invisus.

In certain embodiments, the Consortia comprises a plurality of microbes (e.g., active microbes and supportive community microbes) designated as “Consortia B.” In certain embodiments, Consortia B comprises one or more strains of each of the species Anaerofustis stercorihominis, Anaerostipes hadrus, Bacteroides kribbi, Bacteroides stercoris, Bacteroides thetaiotaomicron, Bacteroides vulgatus, Bacteroides xylanisolvens, Bifidobacterium adolescentis, Bifidobacterium catenulatum, Bifidobacterium dentinum, Bifidobacterium longum, Bifidobacterium pseudocatenulatum, Blautia obeum, Blautia wexlerae, Butyricimonas faecihominis, Clostridium bolteae, Clostridium citroniae, Clostridium scindens, Collinsella aerofaciens, Coprococcus comes, Dorea formicigenerans, Dorea longicatena, Eggerthella lenta, Emergencia timonensis, Eubacterium eligens, Eubacterium hallii, Eubacterium ventriosum, Hungatella hathewayi, Parabacteroides distasonis, Parabacteroides merdae, Roseburia hominis, Ruminococcus faecis, Alistipes onderdonkii, Alistipes senegalensis, Alistipes shahii, Alistipes timonensis, Anaerotruncus massiliensis, Bacteroides caccae, Bacteroides fragilis, Bacteroides ovatus, Bacteroides uniformis, Bifidobacterium bifidum, Bilophila wadsworthia, Catabacter hongkongensis, Clostridium aldenense, Coprococcus eutactus, Dielma fastidiosa, Eubacterium rectale, Eubacterium siraeum, Faecalibacterium prausnitzii, Gordonibacter pamelaeae, Methanobrevibacter smithii, Monoglobus pectinilyticus, Paraprevotella clara, Ruminococcus bromii, Ruthenibacterium lactatiformans, Sutterella wadsworthensis, Akkermansia muciniphila, Bacteroides massiliensis, Dialister succinatiphilus, Eubacterium xylanophilum, Acidaminococcus intestine, Bacteroides faecis, Blautia faecis, Blautia hydrogenotrophica, Blautia massiliensis, Eisenbergiella tayi, Fusicatenibacter saccharivorans, Megasphaera massiliensis, Parasutterella excrementihominis, Phascolarctobacterium faecium, Ruminococcaceae, Sutterella massiliensis, Alistipes putredinis, Holdemanella biformis, Bifidobacterium dentium, Bacteroides finegoldii, Bacteroides nordii, Bacteroides salyersiae, Bacteroides stercorirosoris, Clostridiaceae, Clostridium clostridioforme, Clostridium fessum, Hungatella effluvia, Lachnoclostridium pacaense, Lachnospiraceae, Lactobacillus rogosae, Longicatena caecimuris, Barnesiella intestinihominis, Porphyromonas asaccharolytica, Acutalibacter timonensis, Turicibacter sanguinis, Alistipes sp., Bacteroides coprocola, Dialister invisus.

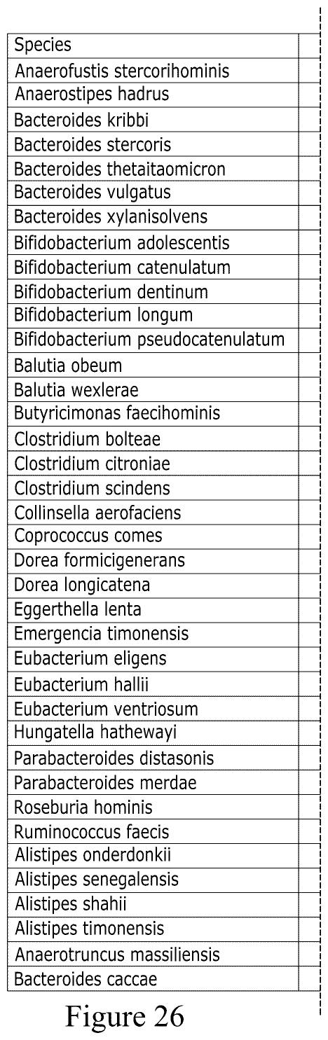

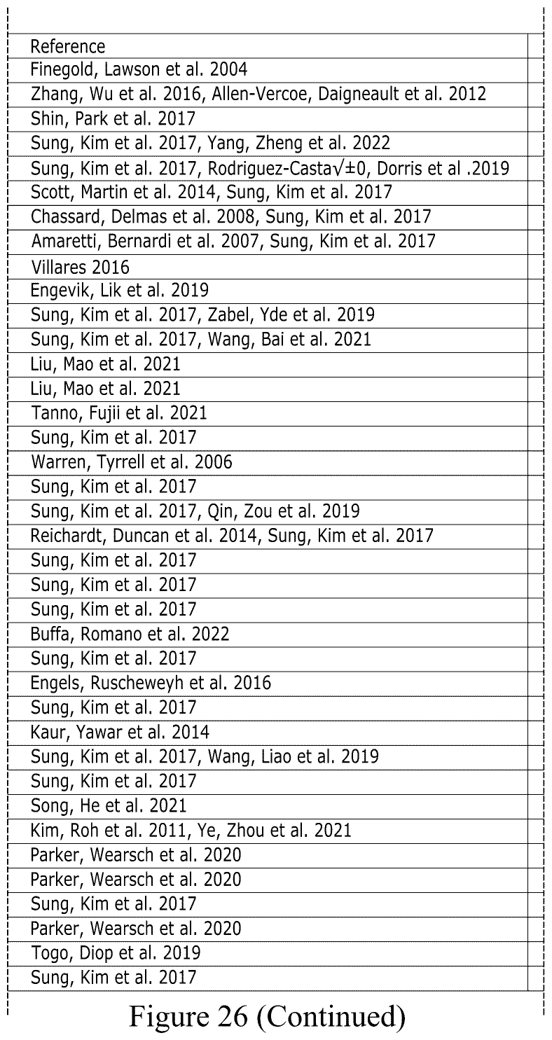

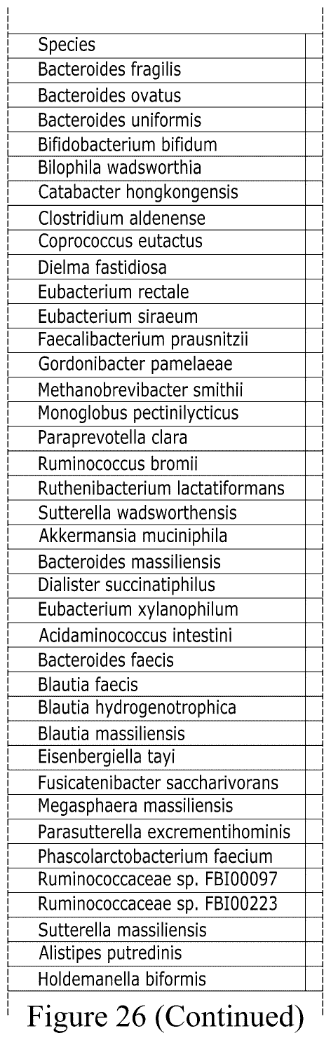

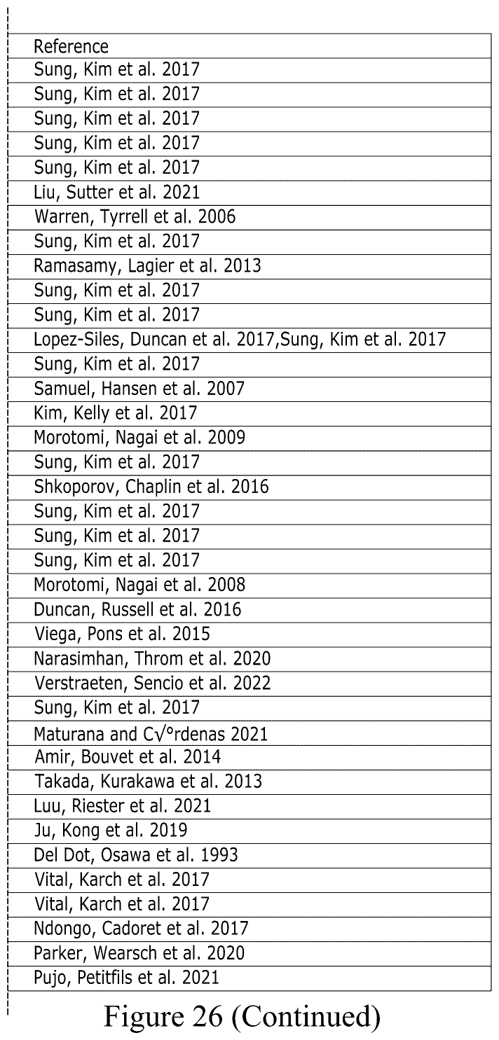

In certain embodiments, Consortia A and Consortia B comprise strains with the function of each species as described in FIG. 26.

In certain embodiments, Consortia A and Consortia B further comprise one or more O. formigenes strains. In certain embodiments, Consortia A and Consortia B comprise strains with the function of each species as described in FIG. 26 and one or more O. formigenes strains.

In certain embodiments, a Consortia comprises a microbial strain having a relative abundance of approximately 90%, 80%, 70%, 60%, 50%, 40%, 30%, 20%, 10%, 5%, 1%, 0.1%, 0.01%, 0.001%, 0.0001%, 0.00001%, or 0.000001% of the total microbial consortium. In certain embodiments, the relative abundance of a microbial strain is determined by metagenomic sequencing and calculated as the percentage of reads that are classified as an identified microbial strain, divided by the genome size. In certain embodiments, the relative abundance of a microbial strain of the present disclosure is determined by metagenomic shotgun sequencing.

### Active and Supportive Community of Microbes

The Consortia described herein comprise a plurality of active microbes.

Furthermore, the Consortia of the present disclosure further comprise a supportive community of microbes that enhances one or more characteristic of the plurality of active microbes. For example, in certain non-limiting embodiments, the supportive community of microbes enhances gastrointestinal engraftment of the plurality of active microbes. In other embodiments, the supportive community of microbes enhances biomass of the plurality of active microbes. In other embodiments, the supportive community of microbes enhances metabolism of the first metabolic substrate by the plurality of active microbes. In other embodiments, the supportive community of microbes enhances longitudinal stability of the plurality of active microbes.

The supportive community of microbes disclosed herein metabolize one or more metabolite produced by the plurality of active microbes, wherein the one or more metabolite inhibits metabolism of the plurality of active microbes. For example, in certain non-limiting embodiments, the supportive community of microbes metabolizes formate produced by the plurality of active microbes, wherein the presence of formate inhibits the metabolism of oxalate by the plurality of active microbes. In certain embodiments, the supportive community of microbes of the current disclosure catalyzes the fermentation of polysaccharides to one or more of the group consisting of acetate, acetoin, 2-oxoglutarate, propionate, 1,3-propanediol, succinate, ethanol, lactate, butyrate, 2,3-butanediol, acetone, butanol, formate, H₂, and CO₂. In certain embodiments, the supportive community of microbes catalyzes the fermentation of amino acids to one or more of the group consisting of acetate, propionate, butanoate, butyrate, isobutyrate, 2-methylbutyrate, isovalerate, isocaproate, 3-phenylpropanoate, phloretate, 3-(1H-indol-3-yl)propanoate, 5-aminopentanoate, H₂, H₂S, and CO₂. In certain embodiments, the supportive community catalyzes the synthesis of one or more of the group consisting of methane from H₂ and CO₂, methane from formate and H₂, acetate from H₂ and CO₂, acetate from formate and H₂, acetate and sulfide from H₂, CO₂, and sulfate, propionate and CO₂ from succinate, succinate from H₂ and fumarate; synthesis of succinate from formate and fumarate, and butyrate, acetate, H₂, and CO₂ from lactate. In certain embodiments, the supportive community of microbes of the current disclosure catalyzes the deconjugation of conjugated bile acids to produce primary bile acids, the conversion of cholic acid (CA) to 7-oxocholic acid, the conversion of 7-oxocholic acid to 7-beta-cholic acid (7betaCA), the conversion of chenodeoxycholic acid (CDCA) to 7-oxochenodeoxycholic acid, and/or the conversion of 7-oxochenodeoxycholic acid to ursodeoxycholic acid (UDCA).

### Consortia Design

In certain embodiments, microbial consortia disclosed herein are designed to meet one or more of the following criteria:
(i) an ability to eliminate or reduce levels of a first metabolic substrate causing or contributing to a disease in an animal;
(ii) an ability to metabolize or convert one or more metabolite produced by the metabolism of the first metabolic substrate;
(iii) an ability to metabolize one or more nutrient typically found in the human diet;
(iv) an ability to fulfill unique and potentially beneficial biological functions in the gastrointestinal (GI) tract (e.g., bile salt hydrolase activity or butyrate production);
(v) an ability to engraft in various biological niches and physical and metabolic compartments of the GI tract of an animal;
(vi) an ability to increase biomass upon engraftment in the GI tract;
(vii) an ability to have longitudinal stability in the GI tract of an animal;
(viii) an ability to increase the flux of a precursor of the first metabolic substrate into a biochemical pathway that converts said precursor into a metabolite that is not the first metabolic substrate;
(ix) diversity of component microbial species across one or more taxonomic phyla; and
(x) natural prevalence of component microbial species in the GI tract of healthy adults.

In certain embodiments, the microbial consortia of the present disclosure are designed to comprise a plurality of active microbes capable of metabolizing a first metabolic substrate that causes or contributes to disease in an animal. In certain embodiments, the first metabolic substrate may be selected from, but not limited to, oxalate and a bile acid (e.g., lithocholic acid (LCA), deoxycholic acid (DCA)). In certain embodiments, the microbial consortium is designed to be capable of metabolizing the first metabolic substrate across a variety of pH ranges found within the GI tract (e.g., pH 4 to 8). In certain embodiments, the microbial consortium is designed to be capable of metabolizing the first metabolic substrate in the presence of various concentrations of first metabolic substrate as they exist in different regions of the GI tract.

In certain embodiments, the Consortia is FB-001 (e.g., disclosed in Table 1) or a functional equivalent thereof. In certain embodiments, FB-001 is defined by its function. In certain embodiments, FB-001 is defined by its function as set forth in Tables 3 and/or 4. In certain embodiments, FB-001 is defined by its function as set forth in Tables 3 and 4. In certain embodiments, FB-001 is defined by its function as set forth in Table 3 or 4. In certain embodiments, FB-001 is defined by its function as set forth in Tables 14, 15, and 16. In certain embodiments, FB-001 is defined by its function as set forth in one or more of Tables 14, 15, and 16. In certain embodiments, FB-001 is defined by its function as set forth in Tables 3, 4, 14, 15, and 16. In certain embodiments, FB-001 is defined by its function as set forth in one or more of Tables 3, 4, 14, 15, and 16. In certain embodiments, methods for determining function of FB-001 are provided in Examples 5 and 6.

In certain embodiments, the Consortia is FB-003 (e.g., disclosed in Table 2) or a functional equivalent thereof. In certain embodiments, FB-003 is defined by its function. In certain embodiments, FB-003 is defined by its function as set forth in Tables 3 and/or 4. In certain embodiments, FB-003 is defined by its function as set forth in Tables 3 and 4. In certain embodiments, FB-003 is defined by its function as set forth in Table 3 or 4. In certain embodiments, FB-003 is defined by its function as set forth in Tables 14, 15, and 16. In certain embodiments, FB-003 is defined by its function as set forth in one or more of Tables 14, 15, and 16. In certain embodiments, FB-003 is defined by its function as set forth in Tables 3, 4, 14, 15, and 16. In certain embodiments, FB-003 is defined by its function as set forth in one or more of Tables 3, 4, 14, 15, and 16.

In certain embodiments, the Consortia is Consortia A or B or a functional equivalent thereof. In certain embodiments, Consortia A and B are defined by its function. In certain embodiments, FB-003 is the consortia of Consortia A or Consortia B. In certain embodiments, Consortia A and B are defined by its function as set forth in Tables 3 and/or 4. In certain embodiments, Consortia A and B are defined by its function as set forth in Tables 3 and 4. In certain embodiments, Consortia A and B are defined by its function as set forth in Table 3 or 4. In certain embodiments, Consortia A and B are defined by its function as set forth in Tables 14, 15, and 16. In certain embodiments, Consortia A and B are defined by its function as set forth in one or more of Tables 14, 15, and 16. In certain embodiments, Consortia A and B are defined by its function as set forth in Tables 3, 4, 14, 15, and 16. In certain embodiments, Consortia A and B are defined by its function as set forth in one or more of Tables 3, 4, 14, 15, and 16.

### Methods of Preparation

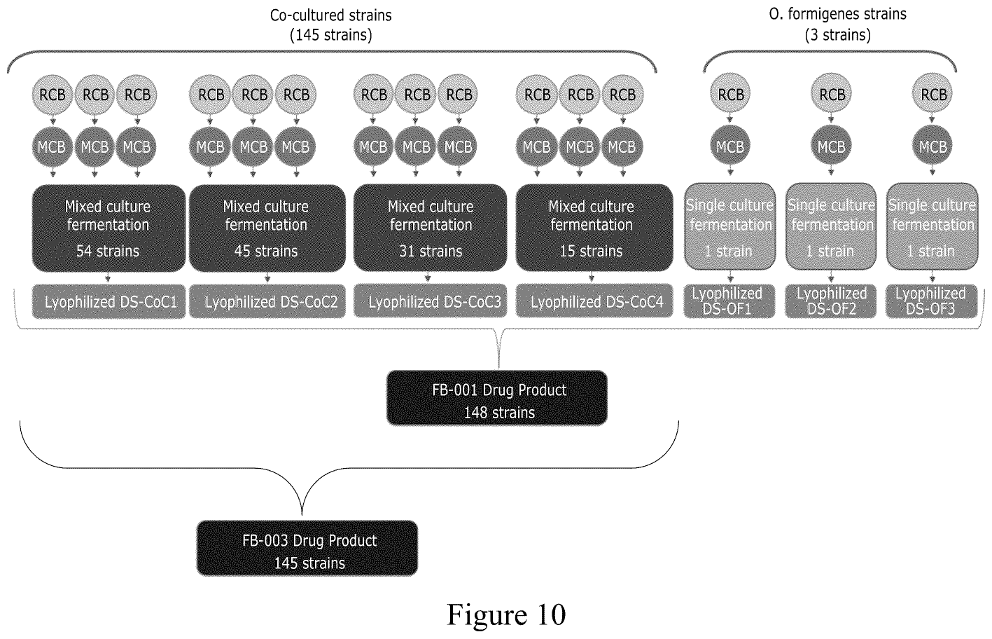

The present disclosure also provides methods for preparing and/or manufacturing the microbial consortia described herein. FIGS. 10-12 illustrate certain methods for the preparation and manufacturing of the microbial consortia described herein.

In certain embodiments, the methods comprise obtaining a donor stool and preparing a stool dilution. In certain embodiments, the stool dilution is plated onto an agar plate. In certain embodiments, the agar plate includes an anaerobic media. In certain embodiments, the agar plate includes colonies. Characterization and quality analysis of these colonies can be performed. For example, but without any limitation, 16s RNA and/or MALDI mass spectrometry could be performed. In certain embodiments, the characterized colonies can be further expanded in a broth culture. After growth and expansion, the microbes can be stored in vials for further use.

In certain embodiments, the microbes can be further expanded in a bioreactor including a cell culture medium. In certain embodiments, the cell culture medium can include:
a) soytone, D-cellobiose, yeast extract, dextrose (glucose), maltose monohydrate, magnesium sulfate heptahydrate, calcium chloride dihydrate, potassium phosphate monobasic, potassium phosphate dibasic, sodium chloride, sodium bicarbonate, volatile fatty acid solution, L-cysteine HCl monohydrate, hemin solution, vitamin solution, or a combination thereof; or
b) soytone, D-cellobiose, yeast extract, dextrose (glucose), maltose monohydrate, magnesium sulfate heptahydrate, calcium chloride dihydrate, potassium phosphate monobasic, potassium phosphate dibasic, sodium chloride, ammonium sulfate, sodium bicarbonate, volatile fatty acid solution, L-cysteine HCl monohydrate, hemin solution, vitamin solution, or a combination thereof.

In certain embodiments, the cell culture medium is YCFAC. In certain embodiments, the cell culture medium further comprises threonine.

In certain embodiments, the microbes can be expanded in a bioreactor in anaerobic conditions. In certain embodiments, the microbes can be expanded in a bioreactor in the presence of gas overlay. In certain embodiments, the microbes can be expanded in a bioreactor in absence of gas sparing.

In certain embodiments, the methods include expanding microbes in mixed cultures.

In certain embodiments, the methods comprise expanding microbes in a first mixed culture or composition comprising:
a) Clostridium citroniae, Bacteroides salyersiae, Blautia obeum, Parabacteroides merdae, Parabacteroides distasonis, Anaerostipes hadrus, Lachnospiraceae sp. FBI00033, Eubacterium eligens, Bifidobacterium dentium, Blautia wexlerae, Fusicatenibacter saccharivorans, Bacteroides nordii, Dorea formicigenerans, Dorea longicatena, Bacteroides stercorirosoris, Bifidobacterium longum, Bacteroides kribbi, Lachnospiraceae sp. FBI00071, Bacteroides thetaiotaomicron, Clostridium clostridioforme, Clostridium scindens, Roseburia hominis, Clostridium fessum, Coprococcus comes, Blautia faecis, Hungatella hathewayi, Bacteroides stercoris, Collinsella aerofaciens, Hungatella effluvii, Bifidobacterium adolescentis, Bifidobacterium catenulatum, Lactobacillus rogosae, Bacteroides faecis, Bacteroides finegoldii, Clostridiaceae sp. FBI00191, Ruminococcus faecis, Lachnoclostridium pacaense, Clostridium bolteae, Longicatena caecimuris, Eggerthella lenta, Blautia massiliensis, Bacteroides xylanisolvens, Bacteroides vulgatus, Megasphaera massiliensis, Butyricimonas faecihominis, Eisenbergiella tayi, Acidaminococcus intestini, Emergencia timonensis, Bifidobacterium pseudocatenulatum, Eubacterium hallii, Anaerofustis stercorihominis, Eubacterium ventriosum, Blautia hydrogenotrophica, and Lachnospiraceae sp. FBI00290, or a functional equivalent thereof; or
b) FBI00001, FBI00002, FBI00010, FBI00013, FBI00029, FBI00032, FBI00033, FBI00034, FBI00043, FBI00044, FBI00048, FBI00050, FBI00051, FBI00057, FBI00059, FBI00060, FBI00070, FBI00071, FBI00076, FBI00079, FBI00087, FBI00093, FBI00102, FBI00109, FBI00117, FBI00120, FBI00125, FBI00127, FBI00128, FBI00145, FBI00162, FBI00174, FBI00184, FBI00190, FBI00191, FBI00194, FBI00198, FBI00199, FBI00200, FBI00201, FBI00205, FBI00206, FBI00211, FBI00220, FBI00221, FBI00236, FBI00245, FBI00248, FBI00251, FBI00254, FBI00267, FBI00278, FBI00288, and FBI00290, or a functional equivalent thereof.

In certain embodiments, the methods comprise expanding microbes in a second mixed culture or composition comprising:
a) Acutalibacter timonensis, Alistipes onderdonkii, Bacteroides uniformis, Eubacterium rectale, Alistipes timonensis, Bacteroides kribbi, Coprococcus eutactus, Bilophila wadsworthia, Bacteroides caccae, Alistipes shahii, Parasutterella excrementihominis, Paraprevotella clara, Sutterella wadsworthensis, Sutterella massiliensis, Porphyromonas asaccharolytica, Ruminococcus bromii, Monoglobus pectinilyticus, Ruminococcaceae sp. FBI00097, Gordonibacter pamelaeae, Bacteroides uniformis, Gordonibacter pamelaeae, Bacteroides fragilis, Phascolarctobacterium faecium, Monoglobus pectinilyticus, Clostridium aldenense, Ruthenibacterium lactatiformans, Bacteroides ovatus, Bifidobacterium bifidum, Anaerotruncus massiliensis, Clostridium aldenense, Sutterella wadsworthensis, Catabacter hongkongensis, Alistipes senegalensis, Ruminococcaceae sp. FBI00233, Alistipes shahii, Dielma fastidiosa, Eubacterium siraeum, Faecalibacterium prausnitzii, Turicibacter sanguinis, Eubacterium rectale, Bacteroides caccae, Methanobrevibacter smithii, Barnesiella intestinihominis, Alistipes onderdonkii, and Methanobrevibacter smithii, or a functional equivalent thereof; or
b) FBI00004, FBI00012, FBI00015, FBI00018, FBI00019, FBI00021, FBI00038, FBI00040, FBI00046, FBI00061, FBI00066, FBI00075, FBI00077, FBI00080, FBI00081, FBI00085, FBI00092, FBI00097, FBI00099, FBI00112, FBI00132, FBI00137, FBI00140, FBI00149, FBI00151, FBI00176, FBI00189, FBI00197, FBI00208, FBI00212, FBI00224, FBI00226, FBI00229, FBI00233, FBI00235, FBI00237, FBI00243, FBI00244, FBI00258, FBI00260, FBI00263, FBI00270, FBI00273, FBI00277, and FBI00292, or a functional equivalent thereof.

In certain embodiments, the methods comprise expanding microbes in a third mixed culture or composition comprising:
a) Bifidobacterium adolescentis, Bifidobacterium longum, Bifidobacterium pseudocatenulatum, Bacteroides thetaiotaomicron, Coprococcus comes, Fusicatenibacter saccharivorans, Eggerthella lenta, Eubacterium eligens, Bacteroides xylanisolvens, Lactobacillus rogosae, Clostridium citroniae, Collinsella aerofaciens, Blautia obeum, Eggerthella lenta, Blautia wexlerae, Lachnoclostridium pacaense, Bacteroides vulgatus, Parabacteroides merdae, Dorea formicigenerans, Ruminococcus faecis, Roseburia hominis, Anaerostipes hadrus, Bifidobacterium adolescentis, Bifidobacterium pseudocatenulatum, Clostridium bolteae, Eisenbergiella tayi, Dorea longicatena, Eggerthella lenta, Bacteroides stercoris, Hungatella hathewayi, and Bacteroides xylanisolvens, or a functional equivalent thereof; or
b) FBI00009, FBI00011, FBI00016, FBI00020, FBI00025, FBI00027, FBI00030, FBI00047, FBI00052, FBI00053, FBI00056, FBI00062, FBI00078, FBI00096, FBI00104, FBI00110, FBI00111, FBI00113, FBI00115, FBI00116, FBI00123, FBI00124, FBI00126, FBI00135, FBI00147, FBI00159, FBI00167, FBI00170, FBI00232, FBI00255, and FBI00271, or a functional equivalent thereof.

In certain embodiments, the methods comprise expanding microbes in a fourth mixed culture or composition comprising:
a) Alistipes putredinis, Dialister succinatiphilus, Akkermansia muciniphila, Ruminococcus bromii, Dialister invisus, Bacteroides massiliensis, Bilophila wadsworthia, Holdemanella biformis, Parasutterella excrementihominis, Alistipes sp. FBI00180, Bacteroides coprocola, Alistipes sp. FBI00238, Alistipes putredinis, Eubacterium xylanophilum, and Senegalimassilia anaerobia, or a functional equivalent thereof; or
b) FBI00022, FBI00049, FBI00068, FBI00069, FBI00152, FBI00165, FBI00171, FBI00175, FBI00177, FBI00180, FBI00182, FBI00238, FBI00269, FBI00274, and FBI00281, or a functional equivalent thereof.

In certain embodiments, the methods include expanding each of the three O. formigenes microbes of FB-001 in single cultures. In certain embodiments, the methods comprise expanding microbes in a first single culture (or fifth composition) comprising a) a first O. formigenes strain; or b) FBI00067 or a functional equivalent thereof. In certain embodiments, the methods comprise expanding microbes in a second single culture (or sixth composition) comprising a) a second O. formigenes strain; or b) FBI00133 or a functional equivalent thereof. In certain embodiments, the methods comprise expanding microbes in a third single culture (or seventh composition) comprising a) a third O. formigenes strain; or b) FBI00289 or a functional equivalent thereof.

In certain embodiments, the methods comprise lyophilizing cultures and compositions described herein. In certain embodiments, the cultures and compositions comprises a lyoprotectant. In certain embodiments, the lyoprotectant comprises maltodextrin. In certain embodiments, the lyoprotectant comprises inulin. In certain embodiments, the lyoprotectant comprises maltodextrin and inulin. In certain embodiments, the maltodextrin is present at a concentration of about 8%. In certain embodiments, the inulin is present at a concentration of about 0.5%.

In certain embodiments, the methods comprise blending and/or mixing lyophilized cultures and compositions outlined above. Additional information on the strains for each composition can be found in Table 1 or Table 2.

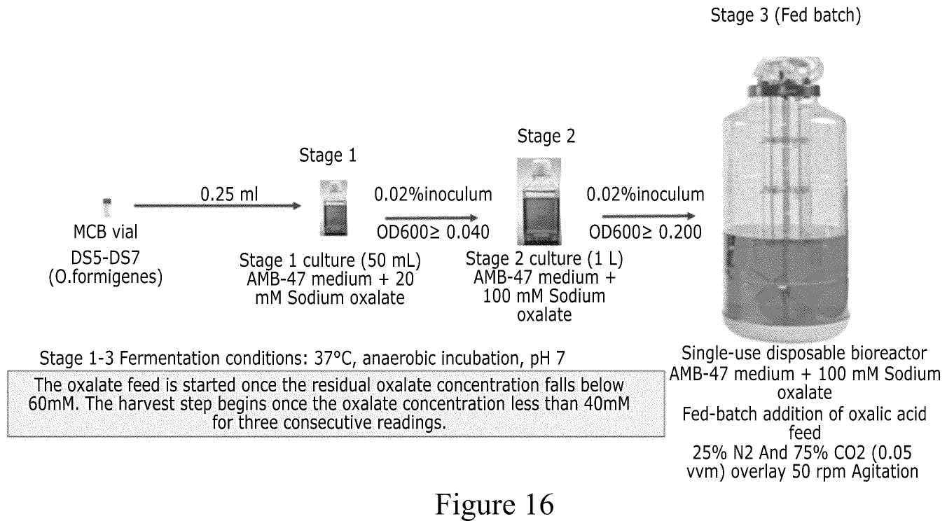

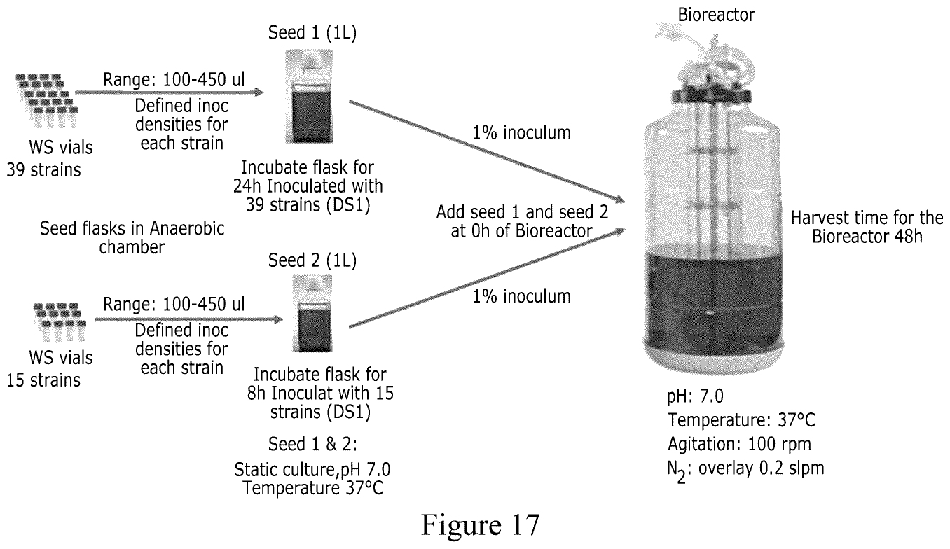

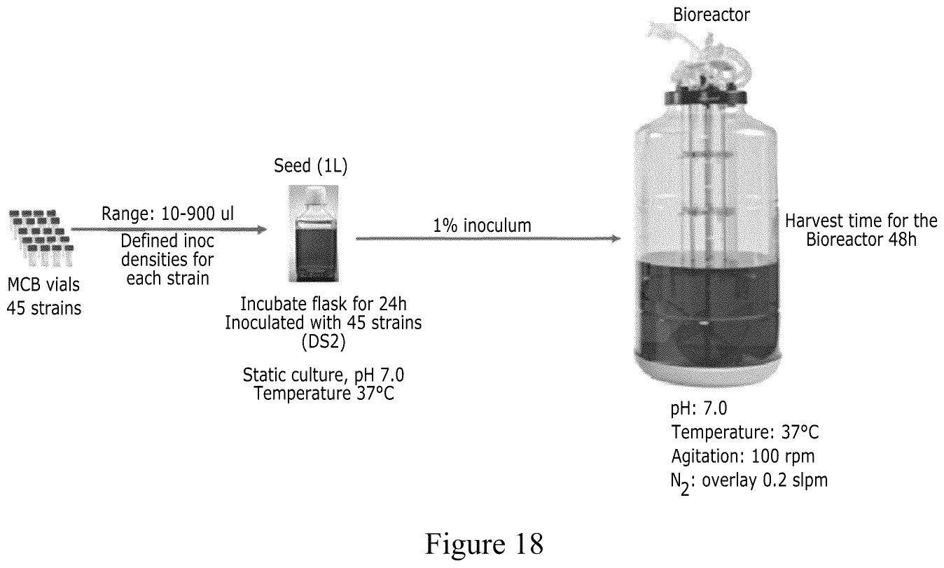

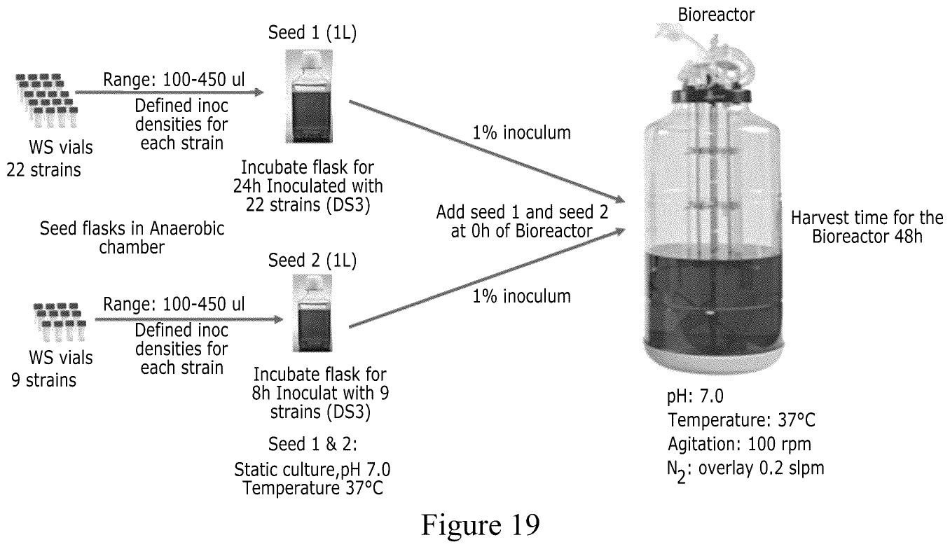

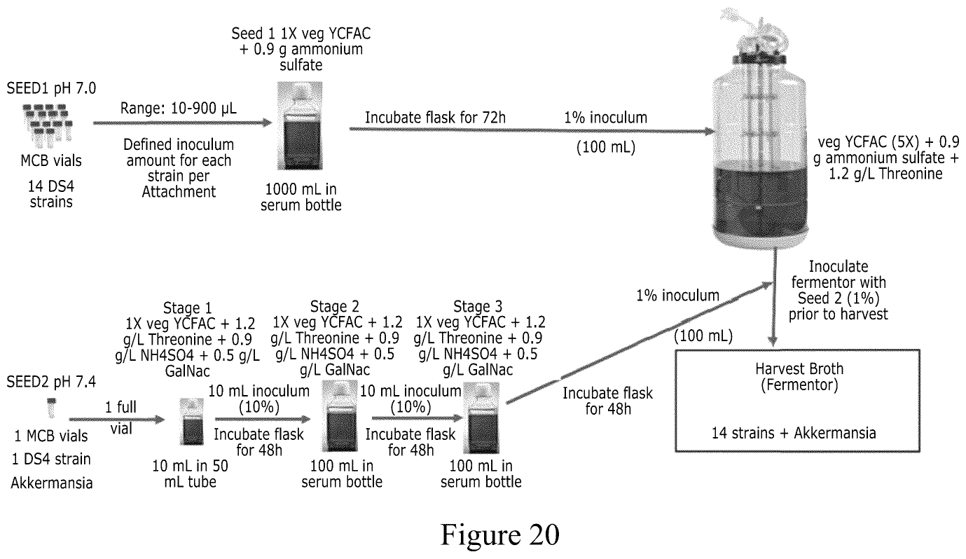

In certain embodiments, DS1 as described in Table 1 is prepared using the method described in FIG. 17. In certain embodiments, DS2 as described in Table 1 is prepared using the method described in FIG. 18. In certain embodiments, DS3 as described in Table 1 is prepared using the method described in FIG. 19. In certain embodiments, DS4 as described in Table 1 is prepared using the method described in FIG. 20. In certain embodiments, for FB-001, DS5-DS7 (i.e., the manufacture of 0.formigenes) as described in Table 21 are prepared using the method described in FIG. 16. In certain embodiments, the manufacture of FB-001 and FB-003 comprise the separate manufacture of each of DS1-DS4 (and DS5-DS7 for FB-001) as described in FIGS. 16-20, followed by blending to achieve a uniform distribution of each of the DSs. In certain embodiments, the blending of DS1-DS4 (for FB-003) and DS1-DS7 (for FB-003) is followed by encapsulation for oral administration.

### Pharmaceutical Compositions

The present disclosure also provides pharmaceutical compositions that contain an effective amount of a microbial consortium described herein. The composition can be formulated for use in a variety of delivery systems. One or more physiologically acceptable buffer(s) or carrier(s) can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

In certain embodiments, the presently disclosed pharmaceutical composition comprises Consortia I. In certain embodiments, the presently disclosed pharmaceutical composition comprises Consortia II. In certain embodiments, the presently disclosed pharmaceutical composition comprises Consortia I. In certain embodiments, the presently disclosed pharmaceutical composition comprises Consortia III. In certain embodiments, the presently disclosed pharmaceutical composition comprises Consortia IV. In certain embodiments, the presently disclosed pharmaceutical composition comprises Consortia V. In certain embodiments, the presently disclosed pharmaceutical composition comprises Consortia VI. In certain embodiments, the presently disclosed pharmaceutical composition comprises Consortia VII. In certain embodiments, the presently disclosed pharmaceutical composition comprises Consortia VIII. In certain embodiments, the presently disclosed pharmaceutical composition comprises Consortia IX. In certain embodiments, the presently disclosed pharmaceutical composition comprises Consortia X. In certain embodiments, the presently disclosed pharmaceutical composition comprises Consortia XI. In certain embodiments, the presently disclosed pharmaceutical composition comprises Consortia XII. In certain embodiments, the presently disclosed pharmaceutical composition comprises Consortia XIII. In certain embodiments, the presently disclosed pharmaceutical composition comprises Consortia XIV. In certain embodiments, the presently disclosed pharmaceutical composition comprises Consortia XV. In certain embodiments, the presently disclosed pharmaceutical composition comprises Consortia XVI. In certain embodiments, the presently disclosed pharmaceutical composition comprises Consortia XVII. In certain embodiments, the presently disclosed pharmaceutical composition comprises Consortia XVIII. In certain embodiments, the presently disclosed pharmaceutical composition comprises Consortia XIX. In certain embodiments, the presently disclosed pharmaceutical composition comprises Consortia A. In certain embodiments, the presently disclosed pharmaceutical composition comprises Consortia B.

In certain embodiments, the presently disclosed pharmaceutical composition comprises FB-001. In certain embodiments, the presently disclosed pharmaceutical composition comprises FB-003.

In certain embodiments, the presently disclosed pharmaceutical composition comprises an amount of at least about 10, at least about 10², at least about 10³, at least about 10⁴, at least about 10⁵, at least about 10⁶, at least about 10⁷, at least about 10⁸, at least about 10⁹, at least about 10¹⁰, at least about 10¹¹, at least about 10¹², or at least about 10¹³ microbes of each strain (e.g., each strain of FB-001 or FB-003). In certain embodiments, the presently disclosed pharmaceutical composition comprises between about 10 and about 10¹³, between about 10² and about 10¹³, between about 10³ and about 10¹³, between about 10⁴ and about 10¹³, between about 10⁵ and about 10¹³, between about 10⁶ and about 10¹³, between about 10⁷ and about 10¹³, between about 10⁸ and about 10¹³, between about 10⁹ and about 10¹³, between about 10¹¹ and about 10¹³, between about 10¹¹ and about 10¹³, between about 10¹² and about 10¹³, between about 10² and about 10¹², between about 10² and about 10¹¹, between about 10² and about 10¹⁰, between about 10² and about 10⁹, between about 10² and about 10⁸, between about 10² and about 10⁷, between about 10² and about 10⁶, between about 10² and about 10⁵, between about 10² and about 10⁴, between about 10² and about 10³, between about 10² and about 10⁹, between about 10³ and about 10⁹, between about 10⁴ and about 10⁹, between about 10⁵ and about 10⁹, between about 10⁶ and about 10⁹, between about 10⁷ and about 10⁹, between about 10⁸ and about 10⁹, between about 10² and about 10⁶, between about 10³ and about 10⁶, between about 10⁴ and about 10⁶, or between about 10⁵ and about 10⁶ microbes for each strain (e.g., each strain of FB-001 or FB-003). In certain embodiments, each strain can be present in different amounts. For example, but without any limitation, a pharmaceutical composition comprising a first strain, a second strain, and a third strain can comprise 10 microbes of the first strain, 10⁷ microbes of the second strain, 10³ microbes of the third strain.

In certain embodiments, the presently disclosed pharmaceutical composition comprises an amount of at least about 10, at least about 10², at least about 10³, at least about 10⁴, at least about 10⁵, at least about 10⁶, at least about 10⁷, at least about 10⁸, at least about 10⁹, at least about 10¹⁰, at least about 10¹¹, at least about 10¹², or at least about 10¹³ CFUs of each strain (e.g., each strain of FB-001 or FB-003). In certain embodiments, the presently disclosed pharmaceutical composition comprises between about 10 and about 10¹³, between about 10² and about 10¹³, between about 10³ and about 10¹³, between about 10⁴ and about 10¹³, between about 10⁵ and about 10¹³, between about 10⁶ and about 10¹³, between about 10⁷ and about 10¹³, between about 10⁸ and about 10¹³, between about 10⁹ and about 10¹³, between about 10¹⁰ and about 10¹³, between about 10¹¹ and about 10¹³, between about 10¹² and about 10¹³, between about 10² and about 10¹², between about 10² and about 10¹¹, between about 10² and about 10¹⁰, between about 10² and about 10⁹, between about 10² and about 10⁸, between about 10² and about 10⁷, between about 10² and about 10⁶, between about 10² and about 10⁵, between about 10² and about 10⁴, between about 10² and about 10³, between about 10² and about 10⁹, between about 10³ and about 10⁹, between about 10⁴ and about 10⁹, between about 10⁵ and about 10⁹, between about 10⁶ and about 10⁹, between about 10⁷ and about 10⁹, between about 10⁸ and about 10⁹, between about 10² and about 10⁶, between about 10³ and about 10⁶, between about 10⁴ and about 10⁶, or between about 10⁵ and about 10⁶ CFUs for each strain (e.g., each strain of FB-001 or FB-003). In certain embodiments, each strain can be present in different amounts. For example, but without any limitation, a pharmaceutical composition comprising a first strain, a second strain, and a third strain can comprise 10 CFUs of the first strain, 10⁷ CFUs of the second strain, 10³ CFUs of the third strain.

In certain embodiments, the presently disclosed pharmaceutical composition comprises an amount of at least about 10⁻¹, at least about 10⁻², at least about 10⁻³, at least about 10⁴, at least about 10⁻⁵, at least about 10⁻⁶, at least about 10⁻⁷, at least about 10⁻⁸, at least about 10⁻⁹, at least about 10⁻¹⁰, at least about 10⁻¹¹, or at least about 10⁻¹², or at least about 10¹³ grams of each strain (e.g., each strain of FB-001 or FB-003). In certain embodiments, each strain can be present in different amounts. For example, but without any limitation, a pharmaceutical composition comprising a first strain, a second strain, and a third strain can comprise 10⁴ grams of the first strain, 10⁻³ grams of the second strain, 10⁻⁶ grams of the third strain.

In certain embodiments, the presently disclosed pharmaceutical composition comprises a total amount of at least about 10, at least about 10², at least about 10³, at least about 10⁴, at least about 10⁵, at least about 10⁶, at least about 10⁷, at least about 10⁸, at least about 10⁹, at least about 10¹⁰, at least about 10¹¹, at least about 10¹², or at least about 10¹³ microbes (e.g., total amount of microbes of FB-001 or total amount of microbes FB-003). In certain embodiments, the presently disclosed pharmaceutical composition comprises a total amount of between about 10 and about 10¹³, between about 10² and about 10¹³, between about 10³ and about 10¹³, between about 10⁴ and about 10¹³, between about 10⁵ and about 10¹³, between about 10⁶ and about 10¹³, between about 10⁷ and about 10¹³, between about 10⁸ and about 10¹³, between about 10⁹ and about 10¹³, between about 10¹⁰ and about 10¹³, between about 10¹¹ and about 10¹³, between about 10¹² and about 10¹³, between about 10² and about 10¹², between about 10² and about 10¹¹, between about 10² and about 10¹⁰, between about 10² and about 10⁹, between about 10² and about 10⁸, between about 10² and about 10⁷, between about 10² and about 10⁶, between about 10² and about 10⁵, between about 10² and about 10⁴, between about 10² and about 10³, between about 10² and about 10⁹, between about 10³ and about 10⁹, between about 10⁴ and about 10⁹, between about 10⁵ and about 10⁹, between about 10⁶ and about 10⁹, between about 10⁷ and about 10⁹, between about 10⁸ and about 10⁹, between about 10² and about 10⁶, between about 10³ and about 10⁶, between about 10⁴ and about 10⁶, or between about 10⁵ and about 10⁶ microbes.

In certain embodiments, the presently disclosed pharmaceutical composition comprises a total amount of at least about 10, at least about 10², at least about 10³, at least about 10⁴, at least about 10⁵, at least about 10⁶, at least about 10⁷, at least about 10⁸, at least about 10⁹, at least about 10¹⁰, at least about 10¹¹, at least about 10¹², or at least about 10¹³ CFUs (e.g., total CFUs of FB-001 or total CFUs of FB-003). In certain embodiments, the presently disclosed pharmaceutical composition comprises a total amount of between about 10 and about 10¹³, between about 10² and about 10¹³, between about 10³ and about 10¹³, between about 10⁴ and about 10¹³, between about 10⁵ and about 10¹³, between about 10⁶ and about 10¹³, between about 10⁷ and about 10¹³, between about 10⁸ and about 10¹³, between about 10⁹ and about 10¹³, between about 10¹⁰ and about 10¹³, between about 10¹¹ and about 10¹³, between about 10¹² and about 10¹³, between about 10² and about 10¹², between about 10² and about 10¹¹, between about 10² and about 10¹⁰, between about 10² and about 10⁹, between about 10² and about 10⁸, between about 10² and about 10⁷, between about 10² and about 10⁶, between about 10² and about 10⁵, between about 10² and about 10⁴, between about 10² and about 10³, between about 10² and about 10⁹, between about 10³ and about 10⁹, between about 10⁴ and about 10⁹, between about 10⁵ and about 10⁹, between about 10⁶ and about 10⁹, between about 10⁷ and about 10⁹, between about 10⁸ and about 10⁹, between about 10² and about 10⁶, between about 10³ and about 10⁶, between about 10⁴ and about 10⁶, or between about 10⁵ and about 10⁶ CFUs. In certain embodiments, the presently disclosed pharmaceutical composition comprises between about 10⁵ and about 10¹³, between about 10⁶ and about 10¹³, between about 10⁷ and about 10¹³, between about 10⁸ and about 10¹³, between about 10⁹ and about 10¹³, between about 10¹⁰ and about 10¹³, between about 10¹¹ and about 10¹³, between about 10¹² and about 10¹³, between about 10⁵ and about 10¹², between about 10⁶ and about 10¹², between about 10⁷ and about 10¹², between about 10⁸ and about 10¹², between about 10⁹ and about 10¹², between about 10¹⁰ and about 10¹², between about 10¹¹ and about 10¹², between about 10⁵ and about 10¹¹, between about 10⁶ and about 10¹¹, between about 10⁷ and about 10¹¹, between about 10⁸ and about 10¹¹, between about 10⁹ and about 10¹¹, between about 10¹⁰ and about 10¹¹, between about 10⁵ and about 10¹⁰, between about 10⁶ and about 10¹⁰, between about 10⁷ and about 10¹⁰, between about 10⁸ and about 10¹⁰, between about 10⁹ and about 10¹⁰, between about 10⁵ and about 10⁹, between about 10⁶ and about 10⁹, between about 10⁷ and about 10⁹, between about 10⁸ and about 10⁹, between about 5×10⁹ and about 5×10¹⁰, between about 5×10⁹ and about 5×10¹¹, between about 5×10⁹ and about 5×10¹², between about 5×10¹⁰ and about 5×10¹², between about 5×10¹¹ and about 5×10¹², or between about 5×10¹⁰ and about 5×10¹¹ viable cells. In certain embodiments, the presently disclosed pharmaceutical composition comprises between about 5×10⁹ and about 5×10¹² viable cells. In certain embodiments, the presently disclosed pharmaceutical composition comprises between about 5×10⁹ and about 5×10¹⁰ viable cells. In certain embodiments, the presently disclosed pharmaceutical composition comprises between about 5×10¹⁰ and about 5×10¹¹ viable cells. In certain embodiments, the presently disclosed pharmaceutical composition comprises between about 5×10¹¹ and about 5×10¹² viable cells.

In certain embodiments, the presently disclosed pharmaceutical composition comprises up to about 10⁵, up to about 10⁶, up to about 10⁷, up to about 10⁸, up to about 10⁹, up to about 10¹⁰, up to about 10¹¹, up to about 10¹², or up to about 10¹³ viable cells. In certain embodiments, the presently disclosed pharmaceutical composition comprises up to about 10¹¹ viable cells. In certain embodiments, the presently disclosed pharmaceutical composition comprises up to about 10¹² viable cells.

In certain embodiments, the presently disclosed pharmaceutical composition can be present in the form of a food product comprising a consortia disclosed herein (e.g., FB-001 or FB-003). As used herein, the term “food product” refers to a composition intended for ingestion by an individual (e.g., a human subject). Non-limiting examples of food products encompassed by the present disclosure include juices, refreshing beverages, tea beverages, drink preparations, jelly beverages, functional beverages, milk, dairy beverages, ice creams, cheeses, yogurts, biscuits, cookies, candies, chewing gums, gummies, jellies, cream caramels, frozen desserts, and instant foods. Further, the examples also include health foods and beverages prepared in the forms of powders, granules, tablets, capsules, liquids, pastes, and jellies. In certain embodiments, the food product comprising a presently disclosed consortia further comprises a prebiotic. As used herein, the term “prebiotic” refers to a substance that can promote the growth of the microbes of the consortia. Non-limiting examples of prebiotic include fructose, galactose, mannose, soy, inulin, dietary fibers, or a combination thereof.

In certain embodiments, microbial cells of the present disclosure are harvested by microfiltration and centrifugation. In certain embodiments, microfiltration is done with a membrane comprising a nonreactive polymer. For example, in certain non-limiting embodiments, said membrane comprises Polyvinylidene fluoride, Polysulfones, or nitrocellulose. In certain embodiments, a membrane for microfiltration has a pore size from about 0.2 μm to about 0.45 μm. In certain embodiments, the cells are centrifuged at from about 1000 g to about 30000 g, from about 5000 g to about 30000 g, from about 10000 g to about 30000 g, from about 15000 g to about 30000 g, from about 20000 g to about 30000 g, from about 25000 g to about 30000 g, from about 1000 g to about 25000 g, from about 5000 g to about 25000 g, from about 10000 g to about 25000 g, from about 15000 g to about 25000 g, from about 20000 g to about 25000 g, from about 1000 g to about 20000 g, from about 5000 g to about 20000 g, from about 10000 g to about 20000 g, from about 15000 g to about 20000 g, from about 1000 g to about 15000 g, from about 5000 g to about 15000 g, from about 10000 g to about 15000 g, from about 1000 g to about 10000 g, from about 5000 g to about 10000 g, or from about 1000 g to about 5000 g force.

In certain embodiments, the cells are concentrated to from about 1×10⁶ CFUs per milliliter to about 1×10¹² CFUs per milliliter, from about 1×10⁷ CFUs per milliliter to about 1×10¹² CFUs per milliliter, from about 1×10⁸ CFUs per milliliter to about 1×10¹² CFUs per milliliter, from about 1×10⁹ CFUs per milliliter to about 1×10¹² CFUs per milliliter, from about 1×10¹⁰ CFUs per milliliter to about 1×10¹² CFUs per milliliter, from about 1×10¹¹ CFUs per milliliter to about 1×10¹² CFUs per milliliter, from about 1×10⁶ CFUs per milliliter to about 1×10¹¹ CFUs per milliliter, from about 1×10⁷ CFUs per milliliter to about 1×10¹¹ CFUs per milliliter, from about 1×10⁸ CFUs per milliliter to about 1×10¹¹ CFUs per milliliter, from about 1×10⁹ CFUs per milliliter to about 1×10¹¹ CFUs per milliliter, from about 1×10¹¹ CFUs per milliliter to about 1×10¹¹ CFUs per milliliter, from about 1×10⁶ CFUs per milliliter to about 1×10¹⁰ CFUs per milliliter, from about 1×10⁷ CFUs per milliliter to about 1×10¹⁰ CFUs per milliliter, from about 1×10⁸ CFUs per milliliter to about 1×10¹⁰ CFUs per milliliter, from about 1×10⁹ CFUs per milliliter to about 1×10¹⁰ CFUs per milliliter, from about 1×10⁶ CFUs per milliliter to about 1×10⁹ CFUs per milliliter, from about 1×10⁷ CFUs per milliliter to about 1×10⁹ CFUs per milliliter, from about 1×10⁸ CFUs per milliliter to about 1×10⁹ CFUs per milliliter, from about 1×10⁶ CFUs per milliliter to about 1×10⁸ CFUs per milliliter, from about 1×10⁷ CFUs per milliliter to about 1×10⁸ CFUs per milliliter, or from about 1×10⁶ CFUs per milliliter to about 1×10⁷ CFUs per milliliter.

In certain embodiments, microbial cells of the present disclosure are frozen. In certain embodiments, the microbial cells of the present disclosure are mixed with one or more cryoprotective agents (CPAs) before freezing. In certain embodiments, the ratio of cells to CPA is approximately 25:1, 10:1, 5:1, 4:1, 3:1, 2:1, 1:1, 1:2, 1:3, 1:4, 1:5, 1:10, or 1:25. In certain embodiments, a CPA comprises one or more of glycerol, maltodextrin, sucrose, inulin, trehalose, and alginate. In certain embodiments, a CPA further comprises one or more antioxidants. In certain embodiments, an antioxidant is selected from the list of cysteine, ascorbic acid, and riboflavin.

In certain embodiments, the microbial cells of the present disclosure are lyophilized. In certain embodiments, the lyophilized cells are used to make an orally-administered dose of the disclosure. In certain embodiments, primary drying is conducted below approximately −20° C. In certain embodiments, primary drying is followed by a secondary drying at a higher temperature, e.g. greater than 0° C., greater than 5° C., or greater than 10° C.

### Functionally Equivalent and Identical Drug Products to FB-001

The strains included in FB-001 are described herein by 16S RNA sequences and functional characteristics. Based on this, equivalent Consortia to FB-001 can be generated by screening multiple of the same strain to find equivalent strains with equivalent function to those that comprise FB-001. Accordingly, identical strains may theoretically have different functions, strains can be screened using 16S RNA and Biolog as described herein to identify functionally identical and equivalent strains from any fecal collection using the methods of collection described herein.

It is important to note that FB-001 was articulately designed to have multiple of the same strain in the Consortia. The reason for this to have redundancy to ensure function; however, such redundancy is not required for equivalent function so long as one of the otherwise redundant strains is included in the final drug product at a sufficient viable cell count amount to achieve in vivo function in a subject. Accordingly, a Consortia that is equivalent or identical to FB-001 may contain all redundancies (see Table 1) or alternatively may contain no or fewer redundancies per strain so long as the included strains achieve in vivo function in a subject.

In an alternative approach to creating a functionally equivalent Consortia to FB-001, one of skill in the art could recreate a consortia of supportive microbes from healthy fecal donors and supplement the supportive microbes with one or more O. formigenes strains. In certain embodiments, the supportive microbes will be supplemented with two or more O. formigenes strains or specifically three O. formigenes strains. The supportive microbes may comprise anywhere between 10 and 200 microbes so long as such supportive community supports and encourages the growth, health, and engraftment of the O. formigenes strain(s) in a subject. FB-001 was designed to have 148 microbes to mimic a complete, healthy microbiome. Accordingly, equivalent Consortia may comprise approximately 148 microbes, including O. formigenes strain(s). However, it is interesting to note that older subjects often have smaller microbiomes; accordingly, a functionally equivalent Consortia to FB-001 may also have far fewer microbes (e.g., 30-40, 40-50, 50-60, 60-70, 70-80, 8-90, 90-100, 100-110, 110-120, 120-130, 130-140, or 140-150 microbes, including O. formigenes strain(s)).

### Functionally Equivalent and Identical Drug Products to FB-003

The strains included in FB-003 are described herein by 16S RNA sequences and functional characteristics. Based on this, equivalent Consortia to FB-003 can be generated by screening multiple of the same strain to find equivalent strains with equivalent function to those that comprise FB-003. Accordingly, identical strains may theoretically have different functions, strains can be screened using 16S RNA and Biolog as described herein to identify functionally identical and equivalent strains from any fecal collection using the methods of collection described herein.

It is important to note that FB-003 was articulately designed to have multiple of the same strain in the Consortia. The reason for this to have redundancy to ensure function; however, such redundancy is not required for equivalent function so long as one of the otherwise redundant strains is included in the final drug product at a sufficient viable cell count amount to achieve in vivo function in a subject. Accordingly, a Consortia that is equivalent or identical to FB-003 may contain all redundancies (see Table 2) or alternatively may contain no or fewer redundancies per strain so long as the included strains achieve in vivo function in a subject.

### Therapeutic Applications

The present disclosure provides Consortia capable of engrafting into one or more niche of a gastrointestinal tract where it is capable of treating dysbiosis that causes or contributes to disease in an animal. In certain embodiments, the animal is a human.

In certain embodiments of the disclosure, when administered to an animal, the animal is pre-treated with one or more antibiotics prior to administration of the Consortium. In certain embodiments, the one or more antibiotics is selected from ampicillin, enrofloxacin, clarithromycin, and metronidazole. In certain embodiments, the animal is pre-treated with a polyethylene glycol bowel-preparation procedure.

In certain embodiments, a Consortia is used to treat a subject having or at risk of developing dysbiosis of the gastrointestinal tract. In certain embodiments, the dysbiosis is caused by or causes irritable bowel disease (IBD), colitis, ulcerative colitis, or Crohn's disease. In certain embodiments, the dysbiosis is caused by or causes IBD. In certain embodiments, the dysbiosis is caused by or causes colitis. In certain embodiments, the dysbiosis is caused by or causes ulcerative colitis. In certain embodiments, the dysbiosis is caused by or causes Crohn's disease.

In certain embodiments, a Consortia significantly alters the profile and/or concentration of bile acids present in an animal. For example, in certain non-limiting embodiments, a Consortia significantly alters the profile and/or concentration of Tβ-MCA, Tα-MCA, TUDCA, THDCA, TCA, 7β-CA, 7-oxo-CA, TCDCA, Tω-MCA, TDCA, α-MCA, β-MCA, ω-MCA, Muro-CA, d4-CA, CA, TLCA, UDCA, HDCA, CDCA, DCA, and LCA in an animal. In certain embodiments, FB-003 alters the profile and/or concentration of one or more of Tβ-MCA, Tα-MCA, TUDCA, THDCA, TCA, 7β-CA, 7-oxo-CA, TCDCA, Tω-MCA, TDCA, α-MCA, β-MCA, ω-MCA, Muro-CA, d4-CA, CA, TLCA, UDCA, HDCA, CDCA, DCA, and LCA in an animal.

In certain embodiments, a high-complexity defined gut microbial community of the present disclosure can be used to treat an animal having a cholestatic disease, such as, for example, primary sclerosing cholangitis, primary biliary cholangitis, progressive familial intrahepatic cholestasis, or nonalcoholic steatohepatitis. For example, in certain non-limiting embodiments, the animal may be a mammal, and more particularly a human.

In certain embodiments, a Consortia can be administered via an enteric route. For example, in certain non-limiting embodiments, a microbial consortium is administered orally, rectally (e.g., by enema, suppository, or colonoscope), or by oral or nasal tube.

In certain embodiments, a Consortia is administered orally. In certain embodiments the oral administration is by a powder. In certain embodiments the oral administration is by a slurry. In certain embodiments the oral administration is by pills or capsules.

In certain embodiments, a Consortia can be administered to a specific location along the gastrointestinal tract. For example, in certain non-limiting embodiments, a microbial consortium can be administered into one or more gastrointestinal location including the mouth, esophagus, stomach, small intestine (duodenum, jejunum, ileum), large intestine (cecum, ascending colon, transverse colon, descending colon), or rectum. In certain embodiments, a microbial consortium can be administered in all regions of the gastrointestinal tract.

In certain embodiments, FB-001 and FB-003 can be administered orally via a pill or capsule.

### Methods of Treating Dysbiosis and IBD

The present disclosure provides methods for preventing and/or treating dysbiosis in a subject. In certain embodiments, the methods can comprise administering an effective amount of a Consortia or a pharmaceutical composition thereof disclosed herein. In certain embodiments, the Consortia is FB-001 or a functional equivalent thereof. In certain embodiments, the Consortia is FB-003 or a functional equivalent thereof. In certain embodiments, the administration of the Consortia or pharmaceutical composition thereof results in the engraftment of the microbes of the Consortia. In certain embodiments, the administration of the Consortia or pharmaceutical composition thereof results in increase in the microbial diversity of the gastrointestinal tract. In certain embodiments, the administration of the Consortia or pharmaceutical composition thereof results in increase of short chain fatty acids (SCFAs) (e.g., in the gastrointestinal tract of the subject). In certain embodiments, the administration of the Consortia or pharmaceutical composition thereof results in increase in secondary bile acids (e.g., in the gastrointestinal tract of the subject). In certain embodiments, the administration of the Consortia or pharmaceutical composition thereof results in decrease of bacterial pathogens (e.g., in the gastrointestinal tract of the subject).

The present disclosure also provides methods for decreasing dysbiosis (e.g., dysbiosis of the gastrointestinal tract) in a subject. In certain embodiments, the methods can comprise administering an effective amount of a Consortia or pharmaceutical composition thereof disclosed herein. In certain embodiments, the Consortia is FB-001 or a functional equivalent thereof. In certain embodiments, the Consortia is FB-003 or a functional equivalent thereof. In certain embodiments, the administration of the Consortia or pharmaceutical composition thereof results in the engraftment of the microbes of the Consortia. In certain embodiments, the administration of the Consortia or pharmaceutical composition thereof results in increase in the microbial diversity of the gastrointestinal tract. In certain embodiments, the administration of the Consortia or pharmaceutical composition thereof results in increase of short chain fatty acids (SCFAs) (e.g., in the gastrointestinal tract of the subject). In certain embodiments, the administration of the Consortia or pharmaceutical composition thereof results in increase in secondary bile acids (e.g., in the gastrointestinal tract of the subject). In certain embodiments, the administration of the Consortia or pharmaceutical composition thereof results in decrease of bacterial pathogens (e.g., in the gastrointestinal tract of the subject).

Further, the present disclosure provides methods for restoring microbiome in a subject. In certain embodiments, the methods can comprise administering an effective amount of a Consortia or pharmaceutical composition thereof disclosed herein. In certain embodiments, the Consortia is FB-001 or a functional equivalent thereof. In certain embodiments, the Consortia is FB-003 or a functional equivalent thereof. In certain embodiments, the administration of the Consortia or pharmaceutical composition thereof results in the engraftment of the microbes of the Consortia. In certain embodiments, the administration of the Consortia or pharmaceutical composition thereof results in increase in the microbial diversity of the gastrointestinal tract. In certain embodiments, the administration of the Consortia or pharmaceutical composition thereof results in increase of short chain fatty acids (SCFAs) (e.g., in the gastrointestinal tract of the subject). In certain embodiments, the administration of the Consortia or pharmaceutical composition thereof results in increase in secondary bile acids (e.g., in the gastrointestinal tract of the subject). In certain embodiments, the administration of the Consortia or pharmaceutical composition thereof results in decrease of bacterial pathogens (e.g., in the gastrointestinal tract of the subject).

Moreover, the present disclosure provides methods for recovery of a healthy microbiome in a subject undergone to a dysbiosis-inducing event. In certain non-limiting embodiments, for example and without any limitation, the dysbiosis-inducing event can be an antibiotic treatment, an infectious disease, or an underlying disease. In certain non-limiting embodiments, the underlying disease can be IBD, colitis, ulcerative colitis, or Crohn's disease. In certain embodiments, the methods can comprise administering an effective amount of a Consortia or pharmaceutical composition thereof disclosed herein. In certain embodiments, the Consortia is FB-001 or a functional equivalent thereof. In certain embodiments, the Consortia is FB-003 or a functional equivalent thereof. In certain embodiments, the administration of the Consortia or pharmaceutical composition thereof results in the engraftment of the microbes of the Consortia. In certain embodiments, the administration of the Consortia or pharmaceutical composition thereof results in increase in the microbial diversity of the gastrointestinal tract. In certain embodiments, the administration of the Consortia or pharmaceutical composition thereof results in increase of short chain fatty acids (SCFAs) (e.g., in the gastrointestinal tract of the subject). In certain embodiments, the administration of the Consortia or pharmaceutical composition thereof results in increase in secondary bile acids (e.g., in the gastrointestinal tract of the subject). In certain embodiments, the administration of the Consortia or pharmaceutical composition thereof results in decrease of bacterial pathogens (e.g., in the gastrointestinal tract of the subject).

The present disclosure further provides methods for reducing a severity of at least one symptom of a gastrointestinal disease (e.g., associated with dysbiosis of the gastrointestinal tract) in a subject. Non-limiting examples of gastrointestinal diseases include irritable bowel syndrome, diarrhea, constipation, celiac disease, and leaky gut syndrome. In certain embodiments, the methods can comprise administering an effective amount of a Consortia or pharmaceutical composition thereof disclosed herein. In certain embodiments, the Consortia is FB-001 or a functional equivalent thereof. In certain embodiments, the Consortia is FB-003 or a functional equivalent thereof. In certain embodiments, the administration of the Consortia or pharmaceutical composition thereof results in the engraftment of the microbes of the Consortia. In certain embodiments, the administration of the Consortia or pharmaceutical composition thereof results in increase in the microbial diversity of the gastrointestinal tract. In certain embodiments, the administration of the Consortia or pharmaceutical composition thereof results in increase of short chain fatty acids (SCFAs) (e.g., in the gastrointestinal tract of the subject). In certain embodiments, the administration of the Consortia or pharmaceutical composition thereof results in increase in secondary bile acids (e.g., in the gastrointestinal tract of the subject). In certain embodiments, the administration of the Consortia or pharmaceutical composition thereof results in decrease of bacterial pathogens (e.g., in the gastrointestinal tract of the subject).

The present disclosure further provides methods for treating a disease in a subject. In certain embodiments, the disease is irritable bowel syndrome, diarrhea, constipation, celiac disease, and leaky gut syndrome, colitis, ulcerative colitis, or Crohn's disease. In certain embodiments, the methods can comprise administering an effective amount of a Consortia or pharmaceutical composition thereof disclosed herein. In certain embodiments, the Consortia is FB-001 or a functional equivalent thereof. In certain embodiments, the Consortia is FB-003 or a functional equivalent thereof. In certain embodiments, the administration of the Consortia or pharmaceutical composition thereof results in the engraftment of the microbes of the Consortia. In certain embodiments, the administration of the Consortia or pharmaceutical composition thereof results in increase in the microbial diversity of the gastrointestinal tract. In certain embodiments, the administration of the Consortia or pharmaceutical composition thereof results in increase of short chain fatty acids (SCFAs) (e.g., in the gastrointestinal tract of the subject). In certain embodiments, the administration of the Consortia or pharmaceutical composition thereof results in increase in secondary bile acids (e.g., in the gastrointestinal tract of the subject). In certain embodiments, the administration of the Consortia or pharmaceutical composition thereof results in decrease of bacterial pathogens (e.g., in the gastrointestinal tract of the subject).

The present disclosure further provides methods for treating Inflammatory Bowel Disease (IBD) in a subject. In certain embodiments, the methods can comprise administering an effective amount of a Consortia or pharmaceutical composition thereof disclosed herein. In certain embodiments, the Consortia is FB-001 or a functional equivalent thereof. In certain embodiments, the Consortia is FB-003 or a functional equivalent thereof. In certain embodiments, the administration of the Consortia or pharmaceutical composition thereof results in the engraftment of the microbes of the Consortia. In certain embodiments, the administration of the Consortia or pharmaceutical composition thereof results in increase in the microbial diversity of the gastrointestinal tract. In certain embodiments, the administration of the Consortia or pharmaceutical composition thereof results in increase of short chain fatty acids (SCFAs) (e.g., in the gastrointestinal tract of the subject). In certain embodiments, the administration of the Consortia or pharmaceutical composition thereof results in increase in secondary bile acids (e.g., in the gastrointestinal tract of the subject). In certain embodiments, the administration of the Consortia or pharmaceutical composition thereof results in decrease of bacterial pathogens (e.g., in the gastrointestinal tract of the subject).

IBD (including colitis, ulcerative colitis, and Crohn's disease) is a debilitating disease and treatment of which remains a high unmet need despite approved marketed products. It is a disorder characterized by chronic inflammation and hyperpermeability of the gut. There are no known pharmacological cures for IBD; rather, current standard of care treatment relies on control of symptoms, maintenance of remission, prevention of relapse, and palliative care. There are more than 1.7 million IBD patients in the US alone as of 2022 and a disease-modifying therapy with a good tolerability profile would be game changing. There are also no therapies to treat the underlying dysbiosis of IBD.

In certain embodiments, FB-001 is a disease-modifying therapy for the treatment of IBD. In certain embodiments, FB-001 treats the underlying dysbiosis of IBD. In certain embodiments, a functional equivalent of FB-001 is a disease-modifying therapy for the treatment of IBD. In certain embodiments, a functional equivalent of FB-001 treats the underlying dysbiosis of IBD.

In certain embodiments, FB-003 is a disease-modifying therapy for the treatment of IBD. In certain embodiments, FB-003 treats the underlying dysbiosis of IBD. In certain embodiments, a functional equivalent of FB-003 is a disease-modifying therapy for the treatment of IBD. In certain embodiments, a functional equivalent of FB-003 treats the underlying dysbiosis of IBD.

As a result of the dysbiosis in IBD patients, the microbial diversity, the short chain fatty acids (SCFAs), and the secondary bile acids are all reduced while there is an increase in the presence of bacterial pathogens. In certain embodiments, FB-001 or a functional equivalent thereof can increase (or restore to normal and/or healthy levels) the microbial diversity in an IBD patient, increase the SCFAs in an IBD patient, increase secondary bile acids in IBD patients, and decrease the presence of bacterial pathogens; thereby restoring the gut ecosystem and promoting heathy barrier responses. In certain embodiments, FB-003 or a functional equivalent thereof can increase (or restore to normal and/or healthy levels) the microbial diversity in an IBD patient, increase the SCFAs in an IBD patient, increase secondary bile acids in IBD patients, and decrease the presence of bacterial pathogens; thereby restoring the gut ecosystem and promoting heathy barrier responses.

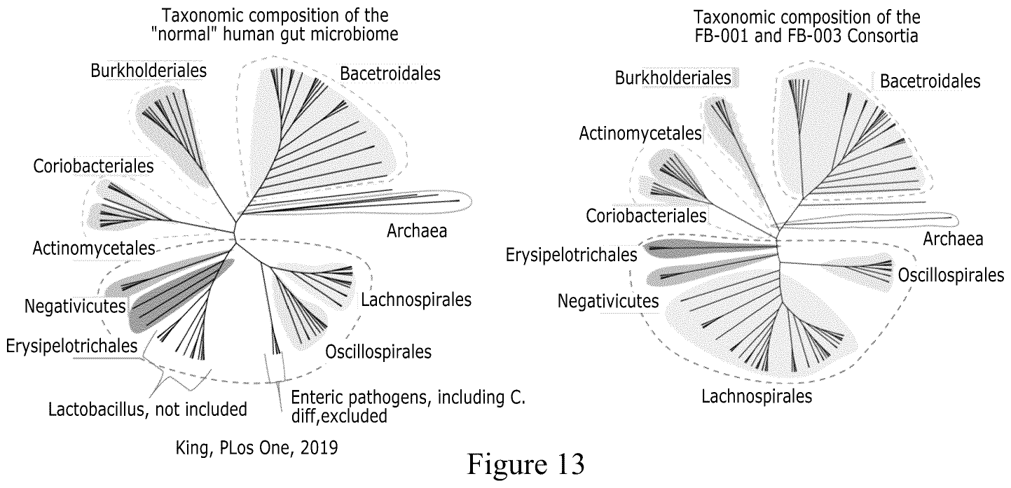

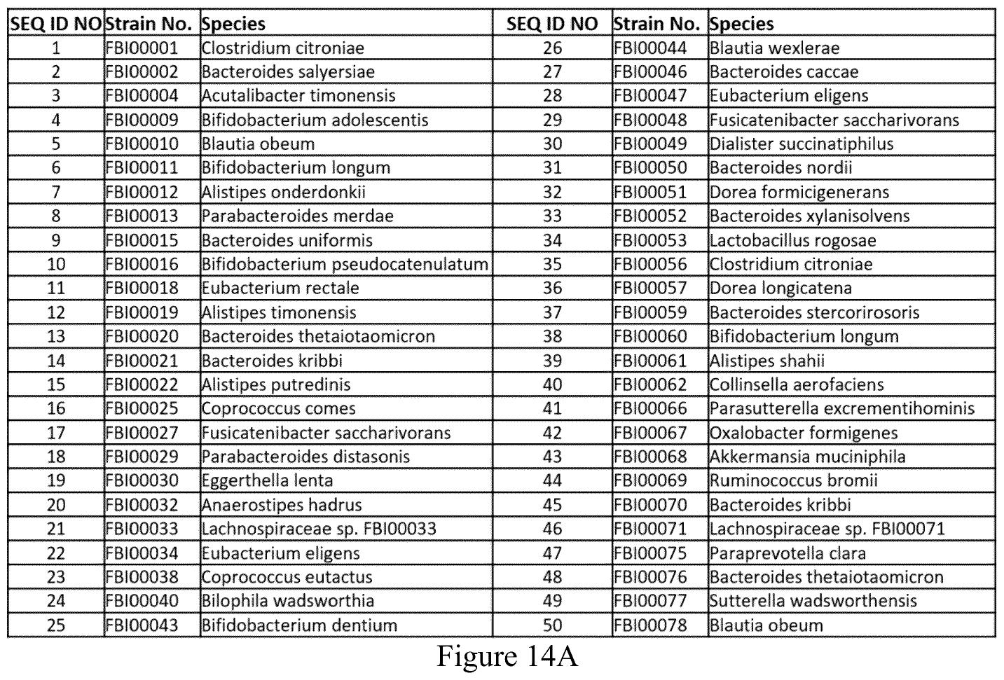

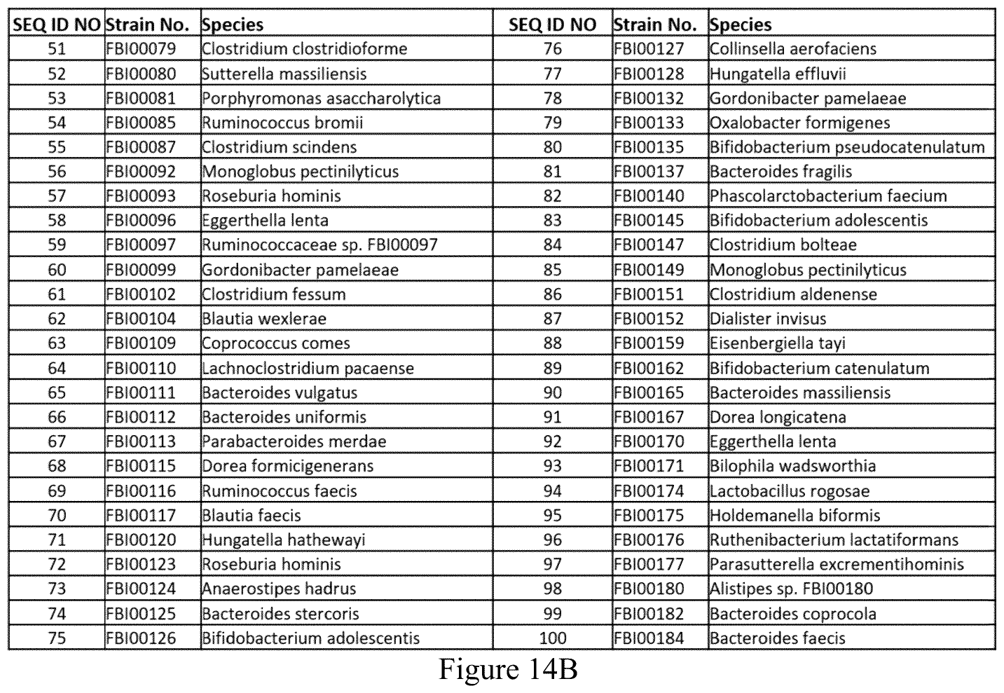

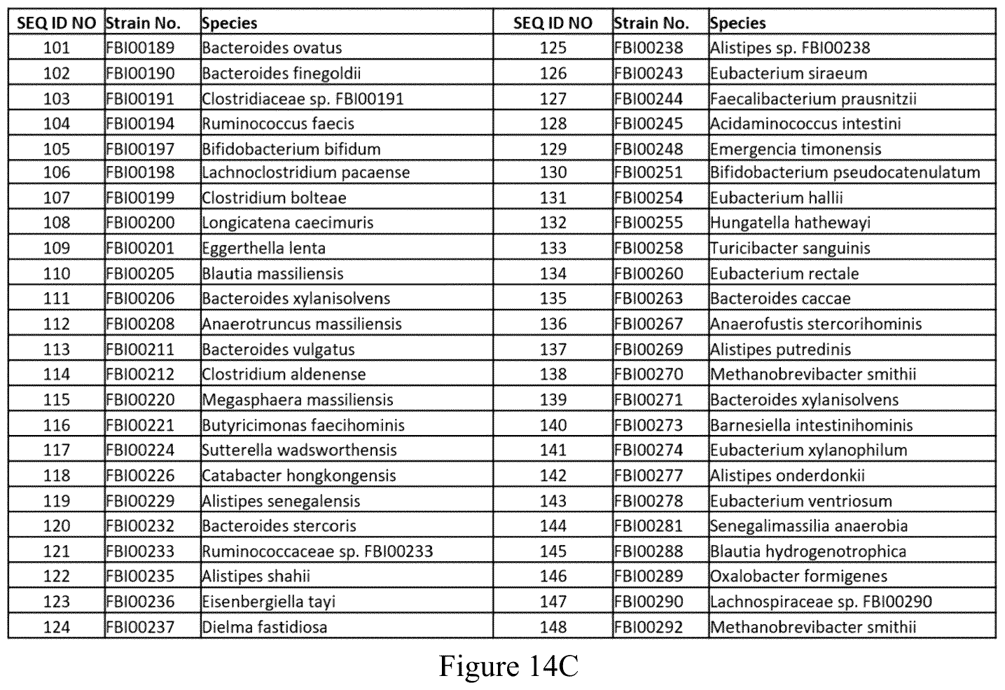

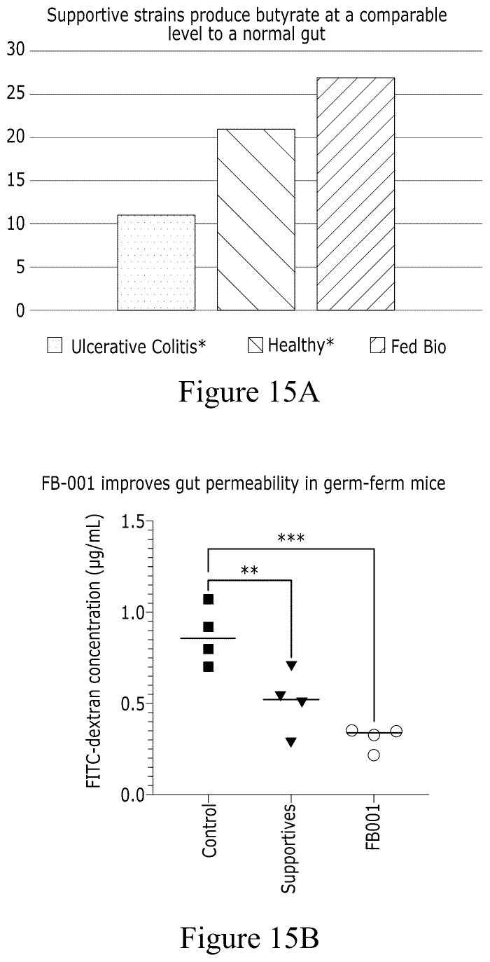

Limitations in the field of IBD microbiome treatments include the low diversity of microbes. For example, certain microbiome treatments for IBD include spore preparations which limit diversity based on the how spores are isolated and manufactured (i.e., the preparation of spores results in a loss of microbial diversity and a complete taxonomic recapitulation of a healthy microbiome is not possible, see e.g., FIG. 13) and small defined consortia of fewer than 20 strains (i.e., limited, if any, microbial diversity and no complete or near complete taxonomic recapitulation of a healthy microbiome). In certain embodiments, the Consortia described herein solve the unmet need of a microbiome therapy with taxonomic and functional diversity for the treatment of IBD. In certain embodiments, the Consortia is FB-001 or a functional equivalent thereof. In certain embodiments, the Consortia is FB-003 or a functional equivalent thereof.

In certain embodiments, FB-001 or a functional equivalent thereof and FB-003 or a functional equivalent thereof engraft in the gastrointestinal tract of patients. In certain embodiments, FB-001 or a functional equivalent thereof and FB-003 or a functional equivalent thereof robustly engraft in the gastrointestinal tract of patients. In certain embodiments, FB-001 or a functional equivalent thereof and FB-003 or a functional equivalent thereof engraft in the gastrointestinal tract of patients and restore microbial diversity. In certain embodiments, FB-001 or a functional equivalent thereof and FB-003 or a functional equivalent thereof engraft in the gastrointestinal tract of patients and restore key functions of a healthy gut microbiome. In certain embodiments, the key functions are 1) increasing microbial diversity, 2) increasing the SCFAs, 3) increasing secondary bile acids, 4) decreasing the presence of bacterial pathogens, and/or 5) promoting heathy barrier responses.

In certain embodiments, a Consortia is used to treat IBD, colitis, ulcerative colitis, and/or Crohn's disease. In certain embodiments, FB-001 is used to treat IBD, colitis, ulcerative colitis, and/or Crohn's disease. In certain embodiments, FB-003 is used to treat IBD, colitis, ulcerative colitis, and/or Crohn's disease.

In certain embodiments, a Consortia is administered as a single dose or as multiple doses. In certain embodiments, a Consortia is administered once a day for 2 days, 3 days, 4 days, 5 days, 6 days, 1 week, 2 weeks 3 weeks, 1 month, 2 months, 3 months, 4 months, 5 months, 6 months, or 1 year. In certain embodiments, a Consortia is administered multiple times daily. In certain embodiments, a Consortia is administered twice daily, three times daily, 4 times daily, or 5 times daily. In certain embodiments, a Consortia is administered intermittently. In certain embodiments, a Consortia is administered once weekly, once monthly, or when a subject is in need thereof. In certain embodiments, the Consortia is FB-001 or FB-003. In certain embodiments, the Consortia is FB-003.

In certain embodiments, a Consortia is administered at an effective dose to allow for engraftment and substrate metabolism. In certain embodiments, a Consortia is administered at an effective dose to allow for engraftment and significant SCFA production. In certain embodiments, a Consortia is administered at an effective dose to allow for engraftment and reduced inflammation of the gastrointestinal tract.

In certain embodiments, a Consortia is administered at a first loading dose and then followed by maintenance doses. In certain embodiments, the first loading dose is administered for 1 day, 2 days, 3 days, 4 days, 5 days, 6 days, 7 days, 8 days, 9 days, or 10 days. In certain embodiments, the loading dose is administered for 1-3 days. In certain embodiments, the loading dose is administered for 2-4 days. In certain embodiments, the loading dose is administered for 2-3 days. In certain embodiments, the loading dose is administered for 3-5 days. In certain embodiments, the loading dose is administered for 4-6 days. In certain embodiments, the loading dose is administered for 5-7 days. In certain embodiments, the loading dose is administered for 1 day. In certain embodiments, the loading dose is administered for 3 days. In certain embodiments, the loading dose is administered for 2 days. In certain embodiments, the maintenance doses are administered for 5-10 days following the last loading dose. In certain embodiments, the maintenance doses are administered for 7-12 days following the last loading dose. In certain embodiments, the maintenance doses are administered for 10-14 days following the last loading dose. In certain embodiments, the maintenance doses are administered for 14-21 days following the last loading dose. In certain embodiments, the maintenance doses are administered for 21-28 days following the last loading dose. In certain embodiments, the maintenance doses are administered for 14 days following the last loading dose. In certain embodiments, the maintenance doses are administered for 21 days following the last loading dose. In certain embodiments, the maintenance doses are administered for 28 days following the last loading dose. In certain embodiments, the maintenance doses are administered for about 8 days following the last loading dose. In certain embodiments, the maintenance doses are administered for about 7 days following the last loading dose. In certain embodiments, the maintenance doses are administered for about 6 days following the last loading dose. In certain embodiments, the maintenance doses are administered for about 9 days following the last loading dose. In certain embodiments, the maintenance doses are administered for about 10 days following the last loading dose. In certain embodiments, the loading dose is administered for 2 days and the maintenance dose is administered for 6 days (for a total of a 8-day course of treatment). In certain embodiments, the loading dose is administered for 2 days and the maintenance dose is administered for 7 days (for a total of a 9-day course of treatment). In certain embodiments, the loading dose is administered for 2 days and the maintenance dose is administered for 8 days (for a total of a 10 day course of treatment). In certain embodiments, the loading dose is administered for 9 days and the maintenance dose is administered for 9 days (for a total of a 11 day course of treatment). In certain embodiments, the loading dose is administered for 2 days and the maintenance dose is administered for 10 days (for a total of a 12 day course of treatment). In certain embodiments, the Consortia is FB-001. In certain embodiments, the loading dose follows the pretreatment with antibiotics as described in the Combination Therapy section below. In certain embodiments, the loading dose follows the pretreatment with a bowel preparation as described in the Combination Therapy section below. In certain embodiments, the loading dose follows the pretreatment with antibiotics and a bowel preparation as described in the Combination Therapy section below.

In certain embodiments, FB-001 is formulated by blending the seven lyophilized DSs containing the 148 microbial species and filling them into coated enteric capsules. In certain embodiments, FB-003 is formulated by blending the four lyophilized DSs containing the 145 microbial species and filling them into coated enteric capsules. In certain embodiments, the capsules of FB-001 or FB-003 are provided in blister packaging or alternative packaging to allow for no or low oxygen exposure (e.g., packaging to sustain the viability of anaerobic microbes). In certain embodiments, each capsule contains a range of 5×10¹⁰ to 5×10¹¹ viable cells/capsule. In certain embodiments, each capsule contains a range of 5×10⁹ to 5×10¹⁰ viable cells/capsule. In certain embodiments, each capsule contains a range of 5×10¹¹ to 5×10¹² viable cells/capsule. In certain embodiments, FB-001 or FB-003 is orally dosed at up to 10¹² viable cells on Days 1 and 2, and up to 10¹¹ viable cells on Days 3 to 10. In certain embodiments, maltodextrin is included as an excipient in the capsules.

In certain embodiments, each capsule of FB-001 or FB-003 contains a range of 5×10¹⁰ to 5×10¹¹ viable cells/capsule and a viable cell count basis and with relative abundance values of the 145 (absent O. formigenes which is present only in FB-001) strains ranging from 18% to 0.015%.

In certain embodiments, the methods disclose herein comprise diagnosing dysbiosis in the subject and then treating the subject with the Consortia or pharmaceutical composition thereof. For example, but not by way of limitation, a method of treating a subject having dysbiosis can include (a) diagnosing the subject with dysbiosis and (b) administering an effective amount of a Consortia (e.g., FB-001 or FB-003) or pharmaceutical composition thereof to the subject. In certain embodiments, the method for diagnosing dysbiosis includes organic acid test, comprehensive digestive stool analysis (CDSA), hydrogen breath test, or a combination thereof.

In certain embodiments, the methods disclose herein comprise diagnosing IBD in the subject and then treating the subject with the Consortia or pharmaceutical composition thereof. For example, but not by way of limitation, a method of treating a subject having IBD can include (a) diagnosing the subject with IBD and (b) administering an effective amount of a Consortia (e.g., FB-001 or FB-003) or pharmaceutical composition thereof to the subject. In certain embodiments, the method for diagnosing IBD includes endoscopy, colonoscopy, flexible sigmoidoscopy, upper endoscopy, capsule endoscopy, analysis of c-reactive protein (CPR) in blood sample, analysis of erythrocyte sedimentation rate in blood sample, detection of calprotectin in stool, detection of lactoferrin in stool or a combination thereof.

In certain embodiments, the Consortia described herein are used to treat dysbiosis caused by chemical insult. In certain embodiments, the Consortia described herein are used to treat a disease associated with dysbiosis caused by chemical insult. In certain embodiments, FB-003, FB-001, Consortia A or Consortia B described herein are used to treat dysbiosis caused by chemical insult. In certain embodiments, FB-003, FB-001, Consortia A or Consortia B described herein are used to treat a disease associated with dysbiosis caused by chemical insult.

In certain embodiments, the Consortia described herein are used to treat dysbiosis caused by inflammation. In certain embodiments, the Consortia described herein are used to treat a disease associated with dysbiosis caused by inflammation. In certain embodiments, FB-003, FB-001, Consortia A or Consortia B described herein are used to treat dysbiosis caused by inflammation. In certain embodiments, FB-003, FB-001, Consortia A or Consortia B described herein are used to treat a disease associated with dysbiosis caused by inflammation.

In certain embodiments, the Consortia described herein are used to treat dysbiosis caused by infectious disease. In certain embodiments, the Consortia described herein are used to treat a disease associated with dysbiosis caused by infectious disease. In certain embodiments, FB-003, FB-001, Consortia A or Consortia B described herein are used to treat dysbiosis caused by infectious disease. In certain embodiments, FB-003, FB-001, Consortia A or Consortia B described herein are used to treat a disease associated with dysbiosis caused by infectious disease.

In certain embodiments, the Consortia described herein are used to treat dysbiosis by improving the epithelial barrier of the intestines and/or colon. In certain embodiments, the Consortia described herein are used to treat dysbiosis by fixing the epithelial barrier of the intestines and/or colon. In certain embodiments, the Consortia described herein are used to treat dysbiosis by transforming the epithelial barrier of the intestines and/or colon to that of a healthy individual. In certain embodiments, the Consortia is FB-003, FB-001, Consortia A or Consortia B.

### Combination Therapy

In certain embodiments, a Consortia can be administered in combination with other agents. In certain embodiments, a Consortia can be administered with an antimicrobial agent, an antifungal agent, an antiviral agent, an antiparasitic agent or a prebiotic. In certain embodiments, a Consortia can be administered subsequent to administration of an antimicrobial agent, an antifungal agent, an antiviral agent, an antiparasitic agent or a prebiotic. In certain embodiments, administration may be sequential over a period of hours or days, or simultaneously.

For example, in certain non-limiting embodiments, a microbial consortium can be administered with, or pre-administered with, one or more antibacterial agent selected from fluoroquinolone antibiotics (ciprofloxacin, Levaquin, floxin, tequin, avelox, and norflox); cephalosporin antibiotics (cephalexin, cefuroxime, cefadroxil, cefazolin, cephalothin, cefaclor, cefamandole, cefoxitin, cefprozil, and ceftobiprole); penicillin antibiotics (amoxicillin, ampicillin, penicillin V, dicloxacillin, carbenicillin, vancomycin, and methicillin); tetracycline antibiotics (tetracycline, minocycline, oxytetracycline, and doxycycline); and carbapenem antibiotics (ertapenem, doripenem, imipenem/cilastatin, and meropenem).

For example, in certain non-limiting embodiments, a microbial consortium can be administered with one or more antiviral agent selected from Abacavir, Acyclovir, Adefovir, Amprenavir, Atazanavir, Cidofovir, Darunavir, Delavirdine, Didanosine, Docosanol, Efavirenz, Elvitegravir, Emtricitabine, Enfuviltide, Etravirine, Famciclovir, Foscamet, Fomivirsen, Ganciclovir, Indinavir, Idoxuridine, Lamivudine, Lopinavir Maraviroc, MK-2048, Nelfinavir, Nevirapine, Penciclovir, Raltegravir, Rilpivirine, Ritonavir, Saquinavir, Stavudine, Tenofovir Trifluridine, Valaciclovir, Valganciclovir, Vidarabine, Ibacitabine, Amantadine, Oseltamivir, Rimantidine, Tipranavir, Zalcitabine, Zanamivir, and Zidovudine.

In certain embodiments, a microbial consortium can be administered with one or more antifungal agent selected from miconazole, ketoconazole, clotrimazole, econazole, omoconazole, bifonazole, butoconazole, fenticonazole, isoconazole, oxiconazole, sertaconazole, sulconazole, and tioconazole; triazole antifungals such as fluconazole, itraconazole, isavuconazole, ravuconazole, posaconazole, voriconazok, terconazole, and albaconazole; thiazole antifungals such as abafungin; allylamine antifungals such as terbinafine, naftifine, and butenafine; and echinocandin antifungals such as anidulafungin, caspofungin, and micafungin; polygodial; benzoic acid; ciclopirox; tolnaftate; undecylenic acid; flucytosine or 5-fluorocytosine; griseofulvin; and haloprogin.

In certain embodiments, a microbial consortium can be administered with one or more anti-inflammatory and/or immunosuppressive agent selected from cyclophosphamide, mycophenolate mofetil, corticosteroids, mesalazine, mesalamine, sulfasalazine, sulfasalazine derivatives, cyclosporin A, mercaptopurine, azathiopurine, prednisone, methotrexate, antihistamines, glucocorticoids, epinephrine, theophylline, cromolyn sodium, anti-leukotrienes, anticholinergics, monoclonal anti-IgE, immunomodulatory peptides, immunomodulatory small molecules, immunomodulatory cytokines, immunomodulatory antibodies, and vaccines.

In certain embodiments, a Consortia can be administered with one or more prebiotic selected from, but not limited to, amino acids, biotin, fructooligosaccharides, galactooligosaccharides, inulin, lactulose, mannan oligosaccharides, oligofructose-enriched inulin, oligofructose, oligodextrose, tagatose, trans-galactooligosaccharide, and xylooligosaccharides.

In certain embodiments, a Consortia described herein is administered in combination with an anti-inflammatory drug.

In certain embodiments, a Consortia described herein is administered in combination with an anti-diarrheal drug.

In certain embodiments, a Consortia described herein is administered in combination with a pain reliever. In certain embodiments, a Consortia described herein is administered in combination with a nonsteroidal anti-inflammatory drug (NSAIDs).

In certain embodiments, a Consortia described herein is administered in combination with vitamins and supplements.

In certain embodiments, a Consortia described herein is administered in combination with aminosalicylates.

In certain embodiments, a Consortia described herein is administered in combination with antibiotics.

In certain embodiments, a Consortia described herein is administered in combination with biologics. In certain embodiments, the biologics interrupt signals from the immune system that cause inflammation.

In certain embodiments, a Consortia described herein is administered in combination with corticosteroids or steroids.

In certain embodiments, a Consortia described herein is administered in combination with immunomodulators.

In certain embodiments, the combination treatment of a Consortia comprises the pretreatment with antibiotics. In certain embodiments, the pretreatment of antibiotics.

In certain embodiments, a bowel preparation (e.g., MiraLax) is administered in the late afternoon or early evening following the final dose of antibiotics.

### Kits

The presently disclosed subject matter provides kits for treating or preventing dysbiosis of the gastrointestinal tract. In certain embodiments, the dysbiosis is caused by or causes IBD, colitis, ulcerative colitis, and/or Crohn's disease. In certain embodiments, the kit comprises an effective amount of presently disclosed Consortia or a pharmaceutical composition comprising thereof.

In certain embodiments, the kit comprises an effective amount of FB-001 or a pharmaceutical composition comprising thereof. In certain embodiments, the kit comprises an effective amount of a functionally equivalent Consortia to FB-001 or a pharmaceutical composition comprising thereof. In certain embodiments, the kit comprises an effective amount of a functionally identical Consortia to FB-001 or a pharmaceutical composition comprising thereof.

In certain embodiments, the kit comprises an effective amount of FB-003 or a pharmaceutical composition comprising thereof. In certain embodiments, the kit comprises an effective amount of a functionally equivalent Consortia to FB-003 or a pharmaceutical composition comprising thereof. In certain embodiments, the kit comprises an effective amount of a functionally identical Consortia to FB-003 or a pharmaceutical composition comprising thereof.

In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes anaerobic containers to hold the Consortia(s) described herein. In certain non-limiting embodiments, the kit includes blister packs to hold the Consortia(s) described herein in the presence of no or limited amounts of oxygen. In certain non-limiting embodiments, the kit includes blister packs with desiccant to hold the Consortia(s) described herein in the presence of no or limited amounts of oxygen. In certain non-limiting embodiments, the kit includes bottles with desiccant to hold the Consortia(s) described herein in the presence of no or limited amounts of oxygen.

In certain embodiments, the kits include instructions for administering the Consortia as described herein. In certain embodiments, the instructions include directions for administering the loading and the maintenance dose.

In certain embodiments, the kits include storage instructions. In certain embodiments, the storage instructions are for storage at approximately −20° C. In certain embodiments, the storage instructions are for storage at less than −5° C. In certain embodiments, the storage instructions are for storage at less than approximately −15 to −20° C., −10 to −20° C., −10 to −15° C., −5 to −10° C., 0 to −5° C., below 0° C., or 0 to −20° C. In certain embodiments, the storage instructions are for storage at less than approximately 4° C. In certain embodiments, the storage instructions are for storage at room temperature.

In certain embodiments, the kits include instructions for maintaining the Consortia in no or low oxygen conditions.

In certain embodiments, the kits include instructions for the subject to remain off all antibiotics during treatment with the Consortia.

In certain embodiments, the kit includes FB-001 and instructions for administering FB-001.

In certain embodiments, the kit includes FB-003 and instructions for administering FB-003.

### EXAMPLES

### Example 1: Design of Consortia

While microbial consortia of two or more microbial strains have been made before, limitations existed that prevent manufacturing and clinical efficacy. Specifically, manufacturing limitations have prevented the design and generation of large consortia that are able to engraft in the gastrointestinal tract and build a functional microbiota system.

Isolation of donor-derived microbial strains. Microbial strains were isolated and identified using the methods described in PCT/US2021/021790.

Generation of Consortia. Using the microbial strains identified using the isolation and identification methods described in PCT/US2021/021790, over 30 large consortia were made and examined for their functional ability to metabolize oxalate, absence of phages, acceptable endotoxin levels, and their ability to be manufactured in multi-strain drug substances. The reason for the large number of experimental large consortia was because it was unknown what combination of microbial strains would be necessary given the considerations above. More so, the combination of microbial strains could not be predicted with algorithms and required wet laboratory work to determine efficacy and manufacturability.

Nineteen exemplary consortia are provided in Consortia I-XIX.

While Consortia V was most effective at oxalate metabolism and degradation (i.e., Consortia V had the lowest concentration of urinary oxalate), additional investigation and modification of the Consortia was needed to design a product for the treatment of disease, specifically a disease that causes or is caused by decrease ability or inability to effectively metabolize and degrade oxalate in the gastrointestinal tract. Accordingly, modifications of Consortia V were made to determine which microbiota provided functional benefits, including but not limited consortia growth, oxalate metabolism and degradation, consortia engraftment, and consortia survival, and which microbiota were either not needed or provided a detriment to the patient receiving the consortia as treatment of the disease or a detriment to the function of the consortia as a whole (including but not limited consortia growth, oxalate metabolism and degradation, consortia engraftment, and consortia survival). Examples of such designed and investigated consortia are Consortia IX-XVI.

Of Consortia IX-XVI that were designed and tested, Consortia IX was selected as the lead for clinical development. Key changes made as variations of the consortia were made to modify for the treatment of disease, specifically a disease that causes or is caused by decrease ability or inability to effectively metabolize and degrade oxalate in the gastrointestinal tract, include removing the Citrobacter freundii strain because through experimentation it was determined to be facultative anaerobes (see e.g., strain removal between Consortia XIII and XV and between Consortia XXIV and XIII and XII), replacement of one Bacteroides kribbi species with a different Bacteroides kribbi species cluster (see e.g., strain replacements between Consortia XV and XVI), replacement of one Blautia faecis species with a different Blautia faecis species (see e.g., strain replacements between Consortia XV and XVI), strains that were determined to be duplicative strains based on Whole Genome Sequencing cluster (see e.g., strain removals between Consortia XVII and XVI), replacement of one Bifidobacterium adolescentis with an alternate Bifidobacterium adolescentis to improve growth in culture (see e.g., strain replacement between Consortia X and XII), replacement of one Bifidobacterium pseudocatenulatum with an alternate Bifidobacterium pseudoactenulatum to improve growth in culture (see e.g., strain replacement between Consortia X and XII), replacement of one Bacteroides xylanisolvens with an alternate Bacteroides xylanisolvens to improve growth in culture (see e.g., strain replacement between Consortia X and XII), replacement of one Clostridium citroniae with an alternate Clostridium citroniae to improve growth in culture (see e.g., strain replacement between Consortia X and XII), replacement of one Blautia faecis with an alternate Blautia faecis in order to identify a Blautia strain that was able to grow sufficiently to produce a master cell bank (see e.g., strain replacement between Consortia X and XII), removal of Holdemanella biformis to eliminate phage risk because while a phage was not detected in co-culture it was detected using bioinformatic methods (see e.g., strain replacement between Consortia X and XII), and removal of Faecalibacterium prasnitzii to eliminate phage risk because while a phage was not detected in co-culture it was detected using bioinformatic methods (see e.g., strain replacement between Consortia X and XII).

### Example 2: Drug Product Design and Manufacture

As shown in Example 1, the Consortia described herein were designed to be a complex community of anaerobic microbiota that can engraft and function in a gastrointestinal tract. However, prior methods known to one of skill in the art were not capable of manufacturing such large consortia. Accordingly, new methods of manufacture were needed in order to grow the microbiota in discrete groups (i.e., drug substances) to then form a final drug product.

Conventionally, Live Biotherapeutic Products (LBPs) are manufactured one strain at a time (i.e., single strain manufacturing). Single strain manufacture necessitates fermentation scale-up of each single strain followed by lyophilization to make individual drug substances (each a “DS”). Thereafter the multiple DSs of individual lyophilized stains are then blended into a mixture and filled into capsules or other suitable packaging/filling to make a final drug product (a “DP”). While this works for small consortia, it is not feasible to grow 100+ strains separately, make 100+DSs, and then blend 100+DSs into a stable DP. In addition to stability limitations, current technology would require 1 or more year(s) to manufacture a single DP. Accordingly, conventional manufacturing using current technology was not an option for a DP comprising 100+ strains, and preferably 145+ strains as provided in Consortia IX.

As the Consortia were designed and modified as described in Examples 1 and 2, manufacturing methods were developed that were capable of manufacturing the 145+ strain consortia that comprise over 90 species, and 4 or more or the 6 taxonomic phyla found in the human gastrointestinal tract microbiome. More so, methods were developed to modify for Consortia IX that comprises approximately 99 species across the taxonomic phyla of Bacteroidetes, Firmicutes, Actinobacteria, Proteobacteria, and Archaea. The methods developed and described herein are mixed co-culture methods that are capable of stably growing greater than 50 strains in one co-culture to generate DSs with greater than 50 strains.

Strains were selected for co-culture by based on growth rates and the manufacturing was initially designed to add strains to the co-culture at different times throughout the manufacturing process in order to achieve growth of each strain. This approach was termed “time of addition” manufacturing. The rationale behind this initial approach was to ensure the strains reanimate in the gastrointestinal tract to increase efficacy of engraftment (i.e., allow for engraftment before the strains are excreted. Reanimation and engraftment of the lyophilized strains require preserving the strains in an “active state” (i.e., active growth state). However, this “time of addition” manufacturing approach was not successful because growth rates of the strains in the Consortia described herein are highly variable which makes it difficult to achieve exponential growth simultaneously for diverse strains in coculture. Accordingly, it was determined that additional experimentation was needed in order to understand each strain's unique growth kinetics to enable binning of strains based on growth rate and further modification of time of addition to the bioreactor. Growth kinetic assays were performed using HTP anaerobic growth kinetic assays on each individual strain in each of Consortia IX-XVI at 8 different inoculation densities.

While experimentation to understand each strain's unique growth kinetics proved helpful with the time of addition manufacturing, ultimately the highly variable nature of growing strains from a lyophilized powder to an active consortia in a bioreactor proved undesirable for the time of addition methods.

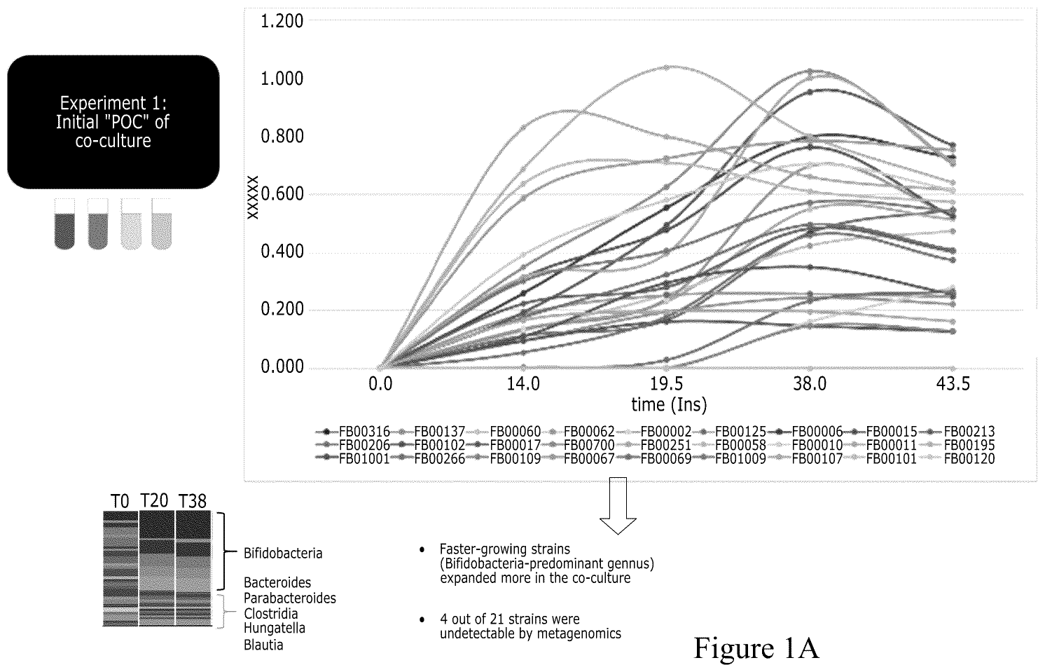

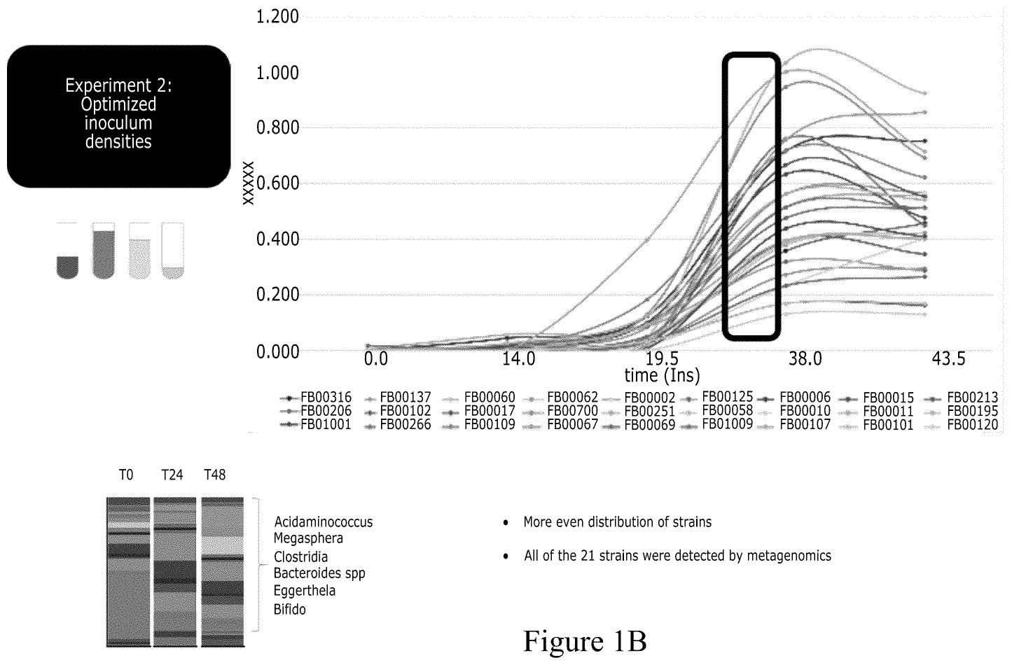

Accordingly, a second approach for coculture was developed. Instead of applying different time of additions, the second approach used inoculation density adjustment for each strain to synchronize growth and control of strain distribution at the time of harvest from the co-culture (“inoculation density” manufacturing). Using the unique growth kinetics determined for each strain in the Consortia, specifically Consortia IX-XVI, growth zones were determined for each strain. In doing so, it was determined that coculture was effective and possible if each strain was added to culture at an initial time point based on inoculum density (i.e., number of cells per strain added to the co-culture) such that higher inoculum densities of certain strains resulted in shorter growth lag time for such strains. Based on this, higher inoculum density of slow growing strains and lower inoculum density of fast growing strains resulted in a synchronized harvest time. As shown by means of example in FIGS. 1A and 1B, modifying inoculation densities of individual strains allowed control over the strain distribution and improved strain recovery in cocultures (i.e., even distribution of strains as well as higher number of strain recovery are achieved by adjusting inoculum densities). FIG. 1A shows an example of a co-culture of 21 fast growing strains where only 4 of the 21 strains were undetectable by metagenomics in the final product. However, it is important to note that even if a strain is not detected in the final product, the strain may still provide a community advantage to allow for more efficient and robust growth of other strains that are detectable in the final product. FIG. 1B shows a further modified experiment of that show in FIG. 1A where the time of harvest and strain detection was modified. As shown the different timing of growth and culture led to a better distribution of strains and detection of all 21 strains.

Further modification of the coculture process was needed to improve fermentation. For example, additional modification was performed to control for pH and to achieve conditions of growth based on the bioreactor container (i.e., the type of container and the size of the container).

Using the methods developed and described herein, Consortia IX-XVI were each manufactured using only 7 DSs. One exemplary 7 DS Drug Product comprises: 3 O. formigenes monocultures, the strains of DS1 (e.g., listed in Table 1), the strains of DS2 (e.g., listed in Table 1), the strains of DS3 (e.g., listed in Table 1), the strains of DS4 (e.g., listed in Table 1).

Using the methods developed and described herein, the microbes listed in Table 2 (FB-003 or FB-003 Consortia) was manufactured using only 4 DSs. One exemplary 4 DS Drug Product comprises: the strains of DS1 (e.g., listed in Table 1), the strains of DS2 (e.g., listed in Table 1), the strains of DS3 (e.g., listed in Table 1), the strains of DS4 (e.g., listed in Table 1).

In order to identify each DS without sequencing the entire genome of all strains and in order to ensure proper growth throughout the coculture process, identifier strains were developed. For DS1, the identifier strains were Bacteroides thetaiotaomicron, Bifidobacterium pseudocatenulatum, and Megasphaera massiliensis. For DS2, the identifier strains were Bacteroides ovatus, Faecalibacterium prausnitzii, and Phascolarctobacterium faecium. For DS3, the identifier strains were Blautia wexlerae, Anaerostipes hadrus, and Clostridium bolteae. For DS4, the identifier strains were Holdemanella biformis, Parasutterella excrementihominis, and Dialister invisus.

As described herein, the number of strains detected at the conclusion of the co-culture may be less than the number of strains added at the beginning of the culture. This may be a result of limited detection methods. Furthermore, while not all strains may be detected at the conclusion of the coculture process, the inclusion of the undetected strains may still be vital for the survival and propagation of other strains that are detected.

In one experiment, DS1 consisted of 54 initial strains and 50 strains were detected at the end of the coculture process; DS2 consisted of 47 initial strains and 39 strains were detected at the end of the coculture process; DS3 consisted of 33 initial strains and 30 strains were detected at the end of the coculture process; and DS4 consisted of 14 initial strains and 11 strains were detected at the end of the coculture process.

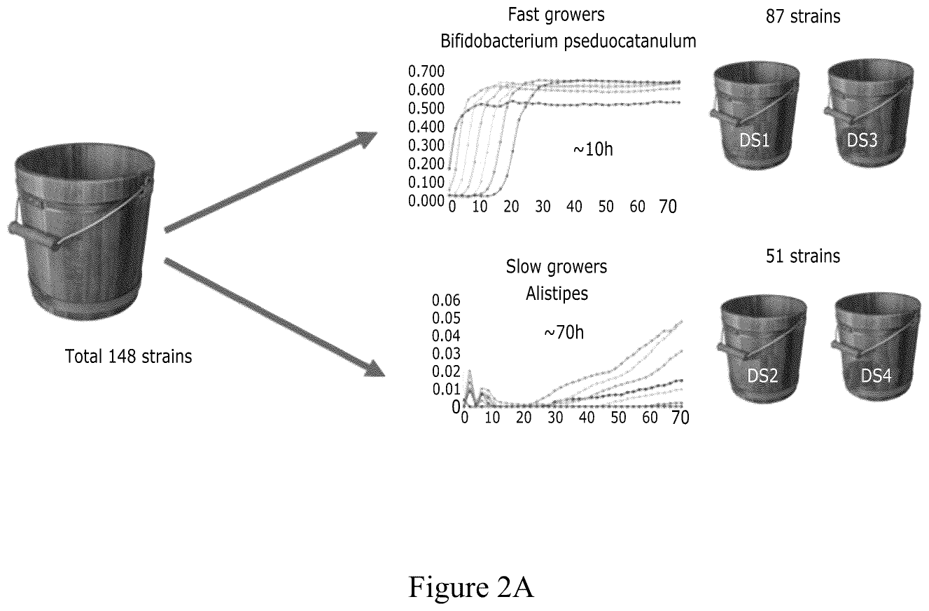

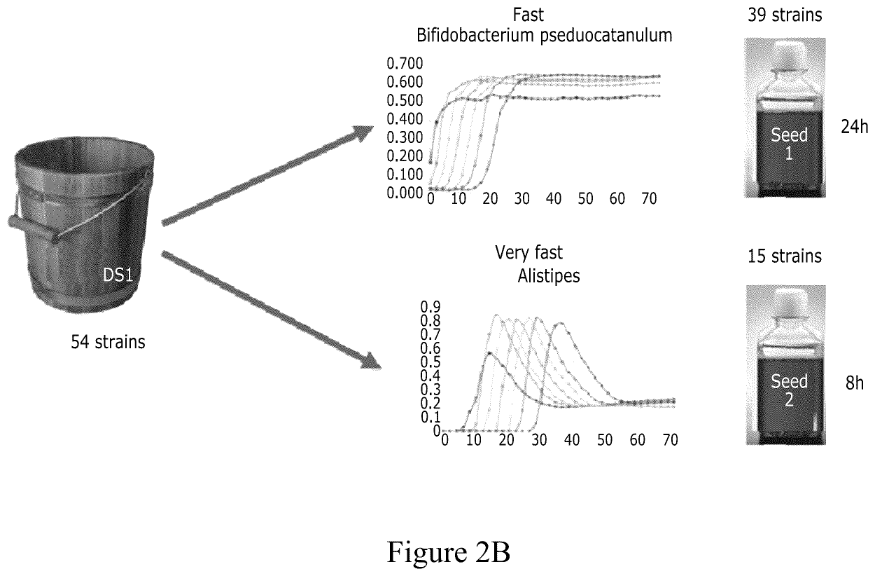

This achievement of the detection of strains was achieved through development of a fermentation process that allowed for growth of diverse strains in coculture. Variables that were investigated include growth kinetics of each strain, nutritional requirements for each strain, competition for nutritional sources in each DS, selection of the starting inoculum concentration to achieve strain growth and distribution in each DS. For example growth curves were performed and used to define DS buckets as well as starting inoculum composition. This is shown in FIGS. 2A and 2B. FIG. 2A shows the design of strain segregation into 4 DS buckets based on slow and fast growing strains. FIG. 2B shows the starting inoculum seed design for fast and very fast growing strains. Using 5 iterations of the strain segregation and inoculum seed design methods, the DS1, for example, was able to increase its yield rate from approximately 35/54 strains detected at the conclusion of the coculture process to 50/54 strains detected at the conclusion of the coculture process.

Additional experimentation was required to successfully manufacture the DSs at large scale. For example, experimentation was performed on sterilization procedures and raw materials used in the media, gas solubility in the bioreactor (i.e., fermenter), shear stress caused by the impeller and gas sparging in the bioreactor, and mass transfer and mixing times. Each of these factors are necessary in order develop a process that could successfully produce a complex consortia such as any of the Consortia described herein. For example, through experimentation, it was determined that nitrogen sparging lead to higher sheering and impacted gas solubility. Accordingly, experiments were performed to adjust the speed of the sparger, location of the sparger, and replacement of sparging to gas overlay. The data showed that gas overlay was the only approach that provided successful coculture of DSs. For example, data from different sparging conditions only allowed for the detection of up to 36 out of 54 strains from DS1 while gas overlay allowed for detection of an additional 11 species at the conclusion of the coculture (i.e., 47/54 strains).

The next step in the manufacturing process that had to be developed was a method of storing the final product in a way that preserved the stability and activity of the strains. Freezing and lyophilization methods were investigated to determine what would preserve the activity and viability of the strains for each DS.

In order to determine if lyophilization would be better than freezing to preserve the activity and viability of the strains in each DS, lyophilization processes had to be developed because none were known in the art for the complexity of the DSs and Consortia provided herein. Key variables that were investigated in order to develop lyophilization process for each DS included but were not limited to: formulation of the broth or alternative microbiota suspension media, methods to prevent oxygen contamination during the lyophilization process, excipient:broth ratio, parameters for freezing the microbiota suspension prior to the lyophilization, cycle parameters for the lyophilization, sterilization requirements, methods for reviving the microbiota following lyophilized storage, buffers for reviving the microbiota, and storage of the lyophilized DS.

By means of example, high throughput, foil covered plates were used as one of the test options for storage of the lyophilized DS. This was presumed to work because the foil cover should prevent oxygen exposure. However, it was determined that foil covered plates in fact did not prevent oxygen contamination because there was no way to partially stopper the plate. Another storage method that was investigated was glass and plastic tray vials with multiplexed stoppers. The theoretical advantage of this approach was hypothesized to be the ability to do high throughput screening without the need to individually stopper each vial because the multiplexed stoppers can be pushed into the vials in a single step. However, this method proved ineffective because oxygen contamination occurred with the removal of the multiplexed stoppers. After exploring additional options for methods of preserving the lyophilized product, it was determined that individual glass vials with individual stoppers allowed for long term storage without oxygen contamination.

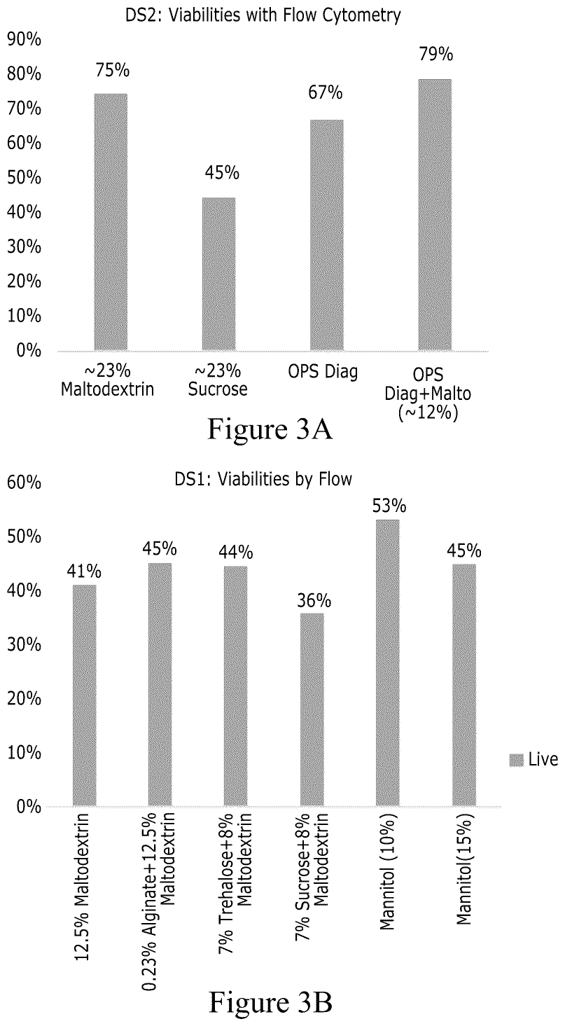

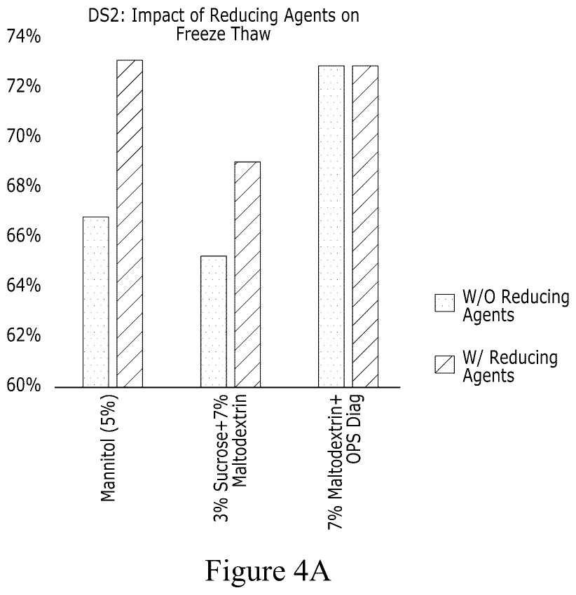

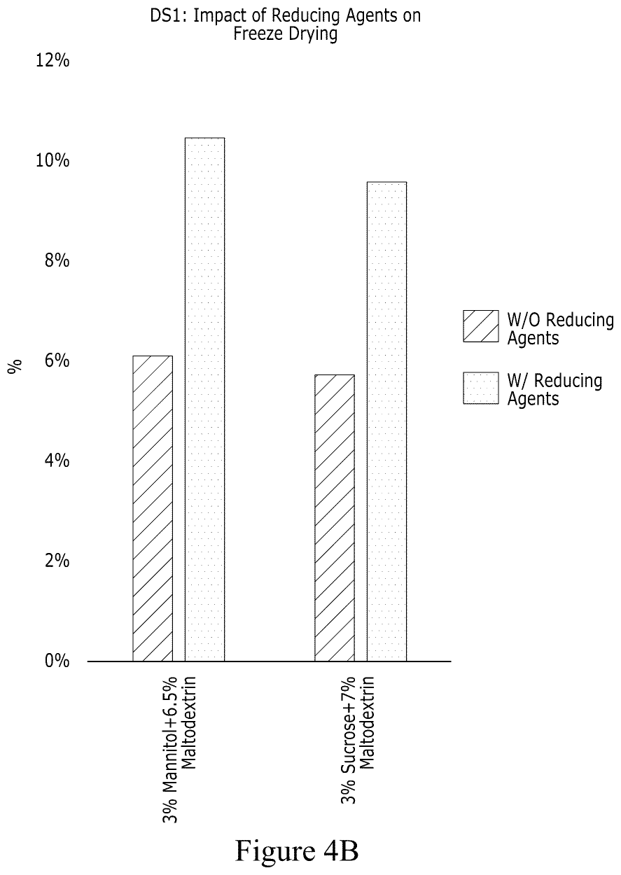

By means of a second example, it was necessary to determine the correct formulation for the lyophilization buffer/media. The following lyoprotectants were investigated to determine the correct formulation for each DS: sorbitol, maltodextrin, OPS diagnostics buffer, sucrose, inulin, alginate, mannitol, trehalose, and skim milk. For example, FIG. 3A shows examples of different viabilities of DS2 based on different lyoprotectants and FIG. 3B shows examples of different viabilities of DS1 based on different lyoprotectants. The addition of reducing agents including but not limited to cysteine HCL and riboflavin were also investigated as shown in FIG. 4A (DS2) and FIG. 4B (DS1). Additional lyophilization formulations that were tested include 8% Maltodextrin+0.5% Inulin+RA, 5% Sucrose+10% Glycerol+0.3% Inulin+RA, 7% Trehalose+8% Maltodextrin+RA, 3% Sucrose+5% Maltodextrin+0.5% Inulin+RA, 5% Maltodextrin+OPS Diag+0.5% Inulin+RA, and 5% maltodextrin+10% Glycerol+0.3% Inulin+RA.

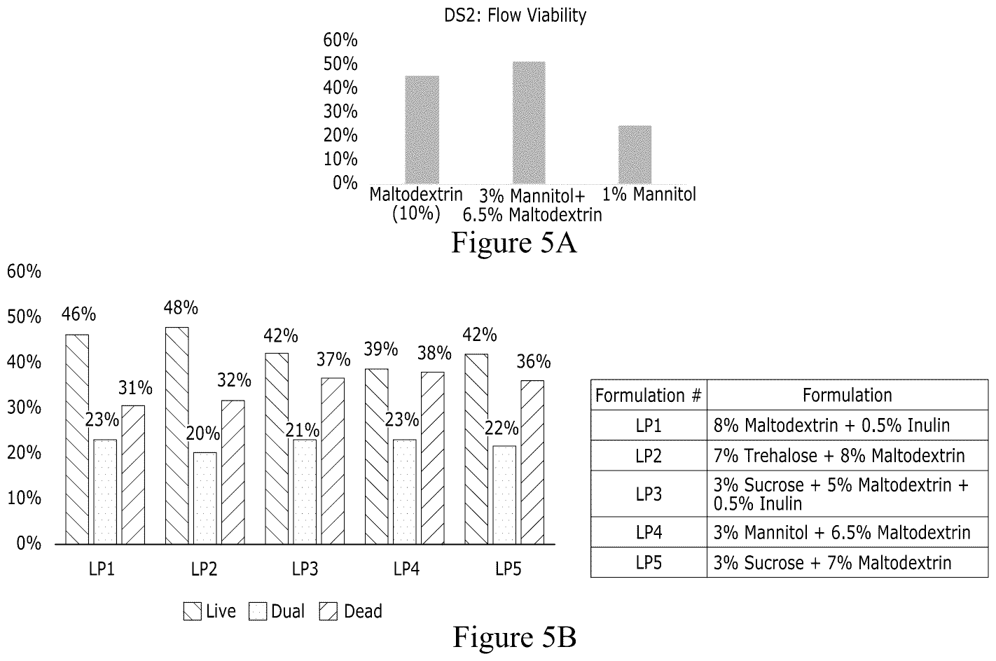

Based on freeze thaw and lyophilization experiments, data suggested that 10-12% solids was the selected dose. However, additional experiments were performed to determine if a lower dose would be possible. One exemplary experiment on DS2 is shown in FIG. 5A and a second exemplary experiment is shown in FIG. 5B.

Assays were then performed to determine the success rates of cell revival. Cell revival was done using the Anaerobe systems YCFAC media and dilution schemes were conducted using 100 fold dilution to the lyophilized powder (e.g., 50 mg (0.05 g) of powder was diluted in 5.0 mL of YCFAC media). Revival was then detected using flow cytometry and the Coulter Counter.

The experiments performed herein and the data generated determined that lyophilized material produced comparable colonization of strains in mice.

### Example 3: Synthetic Consortia

Isolation and Processing. Isolation of bacterial strains to create synthetic consortia: bacterial strains to create consortia were isolated from healthy human stool samples collected under anaerobic conditions, homogenized, and then bacterial species from each sample were identified using whole-genome sequencing (WGS). From there, the bacterial strains and abundance thereof were identified.

Stool samples were then processed and bacterial strains isolated for culture on appropriate culture media (e.g. BHI, blood agar). Isolation of oxalate degrades and strains specific to metabolize EH-related pathways were prioritized along with fastidious and unique strains and strains associated with a healthy gut microbiome. Following culture, strains were purified and sequenced using metagenomics. From the cultured, isolated strains, communities to treat enteric hyperoxaluria were created based on the notion of our bacteria to fill critical functional niches in the gut, support normal GI physiology, support engraftment of specialty strains such as O. formigenes, and degrade oxalate.

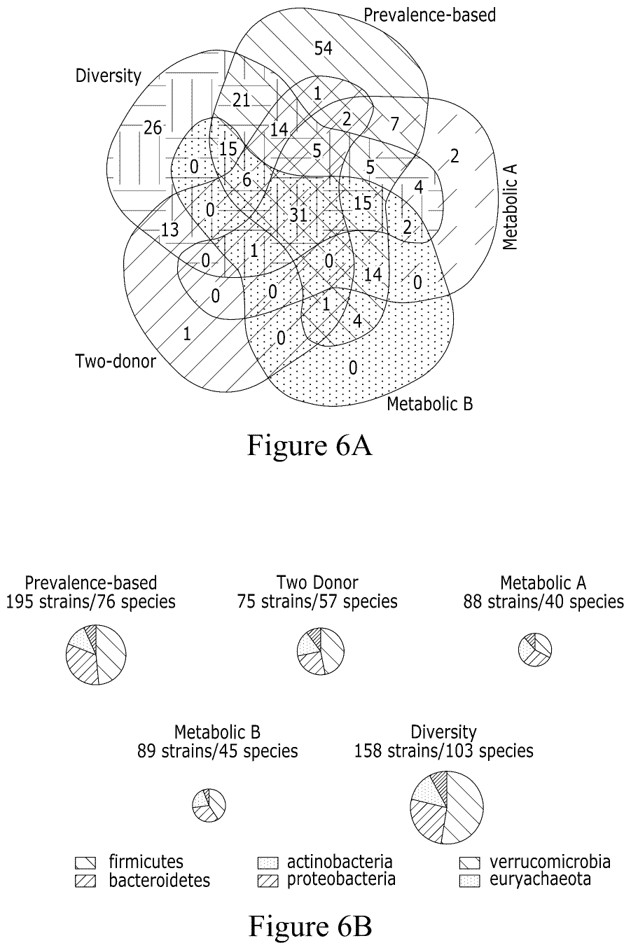

Diversity of synthetic consortia: each Consortium described herein contains unique species and strains to cover various metabolic phenotypes (e.g. bile acid metabolism, short chain fatty acid synthesis, oxalate degradation). A core set of 31 bacterial strains were similar between synthetic consortia and each community had its unique signature as indicated in the Venn diagram. The number of species present in each consortium created ranged from 40 to 103 species and the number of strains ranged from 75 to 195 as shown FIGS. 6A and 6B. The species and strains comprised varying proportions of the phylum-level diversity where the Bacteroidetes to firmicutes ratio ranges from 51% to 96% indicating that the general composition varied.

### Example 4: The Manufacture of Threonine Auxotrophic Microorganisms

Certain microorganisms are auxotrophs. This means that the microorganism is not able to synthesize a particular organic compound required for its growth. One such organic compound that certain microorganisms are incapable of synthesizing themselves is threonine. Furthermore, while some microoganisms are not per se auxotrophs of threonine, they are inefficient producers of threonine which prevent effective growth in commonly used growth medias.

N-Acetylgalactosamine (GalNAc) is an amino sugar derivative of galactose that is typically the first monosaccharide that connects serine or threonine in particular forms of protein O-glycosylation. While it is possible to supplement certain small batch growth medias with GalNAc to grow threonine auxotrophs without the addition of threonine, such supplementation is not preferred for large batch manufacture because GalNAc is costly and large amounts are needed for effective growth of microorganisms that require such galactose derivative. Furthermore, certain medias such as YCFAC media is incapable of effectively growing certain threonine auxotrophs even in the presence of GalNAc.

Accordingly, a method of improving the expansion and growth of inefficient producers of threonine is needed to effectively grow such microorganisms.

One such microorganism included in the consortia described herein is Akkermansia muciniphilia. Akkermansia is not capable of synthesizing threonine itself and thus is not able to effectively expand and grow in culture that is lacking a GalNAc source (or a primary source that can be metabolized into GalNAc). Furthermore, GalNAc is the preferred carbon source for Akkermansia and thus known methods of effectively growing and manufacturing Akkermansia comprise the addition of GalNAc to the growth media.

Accordingly, experiments were designed to identify novel methods of growing Akkermansia in large batches without large amounts of GalNAc. Specifically, three different growth medias were tested: YCFAC+GalNAc, YCFAC+GalNAc+Threonine, and YCFAC+Threonine. Since BHI is an animal-based media that contains threonine, BHI media was used as a positive control (specifically BHI media+GalNAc+Hemin+VitaminK). Because GalNAc is the preferred carbon source for Akkermansia, it was expected to be needed in all medias in order to allow expansion and growth of the microorganism; however, the expected question was how much GalNAc is needed, not whether GalNAc was needed at all, if threonine is also added. Surprisingly, it was determined that 1) YCFAC+0.5 g/L GalNAc did not support Akkermansia growth, 2) YCFAC+0.5 g/L GalNAc+10 mM threonine did support growth, and that 3) YCFAC+10 mM threonine alone supports the growth of Akkermansia. In these experiments, a seed culture containing 0.5 g/L GalNAc in YCFAC was used to initiate cell growth before being transferred to large fermenter for growth and expansion with the 3 medias described above.

However, certain of the consortia described herein comprise more than 100 different microorganisms, Akkermansia being only one of the more than 100 different microorganisms. Furthermore, the manufacturing methods described herein allow for the growth and manufacturing of multiple microorganisms in a single large batch culture (e.g., in a fermenter). The question then became how to grow Akkermansia in a large co-culture when it is the only microorganism that is a threonine auxotroph that has a preferred carbon source of GalNAc. Accordingly, an experiment was designed to determine if it was possible to start a seed culture with Akkermansia alone and then combine it with a second seed culture of multiple microorganisms for the large batch expansion.

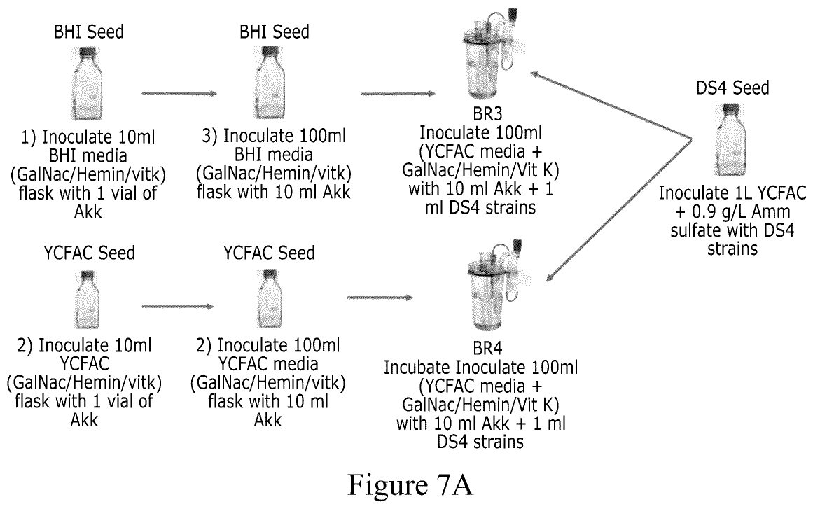

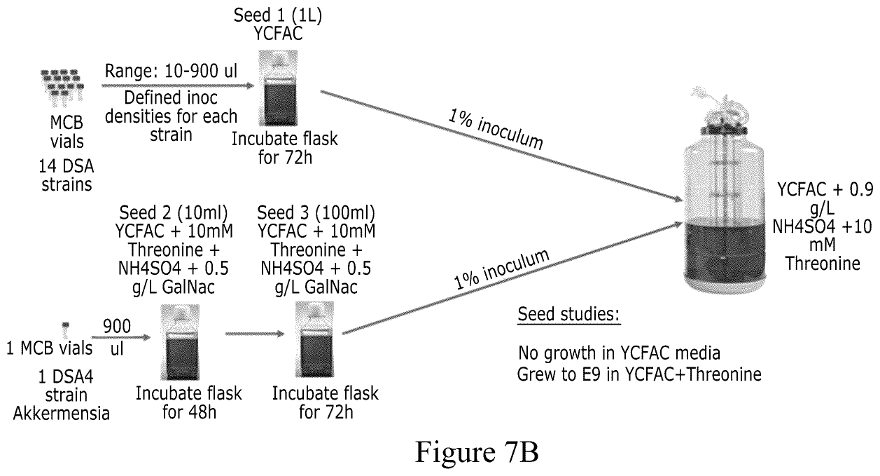

This experiment comprised: 1) a seed culture was first grown to allow the Akkermansia to begin growing in a small culture (i.e., a seed culture) of 10 mL before expansion into a large batch fermenter, 2) concurrently with the Akkermansia seed culture, a second 100 mL seed culture of all other microorganism in the drug substance was separately grown, 3) the 100 mL seed co-culture and the 10 mL Akkermansia seed culture were combined into a large batch fermenter (e.g., 1 L or more), and 4) the strains of the drug substance were detected and the ability of Akkermansia to grow and expand in the co-culture was assessed. A diagram of this experiment is shown in FIG. 7A.

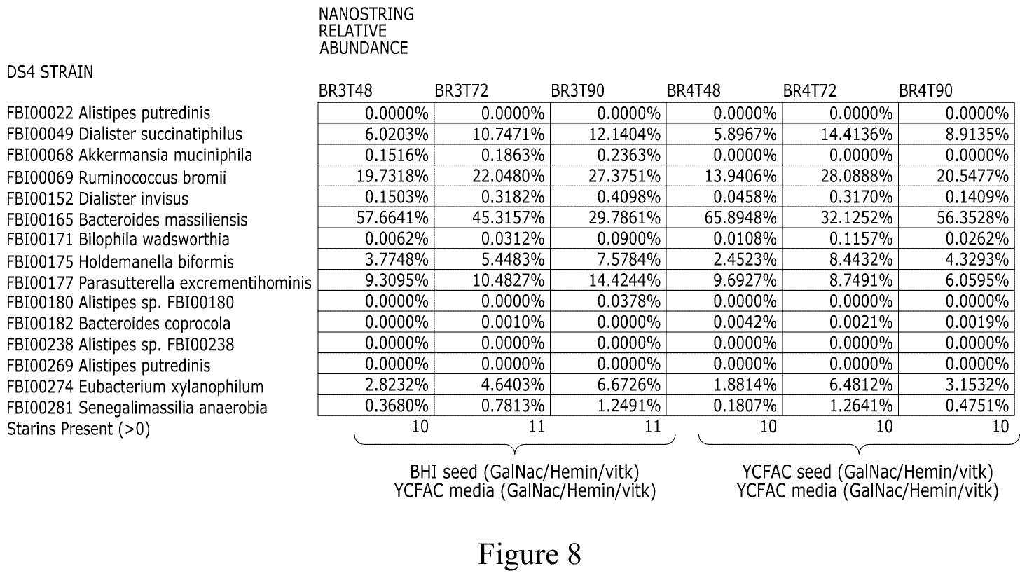

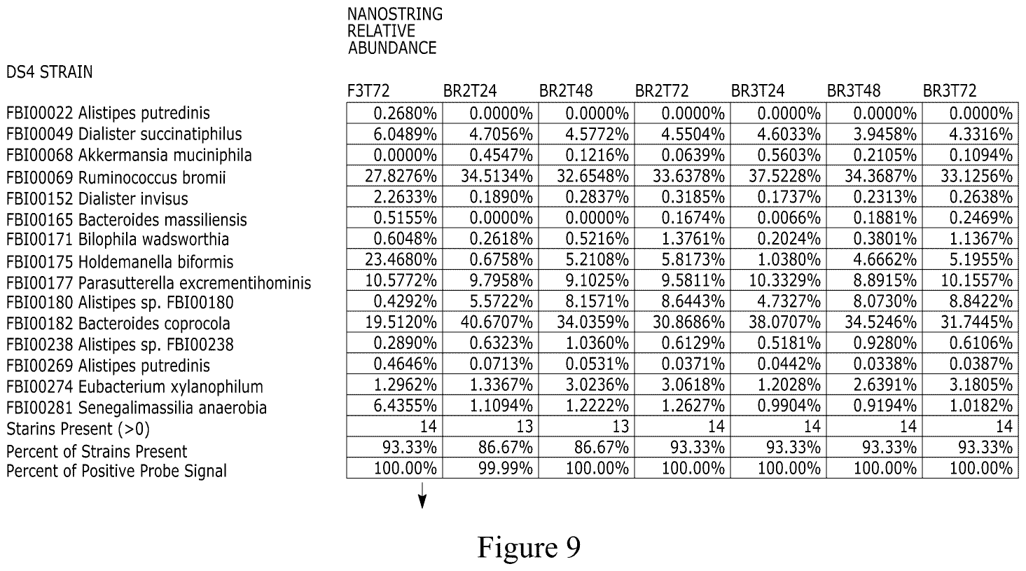

As shown in FIG. 8, it was surprising to see that Akkermansia was unable to grow in YCFAC media that was supplemented with GalNAc, Hemin, and VitaminK (0.0000% Akkermansia detected) compared to BHI media that was supplemented with GalNAc, Hemin, and VitaminK. Accordingly, it was determined that YCFAC+GalNAc cannot support the growth of Akkermansia. The question then became whether the addition of threonine could recover the growth of the Akkermansia.

The next question was whether GalNAc was needed for if threonine was added. Specifically, the question was how Akkermansia would grow in YCFAC+10 mM threonine (72 hr growth) compared to a media comprising YCFAC+10 mM threonine+0.5 g/L GalNAc (48 hr growth). It was surprising to find that the results showed comparable growth with and without the GalNAc (an OD of 0.25 for w/o GalNAc and an OD of 0.35 for w/GalNAc).

A co-culture experiment similar to that described above and shown in FIG. 7A was designed to evaluate the need for GalNAc and threonine. In this experiment, two seed cultures were used: 1) Akkermansia seed grown in YCFAC+10 mM threonine+0.5 g/L GalNAc, and 2) the other microorganisms in the drug substance (14 microorganisms) grown in YCFAC alone. The seed cultures were then combined into a large batch fermenter comprising YCFAC+10 mM threonine (i.e., no GalNAc). See FIG. 7B. This study showed that no GalNAc was needed in the presence of 10 mM threonine in a large batch fermenter in order for Akkermansia to grow in a co-culture with other microorganisms that are not threonine auxotrophs. Furthermore, in the 10 mM threonine YCFAC media, Akkermansia was detected at all growth time points (FIG. 13).

Additional experiments further showed that GalNAc was not even needed in the seed culture in order to achieve Akkermansia growth.

The ability to grow Akkermansia without GalNAc was very surprising given that GalNAc is Akkermansia's preferred carbon source. Furthermore, the ability to grow Akkermansia in a media without GalNAc provides a means of making microbial drug products comprising GalNAc wherein the Akkermansia is grown in a co-culture of multiple microbes.

### Example 5: Clinical Candidate Selection

Consortia IX was selected as the clinical candidate for clinical trials for the treatment of enteric hyperoxaluria and was termed FB-001. FB-003 was selected as the clinical trial candidate for clinical trials for the treatment of IBD, ulcerative colitis, Crohn's disease, and other associated dysbiotic diseases and conditions. FB-001 comprises 148 different anaerobic microbial strains and FB-003 comprises 145 different anaerobic microbial strains. Both FB-001 and FB-003 were designed to emulate the metabolic and phylogenetic diversity of a healthy, human microbiome (FIG. 13) and was split into 7 (for FB-001) and 4 (for FB-003) different drug substances for manufacturing purposes. Tables 1 and 2 show the different drug substances. Species were identified by 16S rRNA gene sequencing and whole genome sequencing of RCBs. The species in the consortium span six of the major phyla found in the GI tracts of healthy adults (King, Desai et al. 2019) with the deliberate exception of Fusobacteria, a phylum generally associated with human infections and enriched for opportunistic pathogens. The strains of both FB-001 and FB-003 encompass 10 distinct classes, 18 orders, 26 families, and 59 genera.

Prior to lyophilization, the cell pellet containing the FB-001 or FB-003 microbial strains were resuspended in YCFAC media with lyoprotectants and then lyophilized. The YCFAC media and lyoprotectants were chosen to stabilize the DS during the lyophilization step. The lyoprotectant combination of 8% maltodextrin+0.5% inulin was chosen for the final DS formulation as it demonstrated high viability of the FB-001 and FB-003 microbial strains in formulation development studies.

Maltodextrin was also added as a filler during DP manufacturing.

The capsules to encapsulate the DP were enteric coated and were chosen to release the DP in the small intestine and resist the gastric acids as they pass through the gastrointestinal tract. The dissolution of these capsules was tested per USP<701> at a pH of 1.2 and showed no disintegration for 2 hours. At a pH of 6.8, the capsules fully disintegrated within 30 minutes, which is the target release pH in the GI tract for FB-001 and FB-003 DP (Hydroxypropyl methylcellulose [HPMC]Capsule COA).

Function Properties of FB-001 and FB-003. FB-001 and FB-003 were manufactured using 7 or 4, respectively, individual drug substances (DS) that contain a total of 148 and 145, respectively, anaerobic microbial strains and is enriched for species performing beneficial or normalizing functions in the human GI tract.

For FB-001, the first of these beneficial or normalizing functions is oxalate degradation, which is the primary EH disease modifying mechanism of FB-001. Oxalobacter formigenes is the principal driver of oxalate degradation in the human GI tract. O. formigenes uses oxalate as its exclusive energy source, metabolizing significant concentrations of oxalate for energy generation and biomass production. The metabolism of oxalate is mediated by a series of enzymatic and transport reactions that ultimately consume oxalate and release CO2 and formate.

Formate, as a by-product of oxalate metabolism, can ultimately inhibit further oxalate metabolism in vitro if it is not removed. Therefore, FB-001 also contains strains capable of formate degradation. These formate-utilizing bacteria help to clear the potentially inhibitory metabolic byproducts of oxalate metabolism.

FB-001 and FB-003 also contain strains that are oxalate resistant, able to grow in the presence of oxalate concentrations that are over a magnitude or higher than the physiologically normal concentrations of oxalate. This enrichment of oxalate-tolerant strains in the FB-001 and FB-003 consortium may support stable engraftment despite potentially elevated levels of free oxalate in the GI lumen of patients with EH, as the abundance of the key oxalotrophs will naturally increase with spikes in oxalate concentration.

The FB-001 and FB-003 Consortia were specifically designed to contain phylogenetically diverse microbial species that function mutualistically to improve the dysbiosis associated with malabsorption. This was achieved by the inclusion of numerous species intended to support the community by restoring essential metabolic functions. The strains that make up the FB-001 and FB-003 Consortia were selected based on their predicted ability to perform a variety of supportive metabolic functions that would contribute to engraftment regardless of differences in patient physiology or diet. Metabolism of macronutrients and dietary molecules that are not digested or utilized by host cells may result in the release of metabolic products that feed other members of the microbiome community.

Other strains in FB-001 and FB-003 were evaluated for unique and potentially beneficial biological functions in the GI tract, including production of short-chain fatty acids (SCFAs), cross-feeding activity, and mucin degradation. SCFAs are absorbed by the host and have been recognized to confer a range of health-promoting functions by acting as key energy substrates for colonocytes, enterocytes, and hepatocytes, while also acting as signaling molecules recognized by specific G-protein couple receptors targeting primarily enteroendocrine and immune cells in the lamina propria of the intestinal mucosa. Strains in FB-001 and FB-003 were evaluated for their cross-feeding activity, a process in which bacteria make by-products that feed other bacteria. Cross-feeding stabilizes the gut microbiome and creates novels niches. Strains in FB-001 and FB-003 were also evaluated for putative protective and/or anti-inflammatory properties.

Table 3 summarizes the number of strains in FB-001 that contribute to each of these functional properties, and characteristics that are associated with each FB-001 species are summarized in Table 4.

TABLE 3
Function Properties of FB-001 and FB-003 DPs

| | | |
|---|---|---|
| | | Number of FB-001 |
| Properties | Classification | DP Strains |

FB-001

| | | |
|---|---|---|
| Oxalate and | Oxalate degradation | 7 |
| formate | Oxalate resistance | 38 |
| metabolism | Formate metabolism | 45 |
| Supportive | Metabolism of macronutrients | 98 |
| metabolic | Production of microbial metabolites | 70 |
| functions | Production of short-chain fatty acids | 131 |
| | Cross-feeding activity | 12 |
| | Mucin degradation | 4 |

| | |
|---|---|
| Putative protection against disease | 22 |
| Prevalence in healthy human gut | 97 |

FB-003

| | | |
|---|---|---|
| Oxalate and | Oxalate degradation | 4 |
| formate | Oxalate resistance | 35 |
| metabolism | Formate metabolism | 45 |
| Supportive | Metabolism of macronutrients | 98 |
| metabolic | Production of microbial metabolites | 70 |
| functions | Production of short-chain fatty acids | 131 |
| | Cross-feeding activity | 12 |
| | Mucin degradation | 4 |

| | |
|---|---|
| Putative protection against disease | 22 |
| Prevalence in healthy human gut | 97 |

TABLE 4
Species Included in FB-001 and FB-003 Drug Products and Characteristics

| | | | | | | | | | | | Known |
|---|---|---|---|---|---|---|---|---|---|---|---|
| | | | | | | | Production | | | Putative | Preva- |
| | | | | | Metabo- | Produc- | of Short- | | | Protec- | lence in |
| | | Oxalate | Oxalate | Formate | lism of | tion of | Chain | Cross- | Mucin | tion | Healthy |
| | # | Degra- | Resis- | Metab- | Macronu- | Microbial | Fatty | Feeding | Degra- | Against | Human |
| Species (by phylum) | strains | dation | tance | olism | trients | Metabolites | Acids | Activity | dation | Disease | Gut |
| Actinobacteria | | | | | | | | | | | |
| Bifidobacterium adolescentis | 3 | | | | ● | ● | ● | | | | ● |
| Bifidobacterium bifidum | 1 | | | | ● | | | ● | ● | | ● |
| Bifidobacterium catenulatum | 1 | | | | ● | ● | ● | | | | ● |
| Bifidobacterium dentium | 1 | ● | | | ● | ● | ● | | | | ● |
| Bifidobacterium longum | 2 | | | | ● | ● | ● | | | ● | ● |
| Bifidobacterium | 3 | ● | | | ● | ● | ● | | | | ● |
| pseudocatenulatum | | | | | | | | | | | |
| Collinsella aerofaciens | 2 | | | ● | ● | ● | ● | | | | ● |
| Eggerthella lenta | 4 | | ● | ● | ● | | ● | | | | ● |
| Gordonibacter pamelaeae | 2 | | | ● | ● | | ● | | | | ● |
| Senegalimassilia anaerobia | 1 | | | ● | | | | | | | |
| Bacteroidetes | | | | | | | | | | | |
| Alistipes onderdonkii | 2 | | | | ● | ● | ● | | | ● | ● |
| Alistipes putredinis | 2 | | | | | ● | ● | | | | ● |
| Alistipes senegalensis | 1 | | | | ● | | | | | | ● |
| Alistipes shahii | 2 | | | | ● | | ● | | | ● | ● |
| Alistipes timonensis | 1 | | | ● | ● | | ● | | | | ● |
| Alistipes sp. FBI00180 | 1 | | | | | | ● | | | | |
| Alistipes sp. FBI00238 | 1 | | | | | | ● | | ● | | |
| Bacteroides caccae | 2 | | | | ● | | ● | | | ● | ● |
| Bacteroides coprocola | 1 | | | | | | | | | | ● |
| Bacteroides faecis | 1 | | | ● | | ● | ● | | | | ● |
| Bacteroides finegoldii | 1 | | | ● | | | | | | | ● |
| Bacteroides fragilis | 1 | | | | ● | | ● | | | | ● |
| Bacteroides kribbi | 2 | | | ● | ● | | ● | | | | |
| Bacteroides massiliensis | 1 | | | | ● | | ● | | | | ● |
| Bacteroides nordii | 1 | | | | | | ● | | | | ● |
| Bacteroides ovatus | 1 | | | ● | ● | | | | | ● | ● |
| Bacteroides salyersiae | 1 | | | ● | | | ● | | | | ● |
| Bacteroides stercorirosoris | 1 | | | ● | | | ● | | | | |
| Bacteroides stercoris | 2 | | | | ● | ● | ● | | | | ● |
| Bacteroides thetaiotaomicron | 2 | | ● | ● | ● | ● | ● | ● | | | ● |
| Bacteroides uniformis | 2 | | | | ● | | ● | ● | | | ● |
| Bacteroides vulgatus | 2 | | | ● | ● | ● | ● | | | | ● |
| Bacteroides xylanisolvens | 3 | | ● | ● | ● | ● | ● | | | | ● |
| Barnesiella intestinihominis | 1 | | | | | | | | | ● | ● |
| Butyricimonas faecihominis | 1 | | | | ● | ● | ● | | | | |
| Parabacteroides distasonis | 1 | | ● | | ● | ● | ● | | | | ● |
| Parabacteroides merdae | 2 | | | | ● | | ● | | | | ● |
| Paraprevotella clara | 1 | | | ● | ● | ● | ● | | | | ● |
| Porphyromonas asaccharolytica | 1 | | | | | | ● | | | | ● |
| Euryarchaeota | | | | | | | | | | | |
| Methanobrevibacter smithii | 2 | | | ● | ● | ● | ● | | | | ● |
| Acidaminococcus intestini | 1 | | | | | ● | ● | | | | ● |
| Acutalibacter timonensis | 1 | | ● | | | | ● | | | | |
| Anaerofustis stercorihominis | 1 | | ● | | ● | ● | ● | | | | ● |
| Anaerostipes hadrus | 2 | | | | ● | ● | ● | ● | | | ● |
| Anaerotruncus massiliensis | 1 | | | ● | ● | | | | | | ● |
| Blautia faecis | 1 | | ● | | | ● | ● | | | | ● |
| Blautia hydrogenotrophica | 1 | | | ● | | ● | ● | | | | ● |
| Blautia massiliensis | 1 | | | ● | | ● | ● | | | | |
| Blautia obeum | 2 | | ● | | ● | ● | ● | | | ● | ● |
| Blautia wexlerae | 2 | | | ● | ● | ● | ● | | | ● | |
| Catabacter hongkongensis | 1 | | ● | | ● | | | | | | ● |
| Clostridiaceae sp. FBI00191 | 1 | | | | | | ● | | | | |
| Clostridium aldenense | 2 | | ● | | ● | | ● | | | | ● |
| Clostridium bolteae | 2 | | ● | ● | ● | | ● | | | | ● |
| Clostridium citroniae | 2 | | ● | | ● | | ● | | | | ● |
| Clostridium clostridioforme | 1 | | | | | | ● | | | | ● |
| Clostridium fessum | 1 | | | | | | ● | | | | |
| Clostridium scindens | 1 | | | | ● | | ● | | | | |
| Coprococcus comes | 2 | | | | ● | ● | ● | | | | ● |
| Coprococcus eutactus | 1 | | | | | | ● | | | | ● |
| Dialister invisus | 1 | | | | | | ● | | | | ● |
| Dialister succinatiphilus | 1 | | ● | | ● | ● | ● | | | | |
| Dielma fastidiosa | 1 | | | | ● | | | | | | |
| Dorea formicigenerans | 2 | | | ● | ● | | ● | | | | ● |
| Dorea longicatena | 2 | | | | ● | | ● | | | | ● |
| Eisenbergiella tayi | 2 | | | | | ● | ● | | | | |
| Emergencia timonensis | 1 | | | | ● | ● | | | | | |
| Eubacterium eligens | 2 | | | | ● | | ● | | | ● | |
| Eubacterium hallii | 1 | | | | ● | ● | ● | | | | ● |
| Eubacterium rectale | 2 | | | | ● | | ● | ● | | | ● |
| Eubacterium siraeum | 1 | | | | ● | | | | | | |
| Eubacterium ventriosum | 1 | | | | ● | | | | | ● | ● |
| Eubacterium xylanophilum | 1 | | | | ● | ● | ● | | | | ● |
| Faecalibacterium prausnitzii | 1 | | | ● | ● | ● | ● | | ● | ● | ● |
| Fusicatenibacter saccharivorans | 2 | | | | | ● | ● | | | | |
| Holdemanella biformis | 1 | | | | | ● | | | | ● | ● |
| Hungatella effluvii | 1 | | | | | | ● | | | | |
| Hungatella hathewayi | 2 | | | | ● | ● | ● | | | | |
| Lachnoclostridium pacaense | 2 | | | | | | ● | | | | |
| Lachnospiraceae sp. FBI00033 | 1 | | | | | | ● | | | | |
| Lachnospiraceae sp. FBI00071 | 1 | | | | | | ● | | | | |
| Lachnospiraceae sp. FBI00290 | 1 | | | | | | ● | | | | |
| Lactobacillus rogosae | 2 | | | | | | ● | | | | |
| Longicatena caecimuris | 1 | | | | | | ● | | | | |
| Megasphaera massiliensis | 1 | | | ● | | ● | ● | | | | |
| Monoglobus pectinilyticus | 2 | | ● | | ● | ● | ● | | | | |
| Phascolarctobacterium faecium | 1 | | ● | | | | | | | | |
| Roseburia hominis | 2 | | ● | | ● | ● | ● | | | ● | ● |
| Ruminococcaceae sp. FBI00097 | 1 | | | | | ● | ● | | | | |
| Ruminococcaceae sp. FBI00233 | 1 | | | | | ● | ● | | | | |
| Ruminococcus bromii | 2 | | ● | | ● | | ● | ● | | | ● |
| Ruminococcus faecis | 2 | | ● | | ● | ● | ● | | | | |
| Ruthenibacterium lactatiformans | 1 | | | | ● | ● | ● | | | | |
| Turicibacter sanguinis | 1 | | | | | | ● | | | ● | |
| Proteobacteria | | | | | | | | | | | |
| Bilophila wadsworthia | 2 | | | ● | ● | | ● | | | | |
| Oxalobacter formigenes* | 3 | ● | ● | | | | | | | | |
| Parasutterella excrementihominis | 2 | | | ● | | ● | ● | | | | ● |
| Sutterella massiliensis | 1 | | ● | ● | | ● | ● | | | | |
| Sutterella wadsworthensis | 2 | | ● | ● | | | ● | | | | ● |
| Verrucomicrobia | | | | | | | | | | | |
| Akkermansia muciniphila | 1 | | | | ● | | ● | ● | ● | | ● |

*not included in FB-003

Formate Metabolism. The FB-001 and FB-003 DP consortium contain formate-utilizing bacteria to maintain maximal carbon flux through the pathway. Symbiotic bacterial species such as methanogens found in the human GI tract can efficiently remove formate via reduction to methane in the presence of hydrogen gas produced by microbial fermenters. Therefore, the FB-001 and FB-003 Consortia includes Methanobrevibacter smithii (DS-CoC2), the most prevalent and abundant archaeal methanogen in the gut, and one that efficiently metabolizes formate, as well as the acetogenic gut commensal Blautia hydrogenotrophica (DS-CoC1), which utilizes formate to generate acetate for short-chain fatty acid (SCFA) synthesis, and a panel of anaerobes (eg, Sutterella and Parasutterella, found in DS-CoC2 and DS-CoC4) that express cytochrome-dependent formate dehydrogenases that oxidize formate to CO₂.

Supportive Metabolic Functions. FB-001 and FB-003 also contain a diverse panel of broadly functional commensals that fulfill unique and potentially beneficial biological functions in the GI tract, including metabolism of macro-nutrients, production of short-chain fatty acids, cross-feeding activity, and mucin degradation.

Composition of FB-001 and FB-003 DPs. FB-001 DP is a highly complex, mixed fermentation of 148 microbial strains, chosen for their potential role in supporting a healthy GI tract. Similarly, FB-003 DP is a highly complex, mixed fermentation of 145 microbial strains, chosen for their potential role in supporting a healthy GI tract.

To support clinical studies, FB-001 DP was characterized for relative abundance of individual species in the final DP using metagenomic sequencing, as well as for total O. formigenes content. In metagenomic sequencing and analysis, strains were first confirmed to be present in the sample by positive identification of pre-specified biomarkers (short sequences of DNA) that are unique to the strain of interest. Then, the results of metagenomic sequencing were reported as the relative abundance of each strain, which approximates the percentage of genome copies that belong to each strain and can range from 0 to 100%. The relative abundance was then calculated by comparing the number and frequency of detected biomarkers to the total number of strain-specific biomarkers and the number of sequencing reads. The percent contribution of each strain in the FB-001 DP comprises a predominant portion of the three O. formigenes strains identified by 16S RNA and carbon source analysis described below as follows: approximately 32% O. formigenes on a relative abundance basis (i.e., approximately 40% on a viable cell count basis) with the other 145 strains having relative abundance values ranging from 18% to 0.015% (distribution of a typical human microbiome).

FB-001 DP was manufactured as a single batch. A single capsule of DP from was collected and stored at −20° C.±5 until DNA extraction. FB-001 DP was sequenced via shotgun metagenomics and the metagenomic sequences of DP were analyzed to determine the composition of FB-001 DP. Results were reported as the relative abundance of each strain. Relative abundance approximates the percentage of FB-001 DP genome copies that belong to each strain and can range from 0 to 100%. A total of 60 of 148 strains were detected at or above their qualified limit of detection, including 21 strains from DS-CoC1, 13 strains from DS-CoC2, 16 strains from DS-CoC3, 7 strains from DS-CoC4, and each of DS-OF1, DS-OF2, and DS-OF3. The absence of detection of a strain should not be interpreted as its absence from the drug substance. The 60 detected strains account for 95.932% of the biomarkers detected in FB-001 DP. The remaining 88 strains therefore account for 4.068% of the biomarkers. The relative abundance profile is expected to vary between batches and data will continue to be collected during development to understand the magnitude of the variability. Furthermore, the exact percentages should not be interpreted as limiting or exclusive; rather each batch of DP may vary in its microbial distribution based on natural growth of bacterial in co-cultures. An example of the relative abundance profile of the microbes in one lot of FB-001 is provided in Table 5.

TABLE 5
Relative Abundance Profile of a FB-001 DP Lot

| | Relative | | Relative | | Relative |
|---|---|---|---|---|---|
| Strain | Abundance (%) | Strain | Abundance (%) | Strain | Abundance (%) |
| FBI00180 | 18 | FBI00245 | 0.076 | FBI00097 | <LoD |
| FBI00289 | 11 | FBI00043 | 0.063 | FBI00099 | <LoD |
| FBI00067 | 11 | FBI00237 | 0.059 | FBI00109 | <LoD |
| FBI00133 | 10 | FBI00206 | 0.052 | FBI00113 | <LoD |
| FBI00038 | 4.3 | FBI00032 | 0.04 | FBI00115 | <LoD |
| FBI00175 | 4.1 | FBI00243 | 0.038 | FBI00117 | <LoD |
| FBI00255 | 3.9 | FBI00116 | 0.035 | FBI00123 | <LoD |
| FBI00120 | 3.3 | FBI00002 | 0.032 | FBI00126 | <LoD |
| FBI00177 | 2.5 | FBI00167 | 0.031 | FBI00127 | <LoD |
| FBI00212 | 2.4 | FBI00068 | 0.015 | FBI00132 | <LoD |
| FBI00025 | 2.2 | FBI00010 | <LoD | FBI00137 | <LoD |
| FBI00060 | 1.8 | FBI00011 | <LoD | FBI00145 | <LoD |
| FBI00048 | 1.6 | FBI00012 | <LoD | FBI00149 | <LoD |
| FBI00104 | 1.5 | FBI00013 | <LoD | FBI00159 | <LoD |
| FBI00151 | 1.5 | FBI00015 | <LoD | FBI00162 | <LoD |
| FBI00220 | 1.1 | FBI00018 | <LoD | FBI00165 | <LoD |
| FBI00004 | 1 | FBI00019 | <LoD | FBI00170 | <LoD |
| FBI00020 | 0.93 | FBI00021 | <LoD | FBI00174 | <LoD |
| FBI00251 | 0.91 | FBI00022 | <LoD | FBI00182 | <LoD |
| FBI00016 | 0.73 | FBI00030 | <LoD | FBI00184 | <LoD |
| FBI00079 | 0.72 | FBI00033 | <LoD | FBI00189 | <LoD |
| FBI00102 | 0.71 | FBI00034 | <LoD | FBI00190 | <LoD |
| FBI00233 | 0.65 | FBI00040 | <LoD | FBI00191 | <LoD |
| FBI00171 | 0.64 | FBI00044 | <LoD | FBI00194 | <LoD |
| FBI00029 | 0.63 | FBI00046 | <LoD | FBI00200 | <LoD |
| FBI00076 | 0.56 | FBI00047 | <LoD | FBI00201 | <LoD |
| FBI00147 | 0.53 | FBI00049 | <LoD | FBI00208 | <LoD |
| FBI00128 | 0.5 | FBI00050 | <LoD | FBI00221 | <LoD |
| FBI00197 | 0.49 | FBI00051 | <LoD | FBI00224 | <LoD |
| FBI00110 | 0.47 | FBI00052 | <LoD | FBI00229 | <LoD |
| FBI00112 | 0.47 | FBI00053 | <LoD | FBI00235 | <LoD |
| FBI00135 | 0.47 | FBI00056 | <LoD | FBI00236 | <LoD |
| FBI00226 | 0.44 | FBI00057 | <LoD | FBI00238 | <LoD |
| FBI00199 | 0.43 | FBI00059 | <LoD | FBI00244 | <LoD |
| FBI00152 | 0.43 | FBI00061 | <LoD | FBI00248 | <LoD |
| FBI00027 | 0.42 | FBI00066 | <LoD | FBI00254 | <LoD |
| FBI00211 | 0.4 | FBI00069 | <LoD | FBI00258 | <LoD |
| FBI00232 | 0.37 | FBI00070 | <LoD | FBI00260 | <LoD |
| FBI00111 | 0.31 | FBI00071 | <LoD | FBI00267 | <LoD |
| FBI00140 | 0.3 | FBI00075 | <LoD | FBI00269 | <LoD |
| FBI00263 | 0.25 | FBI00077 | <LoD | FBI00270 | <LoD |
| FBI00124 | 0.24 | FBI00078 | <LoD | FBI00271 | <LoD |
| FBI00001 | 0.23 | FBI00080 | <LoD | FBI00273 | <LoD |
| FBI00198 | 0.21 | FBI00081 | <LoD | FBI00274 | <LoD |
| FBI00176 | 0.2 | FBI00085 | <LoD | FBI00277 | <LoD |
| FBI00125 | 0.2 | FBI00087 | <LoD | FBI00278 | <LoD |
| FBI00205 | 0.17 | FBI00092 | <LoD | FBI00288 | <LoD |
| FBI00062 | 0.096 | FBI00093 | <LoD | FBI00290 | <LoD |
| FBI00281 | 0.095 | FBI00096 | <LoD | FBI00292 | <LoD |
| FBI00009 | 0.09 | | | | |

Process Development. The blending process during DP manufacture for FB-001 and FB-003 was developed to create a homogenous mixture of the DSs. During the development phase, the blend-sieve-blend technique for mixing the DSs was tested. Using this technique, several of the DSs were blended in a Turbula mixer for 15 minutes at 43 rpm followed by sieving of the material through #50 sieve. The material was again blended for 15 minutes at 43 rpm. An aliquot of blended material from the top, middle and bottom of the container were taken and evaluated for TCC, VCC and strain distribution by relative abundance. The blending study results showed that the DS material was homogenously mixed with blend-sieve-blend mixing technique. For the FB-001 DP, the VCC/g, TCC/g and relative abundance of the three O. formigenes strains in the top, middle and bottom of the mixing container are very similar, which indicates a homogenous blend of DSs in the blending container.

A diagram of the coculture method of manufacture is provided in FIG. 10.

Manufacture of DS1. Yeast casitone fatty acids with carbohydrates (YCFAC) medium, pH 7, was prepared at 1× concentration in batches of 4 L each for Seed 1 fermentation and Seed 2 fermentation. The medium was prepared by adding the components indicated in Table 6 to 3.46 kg of water for injection, boiling for 5 to 10 minutes, then allowing the medium to cool down. Upon reaching a temperature of 50° C. or lower, the medium was sparged with N2 while the rest of the components were added in the following order: sodium bicarbonate, 50× volatile fatty acid solution, L-cysteine H monohydrate, 0.5% hemin solution, and 25× vitamin solution. The pH was adjusted to 7 with 10 N NaOH or sulfuric acid, and the medium was autoclaved at 122.5° C. for 45 minutes. The medium was incubated at 37° C. for a minimum of 24 hours prior to inoculation for a contamination check.

TABLE 6
YCFAC Media

| | Quantity/4.0 L of | | | | |
|---|---|---|---|---|---|
| Reagent Description | Medium | Unit | Final Conc. (1X) | | Addition |
| Soytone | 40.00 | g | 1% | w/w | Boiled for |
| D-cellobiose | 8.00 | g | 0.2% | w/w | 5 to 10 minutes in |
| Yeast extract | 10.00 | g | 0.25% | w/w | 3.46 kg Water for |
| Dextrose (glucose) | 20.00 | g | 0.5% | w/w | Injection (WFI) |
| Maltose monohydrate | 8.00 | g | 0.2% | w/w | |
| Magnesium sulfate | 0.36 | g | 0.009% | w/w | |
| heptahydrate | | | | | |
| Calcium chloride dihydrate | 0.36 | g | 0.009% | w/w | |
| Potassium phosphate | 1.80 | g | 0.045% | w/w | |
| monobasic | | | | | |
| Potassium phosphate dibasic | 1.80 | g | 0.045% | w/w | |
| Sodium chloride | 3.60 | g | 0.09% | w/w | |
| Sodium bicarbonate (7.5%) | 213.0 | mL | 5.325% | w/w | Added after the media |
| Volatile fatty acid solution | 11.56 | mL | 1X volatile fatty acid | | cools down to 50° C. or |
| (50X) | | | solution^{b} | | lower |
| L-cysteine HCl monohydrate | 4.0 | g | 0.1% | w/w | |
| Hemin solution (0.5% w/w) | 8.00 | mL | 0.2% | w/w | |

| | | | |
|---|---|---|---|
| Vitamin solution (25X) | 160.00 | mL | 1X vitamin solution |

A 5× concentration media was also made for use in the main fermentation. The 5× stock was made using the same proportions as described in Table 6, scaled up to 5×. The 5× media was diluted to a 1× concentration before the main fermentation process.

Resuspension medium was also made and comprised YCFAC medium with reducing agents L-cysteine HCO and riboflavin, pH 7. To prepare resuspension medium, 0.6 g of riboflavin and 2.0 g of cysteine-HCl are added per kg of YCFAC medium. The medium is stirred until completely dissolved, then titrated with 10 N NaOH or sulfuric acid to obtain a final pH of 7. The medium is filtered with a 0.22 μm polyethersulfone (PES) filter. The final concentration of Riboflavin was 0.06% and the final concentration of L-cysteine HCL was 0.2%, in YCFAC media.

The volatile fatty acid solution (50×) for the YCFAC media was made and comprised Glacial acetic acid (65.7% w/w for the 50× concentration; 1.31% w/w for the 1× concentration), Propionic acid (24.2% w/w for the 50× concentration; 0.48% w/w for the 1× concentration), Iso-butyric acid (3.1% w/w for the 50× concentration; 0.06% w/w for the 1× concentration), n-Valeric acid (3.5% w/w for the 50× concentration; 0.07% w/w for the 1× concentration), and Iso-valeric acid (3.5% w/w for the 50× concentration; 0.07% w/w for the 1× concentration).

The vitamin solution (25×) for the YCFAC media comprised Biotin powder (1.31 Quantity/6 kg WFI (g)), Folic acid (1.31 Quantity/6 kg WFI (g)), Pyridoxine hydrochloride (6.56 Quantity/6 kg WFI (g)), Thiamine-HCl-2H₂O (3.28 Quantity/6 kg WFI (g)), Riboflavin (0.13 Quantity/6 kg WFI (g)), Nicotinic acid (3.28 Quantity/6 kg WFI (g)), D-calcium pantothenate (3.28 Quantity/6 kg WFI (g)), Vitamin B12 (0.07 Quantity/6 kg WFI (g)), 4-aminobenzoic acid (3.28 Quantity/6 kg WFI (g)), and DL-alfa-lipoic acid (3.28 Quantity/6 kg WFI (g)).

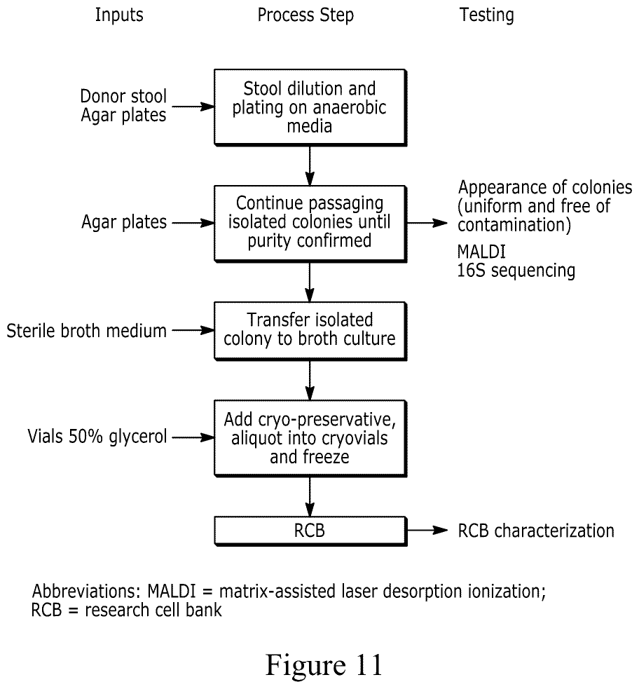

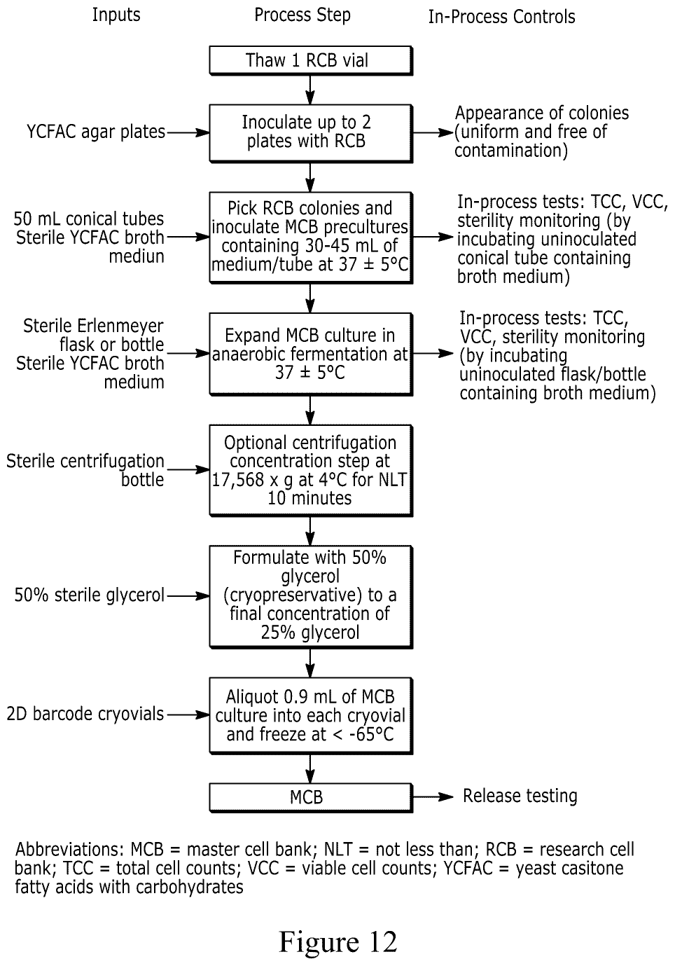

Microbial strains intended for FB-001 and FB-003 DS-CoC1 were isolated from stool samples obtained after extensive donor screening. An overview of the strain isolation and purification process, RCB banking, and RCB identity/purity testing is provided in FIGS. 11 and 12. The entire stool sample homogenization and aliquoting was carried out under anaerobic conditions, starting with transfer of the stool sample to the anaerobic chamber within 15 to 30 minutes of the collection, followed by homogenization and addition of a 1:1 solution of PBS and 50% glycerol prior to aliquoting into 6 to 9 separate cryovials and transferring to <−65° C. for storage until further processing.

To isolate individual strains, fecal samples were serially diluted and then plated onto a variety of agar plates containing anaerobic microbial cultivation media (counted as passage 1). The plates were incubated at 37° C. under anaerobic conditions. Single colonies from these initial growth plates were picked for further isolation on appropriate microbial cultivation agar media plates (counted as passage 2). After incubation at 37° C., if the single-colony plating resulted in isolated colonies with uniform morphology, the culture was further characterized for strain identification. Preliminary strain identification was performed either by 16S rRNA gene sequencing or by creating and analyzing proteomic fingerprinting using high-throughput matrix-assisted laser desorption/ionization-time of flight spectrometry. If the single-colony plating resulted in multiple colony morphologies, each unique colony type was picked from this plating for further isolation on an appropriate cultivation agar plate until uniform colony morphology was achieved (counted as passage 3 or more). The passage history of each strain in FB-001 and FB-003 DS-CoC1 and the agar and broth medias are listed in Table 7.

TABLE 7
Isolation of Research Cell Banks Used in FB-001 and FB-003 DS-CoC1

| | RCB Agar Passaging | | RCB Broth Passaging | |
|---|---|---|---|---|
| FBI Strain | | Passage | | Passage |
| ID | Agar Type | # | Broth Type | # |
| FBI00001 | Bifidobacterium selective agar + 40 mM | 1 | YCFAC, pH 6.0 + 40 mM | 1 |
| | oxalate | | oxalate | |
| | Bifidobacterium selective agar | 2 | | |
| FBI00002 | Bifidobacterium selective agar + 40 mM | 1 | YCFAC, pH 6.0 + 40 mM | 1 |
| | oxalate | | oxalate | |
| | Bifidobacterium selective agar | 2 | | |
| FBI00010 | YCFAC-B agar | 3 | YCFAC, pH 6.0 | 1 |
| FBI00013 | YCFAC-B agar | 3 | YCFAC, pH 6.0 | 1 |
| FBI00029 | Brain heart infusion agar with hemin and | 2 | YCFAC, pH 6.0 | 1 |
| | vitamin K | | | |
| FBI00032 | Bifidobacterium selective agar | 3 | YCFAC, pH 6.0 | 1 |
| | YCFAC agar | 1 | | |
| FBI00033 | YCFAC-B agar + 20 mM oxalate | 1 | YCFAC, pH 6.0 + 20 mM | 1 |
| | YCFAC-BO 40 mM agar | 2 | oxalate | |
| | YCFAC agar | 1 | | |
| FBI00034 | Brain heart infusion agar with hemin and | 2 | YCFAC, pH 6.0 | 1 |
| | vitamin K | | | |
| FBI00043 | Reinforced clostridial agar | 2 | YCFAC, pH 6.0 | 1 |
| FBI00044 | Chocolate agar | 2 | YCFAC, pH 6.0 | 1 |
| FBI00048 | YCFAC-B agar | 3 | YCFAC, pH 6.0 | 1 |
| FBI00050 | Bifidobacterium selective agar + 40 mM | 1 | YCFAC, pH 6.0 + 40 mM | 1 |
| | oxalate | | Oxalate | |
| | YCFAC-BO 40 mM agar | 2 | | |
| FBI00051 | YCFAC-B agar | 2 | YCFAC, pH 6.0 | 1 |
| | YCFAC agar | 1 | | |
| FBI00057 | Reinforced clostridial agar | 2 | YCFAC, pH 6.0 | 1 |
| | YCFAC agar | 1 | | |
| FBI00059 | Columbia agar with 5% sheep blood | 3 | YCFAC, pH 6.0 | 1 |
| FBI00060 | YCFAC-B agar | 3 | YCFAC, pH 6.0 | 1 |
| | YCFAC-BO 40 mM agar | 2 | YCFAC, pH 6.0 + 40 mM | |
| FBI00070 | YCFAC agar | 1 | Oxalate | 1 |
| FBI00071 | YCFAC-B agar | 3 | YCFAC, pH 6.0 | 1 |
| FBI00076 | YCFAC-BO 80 mM agar | 2 | YCFAC, pH 6.0 | 1 |
| | YCFAC agar | 1 | | |
| FBI00079 | Chocolate agar | 2 | YCFAC, pH 6.0 | 1 |
| FBI00087 | Brain heart infusion agar with hemin and | 2 | YCFAC, pH 6.0 | 1 |
| | vitamin K | | | |
| | YCFAC agar | 1 | | |
| FBI00093 | YCFAC-BO 40 mM agar | 2 | YCFAC, pH 6.0 | 1 |
| FBI00102 | YCFAC-BO 40 mM agar | 2 | YCFAC, pH 6.0 | 1 |
| FBI00109 | YCFAC-B agar | 4 | YCFAC, pH 6.0 | 1 |
| FBI00117 | YCFAC-BO 80 mM agar | 2 | YCFAC, pH 6.0 | 2 |
| | YCFAC agar | 1 | | |
| FBI00120 | YCFAC-BO 80 mM agar | 2 | YCFAC, pH 6.0 | 1 |
| FBI00125 | YCFAC-BO 80 mM agar | 2 | YCFAC, pH 6.0 | 1 |
| FBI00127 | YCFAC-B agar | 3 | YCFAC, pH 6.0 | 1 |
| FBI00128 | YCFAC-BO 80 mM agar | 2 | YCFAC, pH 6.0 | 1 |
| | YCFAC-BO 40 mM agar | 1 | | |
| FBI00145 | YCFAC-B agar | 3 | YCFAC, pH 6.0 | 1 |
| FBI00162 | Reinforced clostridial agar | 2 | YCFAC, pH 6.0 | 1 |
| FBI00174 | YCFAC-B agar | 2 | YCFAC, pH 6.0 | 1 |
| FBI00184 | YCFAC-B agar | 3 | YCFAC, pH 6.0 | 1 |
| FBI00190 | Brain heart infusion agar with hemin and | 2 | YCFAC, pH 6.0 | 1 |
| | vitamin K | | | |
| FBI00191 | OxyPras plus brucella blood agar | 2 | YCFAC, pH 6.0 + BBL ™ | 1 |
| | | | Vitamin K1-Hemin Solution | |
| FBI00194 | Brain heart infusion agar with hemin and | 2 | YCFAC, pH 6.0 | 1 |
| | vitamin K | | | |
| FBI00198 | YCFAC-BO 40 mM agar | 3 | YCFAC, pH 6.0 | 1 |
| FBI00199 | YCFAC-BO 40 mM agar | 3 | YCFAC, pH 6.0 | 1 |
| FBI00200 | YCFAC-B agar | 3 | YCFAC, pH 6.0 | 1 |
| FBI00201 | Columbia agar with 5% sheep blood | 3 | YCFAC, pH 6.0 | 1 |
| FBI00205 | YCFAC-BO 40 mM agar | 3 | YCFAC, pH 6.0 | 1 |
| FBI00206 | YCFAC-BO 40 mM agar | 3 | YCFAC, pH 6.0 | 1 |
| FBI00211 | YCFAC-B agar | 3 | YCFAC, pH 6.0 | 1 |
| FBI00220 | Brain heart infusion agar with hemin and | 4 | YCFAC, pH 6.0 | 1 |
| | vitamin K | | | |
| FBI00221 | Brain heart infusion agar with hemin and | 2 | YCFAC, pH 6.0 | 1 |
| | vitamin K | | | |
| FBI00236 | YCFAC-BO 40 mM agar | 2 | YCFAC pH 6.0 | 1 |
| FBI00245 | Columbia agar with 5% sheep blood | 4 | YCFAC, pH 6.0 + BBL ™ | 1 |
| | | | Vitamin K1-Hemin Solution | |
| FBI00248 | Brain heart infusion agar with hemin and | 3 | YCFAC, pH 6.0 | 1 |
| | vitamin K | | | |
| FBI00251 | Reinforced clostridial agar | 2 | YCFAC, pH 6.0 | 1 |
| FBI00254 | Brain heart infusion with hemin and vitamin | 3 | YCFAC, pH 6.0 | 1 |
| | K | | | |
| FBI00267 | YCFAC-BO 80 mM agar | 3 | YCFAC, pH 6.0 | 1 |
| FBI00278 | Brain heart infusion agar with hemin and | 3 | YCFAC, pH 6.0 | 1 |
| | vitamin K | | | |
| FBI00288 | Brain heart infusion agar with hemin and | 3 | YCFAC, pH 6.0 | 2 |
| | vitamin K | | | |
| | YCFAC agar | 3 | | |
| | YCFAC-B agar | 1 | | |
| FBI00290 | Brain heart infusion agar with hemin and | 3 | YCFAC, pH 6.0 | 2 |
| | vitamin K | | | |

Abbreviations:
FBI = Federation Bio isolate;
RCA = reinforced clostridial agar;
RCB = research cell bank;
YCFAC = yeast casitone fatty acids with carbohydrates

To bank the RCBs used in FB-001 and FB-003 DS-CoC1, monocultures were inoculated into culture tubes containing appropriate broth media and incubated under anaerobic conditions at 37° C. until sufficient growth was observed. Sterile glycerol solution was added to achieve a final glycerol concentration of 25% prior to aliquoting approximately 0.2 mL into 2D-barcoded cryo-vials. After removing the cryovials from the anaerobic gas chambers, the 2D bar codes at the bottom of the vials were scanned promptly and the vials were transferred to <−65° C. as the final step in the banking of the RCBs.

After at least 10 hours of freezing, one vial of each purified frozen RCB was retrieved from the freezer and thawed under anaerobic conditions followed by plating on agar plates containing appropriate growth media. The plates were incubated under anaerobic conditions at 37° C. Growth on the plate was observed to confirm revival and uniform colony morphology for each purified isolate. Following confirmation of uniform colony morphology for each RCB, individual colonies were analyzed by 16S rRNA gene sequencing (see Sequence Listing). RCBs were further characterized using whole-genome sequencing followed by genome assembly. Strain-level identification was performed using both 16S rRNA gene sequences and whole-genome assemblies.

An explicit criterion for inclusion of each strain in FB-001 and FB-003 DS-CoC1 was demonstrated susceptibility to at least 2 FDA-approved antibiotics. The anaerobic microbes in the FB-001 DS-CoC1 were tested against multiple FDA-approved, clinically relevant antimicrobials, most of which show especially potent activity against anaerobes. All strains in FB-001 and FB-003 DS-CoC1 were found to demonstrate sensitivity in vitro to 2 or more clinically relevant antibiotics, implying a straightforward means for biological control. Importantly, no strain in the FB-001 or FB-003 DS-CoC1 were resistant to both clindamycin and amoxicillin-clavulanate, suggesting that a combination of the 2 agents could cover all FB-001 and FB-003 DS-CoC1 strains.

MCB generation is described in FIG. 12. The first step of MCB generation for DS-CoC1 strains involved reviving each RCB by plating on YCFAC agar plates followed by incubation under anaerobic conditions at 37° C. Isolated colonies were used for inoculating MCB precultures in 30 to 45 mL of YCFAC broth and were incubated anaerobically at 37° C. Each MCB was passaged 2 to 3 times in YCFAC broth prior to banking. Growth of precultures was monitored using total cell counts and viable cell counts to determine suitable time, inoculation, and culture volumes for MCB cultures. Sterility monitoring was performed by incubating a sterile agar plate or broth during the entire culturing process. A minimum total cell count of 2×10⁸ cells per mL was targeted for the harvest of the MCB culture. When required, cells were harvested by centrifugation to allow concentration of the biomass. Sterile glycerol was added as cryoprotectant to a final concentration of 25% v/v prior to aliquoting cells from MCB culture into 2D barcoded cryovials. The barcodes of cryovials were scanned and entered into an electronic inventory system, then the vials are transferred to long-term storage at <−65° C. All MCBs are stored in at least 2 physically distinct locations.

Manufacture of DS2. The same YCFAC media used for DS1 was used for DS2. Similarly, the same general strain isolation methods were used as described above for DS1. The specific agar types, passages, and broth types used for DS2 strains is provided in Table 8.

TABLE 8
Isolation of Research Cell Banks Used in FB-001 and FB-003 DS-CoC2

| | | | RCB Broth Passaging | |
|---|---|---|---|---|
| | RCB Agar Passaging | | | Passage |
| FBI Strain ID | Agar Type | Passage # | Broth Type | # |
| FBI00004 | YCFAC-B agar + 80 mM oxalate | 1 | YCFAC, pH 6.0 + 80 mM | 1 |
| | YCFAC-BO 80 mM agar | 2 | Oxalate | |
| FBI00012 | Bacteroides bile esculin agar | 2 | YCFAC, pH 6.0 | 1 |
| | YCFAC-B agar | 1 | | |
| FBI00015 | YCFAC-B agar | 2 | YCFAC, pH 6.0 | 1 |
| | YCFAC agar | 1 | | |
| FBI00018 | Bifidobacterium selective agar | 3 | YCFAC, pH 6.0 | 1 |
| FBI00019 | Bacteroides bile esculin agar | 2 | YCFAC, pH 6.0 | 1 |
| | YCFAC-B agar | 1 | | |
| FBI00021 | YCFAC-B agar + 80 mM oxalate | 1 | YCFAC, pH 6.0 + 80 mM | 1 |
| | YCFAC-BO 80 mM agar | 2 | Oxalate | |
| FBI00038 | Chocolate agar | 2 | YCFAC, pH 6.0 | 1 |
| FBI00040 | Bacteroides bile esculin agar | 2 | YCFAC, pH 6.0 | 1 |
| | YCFAC-B agar | 1 | | |
| FBI00046 | YCFAC-B agar | 3 | YCFAC, pH 6.0 | 1 |
| FBI00061 | Bacteroides bile esculin | 2 | YCFAC, pH 6.0 | 1 |
| | Brain heart infusion agar with | 1 | | |
| | hemin and vitamin K | | | |
| FBI00066 | Bacteroides bile esculin | 2 | YCFAC, pH 6.0 | 1 |
| | YCFAC-B agar | 1 | | |
| FBI00075 | YCFAC-B agar | 3 | YCFAC, pH 6.0 | 1 |
| FBI00077 | Lactobacillus MRS agar + 20 mM | 2 | YCFAC, pH 6.0 | 1 |
| | oxalate | | | |
| | YCFAC | 1 | | |
| FBI00080 | Lactobacillus MRS agar + 40 mM | 2 | YCFAC, pH 6.0 | 1 |
| | oxalate | | | |
| | YCFAC agar | 1 | | |
| FBI00081 | Columbia agar with 5% sheep blood | 3 | YCFAC, pH 6.0 | 1 |
| FBI00085 | Reinforced clostridial agar | 2 | YCFAC, pH 6.0 | 1 |
| FBI00092 | YCFAC-BO 40 mM agar | 2 | YCFAC, pH 6.0 | 1 |
| FBI00097 | Brain heart infusion agar with | 2 | YCFAC, pH 6.0 | 2 |
| | hemin and vitamin K | | | |
| | YCFAC agar | 2 | | |
| FBI00099 | Chocolate agar | 2 | YCFAC, pH 6.0 | 1 |
| FBI00112 | Brain heart infusion agar with | 2 | YCFAC, pH 6.0 | 1 |
| | hemin and vitamin K | | | |
| | YCFAC agar | 1 | | |
| FBI00132 | YCFAC-BO 80 mM agar | 2 | YCFAC, pH 6.0 | 1 |
| | YCFAC-BO 80 mM agar | 1 | | |
| FBI00137 | YCFAC-BO 40 mM agar | 2 | YCFAC, pH 6.0 | 1 |
| FBI00140 | YCFAC-BO 160 mM agar | 1 | YCFAC, pH 6.0 + 80 mM | 1 |
| | YCFAC-BO 80 mM agar | 2 | oxalate | |
| FBI00149 | YCFAC-BO 80 mM agar | 1 | YCFAC, pH 6.0 | 1 |
| | YCFAC-BO 80 mM agar | 1 | | |
| | YCFAC-BO 40 mM agar | 1 | | |
| FBI00151 | YCFAC-BO 80 mM agar | 1 | YCFAC, pH 6.0 | 1 |
| | YCFAC-BO 80 mM agar | 1 | | |
| | YCFAC-BO 40 mM agar | 2 | | |
| FBI00176 | Brain heart infusion agar with | 2 | YCFAC, pH 6.0 | 1 |
| | hemin and vitamin K | | | |
| FBI00189 | YCFAC-BO 40 mM agar | 3 | YCFAC, pH 6.0 | 1 |
| | YCFAC-BO 40 mM agar | 1 | | |
| FBI00197 | YCFAC-B agar | 3 | YCFAC, pH 6.0 | 1 |
| FBI00208 | YCFAC-BO 40 mM agar | 3 | YCFAC, pH 6.0 | 1 |
| FBI00212 | YCFAC-B agar | 3 | YCFAC, pH 6.0 | 1 |
| FBI00224 | YCFAC-BO 40 mM agar | 3 | YCFAC, pH 6.0 | 1 |
| FBI00226 | YCFAC-BO 40 mM agar | 3 | YCFAC, pH 6.0 with | 1 |
| | | | BBL ™ Vitamin K1- | |
| | | | Hemin Solution | |
| FBI00229 | Brain heart infusion agar with | 4 | Thioglycollate broth | 1 |
| | hemin and vitamin K | | with hemin and vitamin K | |
| FBI00233 | Brain heart infusion agar with | 4 | YCFAC, pH 6.0 with | 1 |
| | hemin and vitamin K | | BBL ™ Vitamin K1- | |
| | | | Hemin Solution | |
| FBI00235 | Brain heart infusion agar with | 4 | YCFAC, pH 6.0 | 1 |
| | hemin and vitamin K | | | |
| FBI00237 | Brain heart infusion agar with | 3 | YCFAC, pH 6.0 | 1 |
| | hemin and vitamin K | | | |
| FBI00243 | Brain heart infusion agar with | 4 | YCFAC, pH 6.0 | 1 |
| | hemin and vitamin K | | | |
| | YCFAC agar | 1 | | |
| FBI00244 | YCFAC-BO 40 mM agar | 3 | YCFAC, pH 6.0 | 1 |
| | YCFAC-B agar + 40 mM oxalate | 3 | | |
| | YCFAC agar | 1 | | |
| FBI00258 | Modified Eggerth-Gagnon medium | 3 | Thioglycollate broth | 1 |
| | agara | | with hemin and vitamin K | |
| FBI00260 | Brain heart infusion agar with | 3 | YCFAC, pH 6.0 | 1 |
| | hemin and vitamin K | | | |
| FBI00263 | Modified Eggerth-Gagnon medium | 3 | YCFAC, pH 6.0 | 1 |
| | agara | | | |
| FBI00270 | Columbia agar with 5% sheep | 2 | SAB mediac | 1 |
| | blood + antibioticsb | | | |
| | Columbia agar with 5% sheep blood | 3 | | |
| FBI00273 | Brain heart infusion agar with | 3 | YCFAC, pH 6.0 | 1 |
| | hemin and vitamin K | | | |
| FBI00277 | Brain heart infusion agar with | 3 | YCFAC, pH 6.0 | 1 |
| | hemin and vitamin K | | | |
| FBI00292 | Columbia agar with 5% sheep blood | 3 | SAB mediac | 1 |

Abbreviations:
FBI = Federation Bio isolate;
RCB = research cell bank;
YCFAC = yeast casitone fatty acids with carbohydrates,
YCFAC-B = yeast casitone fatty acids with carbohydrates and sheep blood;
YCFAC-BO = yeast casitone fatty acids with carbohydrates, sheep blood, and oxalate
aModified Eggerth-Gagnon medium agar is prepared in house and consists of peptone (1% w/v), Na₂HPO₄ (0.32% w/v), mucin (0.2% w/v), BactoAgar (1.5% w/v), and sheep blood (5% v/v), pH 7.45.
bAntibiotics used for isolation of FBI00270 included vancomycin (100 μg/mL), penicillin 100 units/mL, streptomycin (100 μg/mL), and amphotericin B (0.25 μg/mL).
cSAB media was prepared at described in (Khelaifia, Raoult et al. 2013).

Characterization and banking of the DS2 strains were performed as described above for DS1. It is important to note that while not all DS2 strains were sensitive to both clindamycin and amoxicillin-clavulanate as were the DS1 strains, all strains were still sensitive to at least 2 FDA approved antibiotics.

Manufacture of DS3. The same YCFAC media used for DS1 was used for DS2. Similarly, the same general strain isolation methods were used as described above for DS1. The specific agar types, passages, and broth types used for DS2 strains is provided in Table 9.

TABLE 9
Isolation of Research Cell Banks Used in FB-001 and FB-003 DS-CoC3

| FBI | RCB Agar Passaging | | RCB Broth Passaging | |
|---|---|---|---|---|
| Strain ID | Agar Type | Passage # | Broth Type | Passage # |
| FBI00009 | YCFAC-B agar | 3 | YCFAC, pH 6.0 | 1 |
| FBI00011 | Bifidobacterium selective agar | 3 | YCFAC, pH 6.0 | 1 |
| FBI00016 | YCFAC-B agar | 3 | YCFAC, pH 6.0 | 1 |
| FBI00020 | Bifidobacterium selective agar + | 1 | YCFAC, pH 6.0 with 40 | 1 |
| | 40 mM oxalate | | mM Oxalate | |
| | YCFAC-BO 40 mM agar | 1 | | |
| | YCFAC-BO 40 mM agar | 1 | | |
| FBI00025 | Chocolate agar | 2 | YCFAC, pH 6.0 | 1 |
| FBI00027 | Brain-heart infusion agar with hemin | 2 | YCFAC, pH 6.0 | 1 |
| | and vitamin K | | | |
| FBI00030 | YCFAC-B agar | 3 | YCFAC, pH 6.0 | 1 |
| FBI00047 | YCFAC-B agar | 3 | YCFAC, pH 6.0 | 1 |
| FBI00052 | YCFAC-BO 40 mM agar | 2 | YCFAC, pH 6.0 with 40 | 1 |
| | YCFAC agar | 1 | mM Oxalate | |
| FBI00053 | YCFAC-BO 40 mM agar | 2 | YCFAC, pH 6.0 with 40 | 1 |
| | YCFAC agar | 1 | mM Oxalate | |
| FBI00056 | YCFAC-BO 80 mM agar | 2 | YCFAC, pH 6.0 with 80 | 1 |
| | YCFAC agar | 1 | mM Oxalate | |
| FBI00062 | YCFAC-B agar | 3 | YCFAC, pH 6.0 | 1 |
| FBI00078 | YCFAC-B agar | 2 | YCFAC, pH 6.0 | 1 |
| FBI00096 | Brain-heart infusion agar with hemin | 2 | YCFAC, pH 6.0 | 1 |
| | and vitamin K | | | |
| | YCFAC agar | 1 | | |
| FBI00104 | Brain-heart infusion agar with hemin | 2 | YCFAC, pH 6.0 | 1 |
| | and vitamin K | | | |
| | YCFAC agar | 1 | | |
| FBI00110 | YCFAC-BO 80 mM agar | 2 | YCFAC, pH 6.0 | 1 |
| | YCFAC agar | 1 | | |
| | YCFAC-B agar | 1 | | |
| FBI00111 | YCFAC-BO 80 mM agar | 2 | YCFAC, pH 6.0 | 1 |
| | YCFAC agar | 1 | | |
| | YCFAC-B agar | 1 | | |
| FBI00113 | Brain-heart infusion agar with hemin | 2 | YCFAC, pH 6.0 | 1 |
| | and vitamin K | | | |
| | YCFAC-B agar | 1 | | |
| FBI00115 | Brain-heart infusion agar with hemin | 2 | YCFAC, pH 6.0 | 2 |
| | and vitamin K | | | |
| | YCFAC agar | 3 | | |
| FBI00116 | Bifidobacterium selective agar + | 1 | YCFAC, pH 6.0 with 40 | 1 |
| | 40 mM oxalate | | mM Oxalate | |
| | Bifidobacterium selective agar | 2 | YCFAC, pH 6.0 | 1 |
| FBI00123 | YCFAC-BO 160 mM agar | 1 | YCFAC, pH 6.0 | 1 |
| | YCFAC-B agar | 2 | | |
| FBI00124 | YCFAC-B agar | 3 | YCFAC, pH 6.0 | 1 |
| FBI00126 | YCFAC-BO 40 mM agar | 1 | YCFAC, pH 6.0 | 1 |
| | YCFAC-BO 40 mM agar | 1 | | |
| FBI00135 | YCFAC-B agar | 3 | YCFAC, pH 6.0 | 1 |
| FBI00147 | YCFAC-BO 80 mM agar | 1 | YCFAC, pH 6.0 | 1 |
| | YCFAC-BO 80 mM agar | 1 | | |
| | YCFAC-BO 40 mM agar | 1 | | |
| FBI00159 | YCFAC-BO 160 mM agar | 1 | YCFAC, pH 6.0 | 1 |
| | YCFAC-BO 80 mM agar | 2 | | |
| FBI00167 | YCFAC-B agar | 2 | YCFAC, pH 6.0 | 1 |
| FBI00170 | YCFAC-BO 80 mM agar | 1 | YCFAC, pH 6.0 | 2 |
| | YCFAC-BO 80 mM agar | 1 | | |
| | YCFAC-BO 40 mM agar | 1 | | |
| FBI00232 | Columbia agar with 5% sheep blood | 4 | YCFAC, pH 6.0 | 1 |
| FBI00255 | YCFAC-BO 80 mM agar | 3 | YCFAC, pH 6.0 | 1 |
| FBI00271 | Brain-heart infusion agar with hemin | 3 | YCFAC, pH 6.0 | 1 |
| | and vitamin K | | | |

Abbreviations: FBI = Federation Bio isolate; RCB = research cell bank; YCFAC = yeast casitone fatty acids with carbohydrates, YCFAC-B = yeast casitone fatty acids with carbohydrates and sheep blood; YCFAC-BO = yeast casitone fatty acids with carbohydrates, sheep blood and oxalate.

Characterization and banking of the DS3 strains were performed as described above for DS1. It is important to note that while not all DS2 strains were sensitive to both clindamycin and amoxicillin-clavulanate as were the DS1 strains, all strains were still sensitive to at least 2 FDA approved antibiotics.

Manufacture of DS4. YCFAC media with ammonium sulfate, pH 7 for Seed 1 Fermentation was prepared at 1× concentration in batches of 4 L. The medium is prepared by adding the components indicated in Table 10 to 3.46 kg of water for injection and boiling for 5 to 10 minutes. Then the media was sparged for at least 30 minutes with N2 and allowed to cool down. Upon reaching a temperature of 50° C. or lower, the rest of the components were added in the following order while sparging continues: sodiumbicarbonate, 50× volatile fatty acid solution, L-cysteine HCl monohydrate, and 0.5% hemin solution. The medium was adjusted to a pH of 7 with 10 N NaOH or sulfuric acid and was autoclaved at 122.5° C. for 45 minutes. Vitamin solution (25×) was filtered using a 0.22 μm filter and added post-sterilization. The medium was incubated at 37° C. for a minimum of 24 hours prior to inoculation for a contamination check.

TABLE 10
Yeast Casitone Fatty Acids with Carbohydrates
Medium Composition (1X) For Seed 1 Fermentation

| | Quantity/4.0 L | | | |
|---|---|---|---|---|
| Reagent Description | of Medium | Unit | Final Conc. (1X) | Addition |
| Soytone | 40.00 | g | 1% (w/w) | Boiled for 5 to |
| D-cellobiose | 8.00 | g | 0.2% (w/w) | 10 minutes in 3.46 |
| Yeast extract | 10.00 | g | 0.25% (w/w) | kg WFI; sparging |
| Dextrose (glucose) | 20.00 | g | 0.5% (w/w) | initiated |
| Maltose monohydrate | 8.00 | g | 0.2% (w/w) | |
| Magnesium sulfate heptahydrate | 0.36 | g | 0.009% (w/w) | |
| Calcium chloride dihydrate | 0.36 | g | 0.009% (w/w) | |
| Potassium phosphate monobasic | 1.80 | g | 0.045% (w/w) | |
| Potassium phosphate dibasic | 1.80 | g | 0.045% (w/w) | |
| Sodium chloride | 3.60 | g | 0.09% (w/w) | |
| Ammonium sulfate | 3.60 | g | 0.09% (w/w) | |
| Sodium bicarbonate (7.5% w/w) | 213 | mL | 5.325% (w/w) | Added after the |
| Volatile fatty acid solution | 11.6 | mL | 1X volatile fatty | media cools down |
| (50X) | | | acid solution | to 50° C. or |
| L-cysteine HCl monohydrate | 4.0 | g | 0.1% | lower (sparging |
| Hemin solution (0.5% w/w) | 8.00 | mL | 0.2% w/w hemin | continues) |
| Vitamin solution (25X) | 160.00 | mL | 1X vitamin | Added post- |
| | | | solution | sterilization |

YCFAC medium with ammonium sulfate, threonine, and N-acetylgalactosamine, pH 7.4 for Seed 2 Fermentation (Stage 1 and Stage 2) is prepared at X concentration in batches of 4 L. The medium is prepared by adding the components indicated in Table 11 to 3.46 kg of water for injection and boiling for 5 to 10 minutes. Then the media is sparged for at least 30 minutes with N2 and allowed to cool down. Upon reaching a temperature of 50° C. or lower, the rest of the components are added in the following order while sparging continues: sodium bicarbonate, 50× volatile fatty acid solution, L-cysteine HCl monohydrate, and 0.5% hemin solution. The medium is adjusted to a pH of 7 with 10 N NaH or sulfuric acid and is autoclaved at 122.5° C. for 45 minutes. Sterile 25× vitamin solution (25×), threonine solution, and N-acetylgalactosamine solution are added post-sterilization. The medium is incubated at 37° C. for a minimum of 24 hours prior to inoculation for a contamination check.

TABLE 11
Yeast Casitone Fatty Acids with Carbohydrates
Medium Composition (1X) For Seed 2 Fermentation

| | Quantity/4.0 L | | | |
|---|---|---|---|---|
| Reagent Description | of Medium | Unit | Final Conc. (1X) | Addition |
| Soytone | 40.00 | g | 1% (w/w) | Boiled for 5 to |
| D-cellobiose | 8.00 | g | 0.2% (w/w) | 10 minutes in 3.46 |
| Yeast extract | 10.00 | g | 0.25% (w/w) | kg WFI; sparging |
| Dextrose (glucose) | 20.00 | g | 0.5% (w/w) | initiated |
| Maltose monohydrate | 8.00 | g | 0.2% (w/w) | |
| Magnesium sulfate heptahydrate | 0.36 | g | 0.009% (w/w) | |
| Calcium chloride dihydrate | 0.36 | g | 0.009% (w/w) | |
| Potassium phosphate monobasic | 1.80 | g | 0.045% (w/w) | |
| Potassium phosphate dibasic | 1.80 | g | 0.045% (w/w) | |
| Sodium chloride | 3.60 | g | 0.09% (w/w) | |
| Ammonium sulfate | 3.60 | g | 0.09% (w/w) | |
| Sodium bicarbonate (7.5% w/w) | 213 | mL | 5.325% (w/w) | Added after the |
| Volatile fatty acid solution | 11.6 | mL | 1X volatile fatty | media cools down |
| (50X) | | | acid solution | to 50° C. or |
| L-cysteine HCl monohydrate | 4.0 | g | 0.1% | lower (sparging |
| Hemin solution (0.5% w/w) | 8.00 | mL | 0.2% w/w hemin | continues) |
| Vitamin solution (25X) | 160.00 | mL | 1X vitamin solution | Added post- |
| N-acetylgalactosamine solution | 20 | mL | 0.5% (w/w) | sterilization |
| (1% w/w) | | | | |
| Threonine | 59.6 | g | 1.5% (w/w) | |

YCFAC medium with ammonium sulfate and threonine, pH 7, used for the main fermentation, is prepared at 5×. The 5× medium is prepared by adding the components indicated in Table 12 to 40.0 kg of water for injection, mixing, then autoclaving. The medium is incubated at 37° C. for a minimum of 24 hours prior to inoculation for a contamination check. After pumping the 5× solution into the fermenter, 50× volatile fatty acid solution, threonine solution, 25× vitamin solution, L-cysteine HCO solution, and WFI are added for a final 1× concentration.

TABLE 12
Yeast Casitone Fatty Acids with Carbohydrates
Medium Composition (5X)

| | Quantity/60.0 | | |
|---|---|---|---|
| | kg YCFAC | | Final |
| Reagent Description | (5X) Medium | Unit | Conc. (1X) |
| Soytone | 3.00 | kg | 1% (w/w) |
| D-(+) cellobiose | 600 | g | 0.2% (w/w) |
| Yeast extract | 750 | g | 0.25% (w/w) |
| Dextrose (glucose) | 1.50 | kg | 0.5% (w/w) |
| Maltose monohydrate | 600.0 | g | 0.2% (w/w) |
| Magnesium sulfate heptahydrate | 27.0 | g | 0.009% (w/w) |
| Calcium chloride dihydrate | 27.0 | g | 0.009% (w/w) |
| Potassium phosphate monobasic | 135 | g | 0.045% (w/w) |
| Potassium phosphate dibasic | 135 | g | 0.045% (w/w) |
| Sodium chloride | 270 | g | 0.09% (w/w) |
| Ammonium sulfate | 270 | g | 0.09% (w/w) |
| Sodium bicarbonate (7.5% w/w) | 16.0 | kg | 5.33% (w/w) |
| Hemin solution (0.5% w/w) | 600 | mL | 0.2% (w/w) |

| | |
|---|---|
| Volatile fatty acid solution (50X) | 1X |
| L-cysteine HCl monohydrate solution (3% w/w) | 3.32% (w/w) |
| Vitamin solution (25X) | 1X |
| Threonine solution (7.2% w/w) | 4.0% (w/w) |

The specific agar types, passages, and broth types used for DS2 strains is provided in Table 13.

TABLE 13
Isolation of Research Cell Banks Used in FB-001 and FB-003 DS-CoC4

| | RCB Agar Passaging | | RCB Broth Passaging | |
|---|---|---|---|---|
| FBI | | Passage | | Passage |
| Strain ID | Agar Type | # | Broth Type | # |
| FBI00022 | Bacteroides bile esculin agar | 2 | YCFAC, pH 6.0 | 1 |
| | YCFAC-B agar | 1 | | |
| FBI00049 | YCFAC-B agar | 3 | YCFAC, pH 6.0 | 1 |
| FBI00068 | Bicarbonate-buffered basal medium | 2 | YCFAC, pH 6.0 | 2 |
| | agar^{a} | | | |
| | YCFAC-B agar | 1 | | |
| FBI00069 | Bifidobacterium selective agar + | 1 | YCFAC, pH 6.0 | 1 |
| | 40 mM oxalate | | | |
| | Bifidobacterium selective agar | 2 | | |
| FBI00152 | YCFAC-B agar | 2 | YCFAC, pH 6.0 with BBL ™ | 1 |
| | | | Vitamin K1-Hemin Solution | |
| FBI00165 | Brain-heart infusion agar with | 3 | YCFAC, pH 6.0 | 1 |
| | hemin and vitamin K | | | |
| FBI00171 | YCFAC-BO 80 mM agar | 1 | YCFAC, pH 6.0 | 2 |
| | YCFAC-BO 80 mM agar | 1 | | |
| | YCFAC-BO 40 mM agar | 1 | | |
| FBI00175 | YCFAC-B agar | 2 | YCFAC, pH 6.0 with BBL | 1 |
| | | | Vitamin K1-Hemin Solution | |
| FBI00177 | Bacteroides bile esculin agar | 2 | YCFAC, pH 6.0 | 1 |
| FBI00180 | Bacteroides bile esculin agar | 2 | YCFAC, pH 6.0 | 1 |
| FBI00182 | YCFAC-B agar | 2 | YCFAC, pH 6.0 | 1 |
| FBI00238 | Columbia agar with 5% sheep blood | 3 | YCFAC, pH 6.0 | 1 |
| FBI00269 | Brain-heart infusion agar with | 3 | YCFAC, pH 6.0 | 1 |
| | hemin and vitamin K | | | |
| FBI00274 | YCFAC-BO 80 mM agar | 3 | YCFAC, pH 6.0 | 1 |
| FBI00281 | Reinforced clostridial agar | 3 | YCFAC, pH 6.0 | 1 |

Abbreviations: FBI = Federation Bio isolate; RCA = reinforced clostridial agar; RCB = research cell bank; YCFAC = yeast casitone fatty acids with carbohydrates, YCFAC-B = yeast casitone fatty acids with carbohydrates and sheep blood; YCFAC-BO = yeast casitone fatty acids with carbohydrates, sheep blood, and oxalate; Bicarbonate-buffered basal medium was prepared as described in Derrien 2004 (Derrien, Vaughan et al. 2004).

Characterization and banking of the DS4 strains was performed as described above for DS1. It is important to note that while not all DS2 strains were sensitive to both clindamycin and amoxicillin-clavulanate as were the DS1 strains, all strains were still sensitive to at least 2 FDA approved antibiotics.

### Example 6: Functional Characterization of DS1-7

FB-001 and FB-003 were characterized through 16S sequence identity, macronutrient utilization, metabolite production and Biolog analysis of individual strains. At the species level, FB-001 and FB-003 was characterized by the DNA sequences of 16S rRNA genes which represent 100 species. 16S sequence length varied by strain, from a minimum of 1177 bp (FBI00109, Coprococcus comes) to a maximum of 1532 bp (FBI00087, Clostridium scindens). For FB-001, the 148 16S DNA sequences uniquely identified the majority of the 148 strains within FB-001, with exceptions for closely related strains such as two of the Oxalobacter formigenes strains (FBI00133 and FBI00289) which share identical 16S sequences. To provide phenotypic characterization, Biolog assays were used to characterize the strains in FB-001 and FB-003, as described below.

Biolog phenotype assays were used to determine unique macronutrient signatures for FB-001 and FB-003 strains. These data provide empirical characterization of growth features of each strain. The 148 strains of FB-001 and the 145 strains of FB-003 fit into several broad categories of growth characteristics based on our Biolog analyses: 98 strains showed positive growth signatures; 41 strains did not have positive growth signatures; 9 were not tested using Biolog due to insufficient growth. Table 14 shows the 98 strains with positive growth signatures, with the specific macronutrients that supported growth listed along with the Genus species identification of each strain. Of the 98 strains with positive growth signatures, 60 were tested against the 190 individual carbon and energy sources present in the 96 well plate format of PM1 and 2 plates and the remaining 38 were tested using 2 plates alone. Each 96 well plate contains one negative control well that lacks any additional carbon or energy source. The total number of substrates utilized by any single strain in this assay showed great diversity, ranging from 1 to 59 substrates that yield growth. Furthermore, each of the 98 strains with growth on at least one substrate presented with an entirely unique growth fingerprint, or combination of permissive growth substrates, relative to every other strain in the set.

Table 14. Characterization of Strain-Level Macronutrient Utilization by Biolog Assay In 98 Strains with Positive Growth Signatures.

For each strain, the Biolog PM plates tested are given along with the Genus species identity and the macronutrients that supported growth. Positive growth is defined as an increase of 0.1 or more in optical density at 600 nm above the negative control that contained no supplied carbon and energy source.

| Strain | PM | Macronutrient growth |
|---|---|---|
| FBI00001 | 1, 2 | 2-Deoxy-D-Ribose; D-Fructose; D-Fructose-6-Phosphate; D-Galactose; D- |
| | | Galacturonic Acid; D-Gluconic Acid; D-Glucosamine; D-Glucuronic Acid; D- |
| | | Mannose; D-Ribose; D-Saccharic Acid; D-Trehalose; D-Xylose; Inosine; L- |
| | | Arabinose; L-Galactonic Acid-g-Lactone; Maltose; Mucic Acid; N-Acetyl-D- |
| | | Glucosamine; N-Acetyl-Neuraminic Acid; Sucrose; Thymidine; Uridine; a-D- |
| | | Glucose; b-D-Allose |
| FBI00002 | 1, 2 | D-Galactose; a-D-Glucose; a-D-Lactose |
| FBI00004 | 1, 2 | D-Glucose-6-Phosphate; Maltose; Maltotriose |
| FBI00010 | 1, 2 | D-Arabinose; D-Cellobiose; D-Fructose; D-Fucose; D-Galactose; D-Mannose; D- |
| | | Raffinose; D-Ribose; D-Sorbitol; D-Xylose; L-Arabinose; L-Fucose; L-Rhamnose; |
| | | Maltose; Maltotriose; Stachyose; a-D-Glucose; a-D-Lactose |
| FBI00013 | 1, 2 | Caproic Acid; D-Melibiose; L-Pyroglutamic Acid; Melibionic Acid; N-Acetyl-D- |
| | | Glucosamine; N-Acetyl-L-Glutamic Acid; Oxalic Acid; a-D-Glucose; a-D-Lactose |
| FBI00015 | 1, 2 | 3-Hydroxy 2-Butanone; 3-Methyl Glucose; Amygdalin; Arbutin; D-Cellobiose; D- |
| | | Glucosamine; D-Glucuronic Acid; D-Melibiose; D-Raffinose; Inulin; L-Galactonic |
| | | Acid-g-Lactone; Lactulose; N-Acetyl-D-Galactosamine; N-Acetyl-b-D- |
| | | Mannosamine; Palatinose; Salicin; a-D-Lactose; a-Methyl-D-Galactoside; b- |
| | | Cyclodextrin; b-Methyl-D-Galactoside |
| FBI00025 | 1, 2 | D-Fructose; D-Fucose; D-Galactose; D-Melibiose; D-Raffinose; D-Xylose; Glycogen; |
| | | L-Arabinose; Lactulose; Maltose; Maltotriose; Stachyose; Sucrose; a-D-Glucose; a- |
| | | D-Lactose |
| FBI00027 | 1, 2 | D-Cellobiose; D-Galactose; D-Glucosamine; D-Raffinose; L-Tartaric Acid; |
| | | Lactulose; Maltitol; Maltose; Maltotriose; Palatinose; Pectin; Stachyose; Sucrose; a- |
| | | D-Glucose; a-D-Lactose; a-Methyl-D-Galactoside; a-Methyl-D-Glucoside; b-Methyl- |
| | | D-Galactoside; b-Methyl-D-Glucoside; b-Methyl-D-Xyloside |
| FBI00030 | 1, 2 | D-Mannitol; D-Trehalose; D-Xylose; Glycogen; Palatinose; a-D-Lactose |
| FBI00033 | 1, 2 | Lactulose; a-D-Lactose |
| FBI00044 | 1, 2 | Amygdalin; Arbutin; D-Arabinose; D-Cellobiose; D-Fructose; D-Galactose; D- |
| | | Glucosamine; D-Mannitol; D-Melezitose; D-Melibiose; D-Raffinose; D-Sorbitol; D- |
| | | Trehalose; D-Xylose; Gentiobiose; L-Arabinose; L-Fucose; Lactitol; Lactulose; |
| | | Maltitol; Maltose; Maltotriose; N-Acetyl-Neuraminic Acid; Palatinose; Pectin; |
| | | Salicin; Stachyose; Sucrose; Turanose; a-D-Glucose; a-D-Lactose; b-Methyl-D- |
| | | Galactoside; b-Methyl-D-Glucoside |
| FBI00046 | 1, 2 | D-Arabinose; D-Cellobiose; D-Fructose; D-Galactose; D-Galacturonic Acid; D- |
| | | Glucosamine; D-Glucuronic Acid; D-Mannose; D-Melezitose; D-Melibiose; D- |
| | | Raffinose; D-Ribose; D-Trehalose; D-Xylose; Gentiobiose; L-Arabinose; L-Fucose; |
| | | L-Galactonic Acid-g-Lactone; Lactitol; Lactulose; Maltitol; Maltose; Maltotriose; N- |
| | | Acetyl-D-Galactosamine; N-Acetyl-D-Glucosamine; Palatinose; Pectin; Salicin; |
| | | Stachyose; Sucrose; Turanose; Uridine; a-D-Glucose; a-D-Lactose; a-Methyl-D- |
| | | Glucoside; b-Methyl-D-Galactoside |
| FBI00047 | 1, 2 | D-Gluconic Acid |
| FBI00048 | 1, 2 | Arbutin; D-Cellobiose; D-Fructose; D-Galactose; D-Glucosamine; D-Glucosaminic |
| | | Acid; D-Melibiose; D-Raffinose; Gentiobiose; L-Arabinose; Lactitol; Lactulose; |
| | | Maltitol; Maltose; Maltotriose; Melibionic Acid; Palatinose; Pectin; Salicin; |
| | | Stachyose; Sucrose; Turanose; a-D-Glucose; a-D-Lactose; a-Methyl-D-Glucoside; b- |
| | | Methyl-D-Galactoside; b-Methyl-D-Glucoside |
| FBI00050 | 1, 2 | 3-Methyl Glucose; Amygdalin; Chondroitin Sulfate C; D-Cellobiose; D-Fructose; D- |
| | | Galactose; D-Galacturonic Acid; D-Glucosamine; D-Glucuronic Acid; D-Mannose; |
| | | D-Melibiose; D-Raffinose; D-Xylose; Dextrin; Gentiobiose; Glycogen; Inulin; L- |
| | | Fucose; L-Galactonic Acid-g-Lactone; L-Lyxose; L-Rhamnose; Lactulose; |
| | | Laminarin; Maltose; Maltotriose; N-Acetyl-D-Galactosamine; N-Acetyl-D- |
| | | Glucosamine; N-Acetyl-b-D-Mannosamine; Palatinose; Pectin; Stachyose; Sucrose; |
| | | Thymidine; Uridine; a-Cyclodextrin; a-D-Glucose; a-D-Lactose; b-Cyclodextrin; b- |
| | | Methyl-D-Galactoside; g-Cyclodextrin |
| FBI00051 | 1, 2 | D-Fructose; D-Galactose; D-Galacturonic Acid; D-Sorbitol; Inulin; L-Arabinose; L- |
| | | Galactonic Acid-g-Lactone; L-Tartaric Acid; Maltose; Maltotriose; N-Acetyl- |
| | | Neuraminic Acid; a-D-Glucose |
| FBI00053 | 1, 2 | D-Galacturonic Acid; D-Gluconic Acid; D-Glucuronic Acid; L-Galactonic Acid-g- |
| | | Lactone |
| FBI00057 | 1, 2 | Arbutin; D-Cellobiose; D-Fructose; D-Glucosamine; D-Raffinose; D-Sorbitol; L- |
| | | Arabinose; Maltose; Maltotriose; N-Acetyl-D-Galactosamine; N-Acetyl-Neuraminic |
| | | Acid; Stachyose; Turanose; a-D-Glucose; a-D-Lactose; m-Inositol |
| FBI00059 | 1, 2 | Amygdalin; Arbutin; D-Arabinose; D-Galacturonic Acid; D-Glucosamine; D- |
| | | Glucuronic Acid; D-Ribose; L-Fucose; Lactulose; Laminarin; N-Acetyl-D- |
| | | Glucosamine; Pectin; a-D-Lactose; b-Methyl-D-Galactoside |
| FBI00070 | 1, 2 | 3-0-b-D-Galacto-pyranosyl-D-Arabinose; Amygdalin; Arbutin; Chondroitin Sulfate |
| | | C; D-Arabinose; D-Cellobiose; D-Fructose; D-Fructose-6-Phosphate; D-Galactose; |
| | | D-Galacturonic Acid; D-Glucosamine; D-Glucose-1-Phosphate; D-Glucose-6- |
| | | Phosphate; D-Glucuronic Acid; D-Mannose; D-Melezitose; D-Melibiose; D- |
| | | Raffinose; D-Ribose; D-Trehalose; D-Xylose; Dextrin; Gentiobiose; Glycogen; L- |
| | | Arabinose; L-Fucose; L-Galactonic Acid-g-Lactone; L-Rhamnose; Lactitol; |
| | | Lactulose; Maltitol; Maltose; Maltotriose; N-Acetyl-D-Galactosamine; N-Acetyl-D- |
| | | Glucosamine; N-Acetyl-b-D-Mannosamine; Palatinose; Pectin; Salicin; Stachyose; |
| | | Sucrose; Thymidine; Turanose; Uridine; a-Cyclodextrin; a-D-Glucose; a-D-Lactose; |
| | | a-Methyl-D-Galactoside; a-Methyl-D-Glucoside; a-Methyl-D-Mannoside; b- |
| | | Cyclodextrin; b-Methyl-D-Galactoside; b-Methyl-D-Glucoside; g-Cyclodextrin |
| FBI00078 | 1, 2 | D-Arabinose; D-Cellobiose; D-Fructose; D-Galactose; D-Mannose; D-Melibiose; D- |
| | | Raffinose; D-Ribose; D-Sorbitol; D-Xylose; L-Arabinose; L-Fucose; L-Lyxose; L- |
| | | Rhamnose; L-Sorbose; Lactitol; Lactulose; Maltose; Maltotriose; Pectin; |
| | | Sedoheptulosan; Stachyose; Sucrose; Xylitol; a-D-Glucose; a-D-Lactose; b-Methyl- |
| | | D-Galactoside |
| FBI00079 | 1, 2 | 5-Keto-D-Gluconic Acid; Amygdalin; Arbutin; D-Cellobiose; D-Fructose; D- |
| | | Galactonic Acid-g-Lactone; D-Galactose; D-Gluconic Acid; D-Glucosamine; D- |
| | | Glucose-1-Phosphate; D-Glucuronic Acid; D-Mannose; D-Ribono-1,4-Lactone; D- |
| | | Ribose; D-Saccharic Acid; D-Xylose; Gentiobiose; L-Arabinose; L-Fucose; L- |
| | | Galactonic Acid-g-Lactone; Lactulose; Maltose; Maltotriose; Mucic Acid; N-Acetyl- |
| | | D-Glucosamine; N-Acetyl-Neuraminic Acid; N-Acetyl-b-D-Mannosamine; Pectin; |
| | | Salicin; Sucrose; Thymidine; Uridine; a-D-Glucose; a-D-Lactose; b-Methyl-D- |
| | | Galactoside; b-Methyl-D-Glucoside |
| FBI00087 | 1, 2 | D-Arabitol; D-Fructose; D-Galactose; D-Gluconic Acid; D-Ribose; D-Sorbitol; D- |
| | | Xylose; L-Arabinose; Sucrose; a-D-Glucose |
| FBI00102 | 1, 2 | Amygdalin; Chondroitin Sulfate C; D-Arabinose; D-Cellobiose; D-Fructose; D- |
| | | Galactose; D-Galacturonic Acid; D-Glucosamine; D-Glucuronic Acid; D-Mannose; |
| | | D-Melibiose; D-Raffinose; D-Ribose; D-Trehalose; D-Xylose; Dextrin; Gentiobiose; |
| | | Glycogen; Inulin; L-Arabinose; L-Fucose; L-Galactonic Acid-g-Lactone; L- |
| | | Rhamnose; Lactulose; Laminarin; Maltitol; Maltose; Maltotriose; N-Acetyl-D- |
| | | Galactosamine; N-Acetyl-D-Glucosamine; N-Acetyl-b-D-Mannosamine; Palatinose; |
| | | Pectin; Salicin; Sucrose; Turanose; Uridine; a-Cyclodextrin; a-D-Glucose; a-D- |
| | | Lactose; a-Methyl-D-Glucoside; a-Methyl-D-Mannoside; b-Cyclodextrin; b-Methyl- |
| | | D-Galactoside; g-Cyclodextrin |
| FBI00104 | 1, 2 | Amygdalin; D-Arabinose; D-Cellobiose; D-Fructose; D-Galactose; D-Glucosamine; |
| | | D-Melibiose; D-Raffinose; D-Sorbitol; D-Xylose; L-Arabinose; L-Fucose; L- |
| | | Rhamnose; Maltose; N-Acetyl-Neuraminic Acid; Stachyose; Uridine; a-D-Glucose; a- |
| | | D-Lactose |
| FBI00109 | 1, 2 | Arbutin; D-Fructose; D-Galactose; D-Melibiose; D-Raffinose; D-Sorbitol; Maltose; |
| | | Maltotriose; Salicin; Stachyose; a-D-Glucose; a-D-Lactose; b-Methyl-D-Glucoside |
| FBI00110 | 1, 2 | 5-Keto-D-Gluconic Acid; Arbutin; D-Cellobiose; D-Fructose; D-Fructose-6- |
| | | Phosphate; D-Galactose; D-Gluconic Acid; D-Glucosamine; D-Mannose; D- |
| | | Melezitose; D-Raffinose; D-Ribose; D-Trehalose; D-Xylose; Gentiobiose; Inosine; L- |
| | | Arabinose; L-Fucose; Lactulose; Maltose; Maltotriose; N-Acetyl-D-Glucosamine; |
| | | Palatinose; Salicin; Stachyose; Sucrose; Thymidine; Turanose; a-D-Glucose; b-D- |
| | | Allose; b-Methyl-D-Glucoside |
| FBI00113 | 1, 2 | Arbutin; N-Acetyl-D-Galactosamine; a-Methyl-D-Galactoside |
| FBI00115 | 1, 2 | D-Fructose; D-Galactose; D-Xylose; L-Arabinose; Maltose; Maltotriose; N-Acetyl- |
| | | Neuraminic Acid; Turanose; a-D-Glucose |
| FBI00117 | 1, 2 | Arbutin; Maltotriose |
| FBI00125 | 1, 2 | Chondroitin Sulfate C; D-Fructose; D-Galactose; D-Glucosamine; D-Mannose; |
| | | Gentiobiose; Lactulose; Laminarin; Maltose; Maltotriose; N-Acetyl-D- |
| | | Galactosamine; N-Acetyl-D-Glucosamine; Pectin; Sucrose; a-Cyclodextrin; a-D- |
| | | Glucose; b-Cyclodextrin; b-Methyl-D-Galactoside; g-Cyclodextrin |
| FBI00128 | 1, 2 | 1,2-Propanediol; D-Fructose-6-Phosphate; a-D-Glucose |
| FBI00137 | 1, 2 | D-Fructose; D-Galactose; D-Glucosamine; D-Glucuronic Acid; D-Mannose; D- |
| | | Melibiose; D-Raffinose; D-Xylose; Dextrin; Glycogen; Inulin; L-Fucose; Lactitol; |
| | | Lactulose; Maltose; Maltotriose; N-Acetyl-D-Galactosamine; N-Acetyl-D- |
| | | Glucosamine; N-Acetyl-Neuraminic Acid; Sucrose; a-Cyclodextrin; a-D-Glucose; a- |
| | | D-Lactose; b-Cyclodextrin; b-Methyl-D-Galactoside; g-Cyclodextrin |
| FBI00147 | 1, 2 | 2-Deoxy-D-Ribose; D-Fructose; D-Galactose; D-Gluconic Acid; D-Glucosamine; D- |
| | | Glucuronic Acid; D-Mannose; D-Melibiose; D-Psicose; D-Raffinose; D-Ribose; D- |
| | | Sorbitol; D-Trehalose; D-Xylose; L-Arabinose; Lactulose; Maltose; Maltotriose; N- |
| | | Acetyl-D-Glucosamine; N-Acetyl-Neuraminic Acid; Palatinose; Stachyose; Sucrose; |
| | | Thymidine; Uridine; a-D-Glucose |
| FBI00165 | 1, 2 | N-Acetyl-D-Galactosamine; N-Acetyl-D-Glucosamine; N-Acetyl-Neuraminic Acid |
| FBI00167 | 1, 2 | Amygdalin; Arbutin; D-Cellobiose; D-Fructose; D-Galactose; D-Glucosamine; D- |
| | | Raffinose; D-Sorbitol; Gentiobiose; L-Arabinose; Lactulose; Maltose; Maltotriose; N- |
| | | Acetyl-D-Galactosamine; N-Acetyl-Neuraminic Acid; Salicin; Stachyose; Sucrose; |
| | | Thymidine; Uridine; a-D-Glucose; a-D-Lactose; b-Methyl-D-Glucoside; m-Inositol |
| FBI00174 | 1, 2 | Adenosine; D-Fructose-6-Phosphate; D-Galacturonic Acid; D-Gluconic Acid; D- |
| | | Glucose-1-Phosphate; D-Glucuronic Acid; D-Melezitose; D-Trehalose; Inosine; L- |
| | | Fucose; L-Galactonic Acid-g-Lactone; Laminarin; N-Acetyl-D-Glucosamine; Pectin |
| FBI00180 | 1, 2 | Inulin; b-D-Allose |
| FBI00182 | 1, 2 | Amygdalin; Arbutin; D-Cellobiose; D-Fructose; D-Galactose; D-Galacturonic Acid; |
| | | D-Mannose; D-Melibiose; D-Raffinose; Dextrin; Dihydroxy Acetone; Gentiobiose; |
| | | L-Galactonic Acid-g-Lactone; L-Rhamnose; Lactulose; Maltose; Maltotriose; N- |
| | | Acetyl-D-Glucosamine; Pectin; Salicin; Stachyose; Sucrose; a-D-Glucose; a-D- |
| | | Lactose; a-Methyl-D-Galactoside; b-Methyl-D-Galactoside; g-Cyclodextrin |
| FBI00184 | 1, 2 | Amygdalin; D-Galactose; D-Glucosamine; D-Mannose; D-Melibiose; D-Raffinose; |
| | | D-Trehalose; Gentiobiose; Lactulose; Maltitol; Maltose; Maltotriose; N-Acetyl-D- |
| | | Galactosamine; N-Acetyl-D-Glucosamine; N-Acetyl-b-D-Mannosamine; Palatinose; |
| | | Salicin; Stachyose; Sucrose; Turanose; Uridine; a-Cyclodextrin; a-D-Glucose; a-D- |
| | | Lactose; a-Methyl-D-Glucoside; b-Cyclodextrin; b-Methyl-D-Galactoside; g- |
| | | Cyclodextrin |
| FBI00189 | 1, 2 | Amygdalin; Arbutin; D-Arabinose; D-Cellobiose; D-Galactose; D-Glucosamine; D- |
| | | Mannitol; D-Mannose; D-Melibiose; D-Raffinose; D-Sorbitol; D-Trehalose; |
| | | Gentiobiose; Glycogen; L-Arabinose; L-Fucose; Lactitol; Lactulose; Maltitol; |
| | | Maltose; Maltotriose; N-Acetyl-D-Galactosamine; N-Acetyl-D-Glucosamine; |
| | | Palatinose; Salicin; Stachyose; Sucrose; Turanose; a-Cyclodextrin; a-D-Glucose; a-D- |
| | | Lactose; a-Methyl-D-Glucoside; a-Methyl-D-Mannoside; b-Cyclodextrin; b-Methyl- |
| | | D-Galactoside; b-Methyl-D-Glucoside; g-Cyclodextrin |
| FBI00190 | 1, 2 | Amygdalin; Arbutin; Chondroitin Sulfate C; D-Arabinose; D-Cellobiose; D-Fructose; |
| | | D-Fructose-6-Phosphate; D-Galactose; D-Galacturonic Acid; D-Glucosamine; D- |
| | | Glucose-1-Phosphate; D-Glucose-6-Phosphate; D-Glucuronic Acid; D-Mannose; D- |
| | | Melezitose; D-Melibiose; D-Raffinose; D-Trehalose; D-Xylose; Dextrin; Gentiobiose; |
| | | Glycogen; Glycyl-L-Aspartic Acid; L-Arabinose; L-Fucose; L-Galactonic Acid-g- |
| | | Lactone; L-Rhamnose; Lactitol; Lactulose; Laminarin; Maltitol; Maltose; Maltotriose; |
| | | N-Acetyl-D-Galactosamine; N-Acetyl-D-Glucosamine; N-Acetyl-b-D-Mannosamine; |
| | | Palatinose; Pectin; Salicin; Stachyose; Sucrose; Thymidine; Turanose; Uridine; a-D- |
| | | Glucose; a-D-Lactose; a-Methyl-D-Galactoside; a-Methyl-D-Glucoside; a-Methyl-D- |
| | | Mannoside; b-Methyl-D-Galactoside; b-Methyl-D-Glucoside |
| FBI00191 | 1, 2 | D-Trehalose; L-Arabinose; Maltotriose; N-Acetyl-D-Glucosamine; a-Cyclodextrin; b- |
| | | Cyclodextrin |
| FBI00194 | 1, 2 | Arbutin; D-Arabinose; D-Fructose; D-Galactose; D-Glucosamine; D-Melibiose; D- |
| | | Raffinose; D-Sorbitol; D-Trehalose; Gentiobiose; L-Fucose; Lactulose; Maltose; |
| | | Maltotriose; N-Acetyl-D-Glucosamine; Salicin; Stachyose; Sucrose; Turanose; a-D- |
| | | Glucose; a-D-Lactose |
| FBI00198 | 1, 2 | 2-Deoxy Adenosine; Arbutin; D-Cellobiose; D-Fructose; D-Galactose; D- |
| | | Galacturonic Acid; D-Gluconic Acid; D-Glucosamine; D-Glucuronic Acid; D- |
| | | Mannose; D-Melezitose; D-Melibiose; D-Raffinose; D-Ribose; D-Trehalose; D- |
| | | Xylose; Inosine; L-Arabinose; L-Fucose; Lactulose; Maltose; Maltotriose; N-Acetyl- |
| | | D-Glucosamine; N-Acetyl-Neuraminic Acid; Palatinose; Stachyose; Thymidine; |
| | | Turanose; a-D-Glucose; a-D-Lactose; b-D-Allose |
| FBI00199 | 1, 2 | D-Fructose; D-Galactonic Acid-g-Lactone; D-Galactose; D-Gluconic Acid; D- |
| | | Glucosamine; D-Glucose-1-Phosphate; D-Glucose-6-Phosphate; D-Mannose; D- |
| | | Melibiose; D-Psicose; D-Raffinose; D-Ribose; D-Sorbitol; D-Trehalose; D-Xylose; |
| | | L-Arabinose; L-Serine; L-Tartaric Acid; Lactulose; Maltose; Maltotriose; N-Acetyl- |
| | | D-Glucosamine; N-Acetyl-Neuraminic Acid; Palatinose; Stachyose; Sucrose; Uridine; |
| | | a-D-Glucose |
| FBI00200 | 1, 2 | D-Galacturonic Acid; D-Gluconic Acid; D-Glucuronic Acid; L-Galactonic Acid-g- |
| | | Lactone; Pectin |
| FBI00201 | 1, 2 | Dextrin; Glycogen; Inulin; Laminarin; g-Cyclodextrin |
| FBI00205 | 1, 2 | 3-0-b-D-Galacto-pyranosyl-D-Arabinose; Arbutin; D-Arabinose; D-Fructose; D- |
| | | Galactose; D-Gluconic Acid; D-Mannitol; D-Melibiose; D-Raffinose; D-Sorbitol; D- |
| | | Xylose; L-Fucose; L-Rhamnose; L-Tartaric Acid; Lactitol; Lactulose; Maltitol; |
| | | Maltose; Maltotriose; Melibionic Acid; N-Acetyl-D-Glucosamine; Palatinose; Pectin; |
| | | Salicin; Stachyose; Sucrose; Turanose; a-D-Glucose; a-D-Lactose; a-Methyl-D- |
| | | Galactoside; b-Methyl-D-Galactoside; m-Inositol |
| FBI00220 | 1, 2 | D-Arabitol; D-Fructose; D-Galacturonic Acid; D-Gluconic Acid; D-Mannitol; D- |
| | | Xylose; L-Serine; Maltose; Maltotriose; Pyruvic Acid; Sucrose; a-D-Glucose |
| FBI00232 | 1, 2 | Chondroitin Sulfate C; D-Galactose; Glycogen; Inulin; N-Acetyl-D-Galactosamine; |
| | | N-Acetyl-D-Glucosamine; Pectin; Sucrose; a-Cyclodextrin; b-Cyclodextrin; g- |
| | | Cyclodextrin |
| FBI00235 | 1, 2 | 1,2-Propanediol; 2-Hydroxy Benzoic Acid; 5-Keto-D-Gluconic Acid; Acetic Acid; |
| | | Amygdalin; D-Arabinose; D-Fucose; D-Ribose; D-Tagatose; D-Threonine; L- |
| | | Arabitol; L-Arginine; L-Pyroglutamic Acid; L-Rhamnose; Maltitol; N-Acetyl-L- |
| | | Glutamic Acid; Oxalic Acid; Quinic Acid; Sebacic Acid; Stachyose; Succinamic |
| | | Acid; Turanose; a-D-Lactose; a-Hydroxy Glutaric Acid-g-Lactone; a-Keto-Butyric |
| | | Acid; a-Keto-Valeric Acid; a-Methyl-D-Galactoside; a-Methyl-D-Glucoside; a- |
| | | Methyl-D-Mannoside; b-Methyl-D-Xyloside; d-Amino Valeric Acid; g-Amino |
| | | Butyric Acid; g-Hydroxy Butyric Acid |
| FBI00236 | 1, 2 | D-Galactose; D-Mannose; D-Melibiose; D-Raffinose; D-Trehalose; L-Fucose; |
| | | Maltose; Maltotriose; N-Acetyl-D-Glucosamine; Stachyose; Turanose; a-Methyl-D- |
| | | Galactoside; b-Methyl-D-Galactoside; b-Methyl-D-Glucoside; b-Methyl-D-Xyloside |
| FBI00245 | 1, 2 | Amygdalin; Chondroitin Sulfate C; D-Arabinose; D-Cellobiose; D-Fructose; D- |
| | | Galactose; D-Galacturonic Acid; D-Gluconic Acid; D-Glucosamine; D-Glucuronic |
| | | Acid; D-Mannose; D-Melezitose; D-Melibiose; D-Raffinose; D-Ribose; D-Saccharic |
| | | Acid; D-Trehalose; D-Xylose; Dextrin; Gentiobiose; Glycogen; Inulin; L-Arabinose; |
| | | L-Fucose; L-Galactonic Acid-g-Lactone; L-Glutamine; L-Histidine; L-Pyroglutamic |
| | | Acid; L-Rhamnose; Lactitol; Lactulose; Maltitol; Maltose; Maltotriose; Mannan; |
| | | Mucic Acid; N-Acetyl-D-Galactosamine; N-Acetyl-D-Glucosamine; N-Acetyl-b-D- |
| | | Mannosamine; Palatinose; Pectin; Pyruvic Acid; Salicin; Stachyose; Sucrose; |
| | | Turanose; Uridine; a-Cyclodextrin; a-D-Glucose; a-D-Lactose; a-Keto-Butyric Acid; |
| | | a-Keto-Glutaric Acid; a-Methyl-D-Galactoside; a-Methyl-D-Glucoside; a-Methyl-D- |
| | | Mannoside; b-Cyclodextrin; b-Methyl-D-Galactoside; b-Methyl-D-Glucoside; g- |
| | | Cyclodextrin |
| FBI00263 | 1, 2 | D-Galactose; D-Glucosamine; D-Mannose; D-Melibiose; D-Raffinose; D-Trehalose; |
| | | L-Arabinose; L-Fucose; Lactulose; Maltitol; Maltose; N-Acetyl-D-Galactosamine; N- |
| | | Acetyl-D-Glucosamine; N-Acetyl-Neuraminic Acid; Stachyose; Sucrose; Turanose; |
| | | a-D-Glucose; a-D-Lactose; a-Methyl-D-Glucoside; b-Methyl-D-Galactoside |
| FBI00269 | 1, 2 | D,L-a-Glycerol-Phosphate; D-Gluconic Acid; D-Glucuronic Acid; Dextrin; Gelatin; |
| | | Glycogen; Inulin; Laminarin; g-Cyclodextrin |
| FBI00278 | 1, 2 | Glycogen; Thymidine |
| FBI00281 | 1, 2 | D-Ribose; N-Acetyl-Neuraminic Acid |
| FBI00290 | 1, 2 | Glycogen |
| FBI00009 | 2 | Acetamide; D-Raffinose; g-Cyclodextrin |
| FBI00011 | 2 | 4-Hydroxy Benzoic Acid; Capric Acid; D,L-Carnitine; D,L-Octopamine; D- |
| | | Glucosamine; D-Ribono-1,4-Lactone; Dihydroxy Acetone; Glycine; Inulin; L- |
| | | Alaninamide; L-Arginine; L-Histidine; L-Homoserine; N-Acetyl-L-Glutamic Acid; |
| | | Putrescine; Quinic Acid; Turanose |
| FBI00016 | 2 | D-Arabinose; D-Glucosamine; D-Raffinose; Dextrin; Glycogen; Lactitol; N-Acetyl- |
| | | D-Galactosamine; N-Acetyl-Neuraminic Acid; Palatinose; Stachyose; a-Cyclodextrin; |
| | | b-Cyclodextrin; b-Methyl-D-Galactoside; g-Cyclodextrin |
| FBI00020 | 2 | Amygdalin; D-Arabinose; D-Glucosamine; D-Melezitose; D-Raffinose; Gentiobiose; |
| | | Maltitol; N-Acetyl-D-Galactosamine; Palatinose; Salicin; Stachyose; a-Methyl-D- |
| | | Glucoside; a-Methyl-D-Mannoside; b-Methyl-D-Galactoside; g-Cyclodextrin |
| FBI00021 | 2 | Amygdalin; Arbutin; D-Arabinose; D-Arabitol; D-Glucosamine; D-Melezitose; D- |
| | | Raffinose; Gentiobiose; Glycogen; Lactitol; Maltitol; N-Acetyl-D-Galactosamine; |
| | | Palatinose; Pectin; Salicin; Stachyose; Turanose; a-Cyclodextrin; a-Methyl-D- |
| | | Glucoside; a-Methyl-D-Mannoside; b-Cyclodextrin; b-Methyl-D-Galactoside; g- |
| | | Cyclodextrin |
| FBI00029 | 2 | Amygdalin; Arbutin; D-Glucosamine; D-Melezitose; D-Raffinose; Dextrin; |
| | | Gentiobiose; Inulin; Lactitol; Laminarin; Maltitol; N-Acetyl-D-Galactosamine; N- |
| | | Acetyl-Neuraminic Acid; Palatinose; Salicin; Stachyose; Turanose; a-Cyclodextrin; a- |
| | | Methyl-D-Glucoside; a-Methyl-D-Mannoside; b-Cyclodextrin; b-Methyl-D- |
| | | Galactoside; g-Cyclodextrin |
| FBI00032 | 2 | 5-Keto-D-Gluconic Acid; Arbutin; D-Glucosamine; Inulin; L-Sorbose; L-Tartaric |
| | | Acid; Lactitol; Maltitol; N-Acetyl-D-Glucosaminitol; Palatinose; Pectin; Salicin; |
| | | Turanose; a-Methyl-D-Mannoside |
| FBI00043 | 2 | 2-Hydroxy Benzoic Acid; Amygdalin; D-Arabinose; D-Ribono-1,4-Lactone; Dextrin; |
| | | Dihydroxy Acetone; Glycine; L-Leucine; Maltitol; N-Acetyl-Neuraminic Acid; |
| | | Oxalomalic Acid; Palatinose; Quinic Acid; Sebacic Acid; Stachyose; a-Methyl-D- |
| | | Glucoside; b-Methyl-D-Galactoside |
| FBI00049 | 2 | b-Cyclodextrin; g-Cyclodextrin |
| FBI00052 | 2 | Amygdalin; Arbutin; D-Arabinose; D-Glucosamine; D-Melezitose; D-Raffinose; |
| | | Gentiobiose; Maltitol; N-Acetyl-D-Galactosamine; Palatinose; Salicin; Turanose; a- |
| | | Methyl-D-Glucoside; a-Methyl-D-Mannoside; b-Methyl-D-Galactoside |
| FBI00056 | 2 | Amygdalin; D-Raffinose; Dextrin; Gentiobiose; Maltitol; N-Acetyl-Neuraminic Acid; |
| | | Palatinose; Stachyose; Turanose; b-Methyl-D-Galactoside |
| FBI00060 | 2 | Amygdalin; Arbutin; D-Arabinose; D-Glucosamine; D-Melezitose; D-Raffinose; |
| | | Dextrin; Gentiobiose; Glycogen; Lactitol; Maltitol; N-Acetyl-D-Galactosamine; |
| | | Palatinose; Salicin; Turanose; a-Cyclodextrin; a-Methyl-D-Glucoside; a-Methyl-D- |
| | | Mannoside; b-Cyclodextrin; b-Methyl-D-Galactoside; g-Cyclodextrin |
| FBI00062 | 2 | Dextrin; Lactitol; Maltitol; Palatinose; Salicin; Stachyose; Turanose; b-Methyl-D- |
| | | Galactoside; g-Cyclodextrin |
| FBI00075 | 2 | b-Methyl-D-Galactoside |
| FBI00076 | 2 | Amygdalin; D-Arabinose; D-Glucosamine; D-Melezitose; D-Raffinose; Gentiobiose; |
| | | Glycogen; Inulin; Laminarin; Maltitol; N-Acetyl-D-Galactosamine; Palatinose; |
| | | Pectin; Salicin; Turanose; a-Methyl-D-Glucoside; a-Methyl-D-Mannoside; b-Methyl- |
| | | D-Galactoside; g-Cyclodextrin |
| FBI00080 | 2 | Amygdalin; Arbutin; D-Melezitose; D-Raffinose; Dextrin; L-Arginine; Laminarin; |
| | | Maltitol; N-Acetyl-Neuraminic Acid; Pectin; Stachyose; a-Methyl-D-Mannoside; b- |
| | | Cyclodextrin; b-Methyl-D-Galactoside |
| FBI00111 | 2 | D-Arabinose; D-Glucosamine; D-Raffinose; Stachyose; b-D-Allose |
| FBI00112 | 2 | Amygdalin; Arbutin; D-Arabinose; D-Glucosamine; Inulin; N-Acetyl-D- |
| | | Galactosamine; Salicin; a-Cyclodextrin; b-Cyclodextrin; b-Methyl-D-Galactoside; g- |
| | | Cyclodextrin |
| FBI00116 | 2 | Amygdalin; D-Arabinose; D-Glucosamine; D-Raffinose; Gentiobiose; Palatinose; |
| | | Pectin; Stachyose; Turanose |
| FBI00124 | 2 | D-Glucosamine; D-Raffinose; Lactitol; Pectin; Stachyose; a-Methyl-D-Mannoside |
| FBI00126 | 2 | Arbutin; D-Glucosamine; D-Tagatose; N-Acetyl-D-Galactosamine; Pectin |
| FBI00127 | 2 | Amygdalin; Arbutin; Lactitol; Pectin; Salicin; Turanose; b-Methyl-D-Galactoside |
| FBI00135 | 2 | Amygdalin; D-Glucosamine; N-Acetyl-D-Galactosamine; N-Acetyl-Neuraminic Acid |
| FBI00140 | 2 | Dextrin; Gentiobiose; Glycogen; Laminarin; Stachyose; a-Cyclodextrin; b- |
| | | Cyclodextrin; g-Cyclodextrin |
| FBI00145 | 2 | Amygdalin; Arbutin; D-Raffinose; Dextrin; Gentiobiose; Lactitol; Salicin; Stachyose; |
| | | b-Methyl-D-Galactoside; g-Cyclodextrin |
| FBI00151 | 2 | 2-Deoxy-D-Ribose; D-Arabinose; D-Fructose; D-Gluconic Acid; D-Glucosamine; D- |
| | | Mannose; D-Raffinose; Dextrin; Gentiobiose; Glycogen; L-Isoleucine; Maltitol; |
| | | Maltose; Maltotriose; Mannan; N-Acetyl-D-Glucosamine; Palatinose; Pectin; |
| | | Stachyose; Sucrose; Turanose; a-Cyclodextrin; a-D-Glucose; b-Cyclodextrin; b-D- |
| | | Allose; b-Methyl-D-Galactoside; g-Cyclodextrin |
| FBI00152 | 2 | D-Raffinose; Dextrin; Gentiobiose; Glycogen; Laminarin; Stachyose; a-Cyclodextrin; |
| | | b-Cyclodextrin; g-Cyclodextrin |
| FBI00175 | 2 | D-Arabinose; D-Arabitol |
| FBI00197 | 2 | Amygdalin; Arbutin; Chondroitin Sulfate C; D-Arabinose; D-Glucosamine; D- |
| | | Melezitose; D-Raffinose; Gentiobiose; Lactitol; Laminarin; N-Acetyl-D- |
| | | Galactosamine; N-Acetyl-Neuraminic Acid; Pectin; Salicin; Stachyose; Turanose; a- |
| | | Cyclodextrin; b-Cyclodextrin; b-Methyl-D-Galactoside; b-Methyl-D-Glucuronic |
| | | Acid; b-Methyl-D-Xyloside; g-Cyclodextrin |
| FBI00206 | 2 | Amygdalin; Arbutin; D-Arabinose; D-Glucosamine; D-Melezitose; D-Raffinose; |
| | | Dextrin; Gentiobiose; Glycogen; Inulin; Lactitol; Maltitol; N-Acetyl-D- |
| | | Galactosamine; Palatinose; Pectin; Salicin; Stachyose; Turanose; a-Cyclodextrin; a- |
| | | Methyl-D-Glucoside; a-Methyl-D-Mannoside; b-Cyclodextrin; b-Methyl-D- |
| | | Galactoside; g-Cyclodextrin |
| FBI00211 | 2 | Amygdalin; D-Raffinose; Glycogen; Lactitol; Maltitol; Palatinose; Turanose; b- |
| | | Methyl-D-Galactoside |
| FBI00212 | 2 | 3-Methyl Glucose; 4-Hydroxy Benzoic Acid; Amygdalin; Arbutin; D-Arabinose; D- |
| | | Arabitol; D-Melezitose; Dextrin; Glycogen; Inulin; L-Isoleucine; L-Lysine; L- |
| | | Methionine; Lactitol; Maltitol; Palatinose; Turanose; a-Methyl-D-Glucoside; b-D- |
| | | Allose; b-Methyl-D-Galactoside; g-Cyclodextrin; i-Erythritol |
| FBI00243 | 2 | Sec-Butylamine |
| FBI00251 | 2 | D-Raffinose; Dextrin; Glycogen; Lactitol; Palatinose; Salicin; Turanose; b-Methyl-D- |
| | | Galactoside; g-Cyclodextrin |
| FBI00255 | 2 | Arbutin; D-Arabinose; D-Melezitose; D-Raffinose; Inulin; Stachyose; b-Cyclodextrin; |
| | | b-Methyl-D-Glucuronic Acid; b-Methyl-D-Xyloside; g-Cyclodextrin |
| FBI00267 | 2 | D,L-Octopamine; Sec-Butylamine |
| FBI00271 | 2 | 3-Methyl Glucose; Arbutin; D,L-Carnitine; D-Arabinose; D-Arabitol; D-Raffinose; |
| | | D-Ribono-1,4-Lactone; D-Tartaric Acid; Gentiobiose; Glycogen; Hydroxy-L-Proline; |
| | | Itaconic Acid; L-Histidine; L-Isoleucine; L-Valine; Laminarin; Quinic Acid; Sebacic |
| | | Acid; Sorbic Acid; Stachyose; Succinamic Acid; b-Hydroxy Butyric Acid; b-Methyl- |
| | | D-Galactoside |
| FBI00274 | 2 | D-Ribono-1,4-Lactone; L-Histidine; L-Homoserine; Quinic Acid |

The single carbon source conditions of Biolog plates are restrictive and not expected to promote growth of strains with more complex growth requirements. This was observed with the 41 strains of FB-001 and FB-003 that do not have a positive growth signature in the Biolog assays performed and the 9 strains that were not tested using Biolog due to insufficient growth. Of these combined 50 strains, 23 were tested with just 2 plates, 18 were tested with both PM1 and 2 plates, and 9 fastidious strains failed to reach the turbidity necessary to conduct a Biolog assay and are characterized in more detail below. The 41 strains that reached sufficient growth GD in complex growth media, but did not show positive growth in the Biolog plates tested, are shown in Table 15. These strains are routinely grown on complex YCFAC media and growth data in this medium are provided as the OD600 reached in the time given. Further information on the cultivation of these strains is available in the primary literature and summarized in Table 15 as well. In brief, for each strain we provide known macronutrient utilization, metabolite production and oxalate-formate characters. Macronutrients describe the primary contributors to biomass fora given strain, whereas metabolite production describes excreted small molecules that accumulate during cultivation and oxalate-formate focuses on the ability to degrade or resist the presence of these molecules. In cases where a macronutrient is predicted to be a substrate for growth, but growth was not directly observed in Biolog assays, it is expected that a second nutrient is required such as a vitamin or alternative nitrogen source that can be provided by the YCFAC recipe used for routine growth.

TABLE 15
Characterization of the 41 strains that did not show positive growth signatures by Biolog assay

| | | YCFAC | Incubation | |
|---|---|---|---|---|
| Strain | PM | OD₆₀₀ | time (h) | Macronutrient |
| FBI00068 | 1, 2 | 0.573 | 72 | mucin o-linked glycans |
| FBI00012 | 1, 2 | 0.788 | 68 | indole; mannose; raffinose |
| FBI00277 | 1, 2 | 1.097 | 68 | indole; mannose; raffinose |
| FBI00022 | 1, 2 | 0.342 | 72 | N/A |
| FBI00229 | 1, 2 | 0.186 | 72 | mannose |
| FBI00061 | 1, 2 | 0.047 | 72 | mannose; raffinose |
| FBI00238 | 1, 2 | 0.251 | 72 | N/A |
| FBI00288 | 1, 2 | 0.877 | 66.6 | N/A |
| FBI00038 | 1, 2 | 0.512 | 72 | acetate; cellobiose; fructose; glucose; lactose; maltose; mannose; |
| | | | | raffinose; starch |
| FBI00170 | 1, 2 | 0.067 | 72 | acetate; arginine; secoisolariciresinol diglucoside |
| FBI00096 | 1, 2 | 0.068 | 72 | acetate; arginine; secoisolariciresinol diglucoside |
| FBI00159 | 1, 2 | 0.063 | 68 | N/A |
| FBI00018 | 1, 2 | 0.799 | 72 | acetate; arabinoxylan; arginine; inulin; starch; xos |
| FBI00099 | 1, 2 | 0.094 | 72 | arginine; methionine |
| FBI00081 | 1, 2 | 0.612 | 72 | N/A |
| FBI00071 | 1, 2 | 1.066 | 70.7 | N/A |
| FBI00097 | 1, 2 | 0.179 | 72 | N/A |
| FBI00233 | 1, 2 | 0.432 | 72 | N/A |
| FBI00019 | 2 | 0.12 | 72 | indole |
| FBI00208 | 2 | 0.768 | 70 | inulin; mannose |
| FBI00162 | 2 | 1.146 | 70 | N/A |
| FBI00171 | 2 | 0.164 | 72 | N/A |
| FBI00221 | 2 | 0.963 | 70 | glucose |
| FBI00226 | 2 | 0.097 | 72 | arabinose; glucose; mannose; xylose |
| FBI00040 | 2 | 0.056 | 72 | N/A |
| FBI00237 | 2 | 0.053 | 71.8 | N/A |
| FBI00248 | 2 | 1.13 | 71.8 | N/A |
| FBI00260 | 2 | 1.153 | 72 | acetate; arabinoxylan; arginine; inulin; starch; xos |
| FBI00244 | 2 | 0.114 | 71.8 | acetate; arginine; chondroitin sulfate; fructose; galacturonate; |
| | | | | glucose; inulin; maltose; starch |
| FBI00132 | 2 | 0.154 | 72 | arginine; methionine |
| FBI00120 | 2 | 0.735 | 72 | starch |
| FBI00092 | 2 | 0.15 | 87.8 | arabinose; xylose |
| FBI00149 | 2 | 0.157 | 87.8 | arabinose; xylose |
| FBI00177 | 2 | 0.082 | 87.8 | N/A |
| FBI00066 | 2 | 0.09 | 87.8 | N/A |
| FBI00093 | 2 | 0.896 | 71.8 | starch |
| FBI00123 | 2 | 1.4 | 71.8 | starch |
| FBI00069 | 2 | 0.11 | 72 | starch |
| FBI00085 | 2 | 0.733 | 72 | starch |
| FBI00224 | 2 | 0.047 | 70 | cysteine |
| FBI00077 | 2 | 0.062 | 72 | cysteine |

While most strains show a Biolog or YCFAC signature, the nine most fastidious strains require more characterization, which are provided here. Of these nine strains, two are isolates of Methanobrevibacter smithii (FBI00270 and FBI00292), the only archaeal strains in FB-001. M. smithii grows through methanogenesis (CH₄ production) with utilization of CO₂+H₂, or formate (HCO₂⁻) as macronutrients. Because of these specific growth conditions and phylogeny, M. smithii can be challenging to grow, but is readily identifiable. Another two strains are Oxalobacter formigenes strains FBI0133 and FBI0289, which can be readily grown with YCFAC supplemented with 20 mM Sodium oxalate.

Strain FBI00258 Turicibacter sanguinis, is most easily identified through its distinctive filamentous cell shape, with filamentous growth contributing to a lack of turbidity observed in dispersed culture. For strains FBI00254 Eubacterium hallii, FBI00034 Eubacterium eligens, FBI00176 Ruthenibacterium lactatiformans, and FBI00273 Barnesiella intestinihominis identification was conducted with differential plating on four recipes of complex media (Table 16).

TABLE 16
Seven-day growth scores for strains FBI00176 Ruthenibacterium
lactatiformans and FBI00273 Barnesiella intestinihominis

| Strain ID | YCFAC | YCFAC-B | BHI | CBA |
|---|---|---|---|---|
| FBI00273 | + | − | − | + |
| FBI00176 | + | − | + | + |
| FBI00254 | + | + | − | + |
| FBI00034 | + | + | − | + |

PM1 plates contained the following molecules: L-Arabinose; N-Acetyl-D-Glucosamine; D-Saccharic Acid; Succinic Acid; D-Galactose; L-Aspartic Acid; L-Proline; D-Alanine; D-Trehalose; D-Mannose; Dulcitol; D-Serine; D-Sorbitol; Glycerol; L-Fucose; D-Glucuronic Acid; D-Gluconic Acid; D,L-a-Glycerol-Phosphate; D-Xylose; L-Lactic Acid; Formic Acid; D-Mannitol; L-Glutamic Acid; D-Glucose-6-Phosphate; D-Galactonic Acid-g-Lactone; D,L-Malic Acid; D-Ribose; Tween 20; L-Rhamnose; D-Fructose; Acetic Acid; a-D; Glucose; Maltose; D-Melibiose; Thymidine; L-Asparagine; D-Aspartic Acid; D-Glucosaminic Acid; 1,2-Propanediol; Tween 40; a-Keto-Glutaric Acid; a-Keto-Butyric Acid; a-Methyl-D-Galactoside; a-D-Lactose; Lactulose; Sucrose; Uridine; L-Glutamine; m-Tartaric Acid; D-Glucose-1-Phosphate; D-Fructose-6-Phosphate; Tween 80; a-Hydroxy Glutaric Acid-g-Lactone; a-Hydroxy Butyric Acid; b-Methyl-D-Glucoside; Adonitol; Maltotriose; 2-Deoxy Adenosine; Adenosine; Glycyl-L-Aspartic Acid; Citric Acid; m-Inositol; D-Threonine; Fumaric Acid; Bromo Succinic Acid; Propionic Acid; Mucic Acid; Glycolic Acid; Glyoxylic Acid; D-Cellobiose; InosinevGlycyl-L-Glutamic Acid; Tricarballylic Acid; L-Serine; L-Threonine; L-Alanine; L-Alanyl-Glycine; Acetoacetic Acid; N-Acetyl-b-D-Mannosamine; Mono Methyl Succinate; Methyl Pyruvate; D-Malic Acid; L-Malic Acid; Glycyl-L-Proline; p-Hydroxy Phenyl Acetic Acid; m-Hydroxy Phenyl Acetic Acid; Tyramine; D-Psicose; L-Lyxose; Glucuronamide; Pyruvic Acid; L-Galactonic Acid-g-Lactone; D; Galacturonic Acid; Phenylethyl-amine; 2-Aminoethanol.

PM2 plates contained the following molecules: Chondroitin Sulfate C; a-Cyclodextrin; b-Cyclodextrin; g-Cyclodextrin; Dextrin; Gelatin; Glycogen; Inulin; Laminarin; Mannan; Pectin; N-Acetyl-D-Galactosamine; N-Acetyl-Neuraminic Acid; b-D-Allose; Amygdalin; D-Arabinose; D-Arabitol; L-Arabitol; Arbutin; 2-Deoxy-D-Ribose; i-Erythritol; D-Fucose; 3-0-b-D-Galacto-pyranosyl-D-Arabinose; Gentiobiose; L-Glucose; Lactitol; D-Melezitose; Maltitol; a-Methyl-D-Glucoside; b-Methyl-D-Galactoside; 3-Methyl Glucose; b-Methyl-D-Glucuronic Acid; a-Methyl-D-Mannoside; b-Methyl-D-Xyloside; Palatinose; D-Raffinose; Salicin; Sedoheptulosan; L-Sorbose; Stachyose; D-Tagatose; Turanose; Xylitol; N-Acetyl-D-Glucosaminitol; g-Amino Butyric Acid; d-Amino Valeric Acid; Butyric Acid; Capric Acid; Caproic Acid; Citraconic Acid; Citramalic Acid; D-Glucosamine; 2-Hydroxy Benzoic Acid; 4-Hydroxy Benzoic Acid; b-Hydroxy Butyric Acid; g-Hydroxy Butyric Acid; a-Keto-Valeric Acid; Itaconic Acid; 5-Keto-D-Gluconic Acid; D-Lactic Acid Methyl Ester; Malonic Acid; Melibionic Acid; Oxalic Acid; Oxalomalic Acid; Quinic Acid; D-Ribono-1,4-Lactone; Sebacic Acid; Sorbic Acid; Succinamic Acid; D-Tartaric Acid; L-Tartaric Acid; Acetamide; L-Alaninamide; N-Acetyl-L-Glutamic Acid; L-Arginine; Glycine; L-Histidine; L-Homoserine; Hydroxy-L-Proline; L-Isoleucine; L-Leucine; L-Lysine; L-Methionine; L-Ornithine; L-Phenylalanine; L-Pyroglutamic Acid; L-Valine; D,L-Carnitine; Sec-Butylamine; D.L-Octopamine; Putrescine; Dihydroxy Acetone; 2,3-Butanediol; 2,3-Butanedione; 3-Hydroxy 2-Butanone.

Preparation of cell suspension and PM MicroPlate Inoculation. AN IF-0a Inoculating Fluid (1.2×) was prepared by adding 1.5 ml of 1 M NaHCO₃, 0.15 ml of 0.4 M thioglycolate and 0.15 ml of 1 mM methylene green to a bottle of IF-0a GN/GP base inoculating fluid (1.2×), for a total of 125 ml AN IF-0a Inoculating Fluid (1.2×). The inoculating fluid is confirmed to be fully deoxygenated when colorless as the methylene green indicator changes from the oxidized (green) to the reduced (colorless) form. PM MicroPlates were removed from packaging, placed in an anaerobic chamber. And allowed to equilibrate to the oxygen-free gas mix (5% CO₂, 5% H₂, 90% N₂) for two days to become anaerobic. Preparation of PM inoculating fluids comprised: 1) Prepared a test tube containing 10 ml of 1.2×AN IF-Ga, 2) Prepared inoculating fluids as described below, and 3) Dispensed inoculating fluids into vials.

Inoculation of PM MicroPlates. All the following steps were done in a strictly anaerobic atmosphere containing 5% CO₂, 5% H₂, 90% N₂. Step 1: Prepare Cell Suspensions (a. Strains were re-streaked from Research Cell Banks (RCBs) onto four plates of YCFAC media by streaking heavily and allowing the cells to grow 1-7 days at 37° C. in an atmosphere containing 5% CO₂, 5% H₂, 90% N₂; b. Cells were harvested from agar plates using a sterile swab and transferred into a tube containing 10 ml of 1.2×AN IF-0a. Cell suspensions were gently stirred with the swab to obtain a uniform suspension. Turbidity of the suspension was measured in Turbidimeter, and cells added to achieve a density of 40% T (transmittance)). Step 2: Inoculate PMs 1 and 2 (a. MicroPlates were prepped and labeled for each strain; b. 1.5 ml of cell suspension (Mix A) were added to 22.5 ml of AN PM1,2 inoculating fluid (Mix B) to a total of 24.0 ml. The final cell density is a 1:16 dilution of 40% T; c. PM MicroPlates were inoculated anaerobically from the 24 ml AB mixture by multichannel pipettor, with 100 ml aliquots per well).

Incubation and Data Collection. All cultures were maintained at 37° C. and anerobic conditions throughout the incubation. Growth of cells was measure by reading optical density at 600 nm (OD600) every 2 hours for using an Agilent Biostack microplate reader for 50-90 hours, depending on when stationary phase was reached across the plate.

### Example 7: In Vivo Assessment of FB-003 Engraftment and Metabolic Function

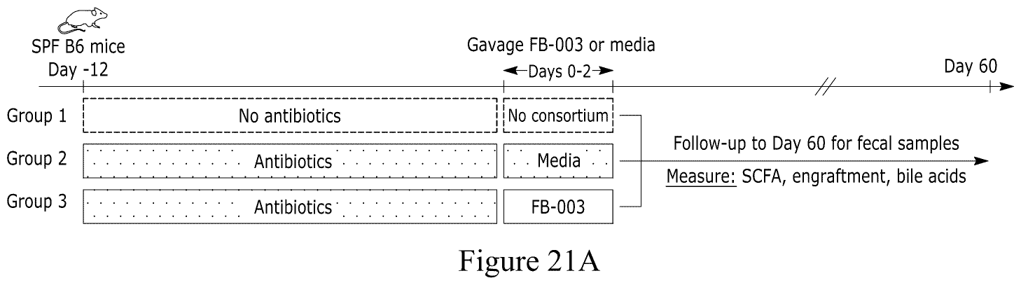

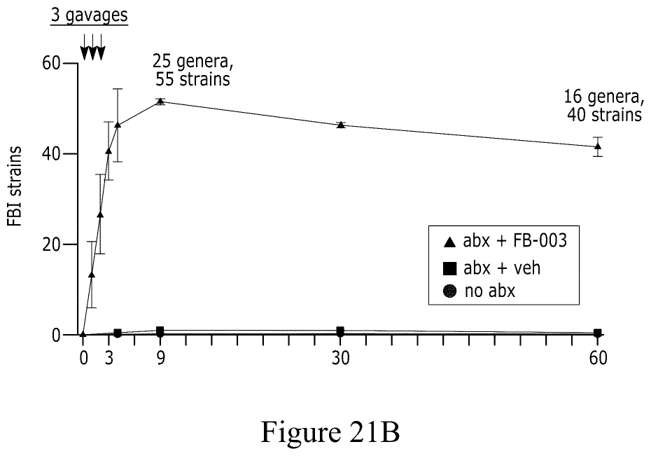

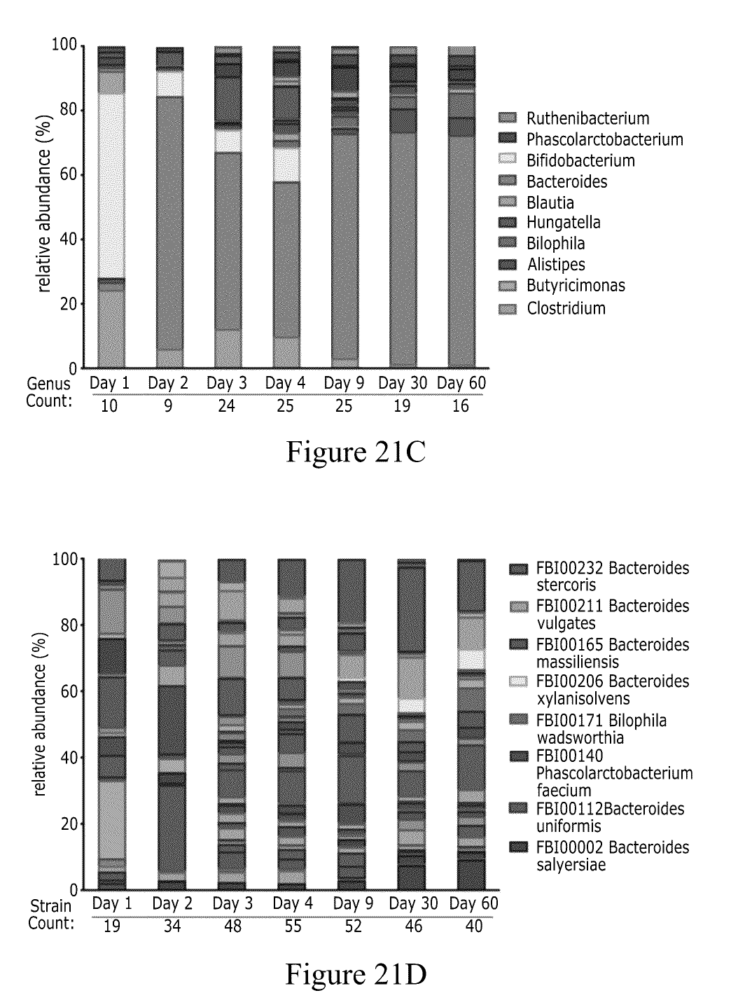

Engraftment of a human commensal consortium in naturally colonized mice is very difficult. In this experiment, SPF mice were treated with antibiotics (0.575 mg/mL enrofloxacin and 1 mg/mL ampicillin) for 12 days and randomized to receive FB-003 or placebo (i.e., “Media” or “veh”). Fecal samples were then taken from the animals over the course of 60 days and microbial engraftment, SCFA production, and bile acid derivatives were measured. FIG. 21A provides a schematic of the experimental design.

FIG. 21B shows robust engraftment in SPF mice. Specifically, FB-003 strains show stable engraftment over 60 days in the SPF mice while the placebo control shows no FB-003 strains detected. Furthermore, the profile of the strains was very stable with variation in abundance and some succession of Bacteroides species.

The engraftment at genus and strain level are shown in FIGS. 21C and 21D, respectively, and proves that not only is there engraftment, but also diverse colonization of FB-003 microbes. This data also shows that FB-003 engraftment stabilizes approximately 1 week after dosing SPF mice and remains stable for at least 60 days.

In addition to microbial diversity, proper SCFA levels are critical for human health. SCFAs are the energy source for the gut epithelium and they improve tight junctions of the intestinal epithelium and increase protective mucin production. SCFAs also balance immune cell function, reducing inflammation, and provide important signaling molecules that connect the gut with central metabolism and the central nervous system (e.g., SCFAs stimulate GLP-1 release, increase insulin release, and decrease appetite).

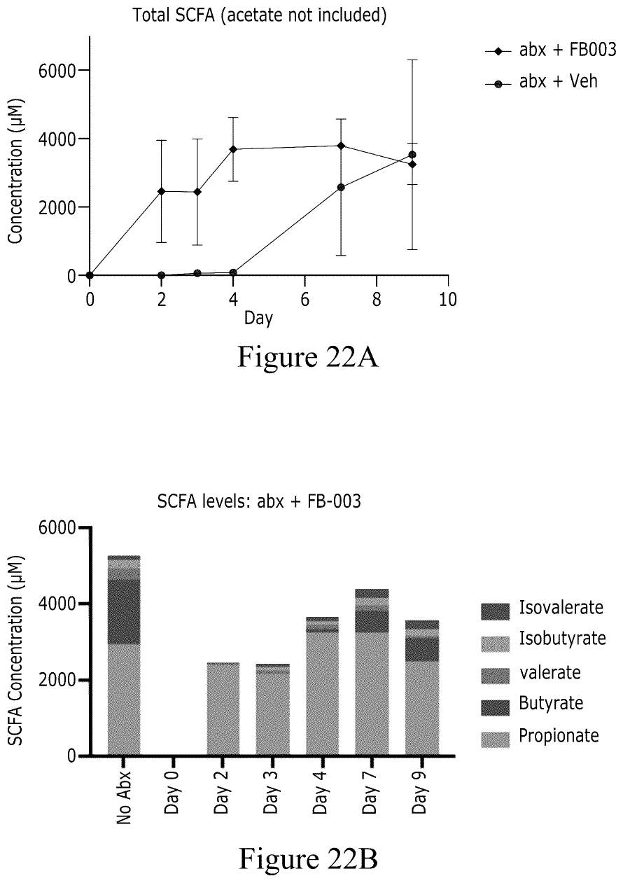

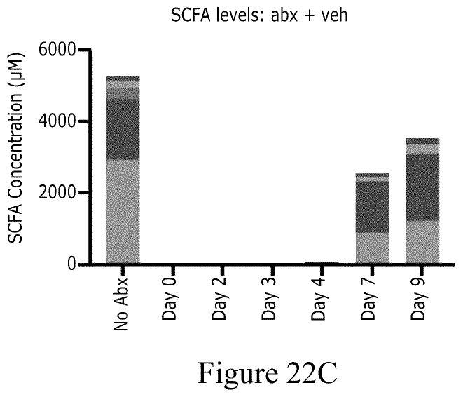

FB-003 treatment rapidly produced a modified SCFA profile in the SPF mice. As shown in FIGS. 22A-22C, the total SCFAs were able to recover faster with FB-003 relative to the vehicle control (i.e., placebo). FB-003 was also shown to be able to control the SCFA profile (see FIG. 22B wherein the relative levels of isovalerate, isobutyrate, valerate, butyrate, and propionate recovered and stabilized). For example, the “No Abx” pre-antibiotic treatment groups shown in FIGS. 22B and 22C show a higher percentage of propionate, followed by butyrate. However, following antibiotic treatment, the vehicle control groups were not able to achieve the health ratio of propionate:butyrate (i.e., butyrate became dominant over propionate which is a sign of dysbiosis) while the FB-003 treatment groups in FIG. 22B regained the healthy propionate:butyrate ratio and did not develop dysbiosis.

The microbiome determines the key signaling and toxicity characteristics of the bile acid pool. Bile acids are toxic molecules made by the liver to solubilize fats and break open the cells of consumed food. However, gut epitheliums and microbiomes are also made up of fat soluble membranes, rendering them susceptible to the toxic effects of bile acids. Accordingly, gut microbiomes have evolved to deconjugate and derivatize the bile acid pool and the properties of the gut microbiome determine whether this makes the bile acid pool more or less toxic to our cells. Furthermore, the microbiome composition can shift the bile acid pool between states. Specifically, bile acids are recycled in a mostly closed loop system between the liver and the gut such that the composition of the microbiome significantly determines the toxicity of the recycled bile acids. Accordingly, “good” bile acid pools are characterized by high solubility, high hydrophilicity, and good signalling properties (collectively resulting in, for example, glucose tolerance, triglyceride metabolism, healthy gut, and cholesterol metabolism) and “bad” bile acid pools are characterized by low solubility, low hydrophilicity (i.e., hydrophobic), and abnormal signalling (collectively resulting in, for example, inflammation and cell damage).

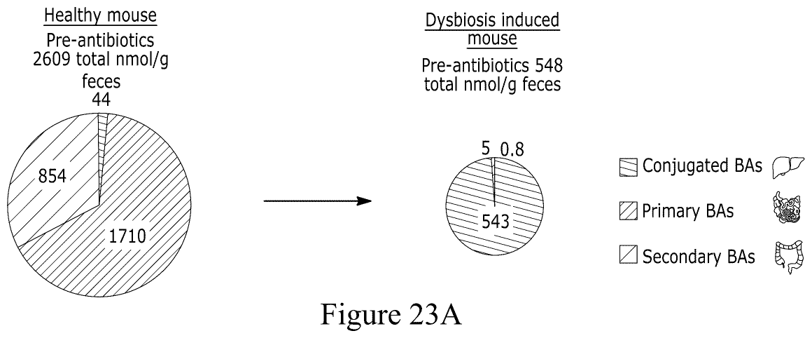

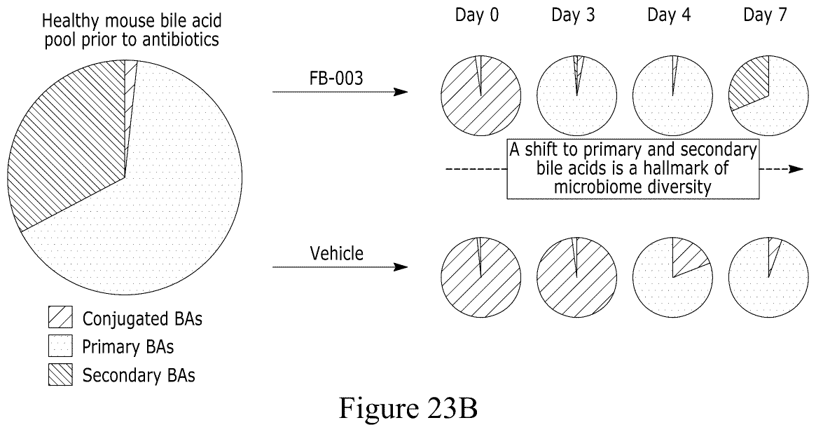

In the experiments described herein, the antibiotic treatment dramatically disrupted the bile acid pool in the SPF mice. As shown in FIG. 23A, prior to antibiotic treatment, colonized mice had very little conjugated bile acids and harbored a diverse array of bacterial produced primary and secondary bile acids. In contrast, antibiotic treatment eliminated nearly all microbiome mediated de-conjugation, and reduced the size of the bile acid pool (likely explained by the complex signaling between host cells and secondary bile acid derivatives that directly impact cholesterol/bile acid synthesis).

FB-003 was shown to rapidly restore the balance of the bile acid pool after antibiotic treatment induced dysbiosis (FIG. 23B). The data in FIG. 23B shows that FB-003 can rapidly engraft and re-balance the bile acid pool after antibiotic induced dysbiosis—by Day 7 after treatment with FB-003, the bile acid pool was restored to similar proportions as the healthy mouse pool before antibiotics. In contrast, without FB-003 treatment, the bile acid pool remained partially conjugated and devoid of secondary bile acids for a week after antibiotic removal. Tissue samples from the small intestines, colon, and cecum were also taken and imaged with probes to identify FB-003 strains that engrafted. The images (data not shown) show that FB-003 robustly engrafts throughout the gut.

### Example 8: FB-003 is Effective at Treating Dysbiosis of the Gut and IBD

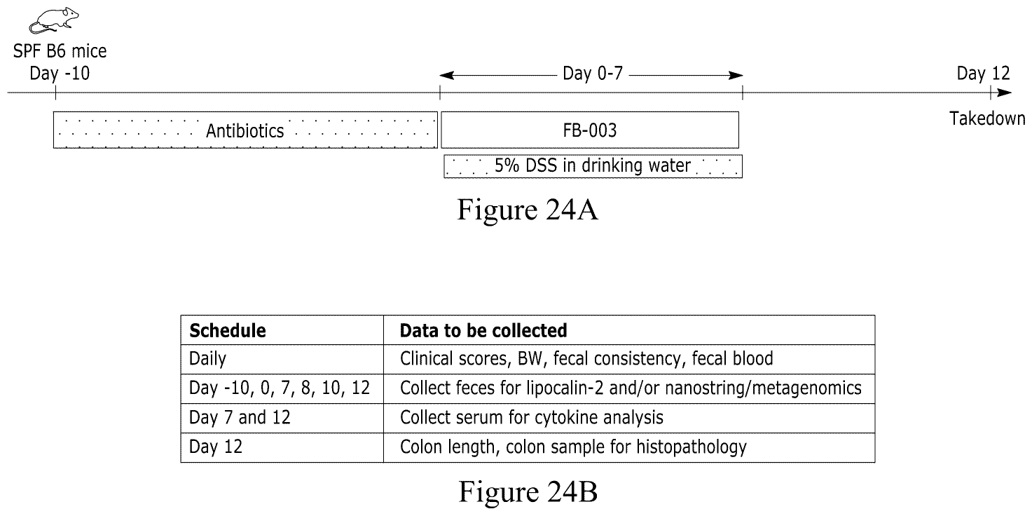

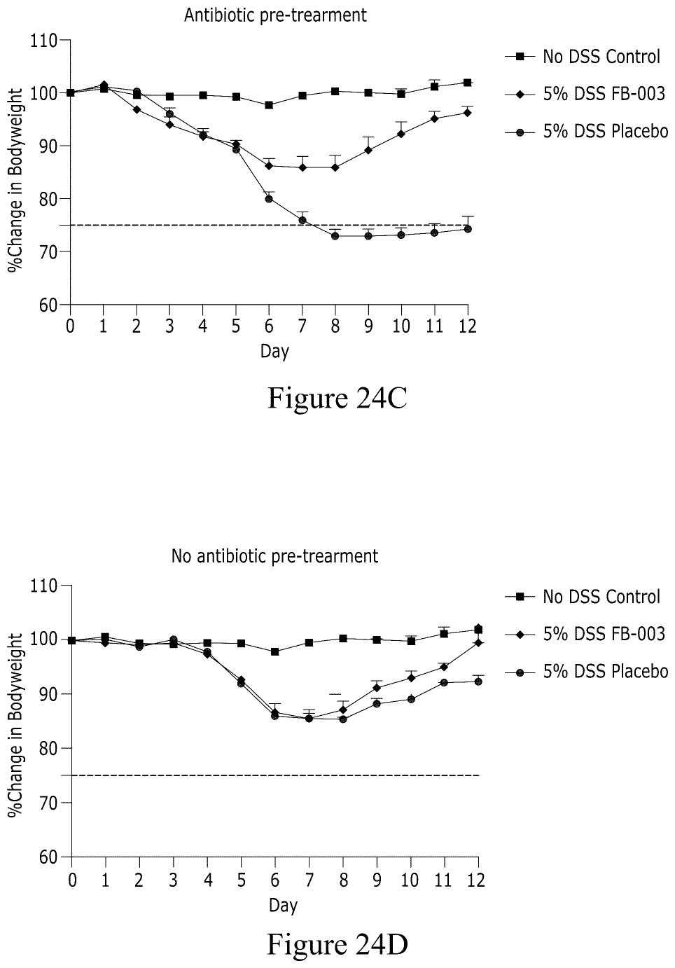

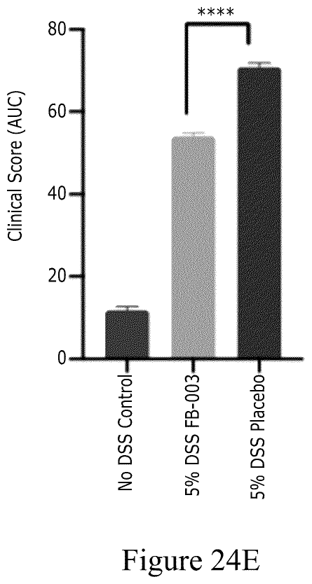

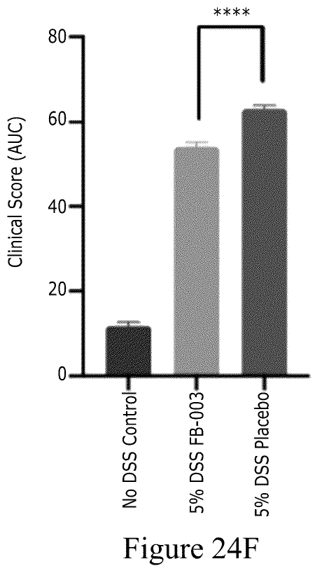

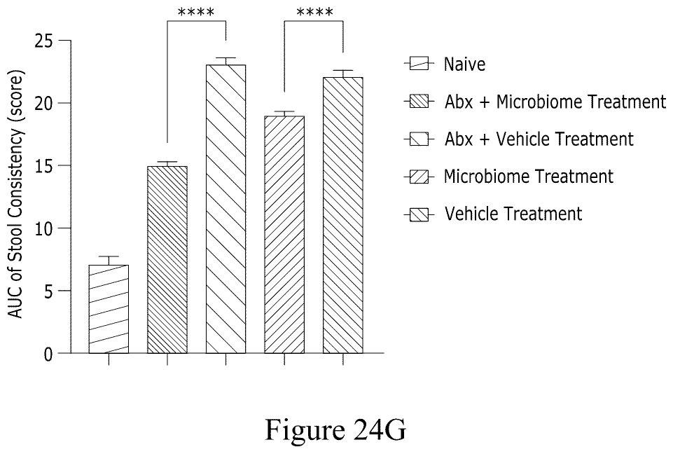

Following the confirmation that FB-003 was able to engraft and restore a healthy and functional gut microbiome, experiments were designed to test FB-003 in a DSS colitis model to determine its safety and efficacy for the treatment of IBD (including colitis, ulcerative colitis, and Crohn's disease). FIGS. 24A and 24B provide a schematic of the DSS colitis model experiments.

Briefly, the study was designed to determine the efficacy of FB-003 in a dextran sulphate sodium (DSS) induced murine model of ulcerative colitis. The experimental conditions of this study were as follows: the study was performed in 42 Adult male C57BL/6J mice and all groups were n=10 except naïve where n=2. Data readouts included morbidity, bodyweight, clinical scores, gross pathology, lipocalin 2 detection, and histopathology. FB-003 was administered orally, once daily. Table 17 provides the experimental conditions for this study.

TABLE 17
Experimental Conditions

| | | Treatments | | | |
|---|---|---|---|---|---|
| Groups | Pre-treatment | Dose | Route | Regimen | Intervention |
| Naïve (n = 2) | NA | NA | NA | NA | NA |
| Abx + microbiome | Ampicillin/Enrofloxacin | 2.5e9 VCC/200 uL | PO | SID from | DSS 5%, PO, |
| treatment | In drinking water | | | Day 0 to 7 | SID, Day 0 |
| Abx + vehicle | | 200 uL vehicle | PO | SID from | to Day 7 |
| treatment | | | | Day 0 7 | |
| Microbiome | NA | 2.5e9 VCC/200 uL | PO | SID from | |
| treatment | | | | Day 0 to 7 | |
| Vehicle | NA | 200 uL vehicle | PO | SID from | |
| treatment | | | | Day 0 to 7 | |

Adult male C57BL/6 mice were randomly allocated experimental groups based on bodyweights and allowed to acclimatize for one week. From Day −10 antibiotics and treatments were be administered in accordance with the experimental conditions outlined in Table 7. On Day 0, sterile drinking water was replaced by 5% Dextran Sulphate Sodium (DSS) in tap water. Animals were given ad libitum access to the DSS solution until Day 5 when it was replaced with tap drinking water for the remainder of the study. From Day 0 until the end of the experiment on Day 12, animals were monitored daily for clinical signs of colitis including; bodyweight loss, loose stools and/or diarrhea and presence of occult or gross blood in the stools. Animals reaching humane-end points were culled immediately. On Day −10 (prior to antibiotics), Day 0 (prior to DSS), Day 1, Day 5, Day 7, Day 11 and Day 12 faecal samples were collected and stored for analysis (Lipocalin-2 for Day 5 &12 samples). On Day 7 in-life blood samples were taken and processed to serum before being stored for optional cytokine analysis by Luminex. On Day 12, remaining animals were culled, terminal bloods were taken, processed to serum and stored for optional cytokine analysis by Luminex. The colon was dissected out and colon length measured. The distal colon was taken from all animals and placed in formalin. After 24 hours formalin was replaced with ethanol and stored at ambient temperature for future histopathology analysis.

Morbidity and mortality were assessed daily. Animals were monitored for clinical signs to include abnormal posture (e.g., hunched), abnormal coat condition (e.g., piloerection), changes to the colour of hairless areas (including cyanosis or jaundice), presence of masses and/or swelling, abnormal breathing, abnormal movements and decrease activity. Cause of death are provided whenever identified. Survival rates are provided. From Day 0, animals were weighed daily. Animals losing 25% or greater of Day 0 bodyweight was culled immediately. From Day 0 until the end of the experiment, animals were monitored daily for clinical signs of colitis to include bodyweight loss, loose stools and/or diarrhea and presence of blood, occult or gross, in the stools. At termination, the colon was dissected out, a picture taken, and length measurements were performed on the digital images using the Image J software. Faecal samples were homogenised in buffer and centrifuged to pellet. The resulting supernatants were added to a murine Lipocalin 2 ELISA and pg/ml levels were determined and corrected for weight of sample.

Results of the body weight analysis is provided in FIGS. 24C and 24D. This data shows that FB-003 reduces weight loss in the DSS colitis model.

The results of the clinical score analysis are provided in FIGS. 24E and 24F. As shown in FIG. 24E, antibiotic treatment increases DSS colitis severity, and FB-03 treatment dramatically improves the clinical score (as well as weight loss as shown in FIG. 24C). In the no antibiotic setting (FIG. 24F), FB-003 still shows statistically significant improvement in the clinical scores of the mice (as well as weight loss as shown in FIG. 24D).

FIG. 24G shows the improved stool consistency with FB-003 treatment (with or without antibiotic treatment).

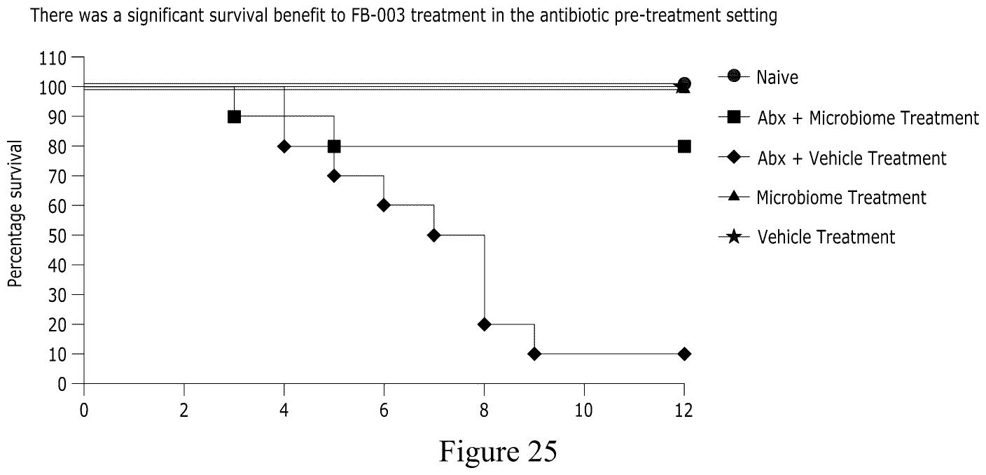

FIG. 25 shows survival data in the form of a Kaplan-Meier graph. As shown, the mice that were administered antibiotics and the vehicle control only showed a markedly reduced survival rate. However, mice that were treated with FB-003 had survival rates identical or on par with the mice received nothing. Furthermore, there was a significant survival benefit to FB-003 treatment in the antibiotic pre-treatment setting.

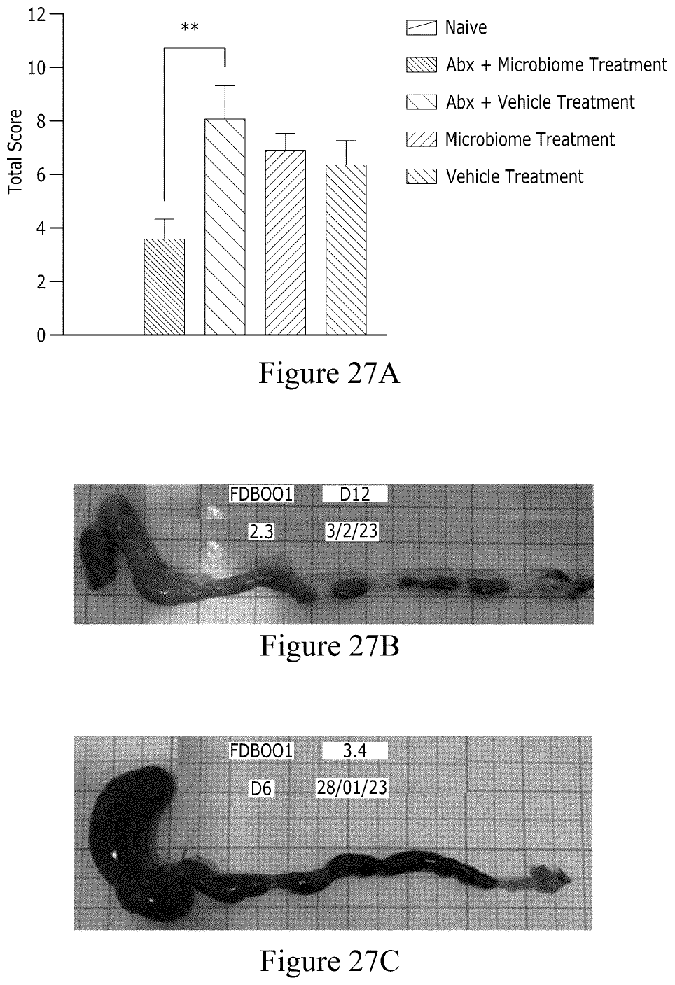

FIGS. 27A-27C show the therapeutic benefit of FB-003 on the health of the colon. Specifically, FIG. 27A shows the Total Histopathology Score of the colon. Total score was calculated based on the following parameters: Mucosal Thickness, Mucosal Ulceration, Mucosal Degeneration, Lamina propria (L.P) Mononuclear infiltration, L.P Granulocyte Infiltration and Crypt Abscesses/Dilation/Distortion. Data was analysed by Ordinary One-Way ANOVA followed by Sidak's multiple comparisons test the mean of each group was compared to the mean of all other treatment groups. Naive animals have been excluded and **<0.001. FIG. 27B shows representative colon images isolated from mice administrated with 5% DSS and then treated with Abx+FB-003. FIG. 27B shows representative colon images isolated from mice administrated with 5% DSS and then treated with Abx+Vehicle Control. As shown, the colon of FIG. 27B is healthy in contrast to the colon in FIG. 27C that is shortened from inflammation with ulcerations and bloody stool.

Furthermore, histopathology showed that mice administered 5% DSS and treated with Abx+FB-003 had a healthy epithelial barrier of the gut in contrast to mice administered 5% DSS and treated with Abx+Vehicle Control that had severe damage to their epithelial barriers of the gut.

Accordingly, FB-003 (and Consortia A and B with substantially similar function to FB-003) significantly improves survival in a DSS colitis model.

### Example 9: FB-003 is Effective at Treating Dysbiosis of the Gut and IBD

FB-003 can be tested in animal models of IBD to assess the ability of the drug product to modulate key pathways in disease. IBD is a complex multifactorial disease, and as such, no single animal model recapitulates all features of human disease. Thus, 3 models were selected to assess the ability of FB-003 to target distinct mechanisms that are central to disease in patients.

As described in Example 8, the dextran sodium sulfate (DSS) colitis model is the most well-characterized and reproducible model to interrogate epithelial barrier function in IBD. Disruption of the intestinal epithelium and entry of luminal bacteria and their products is a disease mechanism that exacerbates colitis in patients. In the DSS chemical model, barrier disruption is mediated by toxicity to the colon epithelium resulting in erosions, ulcers, crypt hyperplasia, and immune infiltration that mimic pathology in UC. Butyrate and other SCFAs are known to mediate colon epithelial health and induce tolerogenic T regs. FB-003 produces butyrate and rapidly corrects SCFA profiles, and efficacy in this model shows that production of these metabolites help restore the intestinal barrier in patients.

Ulcerative colitis (UC) and Crohn's disease (CD) patients have marked gut dysbiosis with increased abundance of pathogenic Proteobacteria (including E. coli, Klebsiella, and Citrobacter). The outgrowth of these pathogenic strains attach to the intestinal epithelium and cause lesions, resulting in damaged barrier integrity and inflammation. Citrobacter rodentium is a mouse-adapted E. coli-like strain that possesses these attaching and effacing properties. It causes a well-established model of infectious colitis in mice that recapitulates the loss of barrier integrity, inflammation, and pathology seen in UC. FB-003 can show that our microbial consortia can 1) displace pathogenic Proteobacteria and 2) protect barrier integrity in IBD patients.

Immune dysregulation and pro-inflammatory cytokines drive disease in both UC and CD. In patients, numerous pro-inflammatory signals are upregulated and there is a defect in the function or tolerogenic anti-inflammatory Tregs. The adoptive T cell transfer model of colitis was designed specifically to interrogate pathogenic pro-inflammatory T cells, in which the induction or transfer of Tregs can reverse pathology and prevent disease. This model captures the clinical pathology of both UC and CD. This model can be used to show that FB-003 induces Tregs and promotes their function, which can be mediated by butyrate and bile acid metabolites.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A method for preventing, decreasing, and/or treating dysbiosis in a subject, comprising administering an effective amount of a microbial consortium or a pharmaceutical composition thereof comprising
a) Clostridium citroniae, Bacteroides salyersiae, Blautia obeum, Parabacteroides merdae, Parabacteroides distasonis, Anaerostipes hadrus, Lachnospiraceae sp. FBI00033, Eubacterium eligens, Bifidobacterium dentium, Blautia wexlerae, Fusicatenibacter saccharivorans, Bacteroides nordii, Dorea formicigenerans, Dorea longicatena, Bacteroides stercorirosoris, Bifidobacterium longum, Bacteroides kribbi, Lachnospiraceae sp. FBI00071, Bacteroides thetaiotaomicron, Clostridium clostridioforme, Clostridium scindens, Roseburia hominis, Clostridium fessum, Coprococcus comes, Blautia faecis, Hungatella hathewayi, Bacteroides stercoris, Collinsella aerofaciens, Hungatella effluvii, Bifidobacterium adolescentis, Bifidobacterium catenulatum, Lactobacillus rogosae, Bacteroides faecis, Bacteroides finegoldii, Clostridiaceae sp. FBI00191, Ruminococcus faecis, Lachnoclostridium pacaense, Clostridium bolteae, Longicatena caecimuris, Eggerthella lenta, Blautia massiliensis, Bacteroides xylanisolvens, Bacteroides vulgatus, Megasphaera massiliensis, Butyricimonas faecihominis, Eisenbergiella tayi, Acidaminococcus intestini, Emergencia timonensis, Bifidobacterium pseudocatenulatum, Eubacterium hallii, Anaerofustis stercorihominis, Eubacterium ventriosum, Blautia hydrogenotrophica, and Lachnospiraceae sp. FBI00290, or a functional equivalent thereof; or
b) FBI00001, FBI00002, FBI00010, FBI00013, FBI00029, FBI00032, FBI00033, FBI00034, FBI00043, FBI00044, FBI00048, FBI00050, FBI00051, FBI00057, FBI00059, FBI00060, FBI00070, FBI00071, FBI00076, FBI00079, FBI00087, FBI00093, FBI00102, FBI00109, FBI00117, FBI00120, FBI00125, FBI00127, FBI00128, FBI00145, FBI00162, FBI00174, FBI00184, FBI00190, FBI00191, FBI00194, FBI00198, FBI00199, FBI00200, FBI00201, FBI00205, FBI00206, FBI00211, FBI00220, FBI00221, FBI00236, FBI00245, FBI00248, FBI00251, FBI00254, FBI00267, FBI00278, FBI00288, and FBI00290, or a functional equivalent thereof.

2. The method of claim 1, wherein the microbial consortium or a pharmaceutical composition thereof further comprises:
a) Acutalibacter timonensis, Alistipes onderdonkii, Bacteroides uniformis, Eubacterium rectale, Alistipes timonensis, Bacteroides kribbi, Coprococcus eutactus, Bilophila wadsworthia, Bacteroides caccae, Alistipes shahii, Parasutterella excrementihominis, Paraprevotella clara, Sutterella wadsworthensis, Sutterella massiliensis, Porphyromonas asaccharolytica, Ruminococcus bromii, Monoglobus pectinilyticus, Ruminococcaceae sp. FBI00097, Gordonibacter pamelaeae, Bacteroides uniformis, Gordonibacter pamelaeae, Bacteroides fragilis, Phascolarctobacterium faecium, Monoglobus pectinilyticus, Clostridium aldenense, Ruthenibacterium lactatiformans, Bacteroides ovatus, Bifidobacterium bifidum, Anaerotruncus massiliensis, Clostridium aldenense, Sutterella wadsworthensis, Catabacter hongkongensis, Alistipes senegalensis, Ruminococcaceae sp. FBI00233, Alistipes shahii, Dielma fastidiosa, Eubacterium siraeum, Faecalibacterium prausnitzii, Turicibacter sanguinis, Eubacterium rectale, Bacteroides caccae, Methanobrevibacter smithii, Barnesiella intestinihominis, Alistipes onderdonkii, and Methanobrevibacter smithii, or a functional equivalent thereof;
b) Bifidobacterium adolescentis, Bifidobacterium longum, Bifidobacterium pseudocatenulatum, Bacteroides thetaiotaomicron, Coprococcus comes, Fusicatenibacter saccharivorans, Eggerthella lenta, Eubacterium eligens, Bacteroides xylanisolvens, Lactobacillus rogosae, Clostridium citroniae, Collinsella aerofaciens, Blautia obeum, Eggerthella lenta, Blautia wexlerae, Lachnoclostridium pacaense, Bacteroides vulgatus, Parabacteroides merdae, Dorea formicigenerans, Ruminococcus faecis, Roseburia hominis, Anaerostipes hadrus, Bifidobacterium adolescentis, Bifidobacterium pseudocatenulatum, Clostridium bolteae, Eisenbergiella tayi, Dorea longicatena, Eggerthella lenta, Bacteroides stercoris, Hungatella hathewayi, and Bacteroides xylanisolvens, or a functional equivalent thereof; and/or
c) Alistipes putredinis, Dialister succinatiphilus, Akkermansia muciniphila, Ruminococcus bromii, Dialister invisus, Bacteroides massiliensis, Bilophila wadsworthia, Holdemanella biformis, Parasutterella excrementihominis, Alistipes sp. FBI00180, Bacteroides coprocola, Alistipes sp. FBI00238, Alistipes putredinis, Eubacterium xylanophilum, and Senegalimassilia anaerobia, or a functional equivalent thereof.

3. The method of claim 1, wherein the microbial consortium or a pharmaceutical composition thereof further comprises:
a) FBI00004, FBI00012, FBI00015, FBI00018, FBI00019, FBI00021, FBI00038, FBI00040, FBI00046, FBI00061, FBI00066, FBI00075, FBI00077, FBI00080, FBI00081, FBI00085, FBI00092, FBI00097, FBI00099, FBI00112, FBI00132, FBI00137, FBI00140, FBI00149, FBI00151, FBI00176, FBI00189, FBI00197, FBI00208, FBI00212, FBI00224, FBI00226, FBI00229, FBI00233, FBI00235, FBI00237, FBI00243, FBI00244, FBI00258, FBI00260, FBI00263, FBI00270, FBI00273, FBI00277, and FBI00292, or a functional equivalent thereof;
b) FBI00009, FBI00011, FBI00016, FBI00020, FBI00025, FBI00027, FBI00030, FBI00047, FBI00052, FBI00053, FBI00056, FBI00062, FBI00078, FBI00096, FBI00104, FBI00110, FBI00111, FBI00113, FBI00115, FBI00116, FBI00123, FBI00124, FBI00126, FBI00135, FBI00147, FBI00159, FBI00167, FBI00170, FBI00232, FBI00255, and FBI00271, or a functional equivalent thereof; and/or
c) FBI00022, FBI00049, FBI00068, FBI00069, FBI00152, FBI00165, FBI00171, FBI00175, FBI00177, FBI00180, FBI00182, FBI00238, FBI00269, FBI00274, and FBI00281, or a functional equivalent thereof.

4. The method of claim 1, wherein the microbial consortium or a pharmaceutical composition thereof comprises:
a) Clostridium citroniae, Bacteroides salyersiae, Blautia obeum, Parabacteroides merdae, Parabacteroides distasonis, Anaerostipes hadrus, Lachnospiraceae sp. FBI00033, Eubacterium eligens, Bifidobacterium dentium, Blautia wexlerae, Fusicatenibacter saccharivorans, Bacteroides nordii, Dorea formicigenerans, Dorea longicatena, Bacteroides stercorirosoris, Bifidobacterium longum, Bacteroides kribbi, Lachnospiraceae sp. FBI00071, Bacteroides thetaiotaomicron, Clostridium clostridioforme, Clostridium scindens, Roseburia hominis, Clostridium fessum, Coprococcus comes, Blautia faecis, Hungatella hathewayi, Bacteroides stercoris, Collinsella aerofaciens, Hungatella effluvii, Bifidobacterium adolescentis, Bifidobacterium catenulatum, Lactobacillus rogosae, Bacteroides faecis, Bacteroides finegoldii, Clostridiaceae sp. FBI00191, Ruminococcus faecis, Lachnoclostridium pacaense, Clostridium bolteae, Longicatena caecimuris, Eggerthella lenta, Blautia massiliensis, Bacteroides xylanisolvens, Bacteroides vulgatus, Megasphaera massiliensis, Butyricimonas faecihominis, Eisenbergiella tayi, Acidaminococcus intestini, Emergencia timonensis, Bifidobacterium pseudocatenulatum, Eubacterium hallii, Anaerofustis stercorihominis, Eubacterium ventriosum, Blautia hydrogenotrophica, and Lachnospiraceae sp. FBI00290, or a functional equivalent thereof;
b) Acutalibacter timonensis, Alistipes onderdonkii, Bacteroides uniformis, Eubacterium rectale, Alistipes timonensis, Bacteroides kribbi, Coprococcus eutactus, Bilophila wadsworthia, Bacteroides caccae, Alistipes shahii, Parasutterella excrementihominis, Paraprevotella clara, Sutterella wadsworthensis, Sutterella massiliensis, Porphyromonas asaccharolytica, Ruminococcus bromii, Monoglobus pectinilyticus, Ruminococcaceae sp. FBI00097, Gordonibacter pamelaeae, Bacteroides uniformis, Gordonibacter pamelaeae, Bacteroides fragilis, Phascolarctobacterium faecium, Monoglobus pectinilyticus, Clostridium aldenense, Ruthenibacterium lactatiformans, Bacteroides ovatus, Bifidobacterium bifidum, Anaerotruncus massiliensis, Clostridium aldenense, Sutterella wadsworthensis, Catabacter hongkongensis, Alistipes senegalensis, Ruminococcaceae sp. FBI00233, Alistipes shahii, Dielma fastidiosa, Eubacterium siraeum, Faecalibacterium prausnitzii, Turicibacter sanguinis, Eubacterium rectale, Bacteroides caccae, Methanobrevibacter smithii, Barnesiella intestinihominis, Alistipes onderdonkii, and Methanobrevibacter smithii, or a functional equivalent thereof;
c) Bifidobacterium adolescentis, Bifidobacterium longum, Bifidobacterium pseudocatenulatum, Bacteroides thetaiotaomicron, Coprococcus comes, Fusicatenibacter saccharivorans, Eggerthella lenta, Eubacterium eligens, Bacteroides xylanisolvens, Lactobacillus rogosae, Clostridium citroniae, Collinsella aerofaciens, Blautia obeum, Eggerthella lenta, Blautia wexlerae, Lachnoclostridium pacaense, Bacteroides vulgatus, Parabacteroides merdae, Dorea formicigenerans, Ruminococcus faecis, Roseburia hominis, Anaerostipes hadrus, Bifidobacterium adolescentis, Bifidobacterium pseudocatenulatum, Clostridium bolteae, Eisenbergiella tayi, Dorea longicatena, Eggerthella lenta, Bacteroides stercoris, Hungatella hathewayi, and Bacteroides xylanisolvens, or a functional equivalent thereof; and
d) Alistipes putredinis, Dialister succinatiphilus, Akkermansia muciniphila, Ruminococcus bromii, Dialister invisus, Bacteroides massiliensis, Bilophila wadsworthia, Holdemanella biformis, Parasutterella excrementihominis, Alistipes sp. FBI00180, Bacteroides coprocola, Alistipes sp. FBI00238, Alistipes putredinis, Eubacterium xylanophilum, and Senegalimassilia anaerobia, or a functional equivalent thereof.

5. The method of claim 1, wherein the microbial consortium or a pharmaceutical composition thereof comprises:
a) FBI00001, FBI00002, FBI00010, FBI00013, FBI00029, FBI00032, FBI00033, FBI00034, FBI00043, FBI00044, FBI00048, FBI00050, FBI00051, FBI00057, FBI00059, FBI00060, FBI00070, FBI00071, FBI00076, FBI00079, FBI00087, FBI00093, FBI00102, FBI00109, FBI00117, FBI00120, FBI00125, FBI00127, FBI00128, FBI00145, FBI00162, FBI00174, FBI00184, FBI00190, FBI00191, FBI00194, FBI00198, FBI00199, FBI00200, FBI00201, FBI00205, FBI00206, FBI00211, FBI00220, FBI00221, FBI00236, FBI00245, FBI00248, FBI00251, FBI00254, FBI00267, FBI00278, FBI00288, and FBI00290, or a functional equivalent thereof;
b) FBI00004, FBI00012, FBI00015, FBI00018, FBI00019, FBI00021, FBI00038, FBI00040, FBI00046, FBI00061, FBI00066, FBI00075, FBI00077, FBI00080, FBI00081, FBI00085, FBI00092, FBI00097, FBI00099, FBI00112, FBI00132, FBI00137, FBI00140, FBI00149, FBI00151, FBI00176, FBI00189, FBI00197, FBI00208, FBI00212, FBI00224, FBI00226, FBI00229, FBI00233, FBI00235, FBI00237, FBI00243, FBI00244, FBI00258, FBI00260, FBI00263, FBI00270, FBI00273, FBI00277, and FBI00292, or a functional equivalent thereof;
c) FBI00009, FBI00011, FBI00016, FBI00020, FBI00025, FBI00027, FBI00030, FBI00047, FBI00052, FBI00053, FBI00056, FBI00062, FBI00078, FBI00096, FBI00104, FBI00110, FBI00111, FBI00113, FBI00115, FBI00116, FBI00123, FBI00124, FBI00126, FBI00135, FBI00147, FBI00159, FBI00167, FBI00170, FBI00232, FBI00255, and FBI00271, or a functional equivalent thereof; and
d) FBI00022, FBI00049, FBI00068, FBI00069, FBI00152, FBI00165, FBI00171, FBI00175, FBI00177, FBI00180, FBI00182, FBI00238, FBI00269, FBI00274, and FBI00281, or a functional equivalent thereof.

6. The method of claim 1, wherein the microbial consortium or a pharmaceutical composition thereof further comprises a first strain of Oxalobacter formigenes or FBI00067, a second strain of Oxalobacter formigenes or FBI00133, and a third strain of Oxalobacter formigenes or FBI00289.

7. The method of claim 1, wherein the microbial consortium or a pharmaceutical composition thereof is FB-001, FB-003, or a functional equivalent thereof.

8. The method of claim 1, wherein (a) the microbial consortium or a pharmaceutical composition thereof increases the microbial diversity of the gastrointestinal tract; (b) the microbial consortium or a pharmaceutical composition thereof increases short chain fatty acids (SCFAs); (c) the microbial consortium or a pharmaceutical composition thereof increases secondary bile acids; and/or (d) the microbial consortium or a pharmaceutical composition thereof decreases bacterial pathogens in the gastrointestinal tract of the subject.

9. The method of claim 1, wherein the microbial consortium or a pharmaceutical composition thereof comprises (a) between about 5×10⁹ and about 5×10¹² viable cells; (b) between about 5×10⁹ and about 5×10¹⁰ viable cells; (c) between about 5×10¹⁰ and about 5×10¹¹ viable cells; (d) comprises between about 5×10¹¹ and about 5×10¹² viable cells; (e) up to about 10¹¹ viable cells; or (f) up to about 10¹² viable cells.

10. The method of claim 1, wherein the method comprises administering a loading dose and one or more maintenance doses.

11. The method of claim 10, wherein the loading dose is administered for 1 day, 2 days, 3 days, 4 days, 5 days, 6 days, 7 days, 8 days, 9 days, or 10 days.

12. The method of claim 10, wherein the one or more maintenance doses are administered for at least 21 days following the last loading dose.

13. The method of claim 1 further comprising administering an antibacterial agent, an antiviral agent, an antifungal agent, anti-inflammatory agent, an immunosuppressive agent, and/or a prebiotic.

14. The method of claim 1, wherein the microbial consortium or pharmaceutical composition thereof is present in a food product.

15. A method for restoring microbiome and/or recovering a healthy microbiome in a subject, comprising administering an effective amount of a microbial consortium or a pharmaceutical composition thereof comprising
a) Clostridium citroniae, Bacteroides salyersiae, Blautia obeum, Parabacteroides merdae, Parabacteroides distasonis, Anaerostipes hadrus, Lachnospiraceae sp. FBI00033, Eubacterium eligens, Bifidobacterium dentium, Blautia wexlerae, Fusicatenibacter saccharivorans, Bacteroides nordii, Dorea formicigenerans, Dorea longicatena, Bacteroides stercorirosoris, Bifidobacterium longum, Bacteroides kribbi, Lachnospiraceae sp. FBI00071, Bacteroides thetaiotaomicron, Clostridium clostridioforme, Clostridium scindens, Roseburia hominis, Clostridium fessum, Coprococcus comes, Blautia faecis, Hungatella hathewayi, Bacteroides stercoris, Collinsella aerofaciens, Hungatella effluvii, Bifidobacterium adolescentis, Bifidobacterium catenulatum, Lactobacillus rogosae, Bacteroides faecis, Bacteroides finegoldii, Clostridiaceae sp. FBI00191, Ruminococcus faecis, Lachnoclostridium pacaense, Clostridium bolteae, Longicatena caecimuris, Eggerthella lenta, Blautia massiliensis, Bacteroides xylanisolvens, Bacteroides vulgatus, Megasphaera massiliensis, Butyricimonas faecihominis, Eisenbergiella tayi, Acidaminococcus intestini, Emergencia timonensis, Bifidobacterium pseudocatenulatum, Eubacterium hallii, Anaerofustis stercorihominis, Eubacterium ventriosum, Blautia hydrogenotrophica, and Lachnospiraceae sp. FBI00290, or a functional equivalent thereof; or
b) FBI00001, FBI00002, FBI00010, FBI00013, FBI00029, FBI00032, FBI00033, FBI00034, FBI00043, FBI00044, FBI00048, FBI00050, FBI00051, FBI00057, FBI00059, FBI00060, FBI00070, FBI00071, FBI00076, FBI00079, FBI00087, FBI00093, FBI00102, FBI00109, FBI00117, FBI00120, FBI00125, FBI00127, FBI00128, FBI00145, FBI00162, FBI00174, FBI00184, FBI00190, FBI00191, FBI00194, FBI00198, FBI00199, FBI00200, FBI00201, FBI00205, FBI00206, FBI00211, FBI00220, FBI00221, FBI00236, FBI00245, FBI00248, FBI00251, FBI00254, FBI00267, FBI00278, FBI00288, and FBI00290, or a functional equivalent thereof.

16. A method for treating a disease in a subject, comprising administering an effective amount of a microbial consortium or a pharmaceutical composition thereof comprising
a) Clostridium citroniae, Bacteroides salyersiae, Blautia obeum, Parabacteroides merdae, Parabacteroides distasonis, Anaerostipes hadrus, Lachnospiraceae sp. FBI00033, Eubacterium eligens, Bifidobacterium dentium, Blautia wexlerae, Fusicatenibacter saccharivorans, Bacteroides nordii, Dorea formicigenerans, Dorea longicatena, Bacteroides stercorirosoris, Bifidobacterium longum, Bacteroides kribbi, Lachnospiraceae sp. FBI00071, Bacteroides thetaiotaomicron, Clostridium clostridioforme, Clostridium scindens, Roseburia hominis, Clostridium fessum, Coprococcus comes, Blautia faecis, Hungatella hathewayi, Bacteroides stercoris, Collinsella aerofaciens, Hungatella effluvii, Bifidobacterium adolescentis, Bifidobacterium catenulatum, Lactobacillus rogosae, Bacteroides faecis, Bacteroides finegoldii, Clostridiaceae sp. FBI00191, Ruminococcus faecis, Lachnoclostridium pacaense, Clostridium bolteae, Longicatena caecimuris, Eggerthella lenta, Blautia massiliensis, Bacteroides xylanisolvens, Bacteroides vulgatus, Megasphaera massiliensis, Butyricimonas faecihominis, Eisenbergiella tayi, Acidaminococcus intestini, Emergencia timonensis, Bifidobacterium pseudocatenulatum, Eubacterium hallii, Anaerofustis stercorihominis, Eubacterium ventriosum, Blautia hydrogenotrophica, and Lachnospiraceae sp. FBI00290, or a functional equivalent thereof; or
b) FBI00001, FBI00002, FBI00010, FBI00013, FBI00029, FBI00032, FBI00033, FBI00034, FBI00043, FBI00044, FBI00048, FBI00050, FBI00051, FBI00057, FBI00059, FBI00060, FBI00070, FBI00071, FBI00076, FBI00079, FBI00087, FBI00093, FBI00102, FBI00109, FBI00117, FBI00120, FBI00125, FBI00127, FBI00128, FBI00145, FBI00162, FBI00174, FBI00184, FBI00190, FBI00191, FBI00194, FBI00198, FBI00199, FBI00200, FBI00201, FBI00205, FBI00206, FBI00211, FBI00220, FBI00221, FBI00236, FBI00245, FBI00248, FBI00251, FBI00254, FBI00267, FBI00278, FBI00288, and FBI00290, or a functional equivalent thereof;
wherein the disease is irritable bowel syndrome, diarrhea, constipation, celiac disease, and leaky gut syndrome, colitis, ulcerative colitis, or Crohn's disease.

17. A method for decreasing dysbiosis in a subject, the method comprising administering to a patient a therapeutically effective amount of a pharmaceutical composition comprising a Consortia to decrease dysbiosis of the gastrointestinal tract in the patient.

18. The method of claim 17, wherein decreasing dysbiosis comprises engraftment of microbes of the Consortia, increase in the microbial diversity of the gastrointestinal tract, increase in the short chain fatty acids (SCFAs), increase in secondary bile acids, and/or decrease of bacterial pathogens.

19. The methods of claim 17, wherein the Consortia is FB-001, FB-003 or a functional equivalent thereof.

20. A method for restoring the microbiome in a patient, increasing the recovery of a healthy microbiome in a patient after a dysbiosis inducing event, and/or treating or reducing a severity of at least one symptom of a gastrointestinal disease associated with a dysbiosis, the method comprising administering to the patient a therapeutically effective amount of a pharmaceutical composition comprising a Consortia.

21. A method of reducing the symptoms associated with or treating IBD, colitis, ulcerative colitis, or Crohn's disease by administering a Consortia.


SEQUENCE LISTING (148 sequences)

SEQ ID NO: 1 - DNA, 1393 bp, synthetic construct
  misc_feature 1..1393: note = Synthetic: FBI00001
  source 1..1393: mol_type = other DNA
gcagtcgnacgaagcaatcagaatgaanttttcggatggatttctggttgactgagtggcggacgggtgagtaacgcgtggataacctgcctcacactgggggataacagttagaaatggctgctaataccgcataagcgcacagtaccgcatggtacggtgtgaaaaactccggtggtgtgagatggatccgcgtctgattagttagttggcggggtaacggcccaccaagacagcgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctggagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggtcagtaaagtgacctttccttcgggacaggggagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcgaccctgcgaaggcaagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcaagtgacccaaccgaaaggaggg

SEQ ID NO: 2 - DNA, 1397 bp, synthetic construct
  misc_feature 1..1397: note = Synthetic: FBI00002
  source 1..1397: mol_type = other DNA
anncntgcaagtcgaggggnagcnnggtgtagcaatacaccgcgtggcgaccggcgcacgggtgagtaacacgtatccaacctgccctttactcggggatagcctttcgaaagaaagattaatacccgatggtataacatgacctcctggttttgttattaaagaatttcggtagaggatggggatgcgttccattaggcagttggcggggtaacggcccaccaaaccttcgatggataggggttctgagaggaaggtcccccacattggaactgagacacggtccaaactcctacgggaggcagcagtgaggaatattggtcaatgggcgagagcctgaaccagccaagtagcgtgaaggatgaccgccctatgggttgtaaacttcttttatatgggaataaagggtgccacgtgtggcattttgtatgtaccatatgaataaggatcggctaactccgtgccagcagccgcggtaatacggaggatccgagcgttatccggatttattgggtttaaagggagcgtaggtggacatgtaagtcagttgtgaaagtttgcggctcaaccgtaaaattgcagttgatactgcgtgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaatgaatatgccggaaacggcatagccgcaaggcatttgtgaaggtgctgcatggttgtcgtcagctcgtgccgtgaggtgtcggcttaagtgccataacgagcgcaacccttatcttcagttactaacaggttatgctgaggactctggagagactgccgtcgtaagatgtgaggaaggtggggatgacgtcaaatcagcacggcccttacgtccggggctacacacgtgttacaatggggggtacagaaggccgctacacagcgatgtgatgccaatccctaaagcccctctcagttcggatcgaagtctgcaacccgacttcgtgaagctggattcgctagtaatcgcgcatcagccacggcgcggtgaatacgttcccgggccttgtacacaccgcccgtcaagccatgggagccgggggtacnngaagtacgtaaccgcaaggagcnnc

SEQ ID NO: 3 - DNA, 1404 bp, synthetic construct
  misc_feature 1..1404: note = Synthetic: FBI00004
  source 1..1404: mol_type = other DNA
tannnntgcagtcgaacggagatagacgctgaaagggagacagcttgctgtaagaatttcttgtttatcttagtggcggacgggtgagtaacgcgtgagtaacctgcctttcagagggggataacgtctggaaacggacgctaataccgcatgagaccacagcttcacatggagcggcggtcaaaggagcaatccgctgaaagatggactcgcgtccgattagatagttggcggggtaacggcccaccaagtcgacgatcggtagccggactgagaggttgaacggccacattgggactgagacacggcccagactcctacgggaggcagcagtgagggatattggtcaatgggggaaaccctgaaccagcaacgccgcgtgagggaagacggttttcggattgtaaacctctgtcctctgtgaagatagtgacggtagcagaggaggaagctccggctaactacgtgccagcagccgcggtaatacgtagggagcgagcgttgtccggatttactgggtgtaaagggtgcgtaggcggctctgcaagtcagaagtgaaatccatgggcttaacccatgaactgcttttgaaaactgtagagcttgagtgaagtagaggtaggcggaattcccggtgtagcggtgaaatgcgtagagatcgggaggaacaccagtgggcgaanggcgnctactgggctttaactgacgctgagcacgaaagcatgggtagcaaacaggattagataccctggtagtccatgccgtaaacgatgattactaggntgtgggggggtctgaccccctccgtgccggagttaacacaataagtaatccaccnggggagtacgaccgcaaggttgaaactcaaaggaattgacgggggcccgcacaagcagtggagtatgtggattaattngaagcaacgcgaaganccttaccaggtcttgacatccaactaacgaagcagagatgcattaggtgcccttcggggaaagttgagacaggtggtgcatggttgtcgtcagctcgtgtcgtgagatgttgggttaagtcccgcaacgagcgcaacccttactgttagttgctacgcaagagcactctagcaggactgccgttgacaaaacggaggaaggtggggacgacgtcaaatcatcatgccccttatgacctgggcctcacacgtactacaatggccattaacagagggaagcaagcccgcgaggtggagcaaaaccctaaaaatggtctcagttcggatcgtaggctgaaacccgcctgcgtgaagttggaattgctagtaatcgcggatcagcatgccgcggtgaatacgttcccgggccttgtacacaccgcccgtcacaccatgggagccggtaatacccgaagtcagtagtctaaccgcaa

SEQ ID NO: 4 - DNA, 1285 bp, synthetic construct
  misc_feature 1..1285: note = Synthetic: FBI00009
  source 1..1285: mol_type = other DNA
tcgacgggatcccaggagcttgctcctgggtgagagtggcgaacgggtgagtaatgcgtgaccgacctgccccatacaccggaatagctcctggaaacgggtggtaatgccggatgctccagttgaccgcatggtcttctgggaaagcttttgcggtatgggatggggtcgcgtcctatcagcttgatggcggggtaacggcccaccatggcttcgacgggtagccggcctgagagggcgaccggccacattgggactgagatacggcccanactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataancaccggctaactacgtgccagcagccgcggtaatacgtanggtgcaagcgttatccggaattattgggcgtaaagggctcgtangcggttcgtcgcgtccggtgtgaaagtccatcgcttaacggtggatccgcgccnggtacgggcgggcttgagtgcggtaggggagactggaattccccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaatgacgggggcccgcacaagcggcggagcatgcggattaattcgatgcaacgcgaagaaccttacctgggcttgacatgttcccgacagccgtagagatacggtctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaagggggggaagacgtcagatcatcatgccccttacgtccagggcttcacgcatgctacaatggccggtacaacgggatgcgacaccgcgaggtggagcgaatcccttaaaaccggtctcagttcggattggagtctgcaaccggactccatgaaggcggagtcgntagtaatggcggatcagcaa

SEQ ID NO: 5 - DNA, 1390 bp, synthetic construct
  misc_feature 1..1390: note = Synthetic: FBI00010
  source 1..1390: mol_type = other DNA
tgcagtcgaacgggaacctttnattgaagcttcggcagatttagctggtttctagtggcggacgggtgagtaacgcgtgggtaacctgccttatacagggggataacaaccagaaatggttgctaataccgcataagcgcacaggaccgcatggtccggtgtgaaaaactccggtggtataagatggacccgcgttggattagctagttggcagggtaacggcctaccaaggcgacgatccatagccggcctgagagggtgaacggccacattgggactgaaacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggactggcaagtctgatgtgaaaggcgggggctcaacccctggactgcattggaaactgttagtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgttggggagcaaagctcttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatccctctgaccgnncntaaccgagactttccttcgggacaggggagacaggtggtgcatggttgtcgtcagctcgtgtcgtgagatgttgggttaagtcccgcaacgagcgcaacccctatccccagtagccagcggtccggccgggcactctgaggagactgccagggataacctggaggaaggcggggatgacgtcaaatcatcatgccccttatgatttgggctacacacgtgctacaatggcgtaaacaaagggaagcaagcctgcgaaggtaagcaaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaactgcaaagaagga

SEQ ID NO: 6 - DNA, 1383 bp, synthetic construct
  misc_feature 1..1383: note = Synthetic: FBI00011
  source 1..1383: mol_type = other DNA
tgcagtcgnncgggatccatcaggctttgcttggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaacccctttgtgg

SEQ ID NO: 7 - DNA, 1400 bp, synthetic construct
  misc_feature 1..1400: note = Synthetic: FBI00012
  source 1..1400: mol_type = other DNA
anncntgcagtcgaggggcatcgggattgaagcttgcttcaattgccggcgaccggcgcacgggtgcgtaacgcgtatgtaacctacctataacaggggcataacactgagaaattggtactaattccccataatattcggagaggcatctctccgggttgaaaactccggtggttatagatggacatgcgttgtattagctagttggtgaggtaacggctcaccaaggcaacgatacatagggggactgagaggttaaccccccacactggtactgagacacggaccagactcctacgggaggcagcagtgaggaatattggtcaatggacgcaagtctgaaccagccatgccgcgtgcaggaagacggctctatgagttgtaaactgcttttgtacgagggtaaactcacctacgtgtaggtgactgaaagtatcgtacgaataaggatcggctaactccgtgccagcagccgcggtaatacggaggattcaagcgttatccggatttattgggtttaaagggtgcgtaggcggtttgataagttagaggtgaaatcccggggcttaactccggaactgcctctaatactgttagactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttactgacgattctggaaacaggatttcccttcggggcaggaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggtcgctactccgtgaggagatgccaatctcgaaagcctatctcagttcggattggaggctgaaacccgcctccatgaagttggattcgctagtaatcgcgcatcagccatggcgcggtgaatacgttcccgggccttgtacacaccgcccgtcaagccatggaagctgggggtgcctgaagttcgtgaccgcaaggagcgnc

SEQ ID NO: 8 - DNA, 1394 bp, synthetic construct
  misc_feature 1..1394: note = Synthetic: FBI00013
  source 1..1394: mol_type = other DNA
cngtcgaggggcagcatgatttgtancnntacanantgatggcgaccggcgcacgggtgagtaacgcgtatgcaacttacctatcagagggggatagcccggcgaaagtcggattaataccccataaaacaggggtcccgcatgggaatatttgttaaagattcatcgctgatagataggcatgcgttccattaggcagttggcggggtaacggcccaccaaaccgacgatggataggggttctgagaggaaggtcccccacattggtactgagacacggaccaaactcaaacgggaggcagcagtgaggaatattggtcaatggccgagaggctgaaccagccaagtcgcgtgaaggaagaaggatctatggtttgtaaacttcttttataggggaataaagtggaggacgtgtccttttttgtatgtaccctatgaataagcatcggctaactccgtgccagcagccgcggtaatacggaggatgcgagcgttatccggatttattgggtttaaagggtgcgtaggtggtgatttaagtcagcggtgaaagtttgtggctcaaccataaaattgccgttgaaactgggttacttgagtgtgtttgaggtaggcggaatgcgtggtgtagcggtgaaatgcatagatatcacgcagaactccgattgcgaaggcagcttactaaaccataactgacactgaagcacgaaagcgtggggatcaaacaggattagataccctggtagtccacgcagtaaacgatgattactaggagtttgcgatacaatgtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtagtctgaccggagtggaaacactccttctagcaatagcagattacgaggtgctgcatggttgtcgtcagctcgtgccgtgaggtgtcggcttaagtgccataacgagcgcaacccttatcactagttactaacaggtgaagctgaggactctggtgagactgccagcgtaagctgtgaggaaggtggggatgacgtcaaatcagcacggcccttacatccggggcgacacacgtgttacaatggcatggacaaagggcagctacctggcgacaggatgctaatctccaaaccatgtctcagttcggatcggagtctgcaactcgactccgtgaagctggattcgctagtaatcgcgcatcagccatggcgcggtgaatacgttcccgggccttgtacacaccgcccgtcaagccatgggagccgggggtacctgaagtccgtaaccgcaaggatcg

SEQ ID NO: 9 - DNA, 1357 bp, synthetic construct
  misc_feature 1..1357: note = Synthetic: FBI00015
  source 1..1357: mol_type = other DNA
gctannnnnnatggcgaccggcgcaggggtgagtaacacgtatgccaacctgccgatgactcggggatagcctttcgaaagaaagattaatacccgatggcataattcttccgcatggtagaactattaaagaatttcggtcatcgatggggatgcgttccattaggttgttggcggggtaacggcccaccaagccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgaggcacgtgtgcctttttgtatgtaccgtatgaataaggatcggctaactccgtgccagcagccgcggtaatacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcttgctggactgtaactgacgctgatgctcgaaagtgtgggtatcaaacaggattagataccctggtagtccacacagtaaacgatgaatactcgctgtttgcgatatacagtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttgaattgcaactgaatgatgtggagacatgtcagccgcaaggcagttgtgaaggtgctgcatggttgtcgtcagctcgtgccgtgaggtgtcggcttaagtgccataacgagcgcaacccttatcgatagttaccatcaggttatgctggggactctgtcgagactgccgtcgtaagatgtgaggaaggtggggatgacgtcaaatcagcacggcccttacgtccggggctacacacgtgttacaatggggggtacagaaggcagctacacggcgacgtgatgctaatccctaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtgcgtaaccgcga

SEQ ID NO: 10 - DNA, 1390 bp, synthetic construct
  misc_feature 1..1390: note = Synthetic: FBI00016
  source 1..1390: mol_type = other DNA
tgcagtcgnacgggatccatcaggctttgcttggtggtgagagtggcgaacgggtgagtaatgcgtgaccgacctgccccatacaccggaatagctcctggaaacgggtggtaatgccggatgctccgactcctcgcatggggtgtcgggaaagatttcatcggtatgggatggggtcgcgtcctatcaggtagtcggcggggtaacggcccaccgagcctacgacgggtagccggcctgagagggcgaccggccacattgggactgaaatacggcccaaactcctacgggaggcagcagtggggaatattgcacaatgggcgcaagcctgatgcagcgacgccgcgtgcgggatgacggccttcgggttgtaaaccgcttttgatcgggagcaagccttcgggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatctgcgccgggtacgggcgggctggagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatatggcctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgtccagggcttcacgcatgctacaatggccggtacaacgggatgcgacacggcgacgtggagcggatccctgaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcctaaccctttgtggangga

SEQ ID NO: 11 - DNA, 1400 bp, synthetic construct
  misc_feature 1..1400: note = Synthetic: FBI00018
  source 1..1400: mol_type = other DNA
annnntgcagtcgaacgaagcactttatttgatttccttcgggactgattattttgtgactgagtggcggacgggtgagtaacgcgtgggtaacctgccttgtacagggggataacagttggaaacggctgctaataccgcataagcgcacagcatcgcatgatgcagtgtgaaaaactccggtggtataagatggacccgcgttggattagctagttggtgaggtaacggcccaccaaggcgacgatccatagccgacctgagagggtgaccggccacattgggactgagacacggcccaaactcctacgggaggcagcagtggggaatattgcacaatgggcgaaagcctgatgcagcgacgccgcgtgagcgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgttgggaagcattgcttctcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggtacttaaccgtatcttctcttcggagcaggagtgacaggtggtgcatggttgtcgtcagctcgtgtcgtgagatgttgggttaagtcccgcaacgagcgcaacccttatctttagtagccagcggttcggccgggcactctagagagactgccagggataacctggaggaaggcggggatgacgtcaaatcatcatgccccttatgacttgggctacacacgtgctacaatggcgtaaacaaagggaagcaaagctgtgaagccgagcaaatctcaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttgggaatgcccgaagccagtgncctaaccgaaaggaagga

SEQ ID NO: 12 - DNA, 1393 bp, synthetic construct
  misc_feature 1..1393: note = Synthetic: FBI00019
  source 1..1393: mol_type = other DNA
tgcagtcgaggggcagcgggnntgaagctntgcttcaatcgccggcgaccggcgcangggtgcgtaacgcgtatgcaacctacccagaacagggggataacactgagaaattggtactaatatcccataacatcataaggggcatcccttttggttgaaaactccggtggttctggatgggcatgcgttgtattagctagttggtgaggtaacggctcaccaaggcaacgatacatagggggactgagaggttaaccccccacattggtactgagacacggaccaaactccnacgggaggcagcagtgaggaatattggtcaatggacgcaagtctgaaccagccatgccgcgtgcaggaagacggctctatgagttgtaaactgcttttgtactagggtaaactcagatacgtgtatctgactgaaagtatagtacgaataaggatcggctaactccgtgccagcagccgcggtaatacggaggattcaagcgttatccggatttattgggtttaaagggtgcgtaggcggtttgataagttagaggtgaaataccggtgcttaacaccggaactgcctctaatactgttgagctagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgctgtcggcgatacacagtcggtggctaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagtgacggatctggaaacaggtcttcccttcggggcacgaaactaggtgctgcatggttgtcgtcagctcgtgccgtgaggtgtcgggttaagtcccataacgagcgcaacccctaccgttagttgccatcaggtcaagctgggcactctgacgggactgccggtgtaagccgagaggaaggtggggatgacgtcaaatcagcacggcccttacgtccggggccacacacgtgttacaatggtaggtacagagggcagctacccagcgatgggatgcgaatctcgaaagcctatctcagttcggatcggaggctgaaacccgcctccgtgaagttggattcgctagtaatcgcgcatcagccatggcgcggtgaatacgttcccgggccttgtacacaccgcccgtcaagccatggaagctgggggtgcctgaagttcgtgaccgcaaggagc

SEQ ID NO: 13 - DNA, 1390 bp, synthetic construct
  misc_feature 1..1390: note = Synthetic: FBI00020
  source 1..1390: mol_type = other DNA
gtcgaggggcagcntttnagttntgcnttgcaaactggagatggcgaccggcgcacgggtgagtaacacgtatccaacctgccgataactcggggatagcctttcgaaagaaagattaatacccgatggtataatcagaccgcatggtcttgttattaaagaatttcggttatcgatggggatgcgttccattaggcagttggtgaggtaacggctcaccaaaccttcgatggataggggttctgagaggaaggtcccccacattggaactgagacacggtccaaactcctacgggaggcagcagtgaggaatattggtcaatgggcgcaggcctgaaccagccaagtagcgtgaaggatgactgccctatgggttgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggacagttaagtcagttgtgaaagtttgcggctcaaccgtaaaattgcagttgatactggctgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaatatattggaaacagtatagtcgtaagacaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctaatcccaaaagcctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggag

SEQ ID NO: 14 - DNA, 1395 bp, synthetic construct
  misc_feature 1..1395: note = Synthetic: FBI00021
  source 1..1395: mol_type = other DNA
cagtcgaggggcagcatttntangnttgcgttgcaaacgngaagatggcgacgggcggncgggtgagtaacacgtatccaacctgccgataactccggaatagcctttcgaaagaaagattaataccggatagcatacgaatatcgcatgatatttttattaaagaatttcggttatcgatggggatgcgttccattagtttgttggcggggtaacggcccaccaagactacgatggataggggttctgagaggaaggtccnccacattggaactgagacacggtccaaactcctacgggaggcagcagtgaggaatattggtcaatgggcgagagcctgaaccagccaagtagcgtgaaggatgaaggctctatgggtcgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgttactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaacagaatatattggaaacagtatagccgtaaggctgttgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcggctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaagnagc

SEQ ID NO: 15 - DNA, 1378 bp, synthetic construct
  misc_feature 1..1378: note = Synthetic: FBI00022
  source 1..1378: mol_type = other DNA
tgcaagtcgaggggcagcataatggatagcaatatctatggtggcgaccggcgcacgggtgcgtaacgcgtatgcaacctacctttaacagggggataacactgagaaattggtactaataccccataatatcatagaaggcatcttttatggttgaaaattccgatggttagagatgggcatgcgttgtattagctagttggtggggtaacggctcaccaaggcgacgatacatagggggactgagaggttaaccccccacactggtactgagacacggaccagactcctacgggaggcagcagtgaggaatattggtcaatggacgcaagtctgaaccagccatgccgcgtgcaggatgacggctctatgagttgtaaactgcttttgtacgagggtaaacgcagatacgtgtatctgtctgaaagtatcgtacgaataaggatcggctaactccgtgccagcagccgcggtaatacggaggattcaagcgttatccggatttattgggtttaaagggtgcgtaggcggtttgataagttagaggtgaaatttcggggctcaaccctgaacgtgcctctaatactgttgagctagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagcgacgattcttgaaagaggatttcccttcggggcgcgaaactaggtgctgcatggttgtcgtcagctcgtgccgtgaggtgtcgggttaagtcccataacgagcgcaacccctaccgttagttgccatcaggtgaagctgggcactctggcgggactgccggtgtaagccgagaggaaggtggggatgacgtcaaatcagcacggcccttacgtccggggctacacacgtgttacaatggtaggtacagagggcagctacccagcgatgggatgcgaatctcgaaagcctatctcagttcggattggaggctgaaacccgcctccatgaagttggattcgctagtaatcgcgcatcagccatggcgcggtgaatacgttcccgggccttgtacacaccgcccgtcaagccatgggagccgggggtgccngaagttcg

SEQ ID NO: 16 - DNA, 1393 bp, synthetic construct
  misc_feature 1..1393: note = Synthetic: FBI00025
  source 1..1393: mol_type = other DNA
gcagtcgnncgaagcacttnnctttgattcttcggatgaagaggtttgtgactgagtggcggacgggtgagtaacgcgtgggtaacctgcctcatacagggggataacagttagaaatgactgctaataccgcataagaccacagagccgcatggctcagagggaaaaactccggtggtatgagatggacccgcgtctgattaggtagttggtggggtaacggcctaccaagccaacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggctgtgtaagtctgaagtgaaagcccggggctcaaccccgggactgctttggaaactatgcagctagagtgtcggagaggtaagtggaattcccagtgtagcggtgaaatgcgtagatattgggaggaacaccagtggcgaaggcggcttactggacgatgactgacgttgaggctcgaaagcgtggggagcaaacaggattagataccctggtagtccacgccgtaaacgatgactactaggtgtcggggagcaaagctcttcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccggtgaccggcgtgtaatgacgccttttcttcggaacaccggtgacaggtggtgcatggttgtcgtcagctcgtgtcgtgagatgttgggttaagtcccgcaacgagcgcaacccttatcttcagtagccagcaattcggatgggcactctggagagactgccagggataacctggaggaaggtggggatgacgtcaaatcatcatgccccttatgagcagggctacacacgtgctacaatggcgtaaacaaagggaagcgagcctgcgagggtaagcaaatctcaaaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggtaacgcccgaagtcagtgacccaaccgtaagganggag

SEQ ID NO: 17 - DNA, 1527 bp, synthetic construct
  misc_feature 1..1527: note = Synthetic: FBI00027
  source 1..1527: mol_type = other DNA
tggagagtttgatcctggctcaggatgaacgctggcggcgtgcttaacacatgcaagtcgagcgaagcrgytargwwgattcttcggatgattytyrrytgactgagcggcggacgggtgagtaacgcgtgggtgacctgccccataccgggggataacagctggaaacggctgctaataccgcataagcgcacagagctgcatggctcrgtgtgaaaaactccggtggtatgggatgggcccgcgtctgattaggcagttggcggggtaacggcccaccaaaccgacgatcagtagccggcctgagagggcgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagataatgacggtacctgactaagaagccccggctaactacgtgccagcagccgcggtaatacgtagggggcaagcgttatccggatttactgggtgtaaagggagcgtagacggcaaggcaagtctgatgtgaaaacccagggcttaaccctgggactgcattggaaactgtctggctcgagtgccggagaggtaagcggaattcctagtgtagcggtgaaatgcgtagatattaggaagaacaccagtggcgaaggcggcttactggacggtaactgacgttgaggctcgaaagcgtggggagcaaacaggattagataccctggtagtccacgccgtaaacgatgaatgctaggtgttggggagcaaagctcttcggtgccgccgcaaacgcattaagcattccacctggggagtacgttcgcaagaatgaaactcaaaggaattgacggggacccgcacaagcggtggagcatgtggtttaattcgaagcaacgcgaagaaccttaccaggtcttgacatcccgatgaycggtycgtaayggrrccttctcttcggagcattggagacaggtggtgcatggttgtcgtcagctcgtgtcgtgagatgttgggttaagtcccgcaacgagcgcaacccttatcctcagtagccagcargtaawgytgggcactctgtggagactgccagggataacctggaggaaggtggggatgacgtcaaatcatcatgccccttatgatctgggctacacacgtgctacaatggcgtaaacaaagggaggcaaagccgcgaggtggagcaaatcccaaaaataacgtctcagttcggactgcagtctgcaactcgactgcacgaagctggaatcgctagtaatcgcgaatcagaatgtcgcggtgaatacgttcccgggtcttgtacacaccgcccgtcacaccatgggagttggtaacgcccgaagtcagtgacccaaccgtttacggagggagctgccgaaggcgggactgataactggggtgaagtcgtaacaaggtagccgtagcggaaggtgcggytggatcacctcctt

SEQ ID NO: 18 - DNA, 1520 bp, synthetic construct
  misc_feature 1..1520: note = Synthetic: FBI00029
  source 1..1520: mol_type = other DNA
tggagagtttgatcctggctcaggatgaacgctagcgacaggcttaacacatgcaagtcgaggggcagcrsrggtagcrataccsgsyggcgaccggcgcacgggtgagtaacgcgtatgcaacttgcctatcagagggggataacccggcgaaagtcggactaataccgcatgaagcagggrycccgcatgggratatttgctaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgagggatgaaggttctatggatcgtaaacctcttttataagggaataaagtgcgggacgtgtcccgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcatagatatcacgcagaaccccgattgcgaaggcagcctgccaagccatgactgacgctgatgcacgaaagcgtggggatcaaacaggattagataccctggtagtccacgcagtaaacgatgatcactagctgtttgcgatacactgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcattcggaccgaggtggaaacaccttttctagcaatagccgtttgcgaggtgctgcatggttgtcgtcagctcgtgccgtgaggtgtcggcttaagtgccataacgagcgcaacccttgccactagttactaacaggtrawgctgaggactctggtgggactgccagcgtaagctgcgaggaaggcggggatgacgtcaaatcagcacggcccttacatccggggcgacacacgtgttacaatggcgtggacaaagggaggccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccgggggtacctgaagtccgtaaccgcgaggatcggcctagggtaaaactggtgactggggctaagtcgtaacaaggtagccgtaccggaaggtgcggytggatcacctcctt

SEQ ID NO: 19 - DNA, 1376 bp, synthetic construct
  misc_feature 1..1376: note = Synthetic: FBI00030
  source 1..1376: mol_type = other DNA
tgcagtcgancgatgaaccgccctcgggcggacatgaagtggcgaacgggtgagtaacacgtgaccaacctgcccccctctccgggacaaccttgggaaaccgaggctaataccggatactccctcccctnntcctgcaggggtcgggaaagcccaggcggagggggatggggtcgcggcccattaggtagtaggcggggtaacggcccacctagcccgcgatgggtagccgggttgagagaccgaccggccacattgggactgagatacggcccagactcctacgggaggcagcagtggggaattttgcgcaatgggggcaaccctgacgcagcaacgccgcgtgcgggacgacggcctttcgggttgtaaaccgctttcagcagggaagaaattcgacggtacctgcagaagaagctccggctaactacgtgccagcagccgcggtaatacgtagggagcgagcgttatccggattcattgggcgtaaagagcgcgtaggcggcctctcaagcgggatctctaatccgagggctcaacccccggccggatcccgaactgggaggctcgagttcggtagaggcaggcggaattcccggtgtagcggtggaatgcgcagatatcgggaagaacaccgatggcgaaggcagcctgctgggccgcaactgacgctgaggcgcgaaagctaggggagcgaacaggattagataccctggtagtcctagccgtaaacgatggatactaggtgtggggggctccgccctccgtgccgcagccaacgcattaagtatcccgcctggggagtacggccgcaaggctaaaactcaaaggaattgacgggggcccgcacaagcagcggagcatgtggcttaattcgaagcaacgcgaagaaccttaccagggcttgacatggacgtgaagccggggaaacccggtggccgagaggagcgtccgcaggtggtgcatggctgtcgtcagctcgtgtcgtgagatgttgggttaagtcccgcaacgagcgcaacccctgccccatgttgccagcattaggttggggactcatgggggactgccggcgtcaagccggaggaaggtggggacgacgtcaagtcatcatgccctttatgccctgggctgcacacgtgctacaatggccggtacaacgggctgcgagaccgcgaggtcgagcgaatccctcaaagccggccccagttcggatcggaggctgcaacccgcctccgtgaagtcggagttgctagtaatcgcggatcagcatgccgcggtgaatacgttcccgggccttgtacacaccgcccgtcacaccacccgagtcgtctgcacccgaagccgccggccgaacccgcaaggggcggaggcnt

SEQ ID NO: 20 - DNA, 1391 bp, synthetic construct
  misc_feature 1..1391: note = Synthetic: FBI00032
  source 1..1391: mol_type = other DNA
tcgaacgaaacaccttatttgattttcttcggaactgaagatttggtgattgagtggcggacgggtgagtaacgcgtgggtaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtagaggcttcggtgccgcagccaacgcagtaagtattccacctggggagtacgttcgcaagaatgaaactcaaaggaattgacggggacccgcacaagcggtggagcatgtggtttaattcgaagcaacgcgaagaaccttacctggtcttgacatccttctgaccggtccttaaccggacctttccttcgggacaggagtgacaggtggtgcatggttgtcgtcagctcgtgtcgtgagatgttgggttaagtcccgcaacgagcgcaacccctatctttagtagccagcatataaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcannnacccaaccgtaaggagggag

SEQ ID NO: 21 - DNA, 1526 bp, synthetic construct
  misc_feature 1..1526: note = Synthetic: FBI00033
  source 1..1526: mol_type = other DNA
tggagagtttgatcctggctcaggatgaacgctggcggcgtgcctaacacatgcaagtcgaacgaggyawwtdrrwtgaagttttcggayrgattyywakwtaccgagtggcggacgggtgagtaacgcgtgggtaacctgcctcatacagggggataacggttagaaatgactgctaataccgcataagcgcacagtaccgcatggtacggtgtgaaaaactccggtggtatgagatggacccgcgtctgattagctagttggtgrggtaacggcccaccaaggcgacgatcagtagccgacctgagagggtgaccggccacattgggactgagacacggcccagactcctacgggaggcagcagtgggggatattgcacaatggaggaaactctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgacgcaagtctgaagtgaaataccygggctcaacctgggaactgctttggaaactgtgttgctagagtgctggagaggtaagcggaattcctagtgtagcggtgaaatgcgtagatattaggaagaacaccagtggcgaaggcggcttactggacagtaactgacgttgaggctcgaaagcgtggggagcaaacaggattagataccctggtagtccacgccgtaaacgatgaatactaggtgttggtgagcaaagctcatcggtgccgccgcaaacgcaataagtattccacctggggagtacgttcgcaagaatgaaactcaaaggaattgacggggacccgcacaagcggtggagcatgtggtttaattcgaagcaacgcgaagaaccttaccaaatcttgacatccctctgaaamryyyttaatcgrgykcctccttcgggacagaggwgacaggtggtgcatggttgtcgtcagctcgtgtcgtgagatgttgggttaagtcccgcaacgagcgcaacccctattgtcagtagccagcaggtmaagctgggcactctgatragactgccagggataacctggaggaaggtggggatgacgtcaaatcatcatgccccttatgatttgggctacacacgtgctacaatggcgtaaacaaagagaagcgagcctgygagggggagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcggaaatgcccgaagccagtgacccaagcgaaagcagggagctgtcgaaggcaggtctgataactggggtgaagtcgtaacaaggtagccgtatcggaaggtgcggytggatcacctcctt

SEQ ID NO: 22 - DNA, 1392 bp, synthetic construct
  misc_feature 1..1392: note = Synthetic: FBI00034
  source 1..1392: mol_type = other DNA
antcgaacgaagcatttacnacagatttcttcggattgaagtnntttatgactgagtggcggacgggtgagtaacgcgtgggtaacctgccttgtactgggggatagcagctggaaacggctggtaataccgcataagcgcacaatgttgcatgacatggtgtgaaaaactccggtggtataagatggacccgcgtctgattagctagttggtgagataacagcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggctcaaccccggaactgcttttgaaactgtaaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccctggtagtccacgccgtaaacgatgaatactaggtgtcggggctcataagngcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgaccggacagtaatgtgtcctttccttcgggacagtggtgacaggtggtgcatggttgtcgtcagctcgtgtcgtgagatgttgggttaagtcccgcaacgagcgcaacccctatccttagtagccagcagtaagatgggcactctagggagactgccagggataacctggaggaaggtggggatgacgtcaaatcatcatgccccttatgacttgggctacacacgtgctacaatggcgtaaacaaagtgaagcgaagtcgtgaggccaagcaaatcacaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgaaaatgcccgaagtcggtgacctaacgtaagaaggagc

SEQ ID NO: 23 - DNA, 1382 bp, synthetic construct
  misc_feature 1..1382: note = Synthetic: FBI00038
  source 1..1382: mol_type = other DNA
tgcagtcgacgagagaagattggtgcttgcactgatccgatcgagtggcggacgggtgagtaacgcgtgggtaacctgcctcatacaggggaataacagttggaaacggctgctaaaaccgcataagcgcacggaatcgcatgatacagtgtgaaaaactccggtggtatgagatggacccgcgtctgattagctagttggtgaggtaacggcccaccaaggcaacgatcagtagccggcctgagagggtgaacggccacattgggactgagacacggcccaaactcctacgggaggcagcagtggggaatattgcacaatgggggaaaccctgatgcagcgacgccgcgtgagtgatgaagtatttcggtatgtaaagctctatcagcagggaagataatgacggtacctgactaagaagcaccggctaaatacgtgccagcagccgcggtaatacgtatggtgcaagcgttatccggatttactgggtgtaaagggtgcgtaggtggtgagacaagtctgaagtgaaaatccggggcttaaccccggaactgctttggaaactgcctgactagagtacaggagaggtaagtggaattcctagtgtagcggtgaaatgcgtagatattaggaggaacaccagtggcgaaggcgacttactggactgctactgacactgaggcacgaaagcgtggggagcaaacaggattagataccctggtagtccacgccgtaaacgatgaatactaggtgtcggggcccaaagggcttcggtgccgcagccaacgcaataagtattccacctggggagtacgttcgcaagaatgaaactcaaaggaattgacggggacccgcacaagcggtggagcatgtggtttaattcgaagcaacgcgaagaaccttaccaagtcttgacatcctgctgaccgttccttaaccggaattttccttcgggacagcagagacaggtggtgcatggttgtcgtcagctcgtgtcgtgagatgttgggttaagtcccgcaacgagcgcaacccctatttccagtagccagcagtcagatgggcactctggagagactgccggggataacccggaggaaggtggggatgacgtcaaatcatcatgccccttatgatttgggctacacacgtgctacaatggcagttacaaagagaagcgaaactgtgaagtggagcaaacctcaaaaaggctgtctcagttcggattgtagtctgcaactcgacnacatgaagctggaatcgctagtaatcgcagatcagaatgctgcggtgaatacgttcccgggtcttgtacacaccgcccgtcacaccatgggagtcggaaacgcccgaagtcagtgacccaaccgcaaggagggagc

SEQ ID NO: 24 - DNA, 1400 bp, synthetic construct
  misc_feature 1..1400: note = Synthetic: FBI00040
  source 1..1400: mol_type = other DNA
gtcgaacgtgaaagtccttcgggacnagtaaagtggcgcacgggtgagtaacgcgtggataatctacccttaagatggggataacggctggaaacggtcgctaataccgaatacgctcccgattttatcattggggggaaagatggcctctgcttgcaagctatcgcttaaggatgagtccgcgtcccattagctagttggcggggtaacggcccaccaaggcaacgatgggtagccggtctgagaggatgaccggccacactggaactggaacacggtccaaactcctacgggaggcagcagtggggaatattgcgcaatgggcgaaagcctgacgcagcgacgccgcgtgagggatgaaggttctcggatcgtaaacctctgtcaggggggaaaaaaccccctcgtgtgaataatgcgagggcttgacggtacccccaaaggaagcaccggctaactccgtgccagcagccgcggtaatacggagggtgcaagcgttaatcggaatcactgggcgtaaagcgcacgtaggcggcttggtaagtcaggggtgaaatcccacagcccaactgtggaactgcctttgatactgccaggcttgagtaccggagagggtggcggaattccaggtgtaggagtgaaatccgtagatatctggaggaacaccggtggcgaaggcggccacctggacggtaactgacgctgaggtgcgaaagcgtgggtagcaaacaggattagataccctggtagtccacgctgtaaacgatgggtgctgggtgctgggatgtatgtctcggtgccgtagctaacgcgataagcaccccgcctggggagtacggtcgcaaggctgaaactcaaagaaattgacgggggcccgcacaagcggtggagtatgtggtttaattcgatgcaacgcgaagaaccttacccaggcttgacatctagggaacccttcggaaatgaaggggtgcccttcggggagccctaagacaggtgctgcatggctgtcgtcagctcgtgccgtgaggtgttgggttaagtcccgcaacgagcgcaacccctatcttcagttgccagcaggtaaggctgggcactctggagagaccgccccggtcaacggggaggaaggtggggacgacgtcaagtcatcatggcccttacgcctggggctacacacgtactacaatggcgcgcacaaagggtagcgagaccgcgaggtggagccaatcccaaaaaacgcgtcccagtccggattggagtctgcaactcgactccatgaagtcggaatcgctagtaattcgagatcagcatgctcgggtgaatgcgttcccgggccttgtacacaccgcccgtcacaccacgaaagtcggttttacccgaagccggtgagctaactntgc

SEQ ID NO: 25 - DNA, 1525 bp, synthetic construct
  misc_feature 1..1525: note = Synthetic: FBI00043
  source 1..1525: mol_type = other DNA
tggagagtttgatcctggctcaggatgaacgctggcggcgtgcttaacacatgcaagtcgaacgggatcccgggggttcgctcccgggtgagagtggcgaacgggtgagtaatgcgtgaccgacctgccccatacaccggaatagctcctggaaacgggtggtaatgccggatgctccggttggatgcatgtccttccgggaaagattccatcggtatgggatggggtcgcgtcctatcagcttgatggcggggtaacggcccaccatggcctcgacgggtagccggcctgagagggcgaccggccacattgggactgagatacggcccagactcctacgggaggcagcagtggggaatattgcacaatgggcgcaagcctgatgcagcgacgccgcgtgcgggatggaggccttcgggttgtaaaccgcttttgatcgggagcaagcccttcggggtgagtgtacccttcgaataagcaccggctaactacgtgccagcagccgcggtaatacgtagggtgcaagcgttatccggaattattgggcgtaaagggctcgtaggcggttcgtcgcgtccggtgtgaaagcccatcgcttaacggtgggtctgcgccgggtacgggcgggctggagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatatggcctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacagcgggatgcgacatggcgacatggagcggatccctgaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggytggatcacctcctt

SEQ ID NO: 26 - DNA, 1525 bp, synthetic construct
  misc_feature 1..1525: note = Synthetic: FBI00044
  source 1..1525: mol_type = other DNA
tggagagtttgatcctggctcaggatgaacgctggcggcgtgcttaacacatgcaagtcgaacgggaawtayttyattgaracttcggtsgatttratytawttctagtggcggacgggtgagtaacgcgtgggtaacctgccttatacagggggataacagtcagaaatggctgctaataccgcataagcgcacagagctgcatggctcagtgtgaaaaactccggtggtataagatggacccgcgttggattagcttgttggtggggtaacggccyaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggkrgcakrgcymttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaartcttgacatccbyctgaccgrwmyttaaycgkwycttyycttcggrrcagrvgwgacaggtggtgcatggttgtcgtcagctcgtgtcgtgagatgttgggttaagtcccgcaacgagcgcaacccctatcctcagtagccagcatttaaggtgggcactctggggagactgccagggataacctggaggaaggcggggatgacgtcaaatcatcatgccccttatgatttgggctacacacgtgctacaatggcgtaaacaaagggaagcgagatcgtgagatggagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacctaactgcaaagaaggagctgccgaaggcgggaccgatgactggggtgaagtcgtaacaaggtagccgtatcggaaggtgcggytggatcacctcctt

SEQ ID NO: 27 - DNA, 1393 bp, synthetic construct
  misc_feature 1..1393: note = Synthetic: FBI00046
  source 1..1393: mol_type = other DNA
tgcagtcgaggggcatcagtttggtttgcttgcaaaccaaagctggcgaccggcgcacgggtgagtaacacgtatccaacctgcctcatactcggggatagcctttcgaaagaaagattaatatccgatagcatatatttcccgcatgggttttatattaaagaaattcggtatgagatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgcgagtctgaaccagccaagtagcgtgaaggatgactgccctatgggttgtaaacttcttttatatgggaataaagtggtccacgtgtggatttttgtatgtaccatatgaataaggatcggctaactccgtgccagcagccgcggtaatacggaggatccgagcgttatccggatttattgggtttaaagggagcgtaggcggattgttaagtcagttgtgaaagtttgcggctcaaccgtaaaattgcagttgatactggcagtcttgagtgcagtagaggtgggcggaattcgtggtgtagcggtgaaatgcttagatatcacgaagaactccgattgcgaaggcagctcactggagtgtaactgacgctgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaatgaattatggggaaacccatacgccgcaaggcatttgtgaaggtgctgcatggttgtcgtcagctcgtgccgtgaggtgtcggcttaagtgccataacgagcgcaacccttatcttcagttactaacaggtcatgctgaggactctggagagactgccgtcgtaagatgtgaggaaggtggggatgacgtcaaatcagcacggcccttacgtccggggctacacacgtgttacaatggggggtacagaaggccgctacctggtgacaggatgccaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagc

SEQ ID NO: 28 - DNA, 1410 bp, synthetic construct
  misc_feature 1..1410: note = Synthetic: FBI00047
  source 1..1410: mol_type = other DNA
anncatgcagtcgaacgaagcatttangaacangattacttcggtgtntgaagttnnttatgactgagtggcggacgggtgagtaacgcgtgggtaacctgccttgtactgggggatagcagctggaaacggctggtaataccgcataagcgcacaatgttgcatgacatggtgtgaaaaactccggtggtataagatggacccgcgtctgattagctagttggtgagataacagcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggctcaaccccggaactgcttttgaaactgtaaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccctggtagtccacgccgtaaacgatgaatactaggtgtcggggcccataagggcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgaccggacagtaatgtgtcctttccttcgggacagtggtgacaggtggtgcatggttgtcgtcagctcgtgtcgtgagatgttgggttaagtcccgcaacgagcgcaacccctatccttagtagccagcagtaagatgggcactctagggagactgccagggataacctggaggaaggtggggatgacgtcaaatcatcatgccccttatgacttgggctacacacgtgctacaatggcgtaaacaaagtgaagcgaagtcgtgaggccaagcaaatcacaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgaaaatgcccgaagtcggtgacctaacgaaagaaggagccgccga

SEQ ID NO: 29 - DNA, 1528 bp, synthetic construct
  misc_feature 1..1528: note = Synthetic: FBI00048
  source 1..1528: mol_type = other DNA
tggagagtttgatcctggctcaggatgaacgctggcggcgtgcttaacacatgcaagtcgagcgaagcrgytargwtgatttcttcggattgattcttractgactgagcggcggacgggtgagtaacgcgtgggtgacctgccccataccgggggataacagctggaaacggctgctaataccgcataagcgcacagagctgcatggctcrgtgtgaaaaactccggtggtatgggatgggcccgcgtctgattaggcagttggcggggtaacggcccaccaaaccgacgatcagtagccggcctgagagggcgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagataatgacggtacctgactaagaagccccggctaactacgtgccagcagccgcggtaatacgtagggggcaagcgttatccggatttaytgggygtaaagggmkcgtagrcggcaaggcaagtctgatgtgaaaacccagggcttaaccctgggactgcattggaaactgtctggctygagtgccggagaggtaagcggaattcctagtgtagcggtgaaatgcgtagatattaggaagaacaccagtggcgaaggcggcttactggacggtaactgacgttgaggctcgaaagcgtggggagcaaacaggattagataccctggtagtccacgccgtaaacgatgaatgctaggtgttggggagcaaagctcttcggtgccgccgcaaacgcattaagcattccacctggggagtacgttcgcaagaatgaaactcaaaggaattgacggggacccgcacaagcggtggagcatgtggtttaattcgaagcaacgcgaagaaccttaccaggtcttgacatcccgatgaccggyycgtaayggrrccttctcttcggagcattggagacaggtggtgcatggttgtcgtcagctcgtgtcgtgagatgttgggttaagtcccgcaacgagcgcaacccttatcctcagtagccagcaggtmaagctgggcactctgtggagactgccagggataacctggaggaaggtggggatgacgtcaaatcatcatgccccttatgatctgggctacacacgtgctacaatggcgtaaacaaagggaggcaaagccgcgaggtggagcaaatcccaaaaataacgtctcagttcggactgcagtctgcaactcgactgcacgaagctggaatcgctagtaatcgcgaatcagaatgtcgcggtgaatacgttcccgggtcttgtacacaccgcccgtcacaccatgggagttggtaacgcccgaagtcagtgacccaacctttyaggagggagctgccgaaggcgggactgataactggggtgaagtcgtaacaaggtagccgtatcggaaggtgcggytggatcacctcctt

SEQ ID NO: 30 - DNA, 1406 bp, synthetic construct
  misc_feature 1..1406: note = Synthetic: FBI00049
  source 1..1406: mol_type = other DNA
cgggaggaaaagagaagcttgcttctttttgaatctagtggcaaacgggtgagtaacacgtaaacaacctgccttcaggatggggacaacagacggaaacgactgctaataccgaatgtgttccggagaccgcatgatttccggaagaaaggatggcctctatttataagctatcgcctgaagaggggtttgcgtctgattaggcagttggtgaggtaacggcccaccaaacctacgatcagtagccggtctgagaggatgaacggccacactggaactgagacacggtccagactcctacgggaggcagcagtggggaatcttccgcaatggacgaaagtctgacggagcaacgccgcgtgagtgatgacggccttcgggttgtaaagctctgtgatcggggacgaatggctggtatgctaataccatatcagagtgacggtacccgaatagcaagccacggctaactacgtgccagcagccgcggtaatacgtaggtggcaagcgttgtccggaattattgggcgtaaagcgcgcgcaggcggcttcttaagtcccatcttaaaagtgcggggcttaaccccgtgatgggatggaaactgggaggctggagtatcggagaggaaagtggaattcctagtgtagcggtgaaatgcgtagagattaggaagaacaccggtggcgaaggcgactttctggacgacaactgacgctgaggcgcgaaagcgtggggagcaaacaggattagataccctggtagtccacgccgtaaacgatgaatactaggtgtaggaggtatcgaccccttctgtgccggagctaacacaataagtattccgcctgggaagtacgatcgcaagattaaaactcaaaggaattgacgggggcccgcacaagcggtggagtatgtggtttaattcgacgcaacgcgaagaaccttaccaggtcttgacattgactgcaatcccgagaaatcgggagttcccttcggggacaggaaaacaggtggtgcacggctgtcgtcagctcgtgtcgtgagatgttgggttaagtcccgcaacgagcgcaacccctatcttatgttgccagcacgtaatggtgggaactcatgagagaccgccgcggacaacgcggaggaaggcggggatgacgtcaagtcatcatgccccttatgacctgggctacacacgtactacaatgggtgtcaacaaagagaagcaatggggcgacccggagcaaacctcaaaaacacacccccagttcagattgcaggctgcaacccgcctgcatgaagcaggaatcgctagtaatcgcgggtcagcataccgcggtgaatacgttcccgggccttgtacacaccgcccgtcacactatgagagtcagaaacacccgaagccggtgaggta

SEQ ID NO: 31 - DNA, 1519 bp, synthetic construct
  misc_feature 1..1519: note = Synthetic: FBI00050
  source 1..1519: mol_type = other DNA
tggagagtttgatcctggctcaggatgaacgctagctacaggcttaacacatgcaagtcgaggggcatcaggatgtagcaatacattgctggcgaccggcgcacgggtgagtaacacgtatccaacctgccttttactcggggataggctttcgaaagaaagattaatacccgatggcataatattyccgcatgggrttattattaaagaatttcggtaaaagatggggatgcgttccattaggcagttggcggggtaacggcccaccaaaccttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttatatgggaataaagtgagccacgtgtggctttttgtatgtaccttatgaataaggatcggctaactccgtgccagcagccgcggtaatacggaggatccgagcgttatccggatttattgggtttaaagggagcgtaggtggacatgtaagtcagttgtgaaagtttgcggctcaaccgtaaaattgcagttgatactgtgtgtcttgagtacagtagaggtgggcggaattcgtggtgtagcggtgaaatgcttagatatcacgaagaactccgattgcgaaggcagctcactggactggtaactgacactgaggctcgaaagtgtgggtatcaaacaggattagataccctggtagtccacacagtaaacgatgaatactcgctgtttgcgatatacagcaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaataatttggaaacagattagccgcaaggcaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacacggcgacgtgatgctaatcccaaaagcctctctcagttcggatcgaagtctgcaacccgacttcgtgaagctggattcgctagtaatcgcgcatcagccacggcgcggtgaatacgttcccgggccttgtacacaccgcccgtcaagccatgggagccgggggtacctgaagtacgtaaccgcaaggagcgtcctagggtaaaactggtgactggggctaagtcgtaacaaggtagccgtaccggaaggtgcggytggatcacctcctt

SEQ ID NO: 32 - DNA, 1526 bp, synthetic construct
  misc_feature 1..1526: note = Synthetic: FBI00051
  source 1..1526: mol_type = other DNA
tggagagtttgatcctggctcaggatgaacgctggcggcgtgcttaacacatgcaagtcgagcgaagcacwtaagtttgattcttcggatgaagacttttgtgactgagcggcggacgggtgagtaacgcgtgggtaacctgcctcatacagggggataacagttagaaatggctgctaataccgcataagaccacrgtactgcatggtacagtggtaaaaactccggtggtatgagatggacccgcgtctgattaggtagttggtgaggtaacggcycaccaagccgacgatcagtagccgacctgagagggtgaccggccacattgggactgagacacggcccagactcctacgggaggcagcagtggggaatattgcacaatgggcgaaagcctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggctgtgcaagtctgaagtgaaaggcatgggctcaacctgtggactgctttggaaactgtgcagctagagtgtcggagaggtaagtggaattcctagtgtagcggtgaaatgcgtagatattaggaggaacaccagtggcgaaggcggcttactggacgatgactgacgttgaggctcgaaagcgtggggagcaaacaggattagataccctggtagtccacgccgtaaacgatgactgctaggtgtcgggtagcaaagctattcggtgccgcagctaaygcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagyttttcttcggaacatcggtgacaggtggtgcatggttgtcgtcagctcgtgtcgtgagatgttgggttaagtcccgcaacgagcgcaacccttatcttcagtagccagcattwaggatgggcactctggagagactgccagggataacctggaggaaggtggggatgacgtcaaatcatcatgccccttatgaccagggctacacacgtgctacaatggcgtaaacaaagggaagcagagccgcgaggccgagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgaaaggagggagctgccgaaggtgggaccgataactggggtgaagtcgtaacaaggtagccgtatcggaaggtgcggytggatcacctcctt

SEQ ID NO: 33 - DNA, 1401 bp, synthetic construct
  misc_feature 1..1401: note = Synthetic: FBI00052
  source 1..1401: mol_type = other DNA
anncatgcagtcgaggggcagcattttagtttgcttgcaaactaaagatggcgaccggcgcacgggtgagtaacacgtatccaacctgccgataactcggggatagcctttcgaaagaaagattaatatccgatagtatattaaaaccgcatggttttactattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtattccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaatatagtggaaacattatagccgcaaggcaaatgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctacctggcgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcnnc

SEQ ID NO: 34 - DNA, 1397 bp, synthetic construct
  misc_feature 1..1397: note = Synthetic: FBI00053
  source 1..1397: mol_type = other DNA
tgcagtcgancgaagcatttaagacggattctttcgggatgaagacttttatgactgagtggcggacgggtgagtaacgcgtgggtaacctgcctcacacagggggatagcagttggaaacggctgataataccgcataagcgcacagtaccgcatggtacagtgtgaaaaactccggtggtgtgagatggacccgcgtctgattagcttgttggcagggtaacggcctaccaaggcaacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggcatcacaagtcagaagtgaaagcccggggctcaaccccgggactgcttttgaaactgtggagctggagtgcaggagaggcaagtggaattcctagtgtagcggtgaaatgcgtagatattaggaggaacaccagtggcgaaaggcggcttgctggactgtaactgacactgaggctcgaaagcgtggggagcaaacaggattagataccctggtagtccacgccgtaaacgatgaatactaggtgtcggggctcataagagcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcctcttgcccggtcagtaatgtgaccttttcttcggaacaagagtgacaggtggtgcatggttgtcgtcagctcgtgtcgtgagatgttgggttaagtcccgcaacgagcgcaacccctattcttagtagccagcatttaaggtgggcactctaggaagactgccagggataacctggaggaaggtggggatgacgtcaaatcatcatgccccttatgacttgggctacacacgtgctacaatggcgtaaacaaagtgaagcgagagtgtgagcttaagcaaatcacaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcggaaatgcccgaagtcggtgacctaacgaaagaaggagc

SEQ ID NO: 35 - DNA, 1387 bp, synthetic construct
  misc_feature 1..1387: note = Synthetic: FBI00056
  source 1..1387: mol_type = other DNA
cntgcagtcgnncgaagcaatcnnaatgaagttttcggatggatttctgattgactgagtggcggacgggtgagtaacgcgtggataacctgcctcacactgggggataacagttagaaatggctgctaataccgcataagcgcacagtaccgcatggtacggtgtgaaaaactccggtggtgtgagatggatccgcgtctgattagttagttggcggggtaacggcccaccaagacagcgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaactgacggtacctgactaagaagccccggctaactacgtgccagcagccgcggtaatacgtagggggcaagcgttatccggatttactgggtgtaaagggagcgtagacgcgaagcaagtctggagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcaataagcattccacctgggggagtacgttcgcaagaatgaaactcaaaggaattgacggggacccgcacaagcggtggagcatgtggtttaattcgaagcaacgcgaagaaccttaccaagtcttgacatccccctgaccggtcagtaaagtgacctttccttcgggacaggggagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcgaccctgcgaaggcaagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgncccaantcg

SEQ ID NO: 36 - DNA, 1399 bp, synthetic construct
  misc_feature 1..1399: note = Synthetic: FBI00057
  source 1..1399: mol_type = other DNA
tgcagtcgagcgaagcacttnngtttgattcttcggatgaagacttttgtgactgagcggcggacgggtgagtaacgcgtgggtaacctgcctcatacagggggataacagttagaaatgactgctaataccgcataagaccacggtaccgcatggtacagtggtaaaaactccggtggtatgagatggacccgcgtctgattaggtagttggtggggtaacggcctaccaagccgacgatcagtagccgacctgagagggtgaccggccacattgggactgagacacggcccagactcctacgggaggcagcagtggggaatattgcacaatggaggaaactctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcacggcaagccagatgtgaaagcccggggctcaaccccgggactgcatttggaactgctgagctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactgctaggtgtcgggtggcaaagccattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagctttttcttcggaacatcggtgacaggtggtgcatggttgtcgtcagctcgtgtcgtgagatgttgggttaagtcccgcaacgagcgcaacccctatcttcagtagccagcaggttaagctgggcactctggagagactgccagggataacctggaggaaggtggggatgacgtcaaatcatcatgccccttatgaccagggctacacacgtgctacaatggcgtaaacaaagagaagcgaactcgcgagggtaagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgtaaggnnnagctgc

SEQ ID NO: 37 - DNA, 1350 bp, synthetic construct
  misc_feature 1..1350: note = Synthetic: FBI00059
  source 1..1350: mol_type = other DNA
gntggcgaccggcgcacgggtgagtaacacgtatccaacctgccgattattccgggatagcctttctaaagaaagattaatactggatagtataacgagaaggcatcttcttgttattaaagaatttcgataattgatggggatgcgttccattagtttgttggcggggtaacggcccaccaagacatcgatggataggggttctgagaggaaggtcccccacattggaactgagacacggaccaaactcatacgggaggcagcagtgaggaatattggtcaatggacgagngnnnnaaccagccaagtagcgtgaaggatgactgccctatgggttgtaaacttcttttatatgggaataaagtgcagtatgtatactgttttgtatgtaccatatgaataaggatcggctaactccgtgccagcagccgcggtaatacggaggatccgagcgttatccggatttattgggtttaaagggagcgtaggcggaagcttaagtcagttgtgaaagtttgcggctcaaccgtaaaattgcagttgatactgggtttcttgagtgcagtagaggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgtttgcgatatacagcaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaactgacgtaggtggaaacatctattccttcgggcagttgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctacacggcgacgtgatgctaatccccaaaacctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtccgtaaccgtaagna

SEQ ID NO: 38 - DNA, 1384 bp, synthetic construct
  misc_feature 1..1384: note = Synthetic: FBI00060
  source 1..1384: mol_type = other DNA
tgcagtcgaacgggatccatcaggctttgcttggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtggga

SEQ ID NO: 39 - DNA, 1361 bp, synthetic construct
  misc_feature 1..1361: note = Synthetic: FBI00061
  source 1..1361: mol_type = other DNA
agcantgcngggtggcgaccggcgcacgggtgcgtaacgcgtatgcaacctacccataacagggggataacactgagaaattggtactaataccccataacatcagaaccggcatcggttttggttgagaactccggtggttatggatgggcatgcgttgtattagctggttggtgaggtaacggctcaccaaggcaacgatacatagggggactgagaggttaaccccccacattggtactgagacacganccaactnnctacgggaggcagcagtgaggaatattggtcaatggacgcaagtctgaaccagccatgccgcgtgcaggaagacggctctatgagttgtaaactgcttttgtacgagggtaaacgcttctacgtgtaggagcctgaaagtatcgtacgaataaggatcggctaactccgtgccagcagccgcggtaatacggaggatccaagcgttatccggatttattgggtttaaagggtgcgtaggcggtttgataagttagaggtgaaataccggtgcttaacaccggaactgcctctaatactgttgaactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgctgtcggcgatacacagtcggcggctaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttactgacgattctggaaacaggatttcccttcggggcaggaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggcagctacccagtgatgggatgcgaatctcgaaagcctatctcagttcggatcggaggctgaaacccgcctccgtgaagttggattcgctagtaatcgcgcatcagccatggcgcggtgaatacgttcccgggccttgtacacaccgcccgtcaagccatggaagctgggggtgcctgaagttcgtgaccgcaagna

SEQ ID NO: 40 - DNA, 1356 bp, synthetic construct
  misc_feature 1..1356: note = Synthetic: FBI00062
  source 1..1356: mol_type = other DNA
cggcacctatcttcggatggaagcgagtggcgaacggctgagtaacacgtggagaacctgccccctcccccgggatagccgcccgaaaggacgggtaataccggataccccggggtgccgcatggcaccccggctaaagccccgacgggaggggatggctccgcggcccatcaggtagacggcggggtgacggcccaccgtgccgacaacgggtagccgggttgagagaccgaccggccagattgggactgagacacggcccagactcctacgggaggcagcagtggggaatcttgcgcaatggggggaaccctgacgcagcgacgccgcgtgcgggacggaggccttcgggtcgtaaaccgctttcagcagggaagagtcaagactgtacctgcagaagaagccccggctaactacgtgccagcagccgcggtaatacgtagggggcgagcgttatccggattcattgggcgtaaagcgcgcgtaggcggcccggcaggccgggggtcgaagcggggggctcaaccccccgaagcccccggaacctccgcggcttgggtccggtaggggagggtggaacacccggtgtagcggtggaatgcgcagatatcgggtggaacaccggtggcgaaggcggccctctgggccgagaccgacgctgaggcgcgaaagctgggggagcgaacaggattagataccctggtagtcccagccgtaaacgatggacgctaggtgtggggggacgatccccccgtgccgcagccaacgcattaagcgtcccgcctggggagtacggccgcaaggctaaaactcaaaggaattgacgggggcccgcacaagcagcggagcatgtggcttaattcgaagcaacgcgaagaaccttaccagggcttgacatatgggtgaagcgggggagaccccgtggccgagaggagcccatacaggtggtggcatggctgtcgtcagctcgtgtcgtgagatgttgggttaagtcccgcaacgagcgcaacccccgccgcgtgttgccatcgggtgatgccgggaacccacgcgggaccgccgccgtcaaggcggaggagggcggggacgacgtcaagtcatcatgccccttatgccctgggctgcacacgtgctacaatggccggtacagagggatgccaccccgcgagggggagcggatcccggaaagccggccccagttcggattgggggctgcaacccgcccccatgaagtcggagttgctagtaatcgcggatcagcatgccgcggtgaatgcgttcccgggccttgtacacaccgcccgtcacaccacccgagtcgtctgcacccgaagtcgcncggcccaaccnnnaggg

SEQ ID NO: 41 - DNA, 1394 bp, synthetic construct
  misc_feature 1..1394: note = Synthetic: FBI00066
  source 1..1394: mol_type = other DNA
ctnncnntgcagtcgtacnggtaaccaagagaagcttgcttctctccggcgacgagtggcgaacgggtgagtaatacatcggaacgtgtccgctcgtgggggacaaccagccgaaaggttggctaataccgcatgagttctacggaagaaagagggggacccgcaagggcctctcgcgagcggagcggccgatgactgattagccggttggtgaggtaacggctcaccaaagcaacgatcagtagctggtctgagaggacgaccagccacactgggactgagacacggcccagactcctacgggaggcagcagtggggaattttggacaatgggcgcaagcctgatccagctattccgcgtgtgggatgaaggccctcgggttgtaaaccacttttgtagagaacgaaaagacaccttttaaaaaagggtgttgctgacggtactctaagaataagcaccggctaactacgtgccagcagccgcggtaatacgtagggtgcgagcgttaatcggaattactgggcgtaaagggtgcgcaggcggttgagtaagacagatgtgaaatccccgagcttaactcgggaatggcatatgtgactgctcgactagagtgtgtcagagggaggtggaattccacgtgtagcagtgaaatgcgtagatatgtggaagaacaccgatggcgaaggcagcctcctgggacataactgacgctcaggcacgaaagcgtggggagcaaacaggattagataccctggtagtccacgccctaaacgatgttaactagttgttgggaagtaaaattctcagtaacgcagccaacgcgagaagttaaccgcctgggaagtacggtcgcaagactaaaactcaaaggaattgacggggacccgcacaagcggtggatgatgtggattaattcgatgcaacgcgaaaaaccttacctacccttgacatgtcaggaagctcttgtaatgagagcgtgcccgcaagggagcctgaacacaggtgctgcatggctgtcgtcagctcgtgtcgtgagatgttgggttaagtcccgcaacgagcgcaacccttgtcactagttgctacgaaagggcactctagtgagactgccggtgacaaaccggaggaaggtggggatgacgtcaagtcctcatggcccttatgggtagggcttcacacgtcatacaatggtcggaacagagggcagcgaagtcgtgagacggagccaatcccagaaaaccgatcgtagtccggattgcagtctgcaactcgactgcatgaagtcggaatcgctagtaatcgcggatcagcatgccgcggtgaatacgttcccgggtcttgtacacaccgcccgtcaaacaatgggagtggtgtttaccagaagtnnntnnga

SEQ ID NO: 42 - DNA, 1375 bp, synthetic construct
  misc_feature 1..1375: note = Synthetic: FBI00067
  source 1..1375: mol_type = other DNA
tcgaacggcagcatgggggcaaccctgatggcgagtggcgaacgggtgagtaatatatcggaacgtacccaagagtgggggacaaccagtcgaaagattggctaataccgcatacgatctaaggatgaaagtgggggaccttcgggcctcacgctcctggagcggccgatagctgattagctagttggtggggtaaaggcctaccaaggcgatgatcagtagctggtctgagaggacgatcagccacactgggactgagacacggcccagactcctacgggaggcagcagtggggaattttggacaatgggggaaaccctgatccagcaatgccgcgtgagtgaagaaggccttcgggttgtaaagctcttttgtcagggaagaaattgattgggctaatatcccgattagatgacggtacctgaagaataagcaccggctaactacgtgccagcagccgcggtaatacgtagggtgcgagcgttaattcggaattactgggcgtaaagcgtgcgcaggcggttgtgtaagacagatgtgaaatgcccgggctcaacctgggaattgcatttgtgactgcacggctagagtgtgtcagaggggggtagaattccacgtgtagcagtgaaatgcgtagatatgtggaggaataccgatggcgaaggcagccccctgggataacactgacgctcatgcacgaaagcgtggggagcaaacaggattagataccctggtagtccacgccctaaacgatgtcgactagttgttgggtcttaattgacttagtaacgcagctaacgcgtgaagtcgaccgcctggggagtacggtcgcaagattaaaactcaaaggaattgacggggacccgcacaagcggtggatgatgtggattaattcgatgcaacgcgaaaaaccttacctacccttgacatgtcaggaatcccgaagagatttgggagtgctcgaaagagagcctgaacacaggtgctgcatggctgtcgtcagctcgtgtcgtgagatgttgggttaagtcccgcaacgagcgcaacccttgtcattagttgctacgaaagggcactctaatgaaactgccggtgacaaaccggaggaaggtggggatgacgtcaagtcctcatggcccttatgggtagggcttcacacgtcatacaatggtgcatacagagggttgccaacccgagagggggagctaatctcagaaagtgcatcgtagtccggattgaagtctgcaactcgacttcatgaagttggaatcgctagtaatcgcggatcagcatgtcgcggtgaatacgttcccgggtcttgtacccaccccccgtcacaccatgggagcgggttttnccagaagtaggtagntta

SEQ ID NO: 43 - DNA, 1380 bp, synthetic construct
  misc_feature 1..1380: note = Synthetic: FBI00068
  source 1..1380: mol_type = other DNA
tgcangtcgaacgagagaattgctagcttgctaataattctctagtggcgcacgggtgagtaacacgtgagtaacctgcccccgagagcgggatagccctgggaaactgggattaataccgcatagtatcgaaagattaaagcagcaatgcgcttggggatgggctcgcggcctattagttagttggtgaggtaacggctcaccaaggcgatgacgggtagccggtctgagaggatgtccggccacactggaactgagacacggtccagacacctacgggtggcagcagtcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatntcgagaggaacactcgtggcgaaggcgggttcctggacattaactgacgctgaggcacgaaggccaggggagcgaaagggattagatacccctgtagtcctggcagtaaacggtgcacgcttggtgtgcggggaatcgaccccctgcgtgccggagctaacgcgttaagcgtgccgcctggggagtacggtcgcaagattaaaactcaaagaaattgacggggacccgcacaagcggtggagtatgtggcttaattcgatgcaacgcgaagaaccttacctgggcttgacatgtaatgaacaacatgtgaaagcatgcgactcttcggaggcgttacacaggtgctgcatggccgtcgtcagctcgtgtcgtgagatgtttggttaagtccagcaacgagcgcaacccctgttgccagttaccagcacgtgaaggtggggactctggcgagactgcccagatcaactgggaggaaggtggggacgacgtcaggtcagtatggcccttatgcccagggctgcacacgtactacaatgcccagtacagagggggccgaagccgcgaggcggaggaaatcctgaaaactgggcccagttcggactgtaggctgcaacccgcctacacgaagccggaatcgctagtaatggcgcatcagctacggcgccgtgaatacgttcccgggtcttgtacacaccgcccgtcacatcatggaagccggtcgcacccgnaagtatnnnnagccaaccgcaaggaggcannnc

SEQ ID NO: 44 - DNA, 1394 bp, synthetic construct
  misc_feature 1..1394: note = Synthetic: FBI00069
  source 1..1394: mol_type = other DNA
tgcngtcgaacggaactgttttgaaagatttcttcggaatgaatttgatttagtttagtggcggacgggtgagtaacgcgtgagtaacctgccttcaagagggggataacattctgaaaagaatgctaataccgcatgacatatcggaaccacatggttttgatatcaaagattttatcgcttgaagatggactcgcgtccgattagttagttggtgaggtaacggctcaccaagaccgcgatcggtagccggactgagaggttgaacggccacattgggactgagacacggcccagactcctacgggaggcagcagtgggggatattgcgcaatgggggcaaccctgacgcagcaacgccgcgtgaaggatgaaggttttcggattgtaaacttcttttattaaggacgaaaattgacggtacttaatgaataagctccggctaactacgtgccagcagccgcggtaatacgtagggagcaagcgttgtccggatttactgggtgtaaagggtgcgtaggcggctttgcaagtcagatgtgaaatctatgggctcaacccataaactgcatttgaaactgtagagcttgagtgaagtagaggcaggcggaattccccgtgtagcggtgaaatgcgtagagatggggaggaacaccagtggcgaaggcggcctgctgggctttaactgacgctgaggcacgaaagcgtgggtagcaaacaggattagataccctggtagtccacgctgtaaacgatgattactaggtgtggggggtctgaccccttccgtgccggagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatccaactaacgaagtagagatacattaggtgcccttcggggaaagttgagacaggtggtgcatggttgtcgtcagctcgtgtcgtgagatgttgggttaagtcccgcaacgagcgcaacccttgctattagttgctacgcaagagcactctaataggactgccgttgacaaaacggaggaaggtggggacgacgtcaaatcatcatgccccttatgacctgggctacacacgtactacaatggatgttaacagagggaagcaagacagcgatgtggagcaaacccctaaaaacattctcagttcagattgcaggctgcaacccgcctgcatgaagatggaattgctagtaatcgcggatcagcatgccgcggtgaatacgttcccgggccttgtacacaccgcccgtcacaccatgggagccggtaatacccgaagtcagtagtccaacctcgtgaggacgc

SEQ ID NO: 45 - DNA, 1392 bp, synthetic construct
  misc_feature 1..1392: note = Synthetic: FBI00070
  source 1..1392: mol_type = other DNA
tgcagtcgaggggcagcattttagtttgcttgcaaactgaagatggcgaccggcgcacgggtgagtaacacgtatccaacctgccgataactccggaatagcctttcgaaagaaagattaataccggatggtatacgaatatcgcatgatatttttattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatgggcgagagcctgaaccagccaagtagcgtgaaggatgaaggctctatgggtcgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgttactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaacagaatatattggaaacagtatagccgtaaggctgttgtgaaggtgctgcatggttgtcgtcagctcgtgccgtgaggtgtcggcttaagtgccataacgagcgcaacccttatctttagttactaacaggtatgctgaggactctagagagactgccgtcgtaagatgtgaggaaggtggggatgacgtcaaatcagcacggcccttacgtccggggctacacacgtgttacaatggggggtacagaaggcggctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagc

SEQ ID NO: 46 - DNA, 1394 bp, synthetic construct
  misc_feature 1..1394: note = Synthetic: FBI00071
  source 1..1394: mol_type = other DNA
tgcagtcgnncgaagcactttattacaatttcttcggaatgacgatttagtgactgagtggcggacgggtgagtaacgcgtgggtaacctgccttatacagggggataacagttagaaatgactgctaataccgcataagcgcacaggattgcatgatctggtgtgaaaaactccggtggtataagatggacccgcgtctgattagctggttggtgaggtaacggcccaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaaaaatgacggtacctgactaagaagccccggctaactacgtgccagcagcccggtaatacgtagggggcaagcgttatccggatttactgggtgtaaagggagcgcaggcggtgcggcaagtctgatgtgaaagcccggggctcaaccccgggactgcattggaaactgtcgtacttgagtatcggagaggtaagtggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgtcgggggacatagtccttcggtgccgcagcaaacgcaataagtattccacctgggggagtacgttcgcaagaatgaaactcaaaggaattgacggggacccgcacaagcggtggagcatgtggtttaattcgaagcaacgcgaagaaccttaccaagtcttgacatcccggtgacaaagtatgtaatgtactctttcttcggaacaccggtgacaggtggtgcatggttgtcgtcagctcgtgtcgtgagatgttgggttaagtcccgcaacgagcgcaacccctgttcttagtagccagcggttcggccgggcactctagggagactgccagggataacctggaggaaggcggggatgacgtcaaatcatcatgccccttatgacttgggctacacacgtgctacaatggcgtaaacaaagggaagcgaaaaggtgacttctagcaaatcccaaaaataacgtcccagttcggactgtagtctgcaactcgactacacgaagctggaatcgctagtaatcgcgaatcagaatgtcgcggtgaatacgttcccgggtcttgtacacaccgcccgtcacaccatgggagtcgggaatgcccgaagccggtgactcaancgaaaggag

SEQ ID NO: 47 - DNA, 1391 bp, synthetic construct
  misc_feature 1..1391: note = Synthetic: FBI00075
  source 1..1391: mol_type = other DNA
tgcagtcgaggggcagcatggacccagctttgctgggtttgatggcgaccggcgcacgggtgagtaacgcgtatccaacctgccctttactccgggatagtctcctgaaagggagtttaataccggatgtgtttgtctttccgcatgggagcgacaaataaagattaattggtaaaggatggggatgcgtcccattagcttgttggcggggtaacggcccaccaaggcgacgatgggtaggggttctgagaggaaggtcccccacattggaactgagacacggtccaaactcctacgggaggcagcagtgaggaatattggtcaatgggcgggagcctgaaccagccaagtagcgtgaaggacgacggccctacgggttgtaaacttcttttataagggaataaagttcgccacgtgtggtgttttgtatgtaccttatgaataagcatcggctaattccgtgccagcagccgcggtaatacggaagatgcgagcgttatccggatttattgggtttaaagggagcgtaggcgggcttttaagtcagcggtcaaatgtcgtggctcaaccatgtcaagccgttgaaactgtaagccttgagtctgcacagggcacatggaattcgtggtgtagcggtgaaatgcttagatatcacgaagaactccgatcgcgaaggcattgtgccggggcataactgacgctgaggctcgaaagtgcgggtatcaaacaggattagataccctggtagtccgcacggtaaacgatgaatgctcgctatgggcgatatattgtccgtggccaagcgaaagcgttaagcattccacctggggagtacgccggcaacggtgaaactcaaaggaattgacgggggcccgcacaagcggaggaacatgtggtttaattcgatgatacgcgaggaaccttacccgggcttgaattgcaggtgcatgagtcagagatgattctttccttcgggactcctgtgaaggtgctgcatggttgtcgtcagctcgtgccgtgaggtgtcggcttaagtgccataacgagcgcaacccttctccccagttgccatcgggtaatgccgggccctctggggacactgccatcgtaagatgcgaggaaggtggggatgacgtcaaatcagcacggcccttacgtccggggccacacacgtgttacaatggggggtacagagggccgctgtccggtgacggtcggccaatccctaaaacccctctcagttcggactggagtctgcaacccgactccacgaagctggattcgctagtaatcgcgcatcagccatggcgcggtgaatacgttcccgggccttgtacacaccgcccgtcaagccatgaaagccgggggtgcctgaagtccgtgaccgcgag

SEQ ID NO: 48 - DNA, 1525 bp, synthetic construct
  misc_feature 1..1525: note = Synthetic: FBI00076
  source 1..1525: mol_type = other DNA
tggagagtttgatcctggctcaggatgaacgctagctacaggcttaacacatgcaagtcgaggggcagcatttcagtttgcttgcaaactggagatggcgaccggcgcacgggtgagtaacacgtatccaacctgccgataactcggggatagcctttcgaaagaaagattaatacccgatggtataatyagaccgcatggtcttgttattaaagaatttcggttatcgatggggatgcgttccattaggcagttggtgaggtaacggctcaccaaaccttcgatggataggggttctgagaggaaggtcccccacattggaactgagacacggtccaaactcctacgggaggcagcagtgaggaatattggtcaatgggcgcaggcctgaaccagccaagtagcgtgaaggatgactgccctatgggttgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggacagttaagtcagttgtgaaagtttgcggctcaaccgtaaaattgcagttgatactggctgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaatatattggaaacagtatagccgtaaggcaaatgtgaaggtgctgcatggttgtcgtcagctcgtgccgtgaggtgtcggcttaagtgccataacgagcgcaacccttatctttagttactaacaggtcatgctgaggactctagagagactgccggtcgtaagatgtgaggaaggtggggatgacgtcaaatcagcacggcccttacgtccggggctacacacgtgttacaatggggggtacagaaggcagctacctggtgacaggatgctaatcccaaaagcctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcgtaacaaggtagccgtaccggaaggtgcggytggatcacctcctt

SEQ ID NO: 49 - DNA, 1397 bp, synthetic construct
  misc_feature 1..1397: note = Synthetic: FBI00077
  source 1..1397: mol_type = other DNA
gcaannnnaacggcagcgcggggagcttgctccctggcggcgagtggcgcacgggtgagtaatacatcggaacgtgtcttctagtgggggataactgcccgaaagggcagctaataccgcatgagacctgagggtgaaagcgggggatcgcaagacctcgcgctggaagagcggccgatgtccgattagctagttggtgaggtaaaggctcaccaaggcgacgatcggtagctggtctgagaggacgaccagccacactgggactgagacacggcccagactcctacgggaggcagcagtggggaattttggacaatgggggcaaccctgatccagccatgccgcgtgcaggatgaaggtcttcggattgtaaactgcttttgtcagggacgaaaagggatgcgataacaccgcattccgctgacggtacctgaagaataagcaccggctaactacgtgccagcagccgcggtaatacgtagggtgcaagcgttaatcggaattactgggcgtaaagcgtgcgcaggcggttctgtaagatagatgtgaaatccccgggctcaacctgggaattgcatatatgactgcaggacttgagtttgtcagaggagggtggaattccacgtgtagcagtgaaatgcgtagatatgtggaagaacaccgatggcgaaggcagccctctgggacatgactgacgctcatgcacgaaagcgtggggagcaaacaggattagataccctggtagtccacgccctaaacgatgtctactagttgttggggacgatagtcnttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctagccttgacatgccaggaaggcctgagagatcaggccgtgcccgcaagggaatctggacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatggctagggcctcacacgtcatacaatggtcggaacagagggaagcgaagccgcgaggtgaagccaatcccagaaaaccgatcgtagtccggattgcagtctgcaactcgactgcatgaagtcggaatcgctagtaatcgcggatcagcatgccgcggtgaatacgttcccgggtcttgtacacaccgcccgtcacaccatgggagtggggttcaccagaagacgtttgcccaaccgaaaggaaggc

SEQ ID NO: 50 - DNA, 1393 bp, synthetic construct
  misc_feature 1..1393: note = Synthetic: FBI00078
  source 1..1393: mol_type = other DNA
tacnntgcagtcgnacgggaaaccttttattgaagcttcggcagatttggctggtttctagtggcggacgggtgagtaacgcgtgggtaacctgccttatacagggggataacaaccagaaatggttgctaataccgcataagcgcacaggaccgcatggtccggtgtgaaaaactccggtggtataagatggacccgcgttggattagctagttggcagggtaacggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggactggcaagtctgatgtgaaaggcgggggctcaacccctggactgcattggaaactgttagtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgttggggagcaaagctcttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatccctctgaccnnnccttaaccgantctttccttcgggacaggggagacaggtggtgcatggttgtcgtcagctcgtgtcgtgagatgttgggttaagtcccgcaacgagcgcaacccctatccccagtagccagcggttcggccgggcactctgaggagactgccagggataacctggaggaaggcggggatgacgtcaaatcatcatgccccttatgatttgggctacacacgtgctacaatggcgtaaacaaagggaagcaagcctgcgaaggtaagcaaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaactgcaaaga

SEQ ID NO: 51 - DNA, 1392 bp, synthetic construct
  misc_feature 1..1392: note = Synthetic: FBI00079
  source 1..1392: mol_type = other DNA
tgcagtcgaacgaagcaattaagatgaagttttcggatggaatcttgattgactgagtggcggacgggtgagtaacgcgtggataacctgcctcacactgggggataacagttagaaatgactgctaataccgcataagcgcacagtgccgcatggcagtgtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgccgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatccccctgacgggccggtaacgcggcctttccttcgggacaggggagacaggtggtgcatggttgtcgtcagctcgtgtcgtgagatgttgggttaagtcccgcaacgagcgcaacccttatccttagtagccagcaggtagagccgggcactctagggagactgccagggataacctggaggaaggtggggatgacgtcaaatcatcatgccccttatgatttgggctacacacgtgctacaatggcgtaaacaaagggaagcgagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaaccgaaaggagg

SEQ ID NO: 52 - DNA, 1389 bp, synthetic construct
  misc_feature 1..1389: note = Synthetic: FBI00080
  source 1..1389: mol_type = other DNA
cgaacggcagcacagggagcttgctcccgggtggcgagtggcgcacgggtgagtaatacatcggaacgtgtcctgttgtgggggataactgctcgaaagggtggctaataccgcatgagacctgagggtgaaagcgggggatcgcaagacctcgcgcaattggagcggccgatgcccgattagctagttggtgaggtaaaggctcaccaaggcgacgatcggtagctggtctgagaggacgaccagccacactgggactgagacacggcccagactcctacgggaggcagcagtggggaattttggacaatgggggcaaccctgatccagccatgccgcgtgcaggatgaaggccttcgggttgtaaactgcttttgtcaggacgaaaaggaccgtgttaataccatggtctgctgacggtacctgaagaataagcaccggctaactacgtgccagcagccgcggtaatacgtagggtgcaagcgttaatcggaattactgggcgtaaagcgtgcgcaggcggttctgtaagacagatgtgaaatccccgggctcaacctgggaattgcatttgtgactgcaggactagagttcatcagaggggggtggaattccaagtgtagcagtgaaatgcgtagatatttggaagaacaccaatggcgaaggcagccccctgggatgcgactgacgctcatgcacgaaagcgtggggagcaaacaggattagataccctggtagtccacgccctaaacgatgtctactggttgttggggttttttaaccttggtaacgaagctaacgcgtgaagtagaccgcctggggagtacggtcgcaagattaaaactcaaaggaattgacggggacccgcacaagcggtggatgatgtggattaattcgatgcaacgcgaaaaaccttacctagccttgacatgccaggaatcctgaagagattcgggagtgcccgcaagggaatctggacacaggtgctgcatggctgtcgtcagctcgtgtcgtgagatgttgggttaagtcccgcaacgagcgcaacccttgtcactagttgctacgcaagagcactctagtgagactgccggtgacaaaccggaggaaggtggggatgacgtcaagtcctcatggcccttatggctagggcctcacacgtcatacaatggtcggaacagagggcagcgaagccgcgaggtggagcaaatcccagaaaaccgatcgtagtccggattgcagtctgcaactcgactgcatgaagtcggaatcgctagtaatcgcggatcagcatgccgcggtgaatacgttcccgggtcttgtacacaccgcccgtcacaccatgggagtggggttcaccagaagacgtttgtttaaccgcaaggagga

SEQ ID NO: 53 - DNA, 1384 bp, synthetic construct
  misc_feature 1..1384: note = Synthetic: FBI00081
  source 1..1384: mol_type = other DNA
gtcgaggggcagcgagatgtagcaatacgtcgtcggcgaccggcgaatgggtgagtaacacgtatgcaacttacctcttagtggtgaataacccgatgaaagtcggactaatacaccatactctccttagatcacatgagaagaggaggaaagattaatcgctaagagatgggcctgcgttccattagctagttggtaaggtaacggcttaccaaggcaacgatggatagggggactgagaggttgaccccccacattgacactgagatacgggtcaaactcctacgggaggcagcagtgaggaatattggtcaatgggcgagagcctgaaccagccaagtcgcgtgaaggaagactgcccgcaagggttgtaaacttcttttgtgtgggattaaagtcacctacgtgtaggtgtttgcagttaccatacgaataagcatcggctaactccgtgccagcagccgcggtaatacggaggatgcgagcgttatccggaattattgggtttaaagggtgcgtaggttgcaagggaagtcaggggtgaaaagctgtagctcaactatggtcttgcctttgaaactctctagctagagtgtattggaggtacgtggaacgtgtggtgtagcggtgaaatgcatagatatcacacagaactccgattgcgcaggcagcgtactacattacaactgacactgaagcacgaaagcgtgggtatcaaacaggattagataccctggtagtccacgcagtaaacgatgaatactagatctatgcgatacacagtatgggtctaagcgaaagcgataagtattccacctgggggagtacgccggcaacggtgaaactcaaagagattggcgggggtccgcacaagcggaggaacatgtggtttaattcgatgatacgcgaggaaccttacccgggattgaaatgtagatgcatgaggctgagaggtctcttcccttcggggcttctatgtaggtgctgcatggttgtcgtcagctcgtgccgtgaggtgtcggcttaagtgccataacgagcgcaacccgcgtcgatagttactaacgagtcatgtcgaggactctatcgagacagccgtcgtaagacgtgaggaaggagcggatgacgtcaaatcagcacggcccttacatccggggcgacacacgtgttacaatggtagggacagcgagcagccatctggtgacagagagctaatctataaaccctatcccagttcggatcggagtctgcaactcgactctgtgaagctggattcgctagtaatcgcgcatcagccatggcgcggtgaatacgttcccggaccttgcacacaccgcccgtcaagccatgggagtcgggggtacctgaagagcgtgaccgtcac

SEQ ID NO: 54 - DNA, 1394 bp, synthetic construct
  misc_feature 1..1394: note = Synthetic: FBI00085
  source 1..1394: mol_type = other DNA
tgcnntcgaacggaactgttttgaaagatttcttcggaatgaatttgatttagtttagtggcggacgggtgagtaacgcgtgagtaacctgccttcaagagggggataacattctgaaaagaatgctaataccgcatgacatatcggaatcacatgattttgatatcaaagattttatcgcttgaagatggactcgcgtccgattagttagttggtgaggtaacggctcaccaagaccgcgatcggtagccggactgagaggttgaacggccacattgggactgagacacggcccagactcctacgggaggcagcagtgggggatattgcgcaatgggggcaaccctgacgcagcaacgccgcgtgaaggatgaaggttttcggattgtaaacttcttttattaaggacgaaacttgacggtacttaatgaataagctccggctaactacgtgccagcagccgcggtaatacgtagggagcaagcgttgtccggatttactgggtgtaaagggtgcgtaggcggctttgcaagtcagatgtgaaatctatgggctcaacccataaactgcatttgaaactgtagagcttgagtgaagtagaggcaggcggaattccccgtgtagcggtgaaatgcgtagagatggggaggaacaccagtggcgaaggcggcctgctgggctttaactgacgctgaggcacgaaagcgtgggtagcaaacaggattagataccctggtagtccacgctgtaaacgatgattactaggtgtggggggtctgaccccttccgtgccggagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatccaactaacgaagtagagatacattaggtgcccttcggggaaagttgagacaggtggtgcatggttgtcgtcagctcgtgtcgtgagatgttgggttaagtcccgcaacgagcgcaacccttgctattagttgctacgcaagagcactctaataggactgccgttgacaaaacggaggaaggtggggacgacgtcaaatcatcatgccccttatgacctgggctacacacgtactacaatggatgttaacagagggaagcaagacagcgatgtggagcaaacccctaaaaacattctcagttcagattgcaggctgcaacccgcctgcatgaagatggaattgctagtaatcgcggatcagcatgccgcggtgaatacgttcccgggccttgtacacaccgcccgtcacaccatgggagccggtaatacccgaagtcagtagtccaacctcgtgaggacgc

SEQ ID NO: 55 - DNA, 1532 bp, synthetic construct
  misc_feature 1..1532: note = Synthetic: FBI00087
  source 1..1532: mol_type = other DNA
tggagagtttgatcctggctcaggatgaacgctggcggcgtgcctaacacatgcaagtcgaacgaagcgcttccgcctgattttcttcggagatgaaggcggctgcgactgagtggcggacgggtgagtaacgcgtgggcaacctgccttgcactgggggataacagccagaaatggctgctaataccgcataagaccgaagcgccgcatggcgcdgcggccaaagccccggcggtgcaagatgggcccgcgtctgattaggtrgttggcggggtaacggcccaccaagccgacgatcagtagccgacctgagagggtgaccggccacattgggactgagacacggcccagactcctacgggaggcagcagtggggaatattgcacaatgggggaaaccctgatgcagcgacgccgcgtgaaggatgaagtatttcggtatgtaaacttctatcagcagggaagaagatgacggtacctgactaagaagccccggctaactacgtgccagcagccgcggtaatacgtagggggcaagcgttatccggatttactgggtgtaaagggagcgtagacggcgatgcaagccagatgtgaaagcccggggctcaaccccgggactgcatttggaactgcgtggctggagtgtcggagaggcaggcggaattcctagtgtagcggtgaaatgcgtagatattaggaggaacaccagtggcgaaggcggcctgctggacgatgactgacgttgaggctcgaaagcgtggggagcaaacaggattagataccctggtagtccacgccgtaaacgatgactactaggtgtcgggtggcaaggccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgatcttgacatcccgatgccaaagcgcgtaacgcgctctttcttcggaacatcggtgacaggtggtgcatggttgtcgtcagctcgtgtcgtgagatgttgggttaagtcccgcaacgagcgcaacccctatcttcagtagccagcatttyggatgggcactctggagagactgccagggacaacctggaggaaggtggggatgacgtcaaatcatcatgccccttatgaccagggctacacacgtgctacaatggcgtaaacaaagggaggcgaacccgcgagggtgggcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagccggtgacccaacccgcaagggagggagccgtcgaaggtgggaccgataactggggtgaagtcgtaacaaggtagccgtatcggaaggtgcggytggatcacctcctt

SEQ ID NO: 56 - DNA, 1389 bp, synthetic construct
  misc_feature 1..1389: note = Synthetic: FBI00092
  source 1..1389: mol_type = other DNA
tgcagtcgagcgagaaatttttaacggattccttcggggagaagataaggatggaaagcggcggacgggtgagtaacgcgtgagtaacctgcctttaggagggggacaacattccgaaagggatgctaataccgcataaaattattgtatcgcatggtatgataatcaaagatttatcgcctaaagatggactcgcgtccgattagctagttggtggggtaaaagcctaccaaggcgacgatcggtagccgaactgagaggttgatcggccacattgggactgagacacggcccagactcctacgggaggcagcagtgggggatattgcgcaatgggggaaaccctgacgcagcaacgccgcgtgaaggaagaaggccttcgggttgtaaacttctttaagtgtggaagataatgacggtacacacagaataagccacggctaactacgtgccagcagccgcggtaatacgtaggtggcaagcgttgtccggatttactgggtgtaaagggcgtgtaggcgggtagacaagtcagatgtgaaataccggggctcaactccggggctgcatttgaaactgtatatcttgagtgtcggagaggaaagcggaattcctagtgtagcggtgaaatgcgtagatattaggaggaacaccagtggcgaaggcggctttctggacgataactgacgctgaggcgcgaaagcgtggggagcaaacaggattagataccctggtagtccacgccgtaaacgatggatactaggtgtaggaggtatcgaccccttctgtgccgcagttaacacaataagtatcccacctggggagtacggtcgcaagattgaaactcaaaggaattgacgggggcccgcacaagcagtggagtatgtggtttaattcgaagcaacgcgaagaaccttaccaggacttgacatcccacgcatagcctagagataggtgaagtcctacgggacgtggagacaggtggtgcatggttgtcgtcagctcgtgtcgtgagatgttgggttaagtcccgcaacgagcgcaacccttactgtcagttaccatcattaagttggggactctggcaggactgccggtgacaaatcggaggaaggtggggacgacgtcaaatcatcatgccccttatgtcctgggctacacacgtactacaatggctgtaaacagagtgaagcaaagcagtgatgtggagcaaaacacaaaaagcagtctcagttcagattgtaggctgaaactcgcctatatgaagtcggaattgctagtaatcgcagatcagcatgctgcggtgaatacgttcccgggccttgtacacaccgcccgtcacaccatgagagtcgataacacccgaagcctgtagcttaaccttaggga

SEQ ID NO: 57 - DNA, 1391 bp, synthetic construct
  misc_feature 1..1391: note = Synthetic: FBI00093
  source 1..1391: mol_type = other DNA
gcaagtcgaacgaagcactttaattgatttcttcggaatgaagtttttgtgactgagtggcggacgggtgagtaacgcgtgggtaacctgcctcatacagggggataacagttggaaacgactgctaataccgcataagcgcacaggattgcatgatccagtgtgaaaaactccggtggtatgagatggacccgcgtctgattagccagttggcggggtaacggcccaccaaagcgacgatcagtagcnnacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaagaatgacggtacctgactaagaagcaccggctaaatacgtgccagcagccgcggtaatacgtatggtgcaagcgttatccggatttactgggtgtaaagggagcgcaggcggtacggcaagtctgatgtgaaatcccggggctcaaccccggtactgcattggaaactgtcggactagagtgtcggaggggtaagtggaattcctagtgtagcggtgaaatgcgtagatattaggaggaacaccagtggcgaaggcggcttactggacgattactgacgctgaggctcgaaagcgtggggagcaaacaggattagataccctggtagtccacgccgtaaacgatgaatactaggtgtcggggagcattgctcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgaccaagtatgtaatgtacttttctcttcggagcagtggtgacaggtggtgcatggttgtcgtcagctcgtgtcgtgagatgttgggttaagtcccgcaacgagcgcaacccctattcttagtagccagcggttcggccgggcactctagggagactgccagggataacctggaggaaggtggggatgacgtcaaatcatcatgccccttatgacttgggctacacacgtgctacaatggcgtaaacaaagggaagcaatcccgcgagggggagcaaatctcaaaaataacgtctcagttcggactgtagtctgcaactcgactacacgaagctggaatcgctagtaatcgcgaatcagaatgtcgcggtgaatacgttcccgggtcttgtacacaccgcccgtcacaccatgggagttggtaatgcccgaagtcagtgacccaaccgcaagga

SEQ ID NO: 58 - DNA, 1371 bp, synthetic construct
  misc_feature 1..1371: note = Synthetic: FBI00096
  source 1..1371: mol_type = other DNA
anncntgcagtcgaacgatgaaaccgccctcgggcggacatgaagtggcgaacgggtgagtaacacgtgaccaacctgcccccctctccgggacaaccttgggaaaccgaggctaataccggatactccctcccctgctcctgcaggggtcgggaaagcccaggcggagggggatggggtcgcggcccattaggtagtaggcggggtaacggcccacctagcccgcgatgggtagccgggttgagagaccgaccggccacattgggactgagatacggcccagactcctacgggaggcagcagtggggaattttgcgcaatgggggcaaccctgacgcagcaacgccgcgtgcgggacgacggccttcgggttgtaaaccgctttcagcagggaagaaattcgacggtacctgcagaagaagctccggctaactacgtgccagcagccgcggtaatacgtagggagcgagcgttatccggattcattgggcgtaaagagcgcgtaggcggcctctcaagcgggatctctaatccgagggctcaacccccggccggatcccgaactgggaggctcgagttcggtagaggcaggcggaattcccggtgtagcggtggaatgcgcagatatcgggaagaacaccgatggcgaaggcagcctgctgggccgcaactgacgctgaggcgcgaaagctaggggagcgaacaggattagataccctggtagtcctagccgtaaacgatggatactaggtgtggggggctccgccctccgtgccgcagccaacgcattaagtatcccgcctggggagtacggccgcaaggctaaaactcaaaggaattgacgggggcccgcacaagcagcggagcatgtggcttaattcgaagcaacgcgaagaaccttaccagggcttgacatggacgtgaagccggggaaacccggtggccgagaggagcgtccgcaggtggtgcatggctgtcgtcagctcgtgtcgtgagatgttgggttaagtcccgcaacgagcgcaacccctgccccatgttgccagcattaggttggggactcatgggggactgccggcgtcaagccggaggaaggtggggacgacgtcaagtcatcatgccctttatgccctgggctgcacacgtgctacaatggccggtacaacgggctgcgagaccgcgaggtcgagcgaatccctcaaagccggccccagttcggatcggaggctgcaacccgcctccgtgaagtcggagttgctagtaatcgcggatcagcatgccgcggtgaatacgttcccgggccttgtacacaccgcccgtcacaccacccgagtcgtctgcacccgaagccgccggccgaacccgcaaggg

SEQ ID NO: 59 - DNA, 1384 bp, synthetic construct
  misc_feature 1..1384: note = Synthetic: FBI00097
  source 1..1384: mol_type = other DNA
tgcagtcgaacggagctattttggcggaacccttcggggggaagctggtttagcttagtggcggacgggtgagtaacacgtgagcaacctgcctttcagagggggataacgtttggaaacgaacgctaataccgcataacgcagcgggccggcatcggcctgctgccaaaggagcaatccgctgaaagatgggctcgcgtccgattagatagttggtgaggtaacggcccaccaagtcgacgatcggtagccggactgagaggttgaacggccacattgggactgagacacggcccagactcctacgggaggcagcagtgggggatattgcacaatgggcgaaagcctgatgcagcgacgccgcgtgagggaagacggccttcgggttgtaaacctctgtctttggggacgataatgacggtacccaaggaggaagctccggctaactacgtgccagcagccgcggtaatacgtagggagcgagcgttgtccggaattactgggtgtaaagggagcgtaggcgggagtgcaagttggatgtgaaaaccacgggctcaacccatggactgcattcaaaactgtatttcttgagtgatgtagaggcaggcggaattcctagtgtagcggtgaaatgcgtagatattaggaggaacaccagtggcgaaggcggcctgctgggcatttactgacgctgaggctcgaaagtgtggggagcaaacaggattagataccctggtagtccacaccgtaaacgatgattactaggtgtgggtgcgtcaagcatccgtgccggagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatcggatgcatagcttagagataagtgaagtccttcgggacatccagacaggtggtgcatggttgtcgtcagctcgtgtcgtgagatgttgggttaagtcccgcaacgagcgcaacccttatgtttagttgctacgcaagagcactctaaagagactgccgttgacaaaacggaggaaggtggggatgacgtcaaatcatcatgccccttatgacctgggccacacacgtactacaatggcgatcaacagagggaagcaaagccgcgaggcagagcaaacccctaaaagtcgtctcagttcggattgcaggctgcaacccgcctgcatgaagccggaattgctagtaatcgcggatcagcatgccgcggtgaatacgttcccgggccttgtacacaccgcccgtcacaccatggaagtcggtaacacccgaagccagtagcccaaccgcaaggag

SEQ ID NO: 60 - DNA, 1379 bp, synthetic construct
  misc_feature 1..1379: note = Synthetic: FBI00099
  source 1..1379: mol_type = other DNA
anncatgcagtcgancgattaagacgccttcgggcgtgcatagagtggcgaacgggtgagtaacacgtgaccaacctgccccctaccccgggataacgcaaggaaacctgcgctaataccggatactccttcccctgctcctgcaggggtcgggaaagcccagacggtaggggatggggtcgcggcccattaggtagacggcggggcaacggcccaccgtgcctgcgatgggtagccgggttgagagaccgaccggccacattgggactgagatacggcccagactcctacgggaggcagcagtggggaattttgcgcaatggggggaaccctgacgcagcaacgccgcgtgcgggatgaaggccttcgggttgtaaaccgctttcagcagggaagatagtgacggtacctgcagaagaagccccggctaactacgtgccagcagccgcggtaatacgtagggggcgagcgttatccggattcattgggcgtaaagcgcgcgtaggcggcccgtcaagcggaacctctaacccgagggctcaacccccggtcgggttccgaactggcaggctcgagtttggtagaggaagatggaattcccggtgtagcggtggaatgcgcagatatcgggaagaacaccgatggcgaaggcagtcttctgggccatcaactgacgctgaggcgcgaaagctgggggagcgaacaggattagataccctggtagtcccagccgtaaacgatgggtgctaggtgtggggggatcatccctccgtgccgcagccaacgcattaagcaccccgcctggggagtacggccgcaaggctaaaactcaaaggaattgacgggggcccgcacaagcagcggagcatgtggcttaattcgaagcaacgcgaagaaccttaccagggcttgacatgctagtgaagccggggaaacccggtggccgagaggagctagcgcaggtggtgcatggctgtcgtcagctcgtgtcgtgagatgttgggttaagtcccgcaacgagcgcaacccctatcgtatgttgccagcattgagttggggactcatacgagactgccggcgtcaagccggaggaaggtggggacgacgtcaagtcatcatgccctttatgccctgggctgcacacgtgctacaatggccggtacaacgggccgcgacctggcgacaggaagcgaatccctcaaagccggccccagttcggatcggaggctgcaacccgcctccgtgaagtcggagttgctagtaatcgcggatcagcatgccgcggtgaatacgttcccgggccttgtacacaccgcccgtcacaccacccgagtcgtctgcacccgaagccgccggccgaacccgcaaggggcggaggc

SEQ ID NO: 61 - DNA, 1408 bp, synthetic construct
  misc_feature 1..1408: note = Synthetic: FBI00102
  source 1..1408: mol_type = other DNA
acncntgcagtcgaacgaagcngatctagcggaagttttcggatggaagttannattgactgagtggcggacgggtgagtaacgcgtgggtaacctgcctcacactgggggacaacagttagaaatgactgctaataccgcataagcgcacaggaccgcatggtccggtgtgaaaaactccggtggtgtgagatggacccgcgtttgattagctagttggtggggtaacggcctaccaaggcgacgatcaatagccgacctgagagggtgaccggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtaaagcaagtctgaagtgaaagcccggggctcaaccccgggactgctttggaaactgtttaactagagtgctggagaggtaagcggaattcctagtgtagcggtgaaatgcgtagatattaggaggaacaccagtggcgaaggcggcttactggacagtaactgacgttgaggctcgaaagcgtggggagcaaacaggattagataccctggtagtccacgccgtaaacgatgaatactaggtgttgggggacaaagtccttcggtgccgtcgcaaacgcaataagtattccacctggggagtacgttcgcaagaatgaaactcaaaggaattgacggggacccgcacaagcggtggagcatgtggtttaattcgaagcaacgcgaagaaccttaccaaatcttgacatccctctgaccggcgagtaatgtcgcctttccttcgggacagaggagacaggtggtgcatggttgtcgtcagctcgtgtcgtgagatgttgggttaagtcccgcaacgagcgcaacccttatcctaagtagccagcagttcggctgggcactcttgggagactgccagggataacctggaggaaggtggggatgacgtcaaatcatcatgccccttatgatttgggctacacacgtgctacaatggcgtaaacaaagagaagcgagaccgcgaggtggagcaaatctcaaaaataacgtctcagttcggactgcaggctgcaactcgcctgcacgaagctggaatcgctagtaatcgcgaatcagaatgtcgcggtgaatacgttcccgggtcttgtacacaccgcccgtcacaccatgggagtcagtaatgcccgaagtcagtgacccaaccgtaaggagggagctgccga

SEQ ID NO: 62 - DNA, 1390 bp, synthetic construct
  misc_feature 1..1390: note = Synthetic: FBI00104
  source 1..1390: mol_type = other DNA
tgcagtcnnnnggaattatcttattgaaacttcggtcgatttaatttaattctagtggcggacgggtgagtaacgcgtgggtaacctgccttatacagggggataacagtcagaaatgactgctaataccgcataagcgcacagagctgcatggctcagtgtgaaaaactccggtggtataagatggacccgcgttggattagcttgttggtggggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacccgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggnngcnnnctttcggtgccgtcgcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccccctgaccggaacttaaccgttcctttccttcgggacaggggagacaggtggtgcatggttgtcgtcagctcgtgtcgtgagatgttgggttaagtcccgcaacgagcgcaacccctatcctcagtagccagcagttcggctgggcactctgtggagactgccagggataacctggaggaaggcggggatgacgtcaaatcatcatgccccttatgatttgggctacacacgtgctacaatggcgtaaacaaagggaagcaagcctgtgaaggtaagcaaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgcaagggaggag

SEQ ID NO: 63 - DNA, 1177 bp, synthetic construct
  misc_feature 1..1177: note = Synthetic: FBI00109
  source 1..1177: mol_type = other DNA
tgcagtcgaacgaagcacttnnnnntgattcttcggatgangnnnnttgtgactgagtggcggacgggtgagtaacgcgtgggtaacctgcctcatacagggggataacagttagaaatgactgctaataccgcataagaccacggagccgcatggctcggtgggaaaaactccggtggtatgagatggacccgcgtctgattaggtagttggtggggtaacggcctaccaagccaacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggctgtgtaagtctgaagtgaaagcccggggctcaaccccgggactgctttggaaactatgcagctagagtgtcggagaggtaagtggaattcccagtgtagcggtgaaatgcgtagatattgggaggaacaccagtggcgaanggcggcttactggacgatgactgacgttgaggctcgaaagcgtggggagcaaacaggattagataccctggtagtccacgccgtaaacgatgactactaggtgtcggggagcaaagctcttcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccggtgaccggcatgtaatgatgccttttcttcggaacaccggtgacaggtggtgcatggttgtcgtcagctcgtgtcgtgagatgttgggttaagtcccgcaacgagcgcaacccttatcttcagtagccagcattttggatgggcactctggagagactgccagggataacctggaggaaggtggggatgacgtcnaatcatcatgcnccttatgagcagggctacacacgtgctacaatggc

SEQ ID NO: 64 - DNA, 1386 bp, synthetic construct
  misc_feature 1..1386: note = Synthetic: FBI00110
  source 1..1386: mol_type = other DNA
tgcagtcgaacgaagcatctataggangttttcggatggaatatgggatgactgagtggcggacgggtgagtaacgcgtggataacctgcctcacactgggggataacagttagaaatggctgctaataccgcaaaagcgcacagtaccgcagggtacggtgtgaaaaacccaggtggtgtgagatggatccgcgtctgattagccagttggcggggtaacggcccaccaaagcgactatagtagccgacctgagagggtgaccggccacattgggactgagacacggcccaaactcctacgggaggcagcagtggggaatattgcacaatgggcgaaagcctgatgcagcgaccccgcgtgagtgaagaagtatctcggtatgtaaagctctatcagcagggaagaaaatgacggtacctgactaanaagccccggctaactacgtgccagcagccgcggtaatacgtagggggcaagcgttatccggatttactgggtgtaaagggagcgtagacggcgatgcaagtctgaagtgaaagcccggggctcaaccccgggactgctttggaaactgtgtggctggagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatgctaggtgtcggggggcaaagcccctcggtgccgccgctaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggacagtaacgtgtcccttccttcgggacaggggagacaggtggtgcatggttgtcgtcagctcgtgtcgtgagatgttgggttaagtcccgcaacgagcgcaacccttatccttagtagccagcatttcgggtgggcactctagggagactgccagggataacctggaggaaggtggggatgacgtcaaatcatcatgccccttatgatttgggctacacacgtgctacaatggcgtaaacaaagggaagcggccctgcgaaggtgagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagcaacgcccgaagtcagtgacccaancgaaagg

SEQ ID NO: 65 - DNA, 1395 bp, synthetic construct
  misc_feature 1..1395: note = Synthetic: FBI00111
  source 1..1395: mol_type = other DNA
tgcagtcgaggggcagcatggtcttagcttgctaaggccgatggcgaccggcgcacgggtgagtaacacgtatccaacctgccgtctactcttggacagccttctgaaaggaagattaatacaagatggcatcatgagtccacatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgcaggcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaatttcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacagcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttccgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatcccaaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcnc

SEQ ID NO: 66 - DNA, 1399 bp, synthetic construct
  misc_feature 1..1399: note = Synthetic: FBI00112
  source 1..1399: mol_type = other DNA
anncntgcagtcgaggggcagcatgaacttagcttgctaagtttgatggcgaccggcgcacgggtgagtaacacgtatccaacctgccgatgactcggggatagcctttcgaaagaaagattaatacccgatggcatagttcttccgcatggtagaactattaaagaatttcggtcatcgatggggatgcgttccattaggttgttggcggggtaacggcccaccaagccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgaggcacgtgtgcctttttgtatgtaccgtatgaataaggatcggctaactccgtgccagcagccgcggtaatacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcttgctggactgtaactgacgctgatgctcgaaagtgtgggtatcaaacaggattagataccctggtagtccacacagtaaacgatgaatactcgctgtttgcgatatacagtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttgaattgcaactgaatgatgtggagacatgtcagccgcaaggcagttgtgaaggtgctgcatggttgtcgtcagctcgtgccgtgaggtgtcggcttaagtgccataacgagcgcaacccttatcgatagttaccatcaggttatgctggggactctgtcgagactgccgtcgtaagatgtgaggaaggtggggatgacgtcaaatcagcacggcccttacgtccggggctacacacgtgttacaatggggggtacagaaggcagctacacggtgacgtgatgctaatcccgaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacnnnnnagtgnntnnccgcaaggagcnc

SEQ ID NO: 67 - DNA, 1395 bp, synthetic construct
  misc_feature 1..1395: note = Synthetic: FBI00113
  source 1..1395: mol_type = other DNA
tgcagtcgaggggcagcatgatttgtagcaatacagattgatggcgaccggcgcacgggtgagtaacgcgtatgcaacttacctatcagagggggatagcccggcgaaagtcggattaataccccataaaacaggggtcccgcatgggaatatttgttaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgaaggaagaaggatctatggtttgtaaacttcttttataggggaataaagtggaggacgtgtccttttttgtatgtaccctatgaataagcatcggctaactccgtgccagcagccgcggtaatacggaggatgcgagcgttatccggatttattgggtttaaagggtgcgtaggtggtgatttaagtcagcggtgaaagtttgtggctcaaccataaaattgccgttgaaactgggttacttgagtgtgtttgaggtaggcggaatgcgtggtgtagcggtgaaatgcatagatatcacgcagaactccgattgcgaaggcagcttactaaaccataactgacactgaagcacgaaagcgtggggatcaaacaggattagataccctggtagtccacgcagtaaacgatgattactaggagtttgcgatacaatgtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtagtctgaccggagtggaaacactctttctagcaatagcagattacgaggtgctgcatggttgtcgtcagctcgtgccgtgaggtgtcggcttaagtgccataacgagcgcaacccttatcactagttactaacaggtgaagctgaggactctggtgagactgccagcgtaagctgtgaggaaggtggggatgacgtcaaatcagcacggcccttacatccggggcgacacacgtgttacaatggcatggacaaagggcagctacctggtgacaggatgctaatctccaaaccatgtctcagttcggatcggagtctgcaactcgactccgtgaagctggattcgctagtaatcgcgcatcagccatggcgcggtgaatacgttcccgggccttgtacacaccgcccgtcaagccatgggagccgggggtacctgaagtccgtaaccgcaaggatc

SEQ ID NO: 68 - DNA, 1387 bp, synthetic construct
  misc_feature 1..1387: note = Synthetic: FBI00115
  source 1..1387: mol_type = other DNA
tgcagtcgagcgaagcacttaagttcgattcttcggatgaagacttttgtgactgagcggcggacgggtgagtaacgcgtgggtaacctgcctcatacagggggataacagttagaaatggctgctaataccgcataagaccacagtactgcatggtacagtggtaaaaactccggtggtatgagatggacccgcgtctgattaggtagttggtgaggtaacggcccaccaagccgacgatcagtagccgacctgagagggtgaccggccacattgggactgagacacggcccagactcctacgggaggcagcagtggggaatattgcacaatgggcgaaagcctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggctgtgcaagtctgaagtgaaaggcatgggctcaacctgtggactgctttggaaactgtgcagctagagtgtcggagaggtaagtggaattcctagtgtagcggtgaaatgcgtagatattaggaggaacaccagtggcgaaggcggcttactggacgatgactgacgttgaggctcgaaagcgtggggagcaaacaggattagataccctggtagtccacgccgtaaacgatgactgctaggtgtcgggtagcaaagctattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagtttttcttcggaacatcggtgacaggtggtgcatggttgtcgtcagctcgtgtcgtgagatgttgggttaagtcccgcaacgagcgcaacccttatcttcagtagccagcatttaagatgggcactctggagagactgccagggataacctggaggaaggtggggatgacgtcaaatcatcatgccccttatgaccagggctacacacgtgctacaatggcgtaaacaaagggaagcagagccgcgaggccgagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaacncgaaa

SEQ ID NO: 69 - DNA, 1526 bp, synthetic construct
  misc_feature 1..1526: note = Synthetic: FBI00116
  source 1..1526: mol_type = other DNA
tggagagtttgatcctggctcaggatgaacgctggcggcgtgcttaacacatgcaagtcgaacgaagcaccttgatttgattcttcggatgaaratcytggtgactgagtggcggacgggtgagtaacgcgtgggtaacctgcctcatacagggggataacagttagaaatgactgctaataccgcataagaccacagcaccgcatggtgcaggggtaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggagtggcaagtctgatgtgaaaacccggggctcaaccccgggactgcattggaaactgtcaatctrgagtaccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgactactaggtgtcgggcagcaaagctgttcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatctccctgaccggcaagtaatgttgcctttccttcgggacagggatgacaggtggtgcatggttgtcgtcagctcgtgtcgtgagatgttgggttaagtcccgcaacgagcgcaacccctatctttagtagccagcggyywggccgggcactctagagagactgccagggataacctggaggaaggtggggatgacgtcaaatcatcatgccccttatgagcagggctacacacgtgctacaatggcgtaaacaaagggaggcagaaccgcgaggtcgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggtgggaccgataactggggtgaagtcgtaacaaggtagccgtatcggaaggtgcggytggatcacctcctt

SEQ ID NO: 70 - DNA, 1526 bp, synthetic construct
  misc_feature 1..1526: note = Synthetic: FBI00117
  source 1..1526: mol_type = other DNA
tggagagtttgatcctggctcaggatgaacgctggcggcgtgcttaacacatgcaagtcgaacgggaaayayttyattgarrcttcggyvgatttrryytrtttctagtggcggacgggtgagtaacgcgtgggtaacctgccttatactgggggataacagccagaaatgrctgctaataccgcataagcgcacagaaccgcatggttcsgtgtgaaaaactccggtggtataagatggacccgcgttggattagctagttggcagggcarcggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcaagcgttatccggatttactgggtgtaaagggagcgtagacggcgcagcaagtctgatgtgaaaggcaggggcttaacccctggactgcattggaaactgctgtgcttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcwrggagcacagctcttwggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaaatcttgacatccctctgaccgggacttaaccgtccctttycttcgggacaggggagacaggtggtgcatggttgtcgtcagctcgtgtcgtgagatgttgggttaagtcccgcaacgagcgcaacccctatccttagtagccagcrsgtmrkgsygggcactctgaggagactgccagggataacctggaggaaggcggggatgacgtcaaatcatcatgccccttatgatttgggctacacacgtgctacaatggcgtaaacaaagggaagcgaaccygygagggtgggcaaatctcaaaaataacgtcccagttcggactgcagtctgcaactcgactgcacraagctggaatcgctagtaatcgcggatcagaatgccgcggtgaatacgttcccgggtcttgtacacaccgcccgtcacaccatgggagtcagtaacgcccgaagtcagtgacctaacygcaargraggagctgccgaaggcgggaccgatgactggggtgaagtcgtaacaaggtagccgtaccggaaggtgcggytggatcacctcctt

SEQ ID NO: 71 - DNA, 1397 bp, synthetic construct
  misc_feature 1..1397: note = Synthetic: FBI00120
  source 1..1397: mol_type = other DNA
tgnnagtcgagcgaagcggntcttcggaagttttcggatggaanaggaattgacttagcggcggacgggtgagtaacgcgtgggtaacctgccttacactgggggataacagttagaaatgactgctaataccgcataagcgcacagggccgcatggcctggtgtgaaaaactccggtggtgtaagatggacccgcgtctgattaggtagttggtggggtaacggcccaccaagccgacgatcagtagccgacctgagagggtgaccggccacattgggactgagacacggcccaaactcctacgggaggcagcagtggggaatattggacaatgggcgaaagcctgatccagcgacgccgcgtgagtgaagaagtatttcggtatgtaaagctctatcagcagggaagaaaaatgacggtacctgactaagaagccccggctaactacgtgccagcagccgcggtaatacgtagggggcaagcgttatccggatttactgggtgtaaagggagcgtagacggtttagcaagtctgaagtgaaagcccggggctcaaccccggtactgctttggaaactgttagacttgagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgtcggggggcaaagcccttcggtgccgccgcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgaaaacactttaaccggtgtccctcttcggagcagtggagacaggtggtgcatggttgtcgtcagctcgtgtcgtgagatgttgggttaagtcccgcaacgagcgcaacccttatccttagtagccagcgagtagagtcgggcactctggggagactgccagggataacctggaggaaggtggggatgacgtcaaatcatcatgccccttatgatttgggctacacacgtgctacaatggcgtaaacaaagggaggcaaaggagcgatctggagcaaaccccaaaaataacgtctcagttcggattgcaggctgcaactcgcctgcatgaagctggaatcgctagtaatcgcgaatcagaatgtcgcggtgaatacgttcccgggtcttgtacacaccgcccgtcacaccatgggagttggtaacgcccgaagtcagtgacccaaccgnaaggagggag

SEQ ID NO: 72 - DNA, 1396 bp, synthetic construct
  misc_feature 1..1396: note = Synthetic: FBI00123
  source 1..1396: mol_type = other DNA
tgcaagtcgaacgaagcactttaattgatttcttcggaatgaagtttttgtgactgagtggcggacgggtgagtaacgcgtgggtaacctgcctcatacagggggataacagttggaaacgactgctaataccgcataagcgcacaggattgcatgatccagtgtgaaaaactccggtggtatgagatggacccgcgtctgattagccagttggcggggtaacggcccaccaaagcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaagaatgacggtacctgactaagaagcaccggctaaatacgtgccagcagccgcggtaatacgtatggtgcaagcgttatccggatttactgggtgtaaagggagcgcaggcggtacggcaagtctgatgtgaaatcccggggctcaaccccggtactgcattggaaactgtcggactagagtgtcggaggggtaagtggaattcctagtgtagcggtgaaatgcgtagatattaggaggaacaccagtggcgaaggcggcttactggacgattactgacgctgaggctcgaaagcgtggggagcaaacaggattagataccctggtagtccacgccgtaaacgatgaatactaggtgtcggggagcattgctcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgacaaagtatgtaatgtactttctcttcggagcagtggtgacaggtggtgcatggttgtcgtcagctcgtgtcgtgagatgttgggttaagtcccgcaacgagcgcaacccctattcttagtagccagcggttcggccgggcactctagggagactgccagggataacctggaggaaggtggggatgacgtcaaatcatcatgccccttatgacttgggctacacacgtgctacaatggcgtaaacaaagggaagcaatcccgcgagggggagcaaatctcaaaaataacgtctcagttcggactgtagtctgcaactcgactacacgaagctggaatcgctagtaatcgcgaatcagaatgtcgcggtgaatacgttcccgggtcttgtacacaccgcccgtcacaccatgggagttggtaatgcccgaagtcagtgacccaaccgcaaggagggag

SEQ ID NO: 73 - DNA, 1382 bp, synthetic construct
  misc_feature 1..1382: note = Synthetic: FBI00124
  source 1..1382: mol_type = other DNA
tcgaacgaaacaccttatttgattttcttcggaactgaagatttggtgattgagtggcggacgggtgagtaacgcgtgggtaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatcttcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtagaggcttcggtgccgcagccaacgcagtaagtattccacctggggagtacgttcgcaagaatgaaactcaaaggaattgacggggacccgcacaagcggtggagcatgtggtttaattcgaagcaacgcgaagaaccttacctggtcttgacatccttctgaccggtccttaaccggacctttccttcgggacaggagagacaggtggtgcatggttgtcgtcagctcgtgtcgtgagatgttgggttaagtcccgcaacgagcgcaacccctatctttagtagccagcatataaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgt

SEQ ID NO: 74 - DNA, 1379 bp, synthetic construct
  misc_feature 1..1379: note = Synthetic: FBI00125
  source 1..1379: mol_type = other DNA
gcaagtcgaggggcagcatcatcaaagcttgctttgatggatggcgaccggcgcacgggtgagtaacacgtatccaacctgccgacaacactgggatagcctttcgaaagaaagattaataccggatggcatagttttcccgcatgggataattattaaagaatttcggttgtcgatggggatgcgttccattaggcagttggcggggtaacggcccaccaaaccaacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgagccacgtgtggctttttgtatgtaccgtatgaataaggatcggctaactccgtgccagcagccgcggtaatacggaggatccgagcgttatccggatttattgggtttaaagggagcgtaggcgggttgttaagtcagttgtgaaagtttgcggctcaaccgtaaaattgcagttgatactggcgaccttgagtgcaacagaggtaggcggaattcgtggtgtagcggtgaaatgcttagatatcacgaagaactccgattgcgaaggcagcttactggattgtaactgacgctgatgctcgaaagtgtgggtatcaaacaggattagataccctggtagtccacacagtaaacgatgaatactcgctgttggcgatatacagtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaactgacggaaccggaaacggttctttcttcggacagttgtgaaggtgctgcatggttgtcgtcagctcgtgccgtgaggtgtcggcttaagtgccataacgagcgcaacccttacgggtagttaccatcaggttatgctggggactctacccggactgccgtcgtaagatgtgaggaaggtggggatgacgtcaaatcagcacggcccttacgtccggggctacacacgtgttacaatggggggtacagaaggcagctacacggcgacgtggtgctaatcccgaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctnaagtacgta

SEQ ID NO: 75 - DNA, 1372 bp, synthetic construct
  misc_feature 1..1372: note = Synthetic: FBI00126
  source 1..1372: mol_type = other DNA
tgcagtcgaacgggancccaggagcttgctcctgggtgagagtggcgaacgggtgagtaatgcgtgaccgacctgccccatacaccggaatagctcctggaaacgggtggtaatgccggatgctccagttggatgcatgtccttctgggaaagattcatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccccagagatggggcctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacactgtgaggtggagcggatcccttaaaaccggtttcagttcggattggagtttgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcc

SEQ ID NO: 76 - DNA, 1190 bp, synthetic construct
  misc_feature 1..1190: note = Synthetic: FBI00127
  source 1..1190: mol_type = other DNA
nnnnnnnnnntgnaagtcgaacggcacctgccctcgggcagaagcgagtggcgaacggctgagtaacacgtggagaacctgccccctcccccgggatagccgcccgaaaggacgggtaataccggataccccggggtgccgcatggcaccccggctaaagccccgacgggaggggatggctccgcggcccatcaggtagacggcggggtgacggcccaccgtgccgacaacgggtagccgggttgagagaccgaccggccagattgggactgagacacggcccagactcctacgggaggcagcagtggggaatcttgcgcaatggggggaaccctgacgcagcgacgccgcgtgcgggacggaggccttcgggtcgtaaaccgctttcagcagggaagagtcaagactgtacctgcagaagaagccccggctaactacgtgccagcagccgcggtaatacgtagggggcgagcgttatccggattcattgggcgtaaagcgcgcgtaggcggcccggcaggccgggggtcgaagcggggggctcaaccccccgaagcccccggaacctccgcggcttgggtccggtaggggagggtggaacacccggtgtagcggtggaatgcgcagatatcgggtggaacaccggtggcgaaggcggccctctgggccgagaccgacgctgaggcgcgaaagctgggggagcgaacaggattagataccctggtagtcccagccgtaaacgatggacgctaggtgtgggggggggnnnatncccccctgncncncncnacncacttnnngncncncccgnggagnnnngncncnnngngtaaantctnannnnttnacngggggncnnncnaannnnngnagnatgtggnttnnntttnnannnnncgnannnnntttnnnnggtnnacatatgggngnnacgggggnnancccnngggnnnnnangnnnccctanngnnggngcanngctnnnncnannnnnnnnnngnnnnnnnnnnggnnnnnnccnnnnnnnnnnnnnnnccnnccncgnngnntnnnnnnnnnggngnnngnncnngnancnnnnnnnnncnnnnccnnnnnnnggnnnanannnnngnnnnnnnnnnntnnnnnnnnnnnnnnnnnnnnnnnnnnnnnnnannnnnnnnnnnnnncnncnnngggannnnnan

SEQ ID NO: 77 - DNA, 1402 bp, synthetic construct
  misc_feature 1..1402: note = Synthetic: FBI00128
  source 1..1402: mol_type = other DNA
annnntgcangtcgagcgaagcggtttcgatgaagttttcggatggatttgaaatcgacttagcggcggacgggtgagtaacgcgtgggtaacctgccttacactgggggataacagttagaaatgactgctaataccgcataagcgcacagggccgcatggtctggtgtgaaaaactccggtggtgtaagatggacccgcgtctgattaggtagttggtggggtaacggcccaccaagccgacgatcagtagccgacctgagagggtgaccggccacattgggactgagacacggcccaaactcctacgggaggcagcagtggggaatattggacaatgggcgaaagcctgatccagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggttaagcaagtctgaagtgaaagcccggggctcaaccccggtactgctttggaaactgtttgacttgagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgtcgggggacaaagtccttcggtgccgccgctaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccattgaaaatcatttaaccgtgatccctcttcggagcaatggagacaggtggtgcatggttgtcgtcagctcgtgtcgtgagatgttgggttaagtcccgcaacgagcgcaacccttatccttagtagccagcaagtaatgttgggcactctggggagactgccagggataacctggaggaaggtggggatgacgtcaaatcatcatgccccttatgatttgggctacacacgtgctacaatggcgtaaacaaagggaagcaaaggagcgatctggagcaaaccccaaaaataacgtctcagttcggattgcaggctgcaactcgcctgcatgaagctggaatcgctagtaatcgcgaatcagaatgtcgcggtgaatacgttcccgggtcttgtacacaccgcccgtcacaccatgggagttggtaacgcccgaagtcagtgacccaaccgtaaggagggagc

SEQ ID NO: 78 - DNA, 1341 bp, synthetic construct
  misc_feature 1..1341: note = Synthetic: FBI00132
  source 1..1341: mol_type = other DNA
tcgaacgattaagacggctccggccgtgaatagagtggcgaacgggtgagtaacacgtgaccaacctgccccctaccccgggataacgcaaggaaacctgcgctaataccggatactccttcccctgctcctgcaggggtcgggaaagcccagacggtaggggatggggtcgcggcccattaggtagacggcggggcaacggcccaccgtgcctgcgatgggtagccgggttgagagaccgaccggccacattgggactgagatacggcccagactcctacgggaggcagcagtggggaattttgcgcaatggggggaaccctgacgcagcaacgccgcgtgcgggatgaaggccttcgggttgtaaaccgctttcagcagggaagatagtgacggtacctgcagaagaagccccggctaactacgtgccagcagctnnggtaatacgtagggggcgagcgttatccggattcattgggcgtaaagcgcgcgtaggcggcccgtcaagcggaacctctaacccgagggctcaacccccggtcgggttccgaactggcaggctcgagtttggtagaggaagatggaattcccggtgtagcggtggaatgcgcagatatcgggaagaacaccgatggcgaaggcagtcttctgggccatcaactgacgctgangcgcgaaagctgggggagcgaacaggattagataccctggtagtcccagccgtaaacgatgggtgctaggtgtggggggatcatccctccgtgccgcagccaacgcattaagcaccccgcctggggagtacggccgcaaggctaaaactcaaaggaattgacgggggcccgcacaagcagcggagcatgtggcttaattcgaagccaacgcgaagaaccttaccagggcttgacatgctagtgaagccgggaaacccggtggccgagaggagctagcgcaggtggtgcatggctgtcgtcagctcgtgtcgtgagatgttgggttaagtcccgcaacgagcgcaacccctatcgtatgttgccagcattgagttggggactcatacgagacngccggcgtcaagccggaggaaggtggggacgacgtcaagtcatcatgccctttatgccctgggctgcacacgtgctacaatggccggtacaacgggccgcgacctggcgacaggaagcgaatccctcaaagccggccccagttcggatcggaggctgcaacccgcctccgtgaagtcggagttgctagtaatcgcggatcagcatgccgcggtgaatacgttcccgggccttgtacacaccgcccgtcacaccacccgagtcgtctgcacccgaagcc

SEQ ID NO: 79 - DNA, 1395 bp, synthetic construct
  misc_feature 1..1395: note = Synthetic: FBI00133
  source 1..1395: mol_type = other DNA
agtcgaacggcagcatgggggcaaccctgatggcgagtggcgaacgggtgagtaatatatcggaacgtacccaagagtgggggacaaccagtcgaaagattggctaataccgcatacgatctaaggatgaaagtgggggaccttcgggcctcacgctcctggagcggccgatagctgattagctagttggtggggtaaaggcctaccaaggcgatgatcagtagctggtctgagaggacgatcagccacactgggactgagacacggcccagactcctacgggaggcagcagtggggaattttggacaatgggggaaaccctgatccagcaatgccgcgtgagtgaagaaggccttcgggttgtaaagctcttttgtcagggaagaaattgattgggctaatatcccgattagatgacggtacctgaagaataagcaccggctaactacgtgccagcagccgcggtaatacgtagggtgcgagcgttaatcggaattactgggcgtaaagcgtgcgcaggcggttgtgtaagacagatgtgaaatgcccgggctcaacctgggaattgcatttgtgactgcacggctagagtgtgtcagaggggggtagaattccacgtgtagcagtgaaatgcgtagatatgtggaggaataccgatggcgaaggcagccccctgggataacactgacgctcatgcacgaaagcgtggggagcaaacaggattagataccctggtagtccacgccctaaacgatgtcgactagttgttgggtcttaattgacttagtaacgcagctaacgcgtgaagtcgaccgcctggggagtacggtcgcaagattaaaactcaaaggaattgacggggacccgcacaagcggtggatgatgtggattaattcgatgcaacgcgaaaaaccttacctacccttgacatgtcaggaatcccgaagagatttgggagtgctcgaaagagagcctgaacacaggtgctgcatggctgtcgtcagctcgtgtcgtgagatgttgggttaagtcccgcaacgagcgcaacccttgtcattagttgctacgaaagggcactctaatgaaactgccggtgacaaaccggaggaaggtggggatgacgtcaagtcctcatggcccttatgggtagggcttcacacgtcatacaatggtgcatacagagggttgccaacccgagagggggagctaatctcagaaagtgcatcgtagtccggattgaagtctgcaactcgacttcatgaagttggaatcgctagtaatcgcggatcagcatgtcgcggtgaatacgttcccgggtcttgtacacaccgcccgtcacaccatgggagcgggttttaccagaagtaggtagcttaaccgtaaggagggcnntnc

SEQ ID NO: 80 - DNA, 1389 bp, synthetic construct
  misc_feature 1..1389: note = Synthetic: FBI00135
  source 1..1389: mol_type = other DNA
agtcgaacgggnnccatcaggctttgcttggtggtgagagtggcgaacgggtgagtaatgcgtgaccgacctgccccatacaccggaatagctcctggaaacgggtggtaatgccggatgctccgactcctcgcatggggtgtcgggaaagattatatcggtatgggatggggtcgcgtcctatcaggtagtcggcggggtaacggcccaccgagcctacgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgatcgggagcaagccttcgggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatctgcgccgggtacgggcgggctggagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatatggcctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgtccagggcttcacgcatgctacaatggccggtacaacgggatgcgacacggcgacgtggagcggatccctgaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcctaaccctttgngganggagc

SEQ ID NO: 81 - DNA, 1384 bp, synthetic construct
  misc_feature 1..1384: note = Synthetic: FBI00137
  source 1..1384: mol_type = other DNA
tcgaggggcatcaggaagaaagcttgctttctttgctggcgaccggcgcacgggtgagtaacacgtatccaacctgccctttactcggggatagcctttcgaaagaaagattaatacccgatagcataatgattccgcatggtttcattattaaaggattccggtaaaggatggggatgcgttccattaggttgttggtgaggtaacggctcaccaagccttcgatggataggggttctgagaggaaggtcccccacattggaactgagacacggtccaaactcctacgggaggcagcagtgaggaatattggtcaatgggcgctagcctgaaccagccaagtagcgtgaaggatgaaggctctatgggtcgtaaacttcttttatataagaataaagtgcagtatgtatactgttttgtatgtattatatgaataaggatcggctaactccgtgccagcagccgcggtaatacggaggatccgagcgttatccggatttattgggtttaaagggagcgtaggtggactggtaagtcagttgtgaaagtttgcggctcaaccgtaaaattgcagttgatactgtcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagtggaatgatgtggaaacatgtcagtgagcaatcaccgctgtgaaggtgctgcatggttgtcgtcagctcgtgccgtgaggtgtcggcttaagtgccataacgagcgcaacccttatcttcagttactaacaggtcatgctgaggactctggagagactgccgtcgtaagatgtgaggaaggtggggatgacgtcaaatcagcacggcccttacgtccggggctacacacgtgttacaatggggggtacagaaggcagctagcgggtgaccgtatgctaatcccaaaatcctctctcagttcggatcgaagtctgcaacccgacttcgtgaagctggattcgctagtaatcgcgcatcagccacggcgcggtgaatacgttcccgggccttgtacacaccgcccgtcaagccatgggagccgggggtacctgaagtacgnaaccgcaag

SEQ ID NO: 82 - DNA, 1414 bp, synthetic construct
  misc_feature 1..1414: note = Synthetic: FBI00140
  source 1..1414: mol_type = other DNA
gcagtcgaacggagaattttatttcggtagaattcttagtggcgaacgggtgagtaacgcgtaggcaacctgccctttagacggggacaacattccgaaaggagtgctaataccggatgtgatcatcttgccgcatggcaggatgaagaaagatggcctctacaagtaagctatcgctaaaggatgggcctgcgtctgattagctagttggtagtgtaacggactaccaaggcgatgatcagtagccggtctgagaggatgaacggccacattgggactgagacacggcccaaactcctacgggaggcagcagtggggaatcttccgcaatggacgaaagtctgacggagcaacgccgcgtgagtgatgaaggatttcggtctgtaaagctctgttgtttatgacgaacgtgcagtgtgtgaacaatgcattgcaatgacggtagtaaacgaggaagccacggctaactacgtgccagcagccgcggtaatacgtaggtggcgagcgttgtccggaattattgggcgtaaagagcatgtaggcggcttaataagtcgagcgtgaaaatgcggggctcaaccccgtatggcgctggaaactgttaggcttgagtgcaggagaggaaaggggaattcccagtgtagcggtgaaatgcgtagatattgggaggaacaccagtggcgaaggcgcctttctggactgtgtctgacgctgagatgcgaaagccagggtagcgaacgggattagataccccggtagtcctggccgtaaacgatgggtactaggtgtaggaggtatcgaccccttctgtgccggagttaacgcaataagtaccccgcctggggagtacggccgcaaggttgaaactcaaaggaattgacgggggcccgcacaagcggtggagtatgtggtttaattcgacgcaacgcgaagaaccttaccaaggcttgacattgattgaacgctctagagatagagctttcccttcggggacaagaaaacaggtggtgcatggctgtcgtcagctcgtgtcgtgagatgttgggttaagtcccgcaacgagcgcaacccctatcctatgttaccagcaagtaaagttggggactcatgggagactgccagggacaacctggaggaaggcggggatgacgtcaagtcatcatgccccttatgtcttgggctacacacgtactacaatggtcggaaacagagggaagcgaagccgcgaggcagagcaaaccccagaaacccgatctcagttcggatcgcaggctgcaacccgcctgcgtgaagtcggaatcgctagtaatcgcaggtcagcatactgcggtgaatacgttcccgggccttgtacacaccgcccgtcacaccacgaaagttggtaacacccgaagccggtgaggtaanctattag

SEQ ID NO: 83 - DNA, 1230 bp, synthetic construct
  misc_feature 1..1230: note = Synthetic: FBI00145
  source 1..1230: mol_type = other DNA
nnnnnnnnnagtcgaacgggancccaggagcttgctcctgggtgagagtggcgaacgggtgagtaatgcgtgaccgacctgccccatacaccggaatagctcctggaaacgggtggtaatgccggatgctccagttggatgcatgtccttctgggaaagattcatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggancatgcggattaattcgatgcaacgcgaagaaccttacctgggcttgacatgttcccgacagcnntagagatnnngtctcccttcggggcgggntcacagnggngcatggncgtcgtcagctcgtgtcgtgagangntgggttaagtcccgcaacgancgcaaccctcgccctnnnnnncnncacgtcnngnnggnnntcacgggggaccnnngggnnactnnnnaaggtggggntnacgtcnnatcatcatncnnnnncgtcaggnntnnncgcanncnacnannnnnnnnnnnnngnnnnngnnannnnnnnnnnccnnaacnngnnnncnnntnnnnnnntnnnnnan

SEQ ID NO: 84 - DNA, 1392 bp, synthetic construct
  misc_feature 1..1392: note = Synthetic: FBI00147
  source 1..1392: mol_type = other DNA
tgcagtcgnncgaagcaattaaaatgaagttttcggatggatttttgattgactgagtggcggacgggtgagtaacgcgtggataacctgcctcacactgggggataacagttagaaatgactgctaataccgcataagcgcacagtaccgcagggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcaagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagaga

SEQ ID NO: 85 - DNA, 1393 bp, synthetic construct
  misc_feature 1..1393: note = Synthetic: FBI00149
  source 1..1393: mol_type = other DNA
tgcagtcgagcgagaaatttttaacggattccttcggggagaagataaggatggaaagcggcggacgggtgagtaacgcgtgagtaacctgcctttaggagggggacaacattccgaaagggatgctaataccgcataaaattattgtatcgcatggtatgataatcaaagatttatcgcctaaagatggactcgcgtccgattagctagttggtggggtaaaagcctaccaaggcgacgatcggtagccgaactgagaggttgatcggccacattgggactgagacacggcccagactcctacgggaggcagcagtgggggatattgcgcaatgggggaaaccctgacgcagcaacgccgcgtgaaggaagaaggccttcgggttgtaaacttctttaagtgtggaagataatgacggtacacacagaataagccacggctaactacgtgccagcagccgcggtaatacgtaggtggcaagcgttgtccggatttactgggtgtaaagggcgtgtaggcgggtagacaagtcagatgtgaaataccggggctcaactccggggctgcatttgaaactgtatatcttgagtgtcggagaggaaagcggaattcctagtgtagcggtgaaatgcgtagatattaggaggaacaccagtggcgaaggcggctttctggacgataactgacgctgaggcgcgaaagcgtggggagcaaacaggattagataccctggtagtccacgccgtaaacgatggatactaggtgtaggaggtatcgaccccttctgtgccgcagttaacacaataagtatcccacctggggagtacggtcgcaagattgaaactcaaaggaattgacgggggcccgcacaagcagtggagtatgtggtttaattcgaagcaacgcgaagaaccttaccaggacttgacatcccacgcatagcctagagataggtgaagtcctacgggacgtggagacaggtggtgcatggttgtcgtcagctcgtgtcgtgagatgttgggttaagtcccgcaacgagcgcaacccttactgtcagttaccatcattaagttggggactctggcaggactgccggtgacaaatcggaggaaggtggggacgacgtcaaatcatcatgccccttatgtcctgggctacacacgtactacaatggctgtaaacagagtgaagcaaagcagtgatgtggagcaaaacacaaaaagcagtctcagttcagattgtaggctgaaactcgcctatatgaagtcggaattgctagtaatcgcagatcagcatgctgcggtgaatacgttcccgggccttgtacacaccgcccgtcacaccatgagagtcgataacacccgaagcctgtagcttaaccttagggagagc

SEQ ID NO: 86 - DNA, 1527 bp, synthetic construct
  misc_feature 1..1527: note = Synthetic: FBI00151
  source 1..1527: mol_type = other DNA
tggagagtttgatcctggctcaggatgaacgctggcggcgtgcctaacacatgcaagtcgaacgaagcgactggaggaagttttcggatggaatccggttgactgagtggcggacgggtgagtaacgcgtggataacctgcctcacactgggggataacagttagaaatggctgctaataccgcataagcgcacagtaccgcatggtacggtgtgaaaaacccaggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcggaagcctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgacgcaagtctggagtgaaagcccggggcccaaccccgggactgctttggaaactgtgctgctggagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatgctaggtgtcggggggcaaagcccttcggtgccgccgctaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggymcgtaacggkgcccttccttcgggacaggggagacaggtggtgcatggttgtcgtcagctcgtgtcgtgagatgttgggttaagtcccgcaacgagcgcaacccttatccttagtagccagcacgtsrtggtgggcactctagggagactgccagggataacctggaggaaggtggggatgacgtcaaatcatcatgccccttatgatttgggctacacacgtgctacaatggcgtaaacaaagggaggcgaccctgcgaaggcaagcaaatcccaaaaataacgtcccagttcggactgtagtctgcaacccgactacacgaagctggaatcgctagtaatcgcgratcagaatgycgcggtgaatacgttcccgggtcttgtacacaccgcccgtcacaccatgggagtcagcaacgcccgaagtcagtgacccaacctkaamrggagggagctgccgaaggcggggcaggtaactggggtgaagtcgtaacaaggtagccgtatcggaaggtgcggytggatcacctcctt

SEQ ID NO: 87 - DNA, 1429 bp, synthetic construct
  misc_feature 1..1429: note = Synthetic: FBI00152
  source 1..1429: mol_type = other DNA
tcgaacgaaaagagggaaagagcttgctctttncngaattgagtggcaaacgggtgagtaacacgtaaacaacctgccttcaggatggggacaacagacggaaacgactgctaataccgaataagttccaagagccgcatggcccatggaagaaaaggtggcctctacctgtaagctatcgcctgaagaggggtttgcgtctgattagctggttggaggggtaacggcccaccaaggcgacgatcagtagccggtctgagaggatgaacggccacactggaactgagacacggtccagactcctacgggaggcagcagtggggaatcttccgcaatgggcgaaagcctgacggagcaacgccgcgtgagtgatgacggccttcgggttgtaaaactctgtgatccgggacgaaaaggcagagtgcgaagaacaaactgcattgacggtaccggaaaagcaagccacggctaactacgtgccagcagccgcggtaatacgtaggtggcaagcgttgtccggaattattgggcgtaaagcgcgcgcaggcggcttcccaagtccctcttaaaagtgcggggcttaaccccgtgatgggaaggaaactgggaagctggagtatcggagaggaaagtggaattcctagtgtagcggtgaaatgcgtagagattaggaagaacaccggtggcgaaggcgactttctggacgaaaactgacgctgaggcgcgaaagcgtggggagcaaacaggattagataccctggtagtccacgccgtaaacgatggatactaggtgtaggaggtatcgacccctcctgtgccggagttaacgcaataagtatcccgcctgggaagtacgatcgcaagattaaaactcaaaggaattgacgggggcccgcacaagcggtggagtatgtggtttaattcgacgcaacgcgaagaaccttaccaggtcttgacattgatcgcgatctgcagaaatgcggagttcttcttcggaagacgagaaaacaggtggtgcacggctgtcgtcagctcgtgtcgtgagatgttgggttaagtcccgcaacgagcgcaacccctatcatttgttgccagcacgtaaaggtggggactcaaatgagaccgccgcggacaacgcggaggaaggcggggacgacgtcaagtcatcatgccccttatgacctgggctacacacgtactacaatgggtgtcaacaaagagaagcgaacccgcgagggagagcaaagctcaaaaacacacccccagttcagatcgcaggctgcaacccgcctgcgtgaagtaggaatcgctagtaatcgcgggtcagcataccgcggtgaatacgttcccgggccttgtacacaccgcccgtcacactatgagagtcagaaacacccgaagccggtgaggtaaccgcaaggagccnncc

SEQ ID NO: 88 - DNA, 1396 bp, synthetic construct
  misc_feature 1..1396: note = Synthetic: FBI00159
  source 1..1396: mol_type = other DNA
tgcagtcgaacgaagttagacagaggaagttttcggatggaatcggtataacttagtggcggacgggtgagtaacgcgtgggaaacctgccctgtaccgggggataacacttagaaataggtgctaataccgcataagcgcacggaaccgcatggttgtgtgtgaaaaactccggtggtacaggatggtcccgcgtctgattagccagttggcagggtaacggcctaccaaagcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaaacccagggctcaaccttgggattgcatttggaactgccaggctggagtgcaggagaggtaagcggaattcctagtgtagcggtgaaatgcgtagatattaggaggaacaccagtggcgaaggcggcttactggactgtaactgacgttgaggctcgaaagcgtggggagcaaacaggattagataccctggtagtccacgcggtaaacgatgattgctaggtgtaggtgggtatggacccatcggtgccgcagctaacgcaataagcaatccacctggggagtacgttcgcaagaatgaaactcaaaggaattgacggggacccgcacaagcggtggagcatgtggtttaattcgaagcaacgcgaagaaccttaccaagtcttgacatccccttgaagatcttgtaaagaggcattcccttcggggcaagggagacaggtggtgcatggttgtcgtcagctcgtgtcgtgagatgttgggttaagtcccgcaacgagcgcaacccttattcttagtagccagcaggtaaagctgggcactctaaggagactgccggggataacccggaggaaggcggggatgacgtcaaatcatcatgccccttatgatttgggctacacacgtgctacaatggcgtaaacaaagggaagcgagacagtgatgtggagcaaatcccagaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggaaatgcccgaagtctgtgacctaaccgaaagggannagc

SEQ ID NO: 89 - DNA, 1395 bp, synthetic construct
  misc_feature 1..1395: note = Synthetic: FBI00162
  source 1..1395: mol_type = other DNA
tgcagtcgaacgggnnccnggcagcttgctgcctggtgagagtggcgaacgggtgagtaatgcgtgaccgacctgccccatacaccggaatagctcctggaaacgggtggtaatgccggatgctccgactcctcgcatggggtgtcgggaaagattatatcggtatgggatggggtcgcgtcctatcaggtagtcggcggggtaacggcccaccgagcctacgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgatcgggagcaagccttcgggtgagtgtacctttcgaataagcaccggctaactacgtgccagcagccgcggtaatacgtagggtgcaagcgttatccggaattattgggcgtaaagggctcgtaggcggtttcgtcgcgtccggtgtgaaagtccatcgcttaacggtggatctgcgccgggtacgggcgggctggagtgcggtaggggagactggaattccccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtgggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatgcggcctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgtccagggcttcacgcatgctacaatggccggtacaacgggatgcgacatggcgacatggagcggatccctgaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcctaaccccttgtggganggagc

SEQ ID NO: 90 - DNA, 1384 bp, synthetic construct
  misc_feature 1..1384: note = Synthetic: FBI00165
  source 1..1384: mol_type = other DNA
tcgaggggcagcatggtcttagcttgctaaggccgatggcgaccggcgcacgggtgagtaacgcgtatccaacctgccttacactcttggacagccttctgaaagggagattaatacaagatgttatcatgagtaagcattttcgcatgattaaaggtttaccggtgtaagatggggatgcgttccattagatagtaggcggggtaacggcccacctagtcttcgatggataggggttctgagaggaaggtcccccacattggaactgagacacggtccaaactcctacgggaggcagcagtgaggaatattggtcaatggacgagagtctgaaccagccaagtagcgtgaaggatgaaggttctatggattgtaaacttcttttatacgggaataaacgaatccacgcgtggatttttgcatgtaccgtatgaataaggatcggctaactccgtgccagcagccgcggtaatacggaggatccgagcgttatccggatttattgggtttaaagggagcgtagatgggttgttaagtcagttgtgaaagtttgcggctcaaccgtaaaattgcaattgatactggcagtcttgagtacagttgaggtaggcggaattcgtggtgtagcggtgaaatgcttagatatcacgaagaactccgattgcgaaggcagcttactaacctgtaactgacattgatgctcgaaagtgtgggtatcaaacaggattagataccctggtagtccacacggtaaacgatgaatactcgctgtaggcgatatacggtctgcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaccgaatatggcggaaacgccatagctagcaatagcggttgtgaaggtgctgcatggttgtcgtcagctcgtgccgtgaggtgtcggcttaagtgccataacgagcgcaacccttgccgatagttactaacaggttatgctgaggactctgtcgggactgccatcgtaagatgtgaggaaggtggggatgacgtcaaatcagcacggcccttacgtccggggctacacacgtgttacaatggggggtacagagggctgctaccacgcaagtggatgccaatcccaaaaacctctctcagttcggattgaagtctgcaacccgacttcatgaagctggattcgctagtaatcgcgcatcagccacggcgcggtgaatacgttcccgggccttgtacacaccgcccgtcaagccatgggagccgggggtacctgaagtgcgtaaccgcaa

SEQ ID NO: 91 - DNA, 1391 bp, synthetic construct
  misc_feature 1..1391: note = Synthetic: FBI00167
  source 1..1391: mol_type = other DNA
tcgagcgaagcacttnnggtnngattcttcggatgannnnntttgtgactgagcggcggacgggtgagtaacgcgtgggtaacctgcctcatacagggggataacagttagaaatgactgctaataccgcataagaccacggtaccgcatggtacagtggtaaaaactccggtggtatgagatggacccgcgtctgattaggtagttggtggggtaacggcctaccaagccgacgatcagtagccgacctgagagggtgaccggccacattgggactgagacacggcccagactcctacgggaggcagcagtggggaatattgcacaatggaggaaactctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcacggcaagccagatgtgaaagcccggggctcaaccccgggactgcatttggaactgctgagctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactgctaggtgtcgggtggcaaagccattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagcttttcttcggaacatcggtgacaggtggtgcatggttgtcgtcagctcgtgtcgtgagatgttgggttaagtcccgcaacgagcgcaacccctatcttcagtagccagcaggtantgttgggcactctggagagactgccagggataacctggaggaaggtggggatgacgtcaaatcatcatgccccttatgaccagggctacacacgtgctacaatggcgtaaacaaagagaagcgaactcgcgagggtaagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgtaaggagggag

SEQ ID NO: 92 - DNA, 1374 bp, synthetic construct
  misc_feature 1..1374: note = Synthetic: FBI00170
  source 1..1374: mol_type = other DNA
tgcagtcnnnnatgaaaccgccctcgggcggacatgaagtggcgaacgggtgagtaacacgtgaccaacctgcccccctctccgggacaaccttgggaaaccgaggctaataccggatactccctcccctgctcctgcaggggtcgggaaagcccaggcggagggggatggggtcgcggcccattaggtagtaggcggggtaacggcccacctagcccgcgatgggtagccgggttgagagaccgaccggccacattgggactgagatacggcccagactcctacgggaggcagcagtggggaattttgcgcaatgggggaaaccctgacgcagcaacgccgcgtgcgggacgacggccttcgggttgtaaaccgctttcagcagggaagaaattcgacggtacctgcagaagaagctccggctaactacgtgccagcagccgcggtaatacgtagggagcgagcgttatccggaatcattgggcgtaaagagcgcgtaggcggcctctcaagcgggatctctaatccgagggctcaaccccccggccggatcccgaactgggaggctcgagttcggtagaggcaggcggaattcccggtgtagcggtggaatgcgcagatatcgggaagaacaccgatggcgaaggcagcctgctgggccgcaactgacgctgaggcgcgaaagctaggggagcgaacaggattagataccctggtagtcctagccgtaaacgatggatactaggtgtgggggggctccgccctccgtgccgcagcccaacgcattaagtatcccgcctggggagtacggccgcaaggctaaaactcaaaggaattgacgggggcccgcacaagcagcggagcatgtggcttaattcgaagcaacgcgaagaaccttaccagggcttgacatggacgtgaagccggggaaacccggtggccgagaggagcgtccgcaggtggtgcatggctgtcgtcagctcgtgtcgtgagatgttgggttaagtcccgcaacgagcgcaacccctgccccatgttgccagcattaggttggggactcatgggggactgccggcgtcaagccggaggaaggtggggacgacgtcaagtcatcatgccctttatgccctgggctgcacacgtgctacaatggccggtacaacgggctgcgagaccgcgaggtcgagcgaatccctcaaagccggccccagttcggatcggaggctgcaacccgcctccgtgaagtcggagttgctagtaatcgcggatcagcatgccgcggtgaatacgttcccgggccttgtacacaccgcccgtcacaccacccgagtcgtctgcacccgaagccgccggccgaacccgcaaggggcggag

SEQ ID NO: 93 - DNA, 1396 bp, synthetic construct
  misc_feature 1..1396: note = Synthetic: FBI00171
  source 1..1396: mol_type = other DNA
tcgaacgtgaaagtccttcgggacgagtaaagtggcgcacgggtgagtaacgcgtggataatctacccttaagatggggataacggctggaaacggtcgctaataccgaatacgctcccgattttatcattggggggaaagatggcctctgcttgcaagctatcgcttaaggatgagtccgcgtcccattagctagttggcggggtaacggcccaccaaggcaacgatgggtagccggtctgagaggatgaccggccacactggaactggaacacggtccagactcctacgggaggcagcagtggggaatattgcgcaatgggcgaaagcctgacgcagcgacgccgcgtgagggatgaaggttctcggatcgtaaacctctgtcaggggggaagaaaccccctcgtgtgaataatgcgagggcttgacggtacccccaaaggaagcaccggctaactccgtgccagcagccgcggtaatacggagggtgcaagcgttaatcggaatcactgggcgtaaagcgcacgtaggcggcttggtaagtcaggggtgaaatcccacagcccaactgtggaactgcctttgatactgccaggcttgagtaccggagagggtggcggaattccaggtgtaggagtgaaatccgtagatatctggaggaacaccggtggcgaaggcggccacctggacggtaactgacgctgaggtgcgaaagcgtgggtagcaaacaggattagataccctggtagtccacgctgtaaacgatgggtgctgggtgctgggatgtatgtctcggtgccgtagctaacgcgataagcaccccgcctggggagtacggtcgcaaggctgaaactcaaagaaattgacggggggcccgcacaagcggtggagtatgtggtttaattcgatgcaacgcgaagaaccttacccaggcttgacatctagggaacccttcggaaatgaaggggtgcccttcggggagccctaagacaggtgctgcatggctgtcgtcagctcgtgccgtgaggtgttgggttaagtcccgcaacgagcgcaacccctatcttcagttgccagcaggtaaggctgggcactctggagagaccgccccggtcaacggggaggaaggtggggacgacgtcaagtcatcatggcccttacgcctggggctacacacgtactacaatggcgcgcacaaagggtagcgagaccgcgaggtggagccaatcccaaaaaacgcgtcccagtccggattggagtctgcaactcgactccatgaagtcggaatcgctagtaattcgagatcagcatgctcgggtgaatgcgttcccgggccttgtacacaccgcccgtcacaccacgaaagtcggttttacccgaagccggtnagctaact

SEQ ID NO: 94 - DNA, 1389 bp, synthetic construct
  misc_feature 1..1389: note = Synthetic: FBI00174
  source 1..1389: mol_type = other DNA
gtcgaacgaagcatttnnnnacggattnnttcggnntgaagnnnnttatgactgagtggcggacgggtgagtaacgcgtgggtaacctgcctcacacagggggatagcagttggaaacggctgataataccgcataagcgcacagtaccgcatggtacagtgtgaaaaactccggtggtgtgagatggacccgcgtctgattagcttgttggcggggtaacggcccaccaaggcaacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggcatcacaagtcagaagtgaaagcccggggctcaaccccgggactgcttttgaaactgtggagctggagtgcaggagaggcaagtggaattcctagtgtagcggtgaaatgcgtagatattaggaggaacaccagtggcgaaggcggcttgctggactgtaactgacactgaggctcgaaagcgtggggagcaaacaggattagataccctggtagtccacgccgtaaacgatgaatactaggtgtcggggctcataagagcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcctcttgaccggtcagtaatgtgaccttttcttcggaacaagagtgacaggtggtgcatggttgtcgtcagctcgtgtcgtgagatgttgggttaagtcccgcaacgagcgcaacccctattcttagtagccagcatataaggtgggcactctaggaagactgccagggataacctggaggaaggtggggatgacgtcaaatcatcatgccccttatgacttgggctacacacgtgctacaatggcgtaaacaaagtgaagcgagagtgtgagcttaagcaaatcacaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcggaaatgcccgaagtcggtgacctaacgaaagaag

SEQ ID NO: 95 - DNA, 1386 bp, synthetic construct
  misc_feature 1..1386: note = Synthetic: FBI00175
  source 1..1386: mol_type = other DNA
gcagtcgnncgagaggaaggnaagcttgcttttctaaatctagtggcgaacgggtgagtaacacgtaggtaacctgcccatgtgcccgggataacttctggaaacggatgctaaaaccggataggtagcagacaagcatttgactgctattaaagtggctaaggccatgaacatggatggacctgcggtgcattagctagttggtgaggtaacggcccaccaaggcgacgatgcatagccggcctgagagggcggacggccacattgggactgagacacggcccaaactcctacgggaggcagcagtagggaattttcgtcaatggggggaaccctgaacgagcaatgccgcgtgagtgaggaaggtcttcggatcgtaaagctctgttgtaagagaaaaacggcactcatagggaatgatgagtgagtgatggtatcttaccagaaagtcacggctaactacgtgccagcagccgcggtaatacgtaggtggcgagcgttatccggaatgattgggcgtaaagggtgcgtaggtggcagatcaagtctggagtaaaaggtatgggctcaacccgtacttgctctggaaactgatcagctagagaacagaagaggacggcggaactccatgtgtagcggtaaaatgcgtagatatatggaagaacaccggtggcgaaggcggccgtctggtctggattctgacactgaagcacgaaagcgtggggagcaaataggattagataccctagtagtccacgccgtaaacgatgagaactaggtgttgggggaataactcagtgccgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatcccttgcagaggtatagagatatatccgaggttatcaaggagacaggtggtgcatggttgtcgtcagctcgtgtcgtgagatgttgggttaagtcccgcaacgagcgcaacccttgtgatatgttactaacattgagttgaggactcatatcagactgccggtgataaaccggaggaaggtggggatgacgtcaaatcatcatgccccttatggcctgggctacacacgtactacaatggcgtctacaaagagcggcaagcctgtgaaggcaagcgaatctcataaaggacgtctcagttcggattgaagtctgcaacccgacttcatgaagctggaatcgctagtaatcgcgaatcagcatgtcgcggtgaatacgttctcgggccttgtacacaccgcccgtcaaaccatgggagttggtaatgcccgaagccggtngnnta

SEQ ID NO: 96 - DNA, 1386 bp, synthetic construct
  misc_feature 1..1386: note = Synthetic: FBI00176
  source 1..1386: mol_type = other DNA
gnnagtcgancggagctgttttctctgaagttttcggatggaagagagttcagcttagtggcgaacgggtgagtaacacgtgagcaacctgcctttcagtgggggacaacatttggaaacgaatgctaataccgcataagaccacagtgtcgcatggcacaggggtcaaaggatttatccgctgaaagatgggctcgcgtccgattagctagatggtgaggtaacggcccaccatggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgtcccaggggacgataatgacggtaccctgggaggaagcaccggctaactacgtgccagcagccgcggtaaaacgtagggtgcaagcgttgtccggaattactgggtgtaaagggagcgcaggcggattggcaagttgggagtgaaatctatgggctcaacccataaattgctttcaaaactgtcagtcttgagtggtgtagaggtaggcggaattcccggtgtagcggtggaatgcgtagatatcgggaggaacaccagtggcgaaggcggcctactgggcactaactgacgctgaggctcgaaagcatgggtagcaaacaggattagataccctggtagtccatgccgtaaacgatgattactaggtgtgggaggattgaccccttccgtgccgcagttaacacaataagtaatccacctgggggagtacgaccgcaaggttgaaactcaaaggaattgacgggggcccgcacaagcagtgggagtatgtggtttaattcgaagcaacgcgaagaaccttaccaggtcttgacatcggatgcatacctaagagattagggaagtccttcgggacatccagacaggtggtgcatggttgtcgtcagctcgtgtcgtgagatgttgggttaagtcccgcaacgagcgcaacccttatcgttagttactacgcaagaggactctagcgagactgccgttgacaaaacggaggaaggtggggatgacgtcaaatcatcatgccctttatgacctgggctacacacgtactacaatggctattaacagagagaagcgataccgcgaggtggagcaaacctcacaaaaatagtctcagttcggatcgcaggctgcaacccgcctgcgtgaagccggaattgctagtaatcgcggatcagcatgccgcggtgaatacgttcccgggccttgtacacaccgcccgtcacaccatgagagccggggggacccgaagtcggtagtctaaccgcaag

SEQ ID NO: 97 - DNA, 1359 bp, synthetic construct
  misc_feature 1..1359: note = Synthetic: FBI00177
  source 1..1359: mol_type = other DNA
agaagcttgcttctcnccggcgacgagtggcgaacgggtgagtaatacatcggaacgtgtccgctcgtgggggacaaccagccgaaaggttggctaataccgcatgagttctacggaagaaagagggggacccgcaagggcctctcgcgagcggagcggccgatgactgattagccggttggtgaggtaacggctcaccaaagcaacgatcagtagctggtctgagaggacgaccagccacactgggactgagacacggcccagactcctacgggaggcagcagtggggaattttggacaatgggcgcaagcctgatccagctattccgcgtgtgggatgaaggccctcgggttgtaaaccacttttgtagagaacgaaaagacaccttcgaataaagggtgttgctgacggtactctaagaataagcaccggctaactacgtgccagcagccgcggtaatacgtagggtgcgagcgttaatcggaattactgggcgtaaagggtgcgcaggcggttgagtaagacagatgtgaaatccccgagcttaactcgggaatggcatatgtgactgctcgactagagtgtgtcagagggaggtggaattccacgtgtagcagtgaaatgcgtagatatgtggaagaacaccgatggcgaaggcagcctcctgggacataactgacgctcaggcacgaaagcgtggggagcaaacaggattagataccctggtagtccacgccctaaacgatgttaactagttgttgggaagtaaaattctcagtaacgcagccaacgcgagaagttaaccgcctgggaagtacggtcgcaagactaaaactcaaaggaattgacggggacccgcacaagcggtggatgatgtggattaattcgatgcaacgcgaaaaaccttacctacccttgacatgtcaggaagctcttgtaatgagagcgtgcccgcaagggagcctgaacacaggtgctgcatggctgtcgtcagctcgtgtcgtgagatgttgggttaagtcccgcaacgagcgcaacccttgtcactagttgctacgaaagggcactctagtgagactgccggtgacaaaccggaggaaggtggggatgacgtcaagtcctcatggcccttatgggtagggcttcacacgtcatacaatggtcggaacagagggcagcgaagtcgtgagacggagccaatcccagaaaaccgatcgtagtccggattgcagtctgcaactcgactgcatgaagtcggaatcgctagtaatcgcggatcagcatgccgcggtgaatacgttcccgggtcttgtacacaccgcccgtcaaacaatgggagtggtgtttaccagaagtcg

SEQ ID NO: 98 - DNA, 1391 bp, synthetic construct
  misc_feature 1..1391: note = Synthetic: FBI00180
  source 1..1391: mol_type = other DNA
tgcnagtcngaggggcagcattcgggtagcaatacctgagatggcgaccggcgcacgggtgcgtaacgcgtatgcaacctacccataacaggggcataacactgagaaattggtactaattccccataacattcgaagaggcatcttttcgggttgaaaactccggtggttatggatgggcatgcgttgtattagctggttggtgaggtaacggctcaccaaggcgacgatacatagggggactgagaggttaaccccccacattggtactgagacacggaccaaactcctacgggaggcagcagtgaggaatattggtcaatggacgcaagtctgaaccagccatgccgcgtgcaggaagacggctctatgagttgtaaactgcttttgtacgagggtaaactcagatacgtgtatctgactgaaagtatcgtacgaataaggatcggctaactccgtgccagcagccgcggtaatacggaggattcaagcgttatccggatttattgggtttaaagggtgcgtaggcggtctgataagttagaggtgaaatcccggggcttaactccggaattgcctctgatactgttgggctagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgctgtcggcgatacacagtcggtggctaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagcgacgattctggaaacaggatttcccttcggggcgcgaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggcagctaccctgcgaagggatgcgaatctcgaaagcctatctcagttcggattggaggctgaaacccgcctccatgaagttggattcgctagtaatcgcgcatcagccatggcgcggtgaatacgttcccgggccttgtacacaccgcccgtcaagccatggaagctgggggtgcctgaagttcgtgaccgncaagga

SEQ ID NO: 99 - DNA, 1392 bp, synthetic construct
  misc_feature 1..1392: note = Synthetic: FBI00182
  source 1..1392: mol_type = other DNA
angtcgaggggcagcatgaacttagctntgctaagtnttgatggcgaccggcgcacgggtgagtaacacgtatccaaccttccgtttactcagggatagcctttcgaaagaaagattaatacctgatagtatggtgagattgcatgatgacaccattaaagatttattggtaaacgatggggatgcgttccattaggtagtaggcggggtaacggcccacctagccggcgatggataggggttctgagaggaaggtcccccacattggaactgagacacggtccaaactcctacgggaggcagcagtgaggaatattggtcaatgggcgagagcctgaaccagccaagtagcgtgaaggatgaaggtcctacggattgtaaacttcttttatacgggaataaagtttcctacgtgtaggattttgtatgtaccgtatgaataagcatcggctaactccgtgccagcagccgcggtaatacggaggatgcgagcgttatccggatttattgggtttaaagggagcgcagacgggagattaagtcagttgtgaaagtttgcggctcaaccgtaaaattgcagttgatactggtttccttgagtgcagttgaggcaggcggaattcgtggtgtagcggtgaaatgcttagatatcacgaagaaccccgattgcgaaggcagcttgctaaactgtaactgacgttcatgctcgaaagtgtgggtatcaaacaggattagataccctggtagtccacacggtaaacgatggatactcgctgttggcgatatactgtcagcggccaagcgaaagcattaagtatcccacctggggagtacgccggcaacggtgaaactcaaaggaattgacgggggcccgcacaagcggaggaacatgtggtttaattcgatgatacgcgaggaaccttacccgggcttaaattgcagacgaattacgaggaaacttgtaagccgcaaggcgtctgtgaaggtgctgcatggttgtcgtcagctcgtgccgtgaggtgtcggcttaagtgccataacgagcgcaaccctcgtggtcagttactaacaggttaagctgaggactctggccagactgccatcgtaagatgtgaggaaggtggggatgacgtcaaatcagcacggcccttacgtccggggctacacacgtgttacaatgggaggtacagaaggccgctacccggcaacgggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgaaagccgggggtacctgaagtgcgtaaccgcaaggagc

SEQ ID NO: 100 - DNA, 1386 bp, synthetic construct
  misc_feature 1..1386: note = Synthetic: FBI00184
  source 1..1386: mol_type = other DNA
gcagtcgaggggcatcatgcttatagcaatataagtgatggcgaccggcgcacgggtgagtaacacgtatccaacctgccgataactcggggatagcctttcgaaagaaagattaatacccgatggcataatagaaccgcacggtttgattattaaagaatttcggttatcgatggggatgcgttccattaggcagttggtggggtaacggcccaccaaaccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtggtccacgtgtggatttttgtatgtaccatatgaataaggatcggctaactccgtgccagcagccgcggtaatacggaggatccgagcgttatccggatttattgggtttaaagggagcgtaggtggacagttaagtcagttgtgaaagtttgcggctcaaccgtaaaattgcagttgatactggctgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaatatattggaaacagtatagtcgtaagacaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctaatcccaaaagcctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaanng

SEQ ID NO: 101 - DNA, 1386 bp, synthetic construct
  misc_feature 1..1386: note = Synthetic: FBI00189
  source 1..1386: mol_type = other DNA
gaggggcagcattttagtttgcttgcaaactgaagatggcgaccggcgcacgggtgagtaacacgtatccaacctgccgataactccggaatagcctttcgaaagaaagattaataccggatagcatacgaatatcgcatgatatttttattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatgggcgagagcctgaaccagccaagtagcgtgaaggatgaaggctctatgggtcgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgtcactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaacagaatatattggaaacagtatagccgtaaggctgttgtgaaggtgctgcatggttgtcgtcagctcgtgccgtgaggtgtcggcttaagtgccataacgagcgcaacccttatctttagttactaacaggtcatgctgaggactctagagagactgccgtcgtaagangtgaggaaggtggggatgacgtcaaatcagcacggcccttacgtccggggctacacacgtgttacaatggggggtacagaaggcggctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagc

SEQ ID NO: 102 - DNA, 1390 bp, synthetic construct
  misc_feature 1..1390: note = Synthetic: FBI00190
  source 1..1390: mol_type = other DNA
agtcgaggggcagcattttagtttgcttgcaaactaaagatggcgaccggcgcacgggtgagtaacacgtatccaacctgccgataactcagggatagcctttcgaaagaaagattaataccggatggcataggattatcgcatgatgatcctattaaagaatttcggttatcgatggggatgcgttccattagtttgttggcggggtaacggcccaccaagacagcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtggtccacgtgtggatttttgtatgtaccgtatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgatactggcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacggtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattacatttgaataatctggaaacaggttagccgtaaggcaaatgtgaaggtgctgcatggttgtcgtcagctcgtgccgtgaggtgtcggcttaagtgccataacgagcgcaacccttatcttcagttactaacagggtcatgctgaggactctggagagactgccgtcgtaagatgtgaggaaggtggggatgacgtcaaatcagcacggcccttacgtccggggctacacacgtgttacaatggggggtacagaaggcagctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcgaggag

SEQ ID NO: 103 - DNA, 1497 bp, synthetic construct
  misc_feature 1..1497: note = Synthetic: FBI00191
  source 1..1497: mol_type = other DNA
tggagagtttgatcctggctcaggacgaacgctggcggcgtgcctaacacatgcaagtcgagcggrsmwcttcrrwkbycagcggcggacgggtgagtaacacgtgggtaacctgcctyrtagagggggatagcctyccgaaaggragattaataccgcatawmmtykcrrtatcgcatgataytkmraksaaaggagyaatctrctayragatggacccgcggcgcattagctagttggtgaggtaagggctcaccaaggcaacgatgcgtagccggcctkagagggtgaacggccacattggaactgagacacggtccagactcctacgggaggcagcagtggggaatattgcacaatgggcgaaagcctgatgcagcaacgccgcgtgagtgatgaaggccttcgggttgtaaagctctgtcttctgggacgataatgacggtaccagaggaggaagccacggctaactacgtgccagcagccgcggtaatacgtaggtggcaagcgttgtccggatttactgggygtaaaggatgcgtaggcggatatttaagtgagatgtgaaaatcccgrgcttaacttgggaactgcatttcaaactgggtatctagagtgcaggagaggaaagcggaattcctagtgtagcggtgaaatgcgtagagattaggaagaacaccagtggcgaaggcggctttctggactgtaactgacgctgaggcatgaaagcgtggggagcaaacaggattagataccctggtagtccacgccgtaaacgatgaatactaggtgtgggrrytgtcatgayytccgtgccgcagttaacacaataagtattccgcctggggagtacggtcgcaagattaaaactcaaaggaattgacgggggcccgcacaagcagcggagcatgtggtttaattcgaagcaacgcgaagaaccttacctagacttgacatctcctgaattactcgtaacggaggaagcccttcggggcaggaagacaggtggtgcatggttgtcgtcagctcgtgtcgtgagatgttgggttaagtcccgcaacgagcgcaacccttgtcgttagttgctaccattaagttgagcactctagcgagactgccgcggttaacgcggaggaaggtggggatgacgtcaaatcatcatgccccttatgtctagggctacacacgtgctacaatggtgagtacaacgagaaccgataccgcaaggtggaggaaaactcaaaaactcatcccagttcggattgcaggctgcaactcgcctgcatgaagctggagttgctagtaatcgcaaatcagaatgttgcggtgaatacgttcccgggccttgtacacaccgcccgtcacaccatgagagttgccaacacccgaagtccgtgaggtaaccgtaaggagccagcggccgaaggcgggggcagcgattggggtgaagtcgtaacaaggtagccgtaggagaacctgcggytggatcacctcctt

SEQ ID NO: 104 - DNA, 1528 bp, synthetic construct
  misc_feature 1..1528: note = Synthetic: FBI00194
  source 1..1528: mol_type = other DNA
tggagagtttgatcctggctcaggatgaacgctggcggcgtgcttaacacatgcaagtcgaacgaagcacyttdwtytgatttcttcggaatgaarawyttrgtgactgagtggcggacgggtgagtaacgcgtgggtaacctgcctcatacagggggataacagttagaaatgactgctaataccgcataagaccacagcrccgcatggtgcaggggtaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggagtggcaagtctgatgtgaaaacccggggctcaaccccgggactgcattggaaactgtcaatctagagtaccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgcygtaaacgatgactactaggtgtcgggcagcaaagctgttcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatctccctgaccggcaagtaatgttgcctttccttcgggacagggatgacaggtggtgcatggttgtcgtcagctcgtgtcgtgagatgttgggttaagtcccgcaacgagcgcaacccctatctttagtagccagcggttcggccgggcactctagagagactgccagggataacctggaggaaggtggggatgacgtcaaatcatcatgccccttatgagcagggctacacacgtgctacaatggcgtaaacaaagggargcagaaccgcgaggtcgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggtgggaccgataactggggtgaagtcgtaacaaggtagccgtatcggaaggtgcggytggatcacctcctt

SEQ ID NO: 105 - DNA, 1367 bp, synthetic construct
  misc_feature 1..1367: note = Synthetic: FBI00197
  source 1..1367: mol_type = other DNA
cggganccntcaagcttgcttggtggtgagagtggcgaacgggtgagtaatgcgtgaccgacctgccccatgctccggaatagctcctggaaacgggtggtaatgccggatgttccacatgatcgcatgtgattgtgggaaagattctatcggcgtgggatggggtcgcgtcctatcagcttgttggtgaggtaacggctcaccaaggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttgtttgggagcaagccttcgggtgagtgtacctttcgaataagcgccggctaactacgtgccagcagccgcggtaatacgtagggcgcaagcgttatccggatttattgggcgtaaagggctcgtaggcggctcgtcgcgtccggtgtgaaagtccatcgcttaacggtggatctgcgccgggtacgggcgggctggagtgcggtaggggagactggaattcccggtgtaacggtggaatgtgtagatatcgggaagaacaccgatggcgaaggcaggtctctgggccgtcactgacgctgaggagcgaaagcgtggggagcgaacaggattagataccctggtagtccacgccgtaaacggtggacgctggatgtggggcacgttccacgtgttccgtgtcggagctaacgcgttaagcgtcccgcctggggagtacggccgcaaggctaaaactcaaagaaattgacgggggcccgcacaagcggcggagcatgcggattaattcgatgcaacgcgaagaaccttacctgggcttgacatgttcccgacgacgccagagatggcgtttcccttcggggcgggttcacaggtggtgcatggtcgtcgtcagctcgtgtcgtgagatgttgggttaagtcccgcaacgagcgcaaccctcgccccgtgttgccagcacgttatggtgggaactcacgggggaccgccggggttaactcggaggaaggtggggatgacgtcagatcatcatgccccttacgtccagggcttcacgcatgctacaatggccggtacagcgggatgcgacatggcgacatggagcggatccctgaaaaccggtctcagttcggatcggagcctgcaacccggctccgtgaaggcggagtcgctagtaatcgcggatcagcaacgccgcggtgaatgcgttcccgggccttgtacacaccgcccgtcaagtcatgaaagtgggcagcacccgaagccggtggcctaaccc

SEQ ID NO: 106 - DNA, 1526 bp, synthetic construct
  misc_feature 1..1526: note = Synthetic: FBI00198
  source 1..1526: mol_type = other DNA
tggagagtttgatcctggctcaggatgaacgctggcggcgtgcctaacacatgcaagtcgaacgaagcatcyyataggaagttttcggatggaatatgggatgactgagtggcggacgggtgagtaacgcgtggataacctgcctcacactgggggataacagttagaaatggctgctaataccgcataagcgcacagtrccgcatggtacggtgtgaaaaacccaggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgatgcaagtctgaagtgaaagcccggggctcaaccccgggactgctttggaaactgtgtggctggagtgcaggagaggtaagtggaattcctagtgtagyggtgaaatgcgtagatattaggaggaacaccagtggcgaaggcggcttactggactgtaactgacgttgaggctcgaaagcgtggggagcaaacaggattagataccctggtagtccacgccgtaaacgatgaatgctaggtgtcggggggcaaagcccctcggtgccgccgctaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggacagtaacgtgtcccttccttcgggacaggggagacaggtggtgcatggttgtcgtcagctcgtgtcgtgagatgttgggttaagtcccgcaacgagcgcaacccttatccttagtagccagcayytcgggtgggcactctagggagactgccagggataacctggaggaaggtggggatgacgtcaaatcatcatgccccttatgatttgggctacacacgtgctacaatggcgtaaacaaagggargcrgccctgcgaaggtgagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagcaacgcccgaagtcagtgacccaaccgdaaggagggagctgccgaaggcggggcaggtaactggggtgaagtcgtaacaaggtagccgtatcggaaggtgcggytggatcacctcctt

SEQ ID NO: 107 - DNA, 1396 bp, synthetic construct
  misc_feature 1..1396: note = Synthetic: FBI00199
  source 1..1396: mol_type = other DNA
tgcagtcgaacgaagcaattaaaatgaagttttcggatggatttttgattgactgagtggcggacgggtgagtaacgcgtggataacctgcctcacactgggggataacagttagaaatgactgctaataccgcataagcgcacagtaccgcagggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttgggggggcaaagcccttcggtgccgtcgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcaagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagg

SEQ ID NO: 108 - DNA, 1397 bp, synthetic construct
  misc_feature 1..1397: note = Synthetic: FBI00200
  source 1..1397: mol_type = other DNA
tcgnacgaagtctttaggangcttgcttccaaagagacttagtggcgaacgggtgagtaacacgtaggtaacctgcccatgtgcccgggataactgctggaaacggtagctaaaaccggataggtatgagggaggcatcttcctcatattaaagcaccttcgggtgtgaacatggatggacctgcggcgcattagctggttggtgaggtaacggcccaccaaggcgatgatgcgtagccgacctgagagggtgaacggccacattgggactgagacacggcccaaactcctacgggaggcagcagtagggaattttcgtcaatggggggaaccctgaacgagcaatgccgcgtgtgtgaagaaggtcttcggatcgtaaagcactgttgtaagtgaagaatgccacatagaggaaatgctatgtgggtgacggtagcttaccagaaagccacggctaactacgtgccagcagccgcggtaatacgtaggtggcaagcgttatccggaatcattgggcgtaaagggtgcgtaggtggcacgataagtctgaagtaaaaggcaacagctcaactgttgtatgctttggaaactgtcgagctagagtgcagaagagggcgatggaattccatgtgtagcggtaaaatgcgtagatatatggaggaacaccagtggcgaaggcggtcgcctggtctgtaactgacactgatgcacgaaagcgtggggagcaaataggattagataccctagtagtccacgccgtaaacgatgagaactaagtgttggagagattcagtgctgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatggatataaatgttctagagatagaaagatagctatatatcacacaggtggtgcatggttgtcgtcagctcgtgtcgtgagatgttgggttaagtcccgcaacgagcgcaacccttgtcttctgttaccagcattaggttggggactcaggagagactgccggtgacaaaccggaggaaggtggggatgacgtcaaatcatcatgccccttatggcctgggctacacacgtactacaatggcgcctacaaagagcagcgacaccgcgaggtggagcgaatctcataaagggcgtctcagttcggattgaagtctgcaactcgacttcatgaagtcggaatcgctagtaatcgcagatcagcatgctgcggtgaatacgttctcgggccttgtacacaccgcccgtcaaaccatgggagttggtaatacccgaagccggtggcataaccgcaaggagngagc

SEQ ID NO: 109 - DNA, 1368 bp, synthetic construct
  misc_feature 1..1368: note = Synthetic: FBI00201
  source 1..1368: mol_type = other DNA
tgcagtcgaacgatgaaaccgccctcgggcggacatgaagtggcgaacgggtgagtaacacgtgaccaacctgcccccctctccgggacaaccttgggaaaccgaggctaataccggatactccctcccctgctcctgcaggggtcgggaaagcccaggcggagggggatggggtcgcggcccattaggtagtaggcggggtaacggcccacctagcccgcgatgggtagccgggttgagagaccgaccggccacattgggactgagatacggcccagactcctacgggaggcagcagtggggaattttgcgcaatgggggaaaccctgacgcagcaacgccgcgtgcgggacgacggccttcgggttgtaaaccgctttcagcagggaagaaattcgacggtacctgcagaagaagctccggctaactacgtgccagcagccgcggtaatacgtagggagcgagcgttatccggattcattgggcgtaaagagcgcgtaggcggcctctcaagcgggatctctaatccgagggctcaacccccggccggatcccgaactgggaggctcgagttcggtagaggcaggcggaattcccggtgtagcggtggaatgcgcagatatcgggaagaacaccgatggcgaaggcagcctgctgggccgcaactgacgctgaggcgcgaaagctaggggagcgaacaggattagataccctggtagtcctagccgtaaacgatggatactaggtgtggggggctccgccctccgtgccgcagccaacgcattaagtatcccgcctggggagtacggccgcaaggctaaaactcaaaggaattgacgggggcccgcacaagcagcggagcatgtggcttaattcgaagcaacgcgaagaaccttaccagggcttgacatggacgtgaagccggggaaacccggtggccgagaggagcgtccgcaggtggtgcatggctgtcgtcagctcgtgtcgtgagatgttgggttaagtcccgcaacgagcgcaacccctgccccatgttgccagcattaggttggggactcatgggggactgccggcgtcaagccggaggaaggtggggacgacgtcaagtcatcatgccctttatgccctgggctgcacacgtgctacaatggccggtacaacgggctgcgagaccgcgaggtcgagcgaatccctcaaagccggccccagttcggatcggaggctgcaacccgcctccgtgaagtcggagttgctagtaatcgcggatcagcatgccgcggtgaatacgttcccgggccttgtacacaccgcccgtcacaccacccgagtcgtctgcacccgaagccgccggccgaacccgcaaggggc

SEQ ID NO: 110 - DNA, 1367 bp, synthetic construct
  misc_feature 1..1367: note = Synthetic: FBI00205
  source 1..1367: mol_type = other DNA
attgaancttcggcngatttnnnttnnnttctagtggcggacgggtgagtaacgcgtgggtaacctgccttatacagggggataacagtcagaaatgactgctaataccgcataagcgcacagggccgcatggctcggtgtgaaaaactccggtggtataagatggacccgcgttggattagctggttggtgaggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggatggacaagtctgatgtgaaaggctggggctcaaccccgggactgcattggaaactgcccgtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgcggtaaacgatgaatgctaggtgtcggggagcaaagctcttcggtgccgccgcaaacgcattaagcattccacctggggagtacgttcgcaagaatgaaactcaaaggaattgacggggacccgcacaagcggtggagcatgtggtttaattcgaagcaacgcgaagaaccttaccaagtcttgacatccctctgaccgnnnnttaaccgnntctttccttcgggacagaggagacaggtggtgcatggttgtcgtcagctcgtgtcgtgagatgttgggttaagtcccgcaacgagcgcaacccctatcctcagtagccagcggntnagccgggcactctggggagactgccagggataacctggaggaaggcggggatgacgtcaaatcatcatgccccttatgatttgggctacacacgtgctacaatggcgtaaacaaagggaagcgaacctgcgagggtgggcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacctaaccgcaaggaa

SEQ ID NO: 111 - DNA, 1364 bp, synthetic construct
  misc_feature 1..1364: note = Synthetic: FBI00206
  source 1..1364: mol_type = other DNA
gcagtcgaggggcagcattttagtttgcttgcaaactaaagatggcgaccggcgcacgggtgagtaacacgtatccaacctgccgataactcggggatagcctttcgaaagaaagattaatatccgatagtatattaaaaccgcatggttttactattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtattccacgtgtgggattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaataatctggaaacaggttagccgcaagcaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggcgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccggggg

SEQ ID NO: 112 - DNA, 1378 bp, synthetic construct
  misc_feature 1..1378: note = Synthetic: FBI00208
  source 1..1378: mol_type = other DNA
tcgancggagtgttttcacggaagttttcggatggaagtggttacacttagtggcggacgggtgagtaacacgtgagcaacctgcctttcagagggggataacagttggaaacgactgctaataccgcatgatattaccgggtcacatggcctggcaatcaaaggagcaatccgctgaaagatgggctcgcgtccgattagccagttggcggggtaatggcccaccaaagcgacgatcggtagccggactgagaggttgaacggccacattgggactgagacacggcccagactcctacgggaggcagcagtgggggatattgcacaatgggcgaaagcctgatgcagcgacgccgcgtgagggaagacggtcttcggattgtaaacctctgtcttaggggaagaaaatgacggtaccctaagaggaagctccggctaactacgtgccagcagccgcggtaatacgtagggagcgagcgttgtccggaattactgggtgtaaagggagcgtaggcgggatgccaagtagaatgttaaatccatcggctcaactggtggcagcgttctaaactggcgttcttgagtgaggtagaggcaggcggaattcctagtgtagcggtgaaatgcgtagatattaggaggaacaccagtggcgaaggcggcctgctgggccttaactgacgctgaggctcgaaagcgtggggagcaaacaggattagataccctggtagtccacgccgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcggatgcataccatagagatatgggaagcccttcggggcatccagacaggtggtgcatggttgtcgtcagctcgtgtcgtgagatgttgggttaagtcccgcaacgagcgcaacccttattattagttgctacgcaagagcactctaatgagactgccgttgacaaaacggaggaaggtggggatgacgtcaaatcatcatgccccttatgacctgggctacacacgtactacaatggcactcaaacagagggaagcgacaccgcgaggtgaagcggatcccaaaaaagtgtctcagttcggatcgcaggctgcaacccgcctgcgtgaagtcggaattgctagtaatcgcggatcagcatgccgcggtgaatacgttcccgggccttgtacacaccgcccgtcacaccatgggagtcggtaacacccgaagccagtagcctaaccgcaagga

SEQ ID NO: 113 - DNA, 1385 bp, synthetic construct
  misc_feature 1..1385: note = Synthetic: FBI00211
  source 1..1385: mol_type = other DNA
tcgaggggcagcatggtcttancttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcganagcctgaaccagccaagtagcgtgaaggatgactgccctatgggttgtaaacttcttttataaaggaataaagtcgggtatgtatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatactgcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttccgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgagga

SEQ ID NO: 114 - DNA, 1379 bp, synthetic construct
  misc_feature 1..1379: note = Synthetic: FBI00212
  source 1..1379: mol_type = other DNA
tgcagtcgaacgaagcgattaggaggaagttttcggatggattcctggttgactgagtggcggacgggtgagtaacgcgtggataacctgcctcacactgggggataacagttagaaatggctgctaataccgcataagcgcacagtaccgcatggtacggtgtgaaaaacccaggtggtgtgagatggatccgcgtctgattagccagttggcggggtaacggcccaccaaagcgacgatcagtagccgacctgagagggtgaccggccacgttgggactgagacagggcccaaactcctaggggaggcagcagtggggaatattgcacaatgggcgcaagcctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgacgcaagtctggagtgaaagcccggggcccaaccccgggactgctttggaaactgtgttgctggagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatgctaggtgtcggggggcaaagcccttcggtgccgccgctaacgcaataagcattccacctggggagtacgttcgcaagaatgaaactcaaaggaattgacggggacccgcacaagcggtggagcatgtggtttaattcgaagcaacgcgaagaaccttaccaagtcttgacatcctnntgaccggcnngtaacggtgcctttccttcgggacaagagagacaggtggtgcatggttgtcgtcagctcgtgtcgtgagatgttgggttaagtcccgcaacgagcgcaacccttatccttagtagccagcacgtcgtggtgggcactctagggagactgccagggataacctggaggaaggtggggatgacgtcaaatcatcatgccccttatgatttgggctacacacgtgctacaatggcgtaaacaaagggaggcgaccctgcgaaggcaagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagcaacgcccgaagtcnagtganc

SEQ ID NO: 115 - DNA, 1417 bp, synthetic construct
  misc_feature 1..1417: note = Synthetic: FBI00220
  source 1..1417: mol_type = other DNA
tcgancgagaagagnngagaagcttgcttcttatnnattcgagtggcaaacgggtgagtaacgcgtaagcaacctgcccttcagatggggacaacagctggaaacggctgctaataccgaatacgttctttccgccgcatgacggaatgaagaaagggaggccttcgggctttcgctggaggaggggcttgcgtctgattagctggttggaggggtaacggcccaccaaggcgacgatcagtagccggtctgagaggatgaacggccacattgggactgagacacggcccagactcctacgggaggcagcantggggaatcttccgcaatggacgaaagtctgacggagcaacgccgcgtgaacgatgacggccttcgggttgtaaagttctgttatatgggacgaacagggcatcggttaatacccggtgtctttgacggtaccgtaagagaaagccacggctaactacgtgccagcagccgcggtaatannnnngtggcaagcgttgtccggaattattgggcgtaaagggcgcgcaggcggcatcgcaagtcggtcttaaaagtgcgggggcttaaccccgtgaggggaccgaaactgtgaagctcgagtgtcggagaggaaagcggaattcctagtgtagcggtgaaatgcgtagatattaggaggaacaccagtggcgaaagcggctttctggacgacaactgacgctgaggcgcgaaagccaggggagcaaacgggattagataccccggtagtcctggccgtaaacgatggatactaggtgtaggaggtatcgactccttctgtgccggagttaacgcaataagtatcccgcctgggggagtacggccgcaaggctgaaactcaaaggaattgacgggggcccgcacaagcggtggagtatgtggtttaattcgacgcaacgcgaagaaccttaccaagccttgacattgattgctacggaaagagatttccggttcttcttcggaagacaagaaaacaggtggtgcacggctgtcgtcagctcgtgtcgtgagatgttgggttaagtcccgcaacgagcgcaacccctatcttctgttgccagcacctcgggtggggactcagaagagactgccgcagacaatgcggaggaaggcggggatgacgtcaagtcatcatgccccttatggcttgggctacacacgtactacaatggctcttaatagagggaagcgaaggagcgatccggagcaaaccccaaaaacagagtcccagttcggattgcaggctgcaactcgcctgcatgaagcaggaatcgctagtaatcgcaggtcagcatactgcggtgaatacgttcccgggccttgtacacaccgcccgtcacaccacgaaagtcattcacacccgaagccggtgaggcaaccgcaagga

SEQ ID NO: 116 - DNA, 1378 bp, synthetic construct
  misc_feature 1..1378: note = Synthetic: FBI00221
  source 1..1378: mol_type = other DNA
cgaggggcagcngnnngtagcaatacttcgccggcgaccggcgcacgggtgagtaacacgtgtgcaaccaaccccgtaccgggagataacccgcggaaacgtggactaacatcccataatactggaggatcgcatgatctttcatttaaaattccggtggtacgggacgggcacgcgcgacattaggtagttggcggggtaacggcccaccaagccgacgatgtctaggggttctgagaggaaggtcccccacactggaactgagacacggtccagactcctacgggaggcagcagtgaggaatattggtcaatgggcgagagcctgaaccagccaagtcgcgtgagggaagaatggtccatggcctgtaaacctcttttgtcaaggaagaataaanngcacgtgtgcnntcttgccagtacttgacgaataagcatcggctaactccgtgccagcagccgcggtaatacgggggatgcgagcgttatccggatttattgggtttaaagggcgcgtaggcgggacgccaagtcagcggtaaaagactgcagctaaactgtagcacgccgttgaaactggcgacctcgagacgagacgagggaggcggaacaagtgaagtagcggtgaaatgcttagatatcacttggaaccccgatagcgaaggcagcttcccaggctcgatctgacgctgatgcgcgagagcgtgggtagcgaacaggattagataccctggtagtccacgccgtaaacgatgctcactggatcttggcgatacactgccagggttcaagcgaaagtattaagtgagccacctggggagtacgtcggcaacgatgaaactcaaaggaattgacgggggcccgcacaagcggaggaacatgtggtttaattcgatgatacgcgaggaaccttacccgggtttaaatgtannntgcatgtnttggaaacaggaattcccttcggggctagatacaaggtgctgcatggttgtcgtcagctcgtgccgtgaggtgtcgggttaagtcccataacgagcgcaacccctatcgccagttgccatcggttgaagccgggcactctgtcgagactgccaccgtaaggtgcgaggaaggcggggatgacgtcaaatcagcacggcccttacacccggggcgacacacgtgttacaatggccggtacagagggcagccacggggtgacccggagcgaatctctaaagccggtcgtagttcggactggagtctgcaacccgactccacgaagttggattcgctagtaatcgcgcatcagccatggcgcggtgaatacgttcccgggccttgtacacaccgcccgtcaagccatggaagccgggagtacctgaagatcgtgaccgcga

SEQ ID NO: 117 - DNA, 1391 bp, synthetic construct
  misc_feature 1..1391: note = Synthetic: FBI00224
  source 1..1391: mol_type = other DNA
tcgaacggcagcgcggggagcttgctccctggcggcgagtggcgcacgggtgagtaatacatcggaacgtgtcttctagtgggggataactgcccgaaagggcagctaataccgcatgagacctgagggtgaaagcgggggatcgcaagacctcgcgctggaagagcggccgatgtccgattagctagttggtgaggtaaaggctcaccaaggcgacgatcggtagctggtctgagaggacgaccagccacactgggactgagacacggcccagactcctacgggaggcagcagtggggaattttggacaatgggggcaaccctgatccagccatgccgcgtgcaggatgaaggtcttcggattgtaaactgcttttgtcagggacgaaaagggatgcgataacaccgcattccgctgacggtacctgaagaataagcaccggctaactacgtgccagcagccgcggtaatacgtagggtgcaagcgttaatcggaattactgggcgtaaagcgtgcgcaggcggttctgtaagatagatgtgaaatccccgggctcaacctgggaattgcatatatgactgcaggacttgagtttgtcagaggagggtggaattccacgtgtagcagtgaaatgcgtagatatgtggaagaacaccgatggcgaaggcagccctctgggacatgactgacgctcatgcacgaaagcgtgggggagcaaacaggattagataccctggtagtccacgccctaaacgatgtctactagttgttggggacgatagtccttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctagccttgacatgccaggaagncntgagagatcaggccgtgcccgcaagggaatctggacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatggctagggcctcacacgtcatacaatggtcggaacagagggaagcgaagccgcgaggtgaagccaatcccagaaaaccgatcgtagtccggattgcagtctgcaactcgactgcatgaagtcggaatcgctagtaatcgcggatcagcatgccgcggtgaatacgttcccgggtcttgtacacaccgcccgtcacaccatgggagtggggttcaccagaagacgtttgcccaaccgaaaggaag

SEQ ID NO: 118 - DNA, 1398 bp, synthetic construct
  misc_feature 1..1398: note = Synthetic: FBI00226
  source 1..1398: mol_type = other DNA
tgcagtcgacganttgctctttgggaagccctcgggtggaactgtgagtatacttagtggcggacgggtgagtaacgcgtgagcaatctgccctgcaatgggggacaacagttggaaacgactgctaataccgcataagaccacgaaaccgcatggtttagaggtaaaaggattcattcgatgcaggatgagctcgcgtcccattagatagttggtgaggtaacggcccaccaagtcaacgatgggtagccgacctgagagggtgatcggccacattggaactgagaaacggtccaaactcctacgggaggcagcagtggggaatattgggcaatgggggaaaccctgacccagcaacgccgcgtgaaggaagaaggccttcgggttgtaaacttttgtcctatgggacgaaaaaaatgacggtaccataggaggaagctccggctaactacgtgccagcagccgcggtaatacgtagggagcaagcgttgtccggaattactgggcgtaaagggtgcgtaggtggccatgtaagtcaggtgtgaaagaccggggctcaaccccggggttgcacttgaaactgtgtggcttgagtacaggagagggaagtggaattcctagtgtagcggtgaaatgcgtagatattaggaggaacaccagtggcgaaggcgactttctggactgtaactgacactgaggcacgaaagcgtggggagcaaacaggattagataccctggtagtccacgccgtaaacgatggatactaggtgtggggggcgatagtcctccgtgccgaagctaacgcattaagtatcccgcctggggagtacgatcgcaaggttgaaactcaaaggaattgacgggggcccgcacaagcagcggagcatgtggtttaattcgaagcaacgcgaagaaccttaccaaggcttgacatcctctgacgcatatagagatatatgttcccttcggggcagagagacaggtggtgcatggttgtcgtcagctcgtgtcgtgagatgttgggttaagtcccgcaacgagcgcaacccttgttgctagttgccagcacgtaaaggtgggaactctagtgagactgccggggacaactcggaggaaggtggggacgacgtcaaatcatcatgccccttatgtcttgggctacacacgtgctacaatggccggtacaaagggcagcgacctcgtaagagtaagcgaatctcaaaaagccggccccagttcggattgtgggctgcaacccgcccacatgaagtcggagttgctagtaatcgcgaatcagcatgtcgcggtgaatgcgttcccgggccttgtacacaccgcccgtcacaccacggaagttgggagcacccgaagccagtggcttaaccgtaaggagagagc

SEQ ID NO: 119 - DNA, 1395 bp, synthetic construct
  misc_feature 1..1395: note = Synthetic: FBI00229
  source 1..1395: mol_type = other DNA
tgcagtnngaggggcagcgnnnnngnngctgngcttcaattgccgnnnaccggcgcacgggtgcgtaacgcgtatgcgaccgtacccataacagggggataacactgagaaattggtactaatatcccataacatcatgaggggcatcccttttggttgaaaactccggtggttatggatgggcatgcgttgtattagctggttggtgaggtaacggctcaccaaggcgacgatacatagggggactgagaggttaaccccccacattggtactgagacacggaccaaactcctacgggaggcagcagtgaggaatattggtcaatggacgcaagtctgaaccagccatgccgcgtgcaggatgacggctctatgagttgtaaactgcttttgtacgagggtaaacccggatacgtgtatccggctgaaagtatcgtacgaataaggatcggctaactccgtgccagcagccgcggtaatacggaggattcaagcgttatccggatttattgggtttaaagggtgcgtaggcggtttgataagttagaggtgaaataccggtgcttaacaccggaactgcctctaatactgttgagctagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgctgtcggcgatacacagtcggtggctaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttactgacgattctggaaacaggatttcccttcggggcaggaaactaggtgntgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggcagctacccagtgatgggatgcaaatctcgaaagcctatctcagttcggattggaggctgaaacccgcctccatgaagttggattcgctagtaatcgcgcatcagccatggcgcggtgaatacgttcccgggccttgtacacaccgcccgtcaagccatggaagctgggggtgcctgaagttcgngaccgcaangagc

SEQ ID NO: 120 - DNA, 1377 bp, synthetic construct
  misc_feature 1..1377: note = Synthetic: FBI00232
  source 1..1377: mol_type = other DNA
gcagtcgaggggcagcatcatcaaagcttgctttgatggatggcgaccggcgcacgggtgagtaacacgtatccaacctgccgacaacactgggatagcctttcgaaagaaagattaataccggatggcatagttttcccgcatgggataattattaaagaatttcggttgtcgatggggatgcgttccattaggcagttggcggggtaacggcccaccaaaccaacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagttagccacgtgtggctttttgtatgtaccgtatgaataaggatcggctaactccgtgccagcagccgcggtaatacggaggatccgagcgttatccggatttattgggtttaaagggagcgtaggcgggttgttaagtcagttgtgaaagtttgcggctcaaccgtaaaattgcagttgatactggcgaccttgagtgcaacagaggtaggcggaattcgtggtgtagcggtgaaatgcttagatatcacgaagaactccgattgcgaaggcagcttactggattgtaactgacgctgatgctcgaaagtgtgggtatcaaacaggattagataccctggtagtccacacagtaaacgatgaatactcgctgttggcgatatacagtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaactgactgaaccggaaacggttctttcttcggacagttgtgaaggtgctgcatggttgtcgtcagctcgtgccgtgaggtgtcggcttaagtgccataacgagcgcaacccttatcgatagttactagcaggtcatgctgaggactctattgagactgccgtcgtaagatgtgaggaaggtggggatgacgtcaaatcagcacggcccttacgtccggggctacacacgtgttacaatggggggtacagaaggcagctacacggcgacgtggtgctaatcccgaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctnaagtacgt

SEQ ID NO: 121 - DNA, 1417 bp, synthetic construct
  misc_feature 1..1417: note = Synthetic: FBI00233
  source 1..1417: mol_type = other DNA
gtcgaacggagacactctgttcactgaatggtagaaggcggcggaaccagaaagggagcgtcggttttctactgttgagtgaatagagtgtcttagtggcggacgggtgagtaacgcgtgaataacctgccttgcagtggggaataacacaccgaaaggtgtgctaataccgcatgatgtaaggggaaggcatcttccccttaccaaagatttatcgctgtgagatggattcgcgtccgattagatagttggtgaggtaacggcccaccaagtcgacgatcggtagccggactgagaggttgaacggccacattgggactgagacacggcccagactcctacgggaggcagcagtgggggatattgcacaatgggcgcaagcctgatgcagcgacgccgcgtgtgggaagacggccctcgggttgtaaaccactggctttggggacgataatgacggtacccaaggaggaagctccggctaactacgtgccagcagccgcggtaatacgtagggagcgagcgttgtccggaattactgggtgtaaagggagcgtaggcgggagtgcaagttgaatgtttaatctatgggctcaacccatatcagcgttcaaaactgcatttcttgagtgaagtagaggttggcggaattcctagtgtagcggtgaaatgcgtagatattaggaggaacaccagtggcgaaggcggccaactgggcttttactgacgctgaggctcgaaagcgtggggagcaaacaggattagataccctggtagtccacgccgtaaacgatgaatactaggtgtggggggactgaccccttccgtgccgcagttaacacaataagtattccacctggggagtacggccgcaaggctgaaactcaaaggaattgacgggggcccgcacaagcagtggattatgtggtttaattcgaagcaacgcgaagaaccttaccaggccttgacatctcctgagtagcctagagataggtgatgcccttcggggcaggaagacaggtggtgcatggttgtcgtcagctcgtgtcgtgagatgttgggttaagtcccgcaacgagcgcaacccttacggatagttgctacgcaagagcactctatcaggactgccgttgacaaaacggaggaaggtggggatgacgtcaaatcatcatgccccttatggcctgggctacacacgtaatacaatggcgtttaacagagggaagcaagaccgcgaggtggagcgaatcctcaaaaggcgtctcagttcagattgcaggctgcaacccgcctgcatgaagtcggaattgctagtaatcgcggatcagcatgccgcggtgaatacgttctcgggccttgtacacaccgcccgtcacaccatggaagtcggtaacacccgaagtcagtagcctaaccgcaa

SEQ ID NO: 122 - DNA, 1387 bp, synthetic construct
  misc_feature 1..1387: note = Synthetic: FBI00235
  source 1..1387: mol_type = other DNA
tgcngtcgaggggcagcacggtgtagcaatacactggtggcgaccggcgcacgggtgcgtaacgcgtatgcaacctacccataacagggggataacactgagaaattggtactaataccccataacatcaggaccggcatcggttctggttgaaaactccggtggttatggatgggcatgcgttgtattagctggttggtgaggtaacggctcaccaaggcaacgatacatagggggactgagaggttaaccccccacattggtactgagacacggaccaaactcctacgggaggcagcagtgaggaatattggtcaatggacgcaagtctgaaccagccatgccgcgtgcaggaagacggctctatgagttgtaaactgcttttgtacgagagtaaacgctcttacgtgtaagagcctgaaagtatcgtacgaataaggatcggctaactccgtgccagcagccgcggtaatacggaggatccaagcgttatccggatttattgggtttaaagggtgcgtaggcggtttgataagttagaggtgaaataccggtgcttaacaccgggactgcctctaatactgttgaactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgctgtcggcgatacacagtcggcggctaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttactgacgattctggaaacaggatttcccttcggggcaggaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggcagctacccagtgatgggatgcgaatctcgaaagcctatctcagttcggatcggaggctgaaacccgcctccgtgaagttggattcgctagtaatcgcgcatcagccatggcgcggtgaatacgttcccgggccttgtacacaccgcccgtcaagccatggaagctgggggtgcctgaagttcgtgaccgcaaggag

SEQ ID NO: 123 - DNA, 1527 bp, synthetic construct
  misc_feature 1..1527: note = Synthetic: FBI00236
  source 1..1527: mol_type = other DNA
tggagagtttgatcctggctcaggatgaacgctggcggcgtgcctaacacatgcaagtcgaacgaagttacacagaggaagttttcggatggaatcrgtataacttagtggcggacgggtgagtaacgcgtgggaaacctgccctgtaccgggggataacacttagaaataggtgctaataccgcataagcgcacrgmwyyrcatgrwkchgtgtgaaaaactccggtggtacaggatggtcccgcgtctgattagccagttggcagggtaacggcctaccaaagcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaaacccagggctcaaccttgggattgcatttggaactgccaggctggagtgcaggagaggtaagcggaattcctagtgtagcggtgaaatgcgtagatattaggaggaacaccagtggcgaaggcggcttactggactgtaactgacgttgaggctcgaaagcgtggggagcaaacaggattagataccctggtagtccacgcggtaaacgatgattgctaggtgtaggtgggtatggacccatcggtgccgcagctaacgcaataagcaatccacctggggagtacgttcgcaagaatgaaactcaaaggaattgacggggacccgcacaagcggtggagcatgtggtttaattcgaagcaacgcgaagaaccttaccaagtcttgacatcccawtgacgyrcctgtaaagaggyrttcccttcggggcawtggagacaggtggtgcatggttgtcgtcagctcgtgtcgtgagatgttgggttaagtcccgcaacgagcgcaacccttattcttagtagccagcaggtaragctgggcactctaaggagactgccggggataacccggaggaaggcggggatgacgtcaaatcatcatgccccttatgatttgggctacacacgtgctacaatggcgtaaacaaagggaagcgagacagtgatgtggagcaaatcccagaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggaaatgcccgaagtctgtgacctaaccgaaagggaggagcagccgaaggcaggtctgataactggggtgaagtcgtaacaaggtagccgtatcggaaggtgcggytggatcacctcctt

SEQ ID NO: 124 - DNA, 1529 bp, synthetic construct
  misc_feature 1..1529: note = Synthetic: FBI00237
  source 1..1529: mol_type = other DNA
tggagagtttgatcctggctcaggatgaacgctggcggcatgcctaatacatgcaagtcgaacgaagtgaagatagcttgctattggaacttagtggcgaacgggtgagtaacacgtagataacctgcctgtatgaccgggataacagttggaaacgactgctaataccggataggcagagaggaggcatctcttctctgttaaagttgggacacaacgcaaacagatggatctgcggtgcattagctagttggtgaggtaacggcccaccaaggcgatgatgcatagccggcctgagagggcgaacggccacattgggactgagacacggcccaaactcctacgggaggcagcagtagggaattttcggcaatgggggaaaccctgaccgagcaatgccgcgtgagtgaagacggccttcgggttgtaaagctctgttgtaagggaagaacggcatagagagggaatgctctatgagtgacggtaccttaccagaaagccacggctaactacgtgccagcagccgcggtaatacgtaggtggcaagcgttatccggaattattgggcgtaaagggtgcgtaggcggcgagataagtctgaggtaaaagcccgtggctcaaccacggtaagccttggaaactgtctggctggagtgcaggagaggacaatggaattccatgtgtagcggtaaaatgcgtagatatatggaggaacaccagtggcgaaggcggttgtctggcctgtaactgacgctgaagcacgaaagcgtggggagcaaataggattagataccctagtagtccacgccgtaaacgatgagaactaagtgttggggaaactcagtgctgcagttaacgcaataagttctccgcctggggagtatgcacgcaagtgtgaaactcaaaggaattgacgggggcccgcacaagcggtggagtatgtggtttaattcgacgcaacgcgaagaaccttaccaggccttgacatggtatcaaaggccctagagatagggagatagttatggtacacacaggtggtgcatggttgtcgtcagctcgtgtcgtgagatgttgggttaagtcccgcaacgagcgcaacccttgtttctagttaccaacagtaagatggggactctagagagactgccggtgacaaaccggaggaaggtggggatgacgtcaaatcatcatgccccttatggcctgggctacacacgtactacaatggcgtctacaaagagcagcgagcaggtgactgtaagcgaatctcataaaggacgtctcagttcggattgaagtctgcaactcgacttcatgaagtcggaatcgctagtaatcgcggatcagcatgccgcggtgaatacgttctcgggccttgtacacaccgcccgtcaaaccatgggagttgataatacccgaagccggtggcctaaccatttatggagggagccgtcgaaggtaggatcgatgactggggttaagtcgtaacaaggtatccctacgggaacgtgcggytggatcacctcctt

SEQ ID NO: 125 - DNA, 1395 bp, synthetic construct
  misc_feature 1..1395: note = Synthetic: FBI00238
  source 1..1395: mol_type = other DNA
anncntgcagtcgaggggcagcatgaagtagcaatactttgatggcgaccggcggacgggtgcgtaacgcgtatgcaacctaccttaaacagggggataacactgagaaattggtactaataccccatattattcgagacggcatcgtcttgggttgaaaactccggtggtttaagatgggcatgcgttgtattagctggttggtgaggtaacggctcaccaaggcgacgatacatagggggactgagaggtcaaccccccacattggtactgagacacggaccaaactcctacgggaggcagcagtgaggaatattggtcaatggacgcaagtctgaaccagccatgccgcgtgcaggaagacggctctatgagttgtaaactgcttttgtacgagggtaacagcatctacgcgtaggtgcatgaaagtatcgtacgaataaggatcggctaactccgtgccagcagccgcggtaatacggaggatccaagcgttatccggatttattgggtttaaagggtgcgtaggcggttttataagttagaggttaaatatcggagcttaactccgttatgcctctaatactgtaggactagagaatagttgcggtaggcggaatgtatggtgtagcggtgaaatgcttagagatcatacagaacaccgattgcgaaggcagcttaccaaactatgactgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttatcgacggacctagaaataggtcttccctacggggcgagaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggccacacacgtgttacaatggtaggtacagagggcagctaccctgcgaagggatgcgaatctcgaaagcctatctcagttcggattggaggctgaaacccgcctccatgaagttggattcgctagtaatcgcgcatcagccatggcgcggtgaatacgttcccgggccttgtacacaccgcccgtcaagccatggaagctgggggtgcctgaagttcgtgaccgcaaggagcgac

SEQ ID NO: 126 - DNA, 1363 bp, synthetic construct
  misc_feature 1..1363: note = Synthetic: FBI00243
  source 1..1363: mol_type = other DNA
tgcagtcgancggtgaagaggagcttgctcctcggancagtggcggacgggtgagtaacacgtgagcaacctggctctaagagggggacaacagttggaaacgactgctaataccgcataacgtatcgggatggcatcttcctgataccaaagattttatcgcttagagatgggctcgcgtctgattagatagttggcggggtaacggcccaccaagtcgacgatcagtagccggactgagaggttgaacggccacattgggactgagacacggcccagactcctacgggaggcagcagtgggggatattggacaatgggggcaaccctgatccagcgacgccgcgtgagggaagaaggttttcggattgtaaacctctgttgacggagaaaaaaatgatggtatccgtttagaaagccacggctaactacgtgccagcagccgcggtaatacgtaggtggcaagcgttgtccggaattactgggtgtaaagggagtgtaggcgggatatcaagtcagaagtgaaaattacgggctcaactcgtaacctgcttttgaaactgacattcttgagtgaagtagaggcaagcggaattcctagtgtagcggtgaaatgcgtagatattaggaggaacaccagtggcgaaggcggcttgctgggcttttactgacgctgaggctcgaaagcgtggggagcaaacaggattagataccctggtagtccacgccgtaaacgatgattactaggtgtggggggattgaccccttccgtgccggagtaaacacaataagtaatccacctggggagtacgaccgcaaggttgaaactcaaaggaattgacgggggcccgcacaagcagtggagtatgtggtttaattcgacgcaacgcgaagaaccttaccaggtcttgacatcgagtgaccgcctaagagattaggctttcccttcggggacacaaagacaggtggtgcatggttgtcgtcagctcgtgtcgtgagatgttgggttaagtcccgcaacgagcgcaacccttatcattagttgctacgcaagagcactctaatgagactgccgttgacaaaacggaggaaggtggggatgacgtcaaatcatcatgccctttatgacctgggctacacacgtactacaatggcgtttaacaaagagaagcaaagccgcgaggcagagcaaatctccaaaaaacgtctcagttcggattgtaggctgcaactcgcctacatgaagtcggaattgctagtaatcgtaggtcagcatactacggtgaatacgttcccgggccttgtacacaccgcccgtcaaaccatgagagttggcaacacccgaagtcggtagtctaancgca

SEQ ID NO: 127 - DNA, 1371 bp, synthetic construct
  misc_feature 1..1371: note = Synthetic: FBI00244
  source 1..1371: mol_type = other DNA
tcgaacgagcgagagagagcttgctttctcgagcgagtggcgaacgggtgagtaacgcgtgaggaacctgcctcaaagagggggacaacagttggaaacgactgctaataccgcataagcccacggcccggcatcgggcagagggaaaaggagcaatccgctttgagatggcctcgcgtccgattagctagttggtgaggtaacggcccaccaaggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttgaggaagataatgacggtactcaacaaggaagtgacggctaactacgtgccagcagccgcggtaaaacgtaggtcacaagcgttgtccggaattactgggtgtaaagggagcgcaggcgggaagacaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacaggattagataccctggtagtccacactgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgacgcaacgcgaagaaccttaccaagtcttgacatcctgcgacggtgctggaaacagtactttccttcgggacgcagagacaggtggtgcatggttgtcgtcagctcgtgtcgtgagatgttgggttaagtcccgcaacgagcgcaacccttatggtcagttactacgcaagaggactctggccagactgccgttgacaaaacggaggaaggtggggatgacgtcaaatcatcatgccctttatgacttgggctacacacgtactacaatggcgttaaacaaagagaagcaagaccgcgaggtggagcaaaactcagaaacaacgtcccagttcggactgcaggctgcaactcgcctgcacgaagtcggaattgctagtaatcgcagatcagcatgctgcggtgaatacgttcccgggccttgtacacaccgcccgtcacaccatgagagccggggggacccgaagtcggtagtctaaccgcaaggaggacncc

SEQ ID NO: 128 - DNA, 1410 bp, synthetic construct
  misc_feature 1..1410: note = Synthetic: FBI00245
  source 1..1410: mol_type = other DNA
agtcgaacggagaacttatttcggtaagttcttagtggcgaacgggtgagtaacgcgtgggcaacctgccctccagttggggacaacattccgaaagggatgctaataccgaatgtcctccctcctccgcatggaggagggaggaaagatggcctctgcttgcaagctatcgctggaagatgggcccgcgtctgattagctagttggtggggtaacggctcaccaaggcgatgatcagtagccggtctgagaggatgaacggccacattgggactgagacacggcccaaactcctacgggaggcagcagtggggaatcttccgcaatggacgaaagtctgacggagcaacgccgcgtgagtgatgaaggtcttcggattgtaaaactctgttgttagggacgaaagcaccgtgttcgaacaggtcatggtgttgacggtacctaacgaggaagccacggctaactacgtgccagcagccgcggtaatacgtaggtggcaagcgttgtccggaattattgggcgtaaagagcatgtaggcgggcttttaagtctgacgtgaaaatgcggggcttaaccccgtatggcgttggatactggaagtcttgagtgcaggagaggaaaggggaattcccagtgtagcggtgaaatgcgtagatattgggaggaacaccagtggcgaaggcgcctttctggactgtgtctgacgctgagatgcgaaagccagggtagcaaacgggattagataccccggtagtcctggccgtaaacgatggatactaggtgtaggaggtatcgaccccttctgtgccggagttaacgcaataagtatcccgcctggggactacgatcgcaagattgaaactcaaaggaattgacgggggcccgcacaagcggtggagtatgtggtttaattcgacgcaacgcgaagaaccttaccaaggcttgacattgagtgaaagacctagagataggtccctcccttcggggacacgaaaacaggtggtgcatggctgtcgtcagctcgtgtcgtgagatgttgggttaagtcccgcaacgagcgcaacccctatcctatgttaccagcgcgtaaaggcggggactcataggagactgccagggataacttggaggaaggcggggatgacgtcaagtcatcatgccccttatgtcttgggctacacacgtactacaatggtcggcaacaaagggcagcgaaaccgcgaggtggagcaaatcccagaaacccgaccccagttcggatcgtaggctgcaacccgcctacgtgaagttggaatcgctagtaatcgcaggtcagcatactgcggtgaatacgttcccgggccttgtacacaccgcccgtcacaccacgaaagttggtaacacccgaagccggtgagataaccttttag

SEQ ID NO: 129 - DNA, 1377 bp, synthetic construct
  misc_feature 1..1377: note = Synthetic: FBI00248
  source 1..1377: mol_type = other DNA
tcnagcgagaagccattgactganacttcggtnagaaggatatggtggaaagcggcggacgggtgagtaacgcgtaggcaacctgccccttacagagggatagccattggaaacgatgattaaaacctcataacgcatcccccccacatggaggggaagccaaagattcatcggtaagggatgggcctgcgtctgattagcttgttggcggggtaacggcccaccaaggcgacgatcagtagccgacctgagagggtgatcggccacattggaactgagacacggtccaaactcctacgggaggcagcagtggggaatattgcacaatgggcgaaagcctgatgcagcaacgccgcgtgagggatgaaggccttcgggtcgtaaacctctgtccttggggaagaaacaaatgacggtacccatggaggaagccccggctaactacgtgccagcagccgcggtaatacgtagggggcgagcgttatccggaattattgggcgtaaagagtgcgtaggtggttacctaagcgcagggtctaaggcaatggctcaaccattgttcgccctgcgaactgggctacttgagtgcaggagaggaaagcggaattcctagtgtagcggtgaaatgcgtagatattaggaggaacaccagtggcgaaggcggctttctggactgttactgacactgaggcacgaaagcgtggggagcaaacaggattagataccctggtagtccacgccgtaaacgatgagcactaggtgtcggggccgcaaggcttcggtgccgcagttaacgcattaagtgctccgcctggggagtacgcacgcaagtgtgaaactcaaaggaattgacggggacccgcacaagcagcggagcatgtggtttaattcgaagcaacgcgaagaaccttaccaggacttgacatcccactgacagatccctaaccggatccttcttcggacagtggagacaggtggtgcatggttgtcgtcagctcgtgtcgtgagatgttgggttaagtcccgcaacgagcgcaacccttgccattagttgccatcattcagttgggcactctaatgggactgccggggacaactcggaggaaggtggggatgacgtcaaatcatcatgccccttatgttctgggctacacacgtgctacaatggccggtacagcaagaagcaaaaccgcgaggtggagcaaatctcaaaaaccggtcccagttcggactgcaggctgaaacccgcctgcacgaagccggagttgctagtaatcgtggatcagaatgccacggtgaatgcgttcccgggtcttgtacacaccgcccgtcacaccatggaagttgggggtgcccgaagccggcagggaaatatgc

SEQ ID NO: 130 - DNA, 1378 bp, synthetic construct
  misc_feature 1..1378: note = Synthetic: FBI00251
  source 1..1378: mol_type = other DNA
gtcgnncgggatccatcaggctttgcttggtggtgagagtggcgaacgggtgagtaatgcgtgaccgacctgccccatacaccggaatagctcctggaaacgggtggtaatgccggatgctccgactcctcgcatggggtgtcgggaaagatttcatcggtatgggatggggtcgcgtcctatcaggtagtcggcggggtaacggcccaccgagcctacgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgatcgggagcaagccttcgggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatctgcgccgggtacgggcgggctggagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatatggcctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgtccagggcttcacgcatgctacaatggccggtacaacgggatgcgacacggcgacgtggagcggatccctgaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcctaaccctttg

SEQ ID NO: 131 - DNA, 1202 bp, synthetic construct
  misc_feature 1..1202: note = Synthetic: FBI00254
  source 1..1202: mol_type = other DNA
gattcttcggatganngnnnggtgactgagtggcggacgggtgagtaacgcgtgggtaacctgccctgtacagggggataacagctggaaacggctgctaataccgcataagcgcacgaggagacatctccttgtgtgaaaaactccggtggtacaggatgggcccgcgtctgattagctggttggcagggtaacggcctaccaaggcaacgatcagtagccggtctgagaggatgaacggccacattggaactgagacacggtccaaactcctacgggaggcagcagtggggaatattgcacaatgggggaaaccctgatgcagcaacgccgcgtgagtgaagaagtatttcggtatgtaaagctctatcagcagggaagataatgacggtacctgactaagaagctccggctaaatacgtgccagcagccgcggtaatacgtatggagcaagcgttatccggatttactgggtgtaaagggtgcgtaggtggcagtgcaagtcagatgtgaaaggccggggctcaaccccggagctgcatttgaaactgctcgggctagagtacaggagaggcaggcggaattcctagtgtagcggtgaaatgcgtagatattaggaggaacaccagtggcgaaggcggcctgctggactgttactgacactgaggcacgaaagcgtggggagcaaacaggattagataccctggtagtccacgccgtaaacgatgaatactaggtgtcggggccgtataggcttcgggtgccgccgctaacgcagtaagtattccacctggggagtacgttcgcaagaatgaaactcaaaggaattgacggggacccgcacaagcggtggagcatgtggtttaattcgaagcaacgcgaaagaaccttaccaggtcttgacatccttctgaccactccgtaatggagttttccttcgggacagaagagacaggtggnntatggttgtcgtcagctcgtgtcgtgagatgttgggttaagtcccgcaacgagcgcaacccctatcttcagtagccagcaggtaaggctgggcactctggagagactgccagggataacctggaggaaggtggggacgacgtcaaatcatcatgccccttatgatctgggcgacacacgtgctacaatggcggtcacagagtgaggcgaacccgggagggggagcaaaccacaacaaggcc

SEQ ID NO: 132 - DNA, 1391 bp, synthetic construct
  misc_feature 1..1391: note = Synthetic: FBI00255
  source 1..1391: mol_type = other DNA
tgcagtcgagcgnngcggtttcnatgaagttttcggatggatttgaaattgacttagcggcggacgggtgagtaacgcgtgggtaacctgccttacactgggggataacagttagaaatgactgctaataccgcataagcgcacagggccgcatggtccggtgtgaaaaactccggtggtgtaagatggacccgcgtctgattaggtagttggcggggtaacggcccaccaagccgacgatcagtagccgacctgagagggtgaccggccacattgggactgagacacggcccaaactcctacgggaggcagcagtggggaatattggacaatgggcgaaagcctgatccagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtttagcaagtctgaagtgaaagcccggggctcaaccccggtactgctttggaaactgttagacttgagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgtcggggggcaaagcccttcggtgccgccgcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccattgaaannnnnttaaccggtgtccctcttcggagcaatggagacaggtggtgcatggttgtcgtcagctcgtgtcgtgagatgttgggttaagtcccgcaacgagcgcaacccttatccttagtagccagcgagtagagtcgggcactctggggagactgccagggataacctggaggaaggtggggatgacgtcaaatcatcatgccccttatgatttgggctacacacgtgctacaatggcgtaaacaaagggaagcaaaggagcgatccggagcaaaccccaaaaataacgtctcagttcggattgcaggctgcaactcgcctgcatgaagctggaatcgctagtaatcgcgaatcagaatgtcgcggtgaatacgttcccgggtcttgtacacaccgcccgtcacaccatgggagttggtaacgcccgaagtcagtgacccaaccgcaagnag

SEQ ID NO: 133 - DNA, 1374 bp, synthetic construct
  misc_feature 1..1374: note = Synthetic: FBI00258
  source 1..1374: mol_type = other DNA
tcgagcgaaccacttcggtggtgagcggcgaacgggtgagtaacacgtaggttatctgcccatcagacggggacaacgattggaaacgatcgctaataccggataggacgaaagtttaaaggtgcttcggcaccactgatggatgagcctgcggcgcattagctagttggtagggtaaaggcctaccaaggcgacgatgcgtagccgacctgagagggtgaacggccacactgggactgagacacggcccagactcctacgggaggcagcagtagggaatcttcggcaatgggcgaaagcctgaccgagcaacgccgcgtgaatgatgaaggccttcgggttgtaaaattctgttataagggaagaatggctctagtaggaaatggctagagtgtgacggtaccttatgagaaagccacggctaactacgtgccagcagccgcggtaatacgtaggtggcgagcgttatccggaattattgggcgtaaagagcgcgcaggtggttgattaagtctgatgtgaaagcccacggcttaacccgtggagggtcattggaaactggtcaacttgagtgcagaagagggaagtggaattccatgtgtagcggtgaaatgcgtagagatatggaggaacaccagtggcgaaggcggcttcctggtctgtaactgacactgaggcgcgaaagcgtggggagcaaacaggattagataccctggtagtccacgccgtaaacgatgagtgctaagtgttgggggtcgaacctcagtgctgaagttaacgcattaagcactccgcctggggagtacggtcgcaagactgaaactcaaaggaattgacggggacccgcacaagcggtggagcatgtggtttaattcgaagcaacgcgaagaaccttaccaggtcttgacataccagtgaccgtcctagagataggattttcccttcggggacaatggatacaggtggtgcatggttgtcgtcagctcgtgtcgtgagatgttgggttaagtcccgcaacgagcgcaacccctgtcgttagttgccagcattcagttggggactctaacgagactgccagtgacaaactggaggaaggtggggatgacgtcaaatcatcatgccccttatgacctgggctacacacgtgctacaatggttggtacaaagagaagcgaagcggtgacgtggagcaaacctcataaagccaatctcagttcggattgtaggctgcaactcgcctacatgaagttggaatcgctagtaatcgcgaatcagcatgtcgcggtgaatacgttcccgggtcttgtacacaccgcccgtcacaccacgagagtttacaacacccgaagtcagtggcctaaccgcaaggaggga

SEQ ID NO: 134 - DNA, 1389 bp, synthetic construct
  misc_feature 1..1389: note = Synthetic: FBI00260
  source 1..1389: mol_type = other DNA
tgcagtcgnacgaagcactttatttgatttccttcgggantgattattttgtgactgagtggcggacgggtgagtaacgcgtgggtaacctgccttgtacagggggataacagttggaaacggctgctaataccgcataagcgcacagcatcgcatgatgcagtgtgaaaaactccggtggtataagatggacccgcgttggattagctagttggtgaggtaacggcccaccaaggcgacgatccatagccgacctgagagggtgaccggccacattgggactgagacacggcccaaactcctacgggaggcagcagtggggaatattgcacaatgggcgaaagcctgatgcagcgacgccgcgtgagcgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgttgggaagcattgcttctcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggtacttaaccgtaccttctcttcggagcaggagtgacaggtggtgcatggttgtcgtcagctcgtgtcgtgagatgttgggttaagtcccgcaacgagcgcaacccttatctttagtagccagcggtttggccgggcactctagagagactgccagggataacctggaggaaggcggggatgacgtcaaatcatcatgccccttatgacttgggctacacacgtgctacaatggcgtaaacaaagggaagcaaagctgtgaagccgagcaaatctcaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttgggaatgcccgaagccagtgacctaaccgaaag

SEQ ID NO: 135 - DNA, 1524 bp, synthetic construct
  misc_feature 1..1524: note = Synthetic: FBI00263
  source 1..1524: mol_type = other DNA
tggagagtttgatcctggctcaggatgaacgctagctacaggcttaacacatgcaagtcgaggggcatcagtttggtttgcttgcaaaccaaagctggcgaccggcgcacgggtgagtaacacgtatccaacctgcctcatactcggggatagcctttcgaaagaaagattaatatccgatagcatatatttcccgcatgggttttatattaaagaaattcggtatgagatggggatgcgttccattagtttgttgggggggtaacggcccaccaagactacgatggataggggttctgagaggaaggtcccccacattggaactgagacacggtccaaactcctacgggaggcagcagtgaggaatattggtcaatggacgcgagtctgaaccagccaagtagcgtgaaggatgactgccctatgggttgtaaacttcttttatatgggaataaagttgtccacgtgtggatttttgtatgtaccatatgaataaggatcggctaactccgtgccagcagccgcggtaatacggaggatccgagcgttatccggatttattgggtttaaagggagcgtaggcggattgttaagtcagttgtgaaagtttgcggctcaaccgtaaaattgcagttgatactggcagtcttgagtgcagtagaggtgggcggaattcgtggtgtagcggtgaaatgcttagatatcacgaagaactccgattgcgaaggcagctcactggagtgtaactgacgctgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaatgaattatggggaaacccatacgccgcaaggcatttgtgaaggtgctgcatggttgtcgtcagctcgtgccgtgaggtgtcggcttaagtgccataacgagcgcaacccttatcttcagttactaacaggtcatgctgaggactctggagagactgccgtcgtaagatgtgaggaaggtggggatgacgtcaaatcagcacggcccttacgtccggggctacacacgtgttacaatggggggtacagaaggccgctacctggtgacaggatgccaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcgtaacaaggtagccgtaccggaaggtgcggytggatcacctcctt

SEQ ID NO: 136 - DNA, 1514 bp, synthetic construct
  misc_feature 1..1514: note = Synthetic: FBI00267
  source 1..1514: mol_type = other DNA
tggagagtttgatcctggctcaggacgaacgctggcggcgtgcttaacacatgcaagtcgaacgagaagcttatgattgattcttcggaagatttcataagcggaaagtggcgaacgggtgagtaacgcgtaggtaaccaacctcatacagggggatagcccagggaaacttggattaaacccgcataagaccacgctatcgcatgataaagtggtaaaaactccggtggtatgagacggacctgcgtcttattaggtagttggtgaggtaacggctcaccaagccgacgatgagtagccgacctgagagggtgatcggccacattgggactgagacacggcccagactcctacgggaggcagcagtggggaatattgcgcaatgggggcaaccctgacgcagcaacgccgcgtgagcgatgaaggttttcggatcgtaaagctctgttctgagggaagataatgacggtacctcaggagaaagccccggctaactacgtgccagcagccgcggtaatacgtagggggcaagcgttgtccggattcactgggcgtaaagagcacgtaggcggttaattaagtcaggtgtgaaagttttcggctcaaccggaaaagtgcacttgaaactggataacttgagtatcggagaggtaagcggaattcctagtgtagcggtgaaatgcgtagagattaggaagaacaccggtggcgaaggcggcttactggacgataactgacgctgaggtgcgaaagcgtggggagcgaacaggattagataccctggtagtccacgccgtaaacgatgaatactaggtgttggggtaactcagtgccgcagttaacacattaagtattccgcctggggagtacgctcgcaagagtgaaactcaaaggaattgacgggggcccgcacaagcagcggagcatgtggtttaattcgaagcaacgcgaagaaccttaccaggtcttgacatcctttgaccgtctaagagattagactttccttcgggacaaagagacaggtggtgcatggttgtcgtcagctcgtgtcgtgagatgttgggttaagtcccgcaacgagcgcaacccttgtgtttagttactaacattcagttgaggactctagacagactgcccttgaaagagggaggaaggtggggacgacgtcaaatcatcatgccccttacgacctgggctacacacgtgctacaatggtctgtacaaagggttgcgaagcagtgatgccaagctaatctcaaaaagcagatctcagttcggattgcaggctgcaactcgcctgcatgaagtcggagttgctagtaatcgcgaatcagaatgtcgcggtgaatgcgttcccgggccttgtacacaccgcccgtcacaccacgagagttggtaacacccgaagccagtgagctaaccgtaaggaggcagctgtcgaaggtgggatcagtaattggggtgaagtcgtaacaaggtagccgtatcggaaggtgcggytggatcacctcctt

SEQ ID NO: 137 - DNA, 1384 bp, synthetic construct
  misc_feature 1..1384: note = Synthetic: FBI00269
  source 1..1384: mol_type = other DNA
tcgaggggcagcataatggatagcaatatctatggtggcgaccggcgcacgggtgcgtaacgcgtatgcaacctacctttaacagggggataacactgagaaattggtactaataccccataatatcatagaaggcatcttttatggttgaaaattccgatggttagagatgggcatgcgttgtattagctagttggtggggtaacggctcaccaaggcgacgatacatagggggactgagaggttaaccccccacactggtactgagacacggaccagactcctacgggaggcagcagtgaggaatattggtcaatggacgcaagtctgaaccagccatgccgcgtgcaggatgacggctctatgagttgtaaactgcttttgtacgagggtaaacgcagatacgtgtatctgtctgaaagtatcgtacgaataaggatcggctaactccgtgccagcagccgcggtaatacggaggattcaagcgttatccggatttattgggtttaaagggtgcgtaggcggtttgataagttagaggtgaaatttcggggctcaaccctgaacgtgcctctaatactgttgagctagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagcgacgattcttgaaagaggatttcccttcggggcgcgaaactaggtgctgcatggttgtcgtcagctcgtgccgtgaggtgtcgggttaagtcccataacgagcgcaacccctaccgttagttgccatcaggtgaagctgggcactctggcgggactgccggtgtaagccgagaggaaggtggggatgacgtcaaatcagcacggcccttacgtccggggctacacacgtgttacaatggtaggtacagagggcagctacccagcgatgggatgcgaatctcgaaagcctatctcagttcggattggaggctgaaacccgcctccatgaagttggattcgctagtaatcgcgcatcagccatggcgcggtgaatacgttcccgggccttgtacacaccgcccgtcaagccatgggagccgggggtgcctgaagttcgtgaccgcaagna

SEQ ID NO: 138 - DNA, 1277 bp, synthetic construct
  misc_feature 1..1277: note = Synthetic: FBI00270
  source 1..1277: mol_type = other DNA
tgggananccctggganctggggataatactggataggcaattattcctgtaatggttttttgtttaaatgttttttcgcctaaggatgggtctgcggccgattaggtagttggttaggtaatggcttaccaagcctttgatcggtacgggttgtgagagcaagagcccggagatggaacctgagacaaggttccaggccctacggggtgcagcaggcgcgaaacctccgcaatgtgagaaatcgcgacggggggatcccaagtgccattcttaacgggatggcttttcattagtgtaaagagcttttggaataagagctgggcaagaccggtgccagccgccgcggtaacaccggcagctctagtggtagcagtttttattgggcctaaagcgtccgtagccggtttaataagtctctggtgaaatcctgcagcttaactgtgggaattgctggagatactattagacttgagatcgggagaggttagaggtactcccagggtagaggtgaaattctgtaatcctgggaggaccgcctgttgcgaaggcgtctgactggaacgattctgacggtgagggacgaaagctaggggcgcgaaccggattagatacccgggtagtcctagctgtaaacgatgcggacttggtgttggggtggctttgagctgtcccagtgccgaagggaagctgttaagtccgccgcctgggaagtacggtcgcaagactgaaacttaaaggaattggcgggggagcaccacaacgcgtggagcctgcggtttaattggattcaacgccggacatctcaccagaggcgacagctgtatgatagccaggttgatgactttgcttgactagctgagaggaggtgcatggccgccgtcagctcgtaccgtgaggcgtcctgttaagtcaggcaacgagcgagacccacgctcttagttaccagcggatccttttttggatgccgggcacactaaggggaccgcctatgataaataggaggaaggagtggacgacggtaggtccgtatgccccgaatcctctgggcaacacgcgggctacaatggctgagacaatgggttccgacgccgaaaggcggaggtaatcctctaaacttagtcgtagttcggattgaggactgtaactcgttctcatgaagctggaatgcgtagtaatcgcgtgtcacaatcgcgcggtgaatacgtccctgctccttgcacacaccgcccgtcacgccacccaaaaagggattggatgaggatgtaatgttttgtna

SEQ ID NO: 139 - DNA, 1390 bp, synthetic construct
  misc_feature 1..1390: note = Synthetic: FBI00271
  source 1..1390: mol_type = other DNA
gcaagtcgaggggcagcattttagtttgcgttgcaaacgtaaagatggcgaccggcgcacgggtgagtaacacgtatccaacctgccgataactcggggatagcctttcgaaagaaagattaatatccgatagtatattaaaaccgcatggttttactattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtattccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaccntacccgggcttaaattgcaatgaatatctggaaacaggttagccgcaaggcatttgtgaaggtgctgcatggtggtcgtcagttcgtcccgtgaggtgtcggcttaagtgccataacgagcgcaacctttttctttagttattaccaggtcatgctgaggattttagagagactcccttcgtaagatgtgaggaaggtggggatgacgtcaaatcagcacggcccttacgtccggggttacacacgtgttccaatggggggtacagaaggcagctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaagga

SEQ ID NO: 140 - DNA, 1404 bp, synthetic construct
  misc_feature 1..1404: note = Synthetic: FBI00273
  source 1..1404: mol_type = other DNA
tgnnagtcgaggggcagcggggaggtagcaatacctttgccggcgaccggcgcacgggtgagtaacacgtatgcaatccacctgtaacagggggataacccggagaaatccggactaataccccataatatgggcgctccgcatggagggttcattaaagagagcaattttggttacngacgagcatgcgctccattagccagttggcggggtaacggcccaccaaagcgacgatggataggggttctgagaggaaggtcccccacattggaactgagacacggtccaaactcctacgggaggcagcagtgaggaatattggtcaatggtcggcagactgaaccagccaagtcgcgtgagggaagacggccctacgggttgtaaacctcttttgtcggagagtaaagtgcgctacgtgtagcgtattgcaagtatccgaagaaaaagcatcggctaactccgtgccagcagccgcggtaatacggaggatgcgagcgttatccggatttattgggtttaaagggtgcgtaggcggcacgccaagtcagcggtgaaatttccgggctcaacccggagtgtgccgttgaaactggcgagctagagtgcacaagaggcaggcggaatgcgtggtgtagcggtgaaatgcatagatatcacgcagaaccccgattgcgaaggcagcctgctagggtgaaacagacgctgaggcacgaaagcgtgggtatcgaacaggattagataccctggtagtccacgcagtaaacgatgaatactaactgtttgcgatacaatgtaagcggtacagcgaaagcgttaagtattccacctggggagtacgccggcaacggtgaaactcaaaggaattgacggggggcccgcacaagcggaggaacatgtggtttaattcgatgatacgcgaggaaccttacccgggctcaaacgcagggggaatgccggtgaaagccggcagctagcaatagtcacctgcgaggtgctgcatggttgtcgtcagctcgtgccgtgaggtgtcggcttaagtgccataacgagcgcaacccctatcgacagttactaacgggtgaagccgaggactctgtcgagactgccggcgcaagccgcgaggaaggtggggatgacgtcaaatcagcacggcccttacgtccggggcgacacacgtgttacaatggcaggtacagaaggcagccagtcagcaatgacgcgcgantcccgaaaacctgtctcagttcggattggagtctgcaacccgactccatgaagctggattcgctagtaatcgcgcatcagccatggcgcggtgaatacgttcccgggccttgtacacaccgcccgtcaagccatggaagccgggagtacctgaagcatgcaaccgcaaggagcgtacga

SEQ ID NO: 141 - DNA, 1405 bp, synthetic construct
  misc_feature 1..1405: note = Synthetic: FBI00274
  source 1..1405: mol_type = other DNA
tgcagtcgaacgaagcacctctcccgaagattgacacagcttgctgtagattgattcatttgaggtgactgagtggcggacgggtgagtaacgcgtgggtaacctgcctcatagagggggacaacagttggaaacgactgctaataccgcatagtaagaaaaattcgcatgttttcttcttgaaagatttatcgctatgagatggacccgcgtctgattagctagttggtaaggtaacggcttaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagataatgacggtacctgactaagaagccccggctaactacgtgccagcagccgcggtaatacgtagggggcaagcgttatccggatttactgggtgtaaagggtgcgtaggtggcaaggcaagtcagatgtgaaagcccggggctcaaccccggtactgcatttgaaactgtctagctagagtgcaggagaggtaagcggaattcctagtgtagcggtgaaatgcgtagatattaggaggaacaccagtggcgaaggcggcttactggactgtaactgacactgaggcacgaaagcgtggggagcaaacaggattagataccctggtagtccacgccgtaaacgatgaatactaggtgtcggggcccacagggcttcggtgccgcagcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatccttctgaccgttccttagccggaacttnccttcggagcagaagtgacaggtggtgcatggttgtcgtcagctcgtgtcgtgagatgttgggttaagtcccgcaacgagcgcaacccttatccttagtagccagcggttcggccgggcactctggggagactgccggagataatccggaggaaggtggggatgacgtcaaatcatcatgccccttatgacttgggctacacacgtgctacaatggcggtaacaaagggaagcagcctcgcgagagtgagcaaaccccaaaaatgccgtctcagttcggattgtagtctgcaactcgactacatgaagctggaatcgctagtaatcgcagatcagaatgctgcggtgaatacgttcccgggtcttgtacacaccgcccgtcacaccatgggagtcggatatgcccgaagccagtgacccaacnnnaagga

SEQ ID NO: 142 - DNA, 1388 bp, synthetic construct
  misc_feature 1..1388: note = Synthetic: FBI00277
  source 1..1388: mol_type = other DNA
tgcagtcgaggggcatcacgaggtagcaatactttggtggcgaccggcgcacgggtgcgtaacgcgtatgtaacctacctataacaggggcataacactgagaaattggtactaattccccataatattcggagaggcatctctccgggttgaaaactccggtggttatagatggacatgcgttgtattagctagttggtgaggtaacggctcaccaaggcaacgatacatagggggactgagaggttaaccccccacactggtactgagacacggaccagactcctacgggaggcagcagtgaggaatattggtcaatggacgcaagtctgaaccagccatgccgcgtgcaggaagacggctctatgagttgtaaactgcttttgtacgagggtaaactcacctacgcgtaggtgactgaaagtatcgtacgaataaggatcggctaactccgtgccagcagccgcggtaatacggaggattcaagcgttatccggatttattgggtttaaagggtgcgtaggcggtttgataagttagaggtgaaatcccggggcttaactccggaactgcctctaatactgttagactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttactgacgattctggaaacaggatttcccttcggggcaggaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggtcgctactccgtgaggagatgccaatctcgaaagcctatctcagttcggattggaggctgaaacccgcctccatgaagttggattcgctagtaatcgcgcatcagccatggcgcggtgaatacgttcccgggccttgtacacaccgcccgtcaagccatggaagctgggggtgcctgaagttcgtgaccgcaangagc

SEQ ID NO: 143 - DNA, 1398 bp, synthetic construct
  misc_feature 1..1398: note = Synthetic: FBI00278
  source 1..1398: mol_type = other DNA
tgcagtcgancgaagcaccnttgngacagannnnttcgganggacagactntggtgacttagtggcggacgggtgagtaacgcgtgggtaacctgccctgtactgggggacaacagttggaaacgactgctaataccgcataagcgcacagcatcgcatgatgcagtgtgaaaaactccggtggtacaggatggacccgcgtctgattagctagttggtgaggtaacggcttaccaaggcaacgatcagtagccgacttgagagagtgatcggccacattgggactgagacacggcccaaactcctacgggaggcagcagtggggaatattgcacaatgggggaaaccctgatgcagcgacgccgcgtgaaggaagaagtatttcggtatgtaaacttctatcagcaaggaagaaaatgacggtacttgactaagaagccccggctaaatacgtgccagcagccgcggtaatacgtatggggcaagcgttatccggatttactgggtgtaaagggagcgtaggcggtaagacaagtcagaagtgaaaggctggggctcaaccctgggactgcttttgaaactgtctaactagagtgcaggagaggtaagtggaattcctagtgtagcggtgaaatgcgtagatattaggaggaacaccagtggcgaaggcggcttactggactgtaactgacgctgaggctcgaaagcgtggggagcaaacaggattagataccctggtagtccacgccgtaaacgatgaatactaggtgttgggggaacaatagttcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctgctcttgacatcccactgaccggtcagtaatgtgaccttttcttcggaacagtggagacaggtggtgcatggttgtcgtcagctcgtgtcgtgagatgttgggttaagtcccgcaacgagcgcaacccttatctttagtagccagcatttaagatgggcactctagagagactgccagggataacctggaggaaggtggggatgacgtcaaatcatcatgccccttatgagcagggctacacacgtgctacaatggcgtaaacaaagggaagcgagaccgtgaggtggagccaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagttggatatgcccgaagtcagtgacccaaccgtaaggagg

SEQ ID NO: 144 - DNA, 1379 bp, synthetic construct
  misc_feature 1..1379: note = Synthetic: FBI00281
  source 1..1379: mol_type = other DNA
tgcagtcgaacgatgaaaccnancctcgggcggacatgaagtggcgaacgggtgagtaacacgtgaccgacctgccccccgcccagggacagccccccgaaagggggattaatacctggtactccggaagcggcgcatgccgcggccgggaaagctccggcggcgggggatggggtcgcggcccatcaggtagacggcggggcagaggcccaccgtgccgacgacgggtagccgggttgagagaccgaccggccacattgggactgagatacggcccagactcctacgggaggcagcagtggggaattttgcgcaatgggggcaaccctgacgcagcaacgccgcgtgcgggacgaaggcgtccgcgtcgtaaaccgctttcagcggggaacacttgaacgagggtacccgcagaagaagccccggctaaatacgtgccagcagccgcggtaatacgtatggggcgagcgttatccggattcattgggcgtaaagcgcgcgtaggcggagcgctaagcgggacctctaacccgagggctcaacccccggccgggtcccgaactggcgctctcgagtgcggtaggggagagcggaattcccggtgtagcggtggaatgcgcagatatcgggaagaacaccgacggcgaaggcagctctctgggccgaaactgacgctgaggcgcgaaagctgggggagcgaacaggattagataccctggtagtcccagccgtaaacgatgggcgctaggtgtgggggagcagatcccccgtgccgcagccaacgcattaagcgccccgcctggggagtacggccgcaaggctaaaactcaaaggaattgacgggggcccgcacaagcagcggagcatgtggcttaattcgaagcaacgcgaagaaccttaccagggcttgacatgcaggtgaagcggcggagacgccgtggccgagaggagcctgcacaggtggtgcatggctgtcgtcagctcgtgtcgtgagatgttgggttaagtcccgcaacgagcgcaacccctgccccgtgttgccagcattcagttggggactcgcgggggactgccggcgtcaagccggaggaaggtggggacgacgtcaagtcatcatgccccttatgccctgggccgcacacgtgctacaatggccggtacagagggttgcgaccccgcgagggggagcggatcccgcaaagccggccccagttcggatcggaggctgcaacccgcctccgtgaagccggagttgctagtaatcgcggatcagcacgccgcggtgaatacgttcccgggccttgtacacaccgcccgtcacaccacccgagtcgtctgcacccgaagccgccnnnnnnaacccccctgggggcggaggc

SEQ ID NO: 145 - DNA, 1398 bp, synthetic construct
  misc_feature 1..1398: note = Synthetic: FBI00288
  source 1..1398: mol_type = other DNA
tgcagtcgaacgaagcgatggagaacggagatttcggttgaagttttctattgactgagtggcggacgggtgagtaacgcgtgggtaacctgccctatacagggggataacagttagaaatgactgctaataccgcataagcgcacagcttcgcatgaagcggtgtgaaaaactgaggtggtataggatggacccgcgttggattagctagttggtgaggtaacggcccaccaaggcgacgatccatagccggcctgagagggtgaacggccacattgggactgagacacggcccaaactcctacgggaggcagcagtggggaatattgcacaatgggggaaaccctgatgcagcgacgccgcgtgaaggaagaagtatctcggtatgtaaacttctatcagcagggaagaaagtgacggtacctgactaagaagccccggctaattacgtgccagcagccgcggtaatacgtaaggggcaagcgttatccggatttactgggtgtaaagggagcgtagacggtttggcaagtctgatgtgaaaggcatgggctcaacctgtggactgcattggaaactgtcagacttgagtgccggagaggcaagcggaattcctagtgtagcggtgaaatgcgtagatattaggaggaacaccagtggcgaaggcggcctgctggacggtaactgacgttgaggctcgaaagcgtggggagcaaacaggattagataccctggtagtccacgctgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaaatcttgacatccctctgaccgggaagtaatgttcccttttcttcggaacagaggagacaggtggtgcatggttgtcgtcagctcgtgtcgtgagatgttgggttaagtcccgcaacgagcgcaacccttattcttagtagccagcaggtaaagctgggcactctagggagactgccagggataacctggaggaaggtggggatgacgtcaaatcatcatgccccttatgatttgggctacacacgtgctacaatggcgtaaacaaagggaagcgaaggggtgacctggagcaaatctcaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgaaagganggagc

SEQ ID NO: 146 - DNA, 1388 bp, synthetic construct
  misc_feature 1..1388: note = Synthetic: FBI00289
  source 1..1388: mol_type = other DNA
aacggcagcatgggggcaaccctgatggcgagtggcgaacgggtgagtaatatatcggaacgtacccaagagtgggggacaaccagtcgaaagattggctaataccgcatacgatctaaggatgaaagtgggggaccttcgggcctcacgctcctggagcggccgatagctgattagctagttggtggggtaaaggcctaccaaggcgatgatcagtagctggtctgagaggacgatcagccacactgggactgagacacggcccagactcctacgggaggcagcagtggggaattttggacaatgggggaaaccctgatccagcaatgccgcgtgagtgaagaaggccttcgggttgtaaagctcttttgtcagggaagaaattgattgggctaatatcccgattagatgacggtacctgaagaataagcaccggctaactacgtgccagcagccgcggtaatacgtagggtgcgagcgttaatcggaattactgggcgtaaagcgtgcgcaggcggttgtgtaagacagatgtgaaatgcccgggctcaacctgggaattgcatttgtgactgcacggctagagtgtgtcagaggggggtagaattccacgtgtagcagtgaaatgcgtagatatgtggaggaataccgatggcgaaggcagccccctgggataacactgacgctcatgcacgaaagcgtggggagcaaacaggattagataccctggtagtccacgccctaaacgatgtcgactagttgttgggtcttaattgacttagtaacgcagctaacgcgtgaagtcgaccgcctggggagtacggtcgcaagattaaaactcaaaggaattgacggggacccgcacaagcggtggatgatgtggattaattcgatgcaacgcgaaaaaccttacctacccttgacatgtcaggaatcccgaagagatttgggagtgctcgaaagagagcctgaacacaggtgctgcatggctgtcgtcagctcgtgtcgtgagatgttgggttaagtcccgcaacgagcgcaacccttgtcattagttgctacgaaagggcactctaatgaaactgccggtgacaaaccggaggaaggtggggatgacgtcaagtcctcatggcccttatgggtagggcttcacacgtcatacaatggtgcatacagagggttgccaacccgagagggggagctaatctcagaaagtgcatcgtagtccggattgaagtctgcaactcgacttcatgaagttggaatcgctagtaatcgcggatcagcatgtcgcggtgaatacgttcccgggtcttgtacacaccgcccgtcacaccatgggagcgggttttaccagaagtaggtagcttaaccgtaaggagggcgct

SEQ ID NO: 147 - DNA, 1381 bp, synthetic construct
  misc_feature 1..1381: note = Synthetic: FBI00290
  source 1..1381: mol_type = other DNA
ttannnntgcagtcgaacgaagacnntnctngagtgcttgcacttggannggactgagtggcggacgggtgagtaacgcgtgggtaacctgccttatacagggggataacagttggaaacgactgctaataccgcataagcgcacggtatcgcatgatacagtgtgaaaaactccggtggtataagatggacccgcgtctgattagcttgttggtgaggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgagtgatgaagtatttcggtatgtaaaactctatcagcagggaagataatgacggtacctgactaagaagcaccggctaaatacgtgccagcagccgcggtaatacgtatggtgcaagcgttatccggatttactgggtgtaaagggtgcgtaggtggtatggcaagtcagaagtgaaaggctggggctcaacccccgggactgcttttgaaactgtcaaactagagtacaggagaggaaaagcggaattcctagtgtagcggtgaaatgcgtagatattaggaggaacaccagtggcgaaggcggctttctggactgaaactgacactgaggcacgaaagcgtggggagcaaacaggattagataccctggtagtccacgccgtaaacgatgaatactaggtgtcgggcagcatagctgttcggtgccgcagcaaacgcattaagtattccacctggggagtacgttcgcaagaatgaaactcaaaggaattgacggggacccgcacaagcggtggagcatgtggtttaattcgaagcaacgcgaagaaccttaccaggtcttgacatcccgatgaccgcttcttaatcgaaacttttcttcggaacatcggtgacaggtggtgcatggttgtcgtcagctcgtgtcgtgagatgttgggttaagtcccgcaacgagcgcaacccctattttcagtagccagcattttggatgggcactctggagagactgcccgggataaccgggaggaaggtggggatgacgtcaaatcatcatgccccttatgatctgggctacacacgtgctacaatggctgttacagagagaagcgaatccgcgaggaagagcaaacctcaaaaaggcagtctcagttcggattgtagtctgcaactcgactacatgaagctggaatcgctagtaatcgcgaatcagaatgtcgcggtgaatacgttcccgggtcttgtacacaccgcccgtcacaccatgggagttggaaatgcccgaagtcagtgaccc

SEQ ID NO: 148 - DNA, 1262 bp, synthetic construct
  misc_feature 1..1262: note = Synthetic: FBI00292
  source 1..1262: mol_type = other DNA
ggnnngggatnnccctgggaactggggataatactggataggcaattattcctgtaatggttttttgtttaaatgttttttcgcctaaggatgggtctgcggccgattaggtagttggttaggtaatggcttaccaagcctttgatcggtacgggttgtgagagcaagagcccggagatggaacctgagacaaggttccaggccctacggggtgcagcaggcgcgaaacctccgcaatgtgagaaatcgcgacggggggatcccaagtgccattcttaacgggatggcttttcattagtgtaaagagcttttggaataagagctgggcaagaccggtgccagccgccgcggtaacaccggcagctctagtggtagcagtttttattgggcctaaagcgtccgtagccggtttaataagtctctggtgaaatcctgcagcttaactgtgggaattgctggagatactattagacttgagatcgggagaggttagaggtactcccagggtagaggtgaaattctgtaatcctgggaggaccgcctgttgcgaaggcgtctgactggaacgattctgacggtgagggacgaaagctaggggcgcgaaccggattagatacccgggtagtcctagctgtaaacgatgcggacttggtgttggggtggctttgagctgtcccagtgccgaagggaagctgttaagtccgccgcctgggaagtacggtcgcaagactgaaacttaaaggaattggcgggggagcaccacaacgcgtggagcctgcggtttaattggattcaacgccggacatctcaccagaggcgacagctgtatgatagccaggttgatgactttgcttgactagctgagaggaggtgcatggccgccgtcagctcgtaccgtgaggcgtcctgttaagtcaggcaacgagcgagacccacgcccttagttaccagcggatccttttggatgccgggcacactaaggggaccgcctatgataaataggaggaaggagtggacgacggtaggtccgtatgccccgaatcctctgggcaacacgcgggctacaatggctgagacaatgggttccgacgccgaaaggcggaggtaatcctctaaacttagtcgtagttcggattgaggactgtaactcgttctcatgaagctggaatgcgtagtaatcgcgtgtcacaatcgcgcggtgaatacgtccctgctccttgcacacaccgcccgtcacgccacccaaaaagggattgngatga